(19) United States
(12) Patent Application Publication
(10) Pub. No.: US 2003/0175761 A1
(43) Pub. Date: Sep. 18, 2003

(54) Identification of genes whose expression patterns distinguish benign lymphoid tissue and mantle cell, follicular, and small lymphocytic lymphoma

(76) Inventors: Daniel E. Sabath, Seattle, WA (US); Stephen C. Schmechel, Seattle, WA (US); Robert J. LeVasseur, Seattle, WA (US); Kathleen H. Yang, Woodinville, WA (US); Karen M. Koehler, Brier, WA (US)

Correspondence Address: GREENLEE WINNER AND SULLIVAN P C, 5370 MANHATTAN CIRCLE, SUITE 201, BOULDER, CO, 80303, US

(21) Appl. No.: 10/313,669

(22) Filed: Dec. 6, 2002

Related U.S. Application Data
(60) Provisional application No. 60/337,862, filed on Dec. 7, 2001.

(51) Int. Cl. C12Q 1/68

(52) U.S. Cl. 435/6

(57) ABSTRACT

Provided are genes whose expression patterns allow differentiation between benign lymph node tissue, follicular lymphoma tissue, mantle cell lymphoma tissue and small lymphocytic lymphoma tissue. These genes are useful as diagnostic markers for lymphoma. The protein products of these genes are useful in diagnostic and therapeutic applications, including monoclonal antibodies, lymphoma-specific chemotherapeutic agents, and gene therapies.

### CROSS-REFERENCE TO RELATED APPLICATIONS

This application takes priority to U.S. provisional application Serial No. 60/337,862, filed Dec. 7, 2001 which is incorporated herein to the extent not inconsistent with the disclosure herewith.

### BACKGROUND OF THE INVENTION

The identification of genes whose patterns of expression are cancer-specific will lead to better management of cancer patients. New tests fall into four areas: (a) tests designed to classify a patient's cancer (diagnosis), (b) tests designed to predict a patient's clinical course (prognosis), (c) tests designed to determine which subset of patients with a particular type of cancer will respond to particular drugs (pharmacogenomics), and (c) tests to monitor patient response to therapy (monitoring). Genes identified as over- or under-expressed in cancer also serve as important targets for the process of drug discovery and have utility as gene therapy agents.

DNA array technology (Schena, Shalon et al. 1995; Wodicka, Dong et al. 1997) provides the means to analyze the expression of hundreds to thousands of biomarkers in parallel. A DNA array is a device containing probes on a solid support that are designed to detect a large number of different DNA sequences. These devices allow quantification of the expression of thousands of distinct genes. Each gene generally encodes a single protein, which is the functional product of the gene. In the process of gene expression, each gene is copied into an intermediate form known as messenger RNA (mRNA). Genes that are expressed at high levels give rise to many copies of mRNA, whereas genes that are not expressed or expressed at low levels express few to no mRNA copies. DNA arrays facilitate the quantitative measurement of thousands of different mRNAs simultaneously.

The biological behavior of tissues, such as cancerous tissues, reflects the quantities and activities of the gene products that the tissue is expressing. Thus, by using DNA arrays to measure mRNAs as surrogates for measuring protein levels directly, one can obtain quantitative information about the biology of cells and tissues. For this reason, the use of DNA arrays containing probes directed against appropriate cancer-specific genes has been suggested as a way of augmenting current diagnostic methods (Brugarolas, Haynes et al. 2001). Currently available DNA arrays contain probes for thousands of human genes (a significant fraction of all human genes). This nearly comprehensive representation of the human genome on arrays has facilitated the search for genes whose patterns of expression are cancer-specific. Once cancer-specific genes are identified, dedicated customized DNA arrays can be designed to measure the expression of selected genes. Diagnostically useful genes may include genes whose up- or down-regulation is the result of oncogene mutation. Biomarker genes may correlate with the presence of specific chromosomal translocations. Finally, genes not previously known to be associated with a molecular determinant of cancer may serve as useful cancer-specific biomarker genes.

In the area of lymphoma biology, DNA array technology has been used to study novel lymphoma markers (Chan and Huang 2001) (Husson, Carideo et al. 2002) (Aalto, El-Rifa et al. 2001) (Stratowa, Loffler et al. 2001) (Hofmann, de Vos et al. 2001), to study distinct subtypes of diffuse large B-cell lymphoma (DLBCL) (Alizadeh, Eisen et al. 2000) (Shipp, Ross et al. 2002), to study molecular pathways potentially involved in lymphoma pathogenesis (Davis, Brown et al. 2001), and to predict the survival of DLBCL lymphoma patients after chemotherapy (Rosenwald, Wright et al. 2002).

There is particular need to discover cancer-specific genes expressed in hematologic cancers (leukemias and lymphomas) since this group of diseases is not optimally diagnosed or treated using current methods. Numerous classification schemes have been used in the diagnosis of hematologic cancers. The recently adopted World Health Organization (WHO) classification of hematopoietic neoplasms, which divides lymphomas into more than 40 distinct entities (Harris, Jaffe et al. 1999), underscores the diversity of these cancers. Indeed, within individual WHO diagnostic categories, cancers vary greatly in prognosis and in response to therapy due to inherent but poorly characterized biological heterogeneity (Cousar, Sawyers et al. 1999). It is likely that each WHO category encompasses multiple biologically distinct disease processes with different natural histories and responses to therapy. Currently, hematologic diagnoses are made using (a) gross and microscopic morphological examination, (b) detection of characteristic chromosomal rearrangements using nucleic acid hybridization, polymerase chain reaction (PCR), reverse transcription-PCR (RT-PCR), and cytogenetic analysis, and (c) detection of aberrant gene expression using PCR, RT-PCR, nucleic acid hybridization, and monoclonal antibodies. Due to the complexity of these diagnostic tests and subjectivity involved in test interpretation, obtaining accurate hematopathologic diagnoses is challenging for pathologists and clinicians. It may be relatively common, for example, that the same lymphoma would be categorized differently by different pathologists (listed 1997). Further, some cases of lymphoma lack features that allow them to fit neatly into any classification scheme.

Approximately 53,900 new cases of non-Hodgkin's lymphoma are diagnosed in the U.S. annually (Jemal, Thomas et al. 2002). Together, low-grade B cell lymphomas (LGBCLs), including follicular lymphoma (FL), mantle cell lymphoma (MCL), and chronic lymphocytic lymphoma/small lymphocytic lymphoma (CLL/SLL), comprise one-third of cases (Ries, Miller et al. 1994). LGBCLs are indolent but generally not curable (Voliotis and Diehl 2002). The time from diagnosis to death is quite variable, ranging from months to 20 years (Homing 2000). Advances in understanding the biological basis, clinical behavior, and treatment of LGBCL rely on accurate diagnoses. The currently used WHO lymphoma classification scheme is based on tumor morphology, molecular abnormalities, and the measurement of a limited number of immunocytochemical markers (Harris, Jaffe et al. 1999). It is likely that current LGBCL diagnostic categories encompass multiple molecularly distinct subtypes of disease with different clinical features and responses to therapy. Tools that allow the measurement of a larger number of relevant markers will lead to improvements in diagnostic classification.

FL is characterized histologically by the replacement of normal lymph node architecture with nodular collections of small cleaved and large non-cleaved neoplastic B cells. By flow cytometry, FL cells are typically CD5−, CD10+/−, CD23+/−, and CD43− (Elenitoba-Johnson and Kjeldsberg 2000). The clinical course of patients with FL is highly variable. Survival depends on the histological type and other unknown factors (Homing 2000) (Seng and Peterson 1997). In approximately 30-40% of patients, low-grade FL undergoes transformation to clinically aggressive diffuse large B-cell lymphoma (DLBCL) and survival after transformation is often less than one year (Knutsen 1997). The t(14;18) chromosomal translocation is seen in 80-90% of FL cases (Dalla-Favera and Gaidano 2001). This translocation joins the BCL-2 gene with immunoglobulin (Ig) heavy chain locus, resulting in over-expression of the anti-apoptotic BCL-2 protein and extended cell survival (Hockenbery, Nunez et al. 1990) (Vaux, Cory et al. 1988). However, BCL-2 overexpression is necessary but not sufficient to cause FL (Limpens, de Jong et al. 1991) (Limpens, Stad et al. 1995) (Liu, Hernandez et al. 1994) (Strasser, Harris et al. 1993). Accordingly, few cases of FL exhibit t(14; 18) as the only clonal chromosomal abnormality (Knutsen 1997). Clinical heterogeneity of FL may reflect the variety of molecular abnormalities that synergize with BCL-2 over-expression (Dalla-Favera and Gaidano 2001).

MCL is characterized histologically by the accumulation of neoplastic cells that either diffusely efface lymph nodes or form expanded nodules surrounding germinal centers. MCL cells are typically CD5+, CD10−/+, CD23−, CD43+, and cyclin D1+. The expression of CD5 and the absence of CD23 expression are useful in distinguishing this tumor from FL (which is CD5−) and CLL/SLL (which is CD23+) (Elenitoba-Johnson and Kjeldsberg 2000). MCL carries a median survival of 3-4 years. However, there is considerable variability in survival time, ranging from 1 to 185 months (Norton, Matthews et al. 1995). The hallmark genetic lesion in MCL is the t(11;14) translocation that brings the CCND1 gene under control of the Ig heavy chain (IgH) locus, resulting in over-expression of cyclin D1. Cyclin D1 mediates progression through the cell cycle (Adams, Harris et al. 1999). As with t(14;18) in FL, the t(11;14) chromosomal translocation is apparently not sufficient to cause MCL (Bodrug, Warner et al. 1994) (Lovec, Grzeschiczek et al. 1994). Differences in clinical outcome may result from a variety of molecular defects that synergize with cyclin D1 overexpression to cause MCL.

CLL/SLL is a neoplasm of small round B lymphocytes found in the peripheral blood and lymph nodes. The most common immunophenotype is CD5+, CD10−, CD23+, and CD43+. The expression of CD23 and CD43 is useful in distinguishing this tumor from MCL (which is CD23−) and FL (which is CD43−) (Elenitoba-Johnson and Kjeldsberg 2000). The clinical course of patients with CLL/SLL is highly variable. In some patients, the disease does not alter life expectancy, whereas in others survival is less than 5 years (E Montserrat F Bosch J Internal Med 242 (Supp 74): 63) (Montserrat, Bosch et al. 1997). In 5% of CLL/SLL cases, neoplastic cells undergo transformation to DLBCL (known as Richter's syndrome), leading to rapid clinical deterioration (Montserrat, Bosch et al. 1997). It appears that the diagnostic category of CLL/SLL encompasses at least two distinct disease subtypes: (a) relatively good prognosis neoplasms arising from B-cells that have transited through the lymph node germinal center (GC) as evidenced by hypermutated Ig variable region genes, and (b) relatively poor prognosis neoplasms arising from pre-GC B-cells that lack hypermutated Ig variable region genes (Naylor and Capra 1999) (Hamblin, Davis et al. 1999). The molecular events leading to the development of CLL/SLL and the basis of clinical heterogeneity among CLL/SLL cases remain elusive (Capello and Gaidano 2000).

Molecular and clinical variability within current LGBCL classifications reflects the complex pathogenesis of cancer. The concerted effects of multiple gene products, which may have partially overlapping functions, regulate the proliferation, maintenance, senescence, and elimination of cells. Closely related cancers may contain various constellations of accumulated genetic defects resulting in different biological behavior (Klein 1993).

There is a need in the art for an improved classification scheme for LGBCLs and methods to differentiate between types of LGBCLs.

### SUMMARY OF THE INVENTION

This invention provides a library of genes that allow differentiation between benign reactive lymph node tissue (RN), follicular lymphoma (FL), mantle cell lymphoma (MCL) and chronic lymphocytic lymphoma/small lymphocytic lymphoma (CLL/SLL). This invention also provides arrays using this set of genes, methods for making such arrays, and methods of using such arrays. The arrays of this invention are useful for determining gene expression profiles. Gene expression profiles are useful for determining expression profiles diagnostic of physiological conditions; diagnosing physiological conditions; identifying biochemical pathways, genes, and mutations involved in physiological conditions; identifying therapeutic agents useful for preventing and/or treating such physiological conditions; evaluating and/or monitoring the efficacy of such therapies, and creating and identifying animal models of human physiologic conditions. Arrays containing probes for all genes known to be useful in differentiating between benign reactive lymph node tissue (RN), follicular lymphoma (FL), mantle cell lymphoma (MCL) and chronic lymphocytic lymphoma/small lymphocytic lymphoma (CLL/SLL) are provided, as well as arrays containing subsets of such probes.

Also provided is an array comprising at least two isolated nucleotide molecules, each molecule having a sequence capable of uniquely hybridizing to a nucleic acid molecule having a sequence with at least 70% homology to a sequence listed in Table 2. Also provided is an array comprising at least two isolated nucleotide molecules, each molecule having a sequence capable of uniquely hybridizing to a nucleic acid molecule having a sequence with at least 80% homology to a sequence listed in Table 2. Also provided is an array comprising at least two isolated nucleotide molecules, each molecule having a sequence capable of uniquely hybridizing to a nucleic acid molecule having a sequence with at least 90% homology to a sequence listed in Table 2. Also provided is an array comprising at least two nucleic acid molecules or spots, each spot comprising a plurality of isolated nucleic acid molecules, each molecule having a sequence consisting essentially of a sequence listed in Table 2. Also provided is an array comprising at least ten nucleic acid molecules or spots, each spot comprising a plurality of isolated nucleic acid molecules, each molecule having a sequence consisting essentially of a sequence listed in Table 2. Also provided is an array comprising at least twenty nucleic acid molecules or spots, each spot comprising a plurality of isolated nucleic acid molecules, each molecule having a sequence consisting essentially of a sequence listed in Table 2. Also provided is an array comprising at least fifty nucleic acid molecules or spots, each spot comprising a plurality of isolated nucleic acid molecules, each molecule having a sequence consisting essentially of a sequence listed in Table 2. Also provided is an array comprising 120 nucleic acid molecules or spots, each spot comprising a plurality of isolated nucleic acid molecules, each molecule having a sequence consisting essentially of a sequence listed in Table 2.

Also provided is a method of selecting marker genes that distinguish between benign lymph node tissue, follicular lymphoma tissue, mantle cell lymphoma tissue and small lymphocytic lymphoma tissue comprising: preparing an array of probe genes; hybridizing labeled benign lymph node tissue, follicular lymphoma tissue, mantle cell lymphoma tissue and small lymphocytic lymphoma tissue with the array; analyzing the expression of the probe genes; selecting the marker genes that are differentially expressed by benign lymph node tissue, follicular lymphoma tissue, mantle cell lymphoma tissue and small lymphocytic lymphoma tissue.

Also provided is a method of determining an expression profile of a sample containing nucleic acid, comprising: providing the sample; providing an array of the invention; contacting said array with said sample under conditions allowing selective hybridization; and measuring hybridization of nucleic acid in said sample to said array to produce an expression profile. Two expression profiles may be generated and compared, for example, the expression profile of a sample known to correspond to a specific physiological condition may be compared with the expression profile of a sample taken from an organism to determine if the organism has the specific physiological condition.

The utility of this set of genes includes both diagnostic applications as well as the development of improved therapeutics. This gene set, or portions thereof, is used in a diagnostic DNA microarray that can diagnose various types of lymphoma. In this diagnostic DNA microarray, oligonucleotide probes for the lymphoma gene set are preferably used in a microarray format, where RNA extracted from patient tissue samples is labeled (preferably fluorescently, but other labels may be used, as known in the art) and applied to this microarray. Other testing methods such as kits containing the selected genes may be used, as known in the art. The amount of information gained from using a microarray for lymphoma diagnosis far exceeds that which can currently be gained from immunohistochemistry or flow cytometry, and the cost is reduced as well.

The protein targets of these genes can be used to generate monoclonal antibodies, as known in the art. These antibodies are useful to detect circulating lymphoma proteins for disease monitoring. The genes are also useful as targets for the development of lymphoma-specific pharmaceuticals. Drugs can be designed in a rational fashion to affect lymphoma cells without affecting normal tissues in a deleterious way, as known in the art. Finally, these genes can be used to combat lymphoma with gene therapy/gene transfer techniques. The expression of genes required for lymphoma cell proliferation can be specifically inhibited, and genes that interfere with the proliferation of lymphoma cells can be activated, as known in the art.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. FIG. 1 shows 48 genes differentially expressed among multiple tissue types. PolyA(+) RNA was pooled from 17 RN, 21 FL, 9 MCL, and 25 SLL specimens, respectively. Fluorescently labeled cDNA was generated from RNA and hybridized to microarrays containing cDNA probes for 14,976 IMAGE clones. Genes were selected based on ≧4-fold differential expression (p≦0.05 based on t-test analysis) between tissue types. Fold differential expression between the tissue types indicated in the figure legend is shown on the x-axis. Gene names and summary functions are shown to the left of y-axes.

FIG. 2 shows 72 genes differentially expressed only among two tissue types. PolyA(+) RNA was pooled from RN, FL, MCL, and SLL specimens. Fluorescently labeled cDNA was generated from RNA and hybridized to spotted cDNA microarrays. Genes were selected based on ≧4-fold differential expression (p≦0.05 based on t-test analysis) between tissue types. The x-axis shows fold differential expression between the tissue types indicated in the figure legends to the right of each graph. Gene names and summary functions are shown to the left of y-axes.

FIG. 3 is a summary flow chart depicting the comparison between gene expression data from microarray and qRT-PCR analyses. The expression of 39 of 120 genes identified by microarray analysis to be ≧4-fold differentially expressed was quantified by qRT-PCR. Using a threshold of ≧2-fold differential expression by qRT-PCR analysis, the expression patterns of 23 of the 39 genes were confirmed to be similar both by microarray and qRT-PCR methods.

FIG. 4 shows a graphical depiction of expression data for 23 genes whose gene expression profiles were similar by microarray and qRT-PCR analysis. The expression measurements for each gene in RN, FL, MCL, and SLL tissues were normalized to the expression level in a reference (tonsil) RNA sample. Each gene (identified at right) is represented by a single row of colored boxes; each tissue type is represented by a single column. Intensity of red indicates the degree of over-expression whereas intensity of blue indicates the degree of under-expression.

FIG. 5 shows that individual specimens of RN, FL, MCL, and SLL vary markedly in gene expression. The expression of (A) cyclin D1, (B) 13cDNA73, and (C) KIAA1407 in tonsil, 10 RN, 9 FL, 9 MCL, and 10 SLL individual specimens was quantified using qRT-PCR. Expression data for each gene was normalized to the level of cyclophilin expression. Tissue specimens are identified on the x-axes; relative expression is indicated on the y-axis. Two independent experiments were performed in duplicate. The results are presented as mean values (solid and gray bars for experiments 1 and 2) and SDs (error bars).

### DETAILED DESCRIPTION OF THE INVENTION

New biomarkers that are useful in distinguishing currently-defined types of LGBCL and currently unrecognized subtypes of FL, MCL and SLL have been discovered. DNA arrays have been used to identify 120 genes whose patterns of expression distinguish among FL, MCL, SLL, and benign lymph node tissue. Two of these genes, 13cDNA73 and KIAA1407, show distinct expression among individual FL, MCL, and SLL specimens.

Gene expression has been extensively studied. Although the regulation of mRNA abundance by-changes in transcription or RNA degradation is by no means the only mechanism that regulates protein levels in a cell, virtually all differences in cell type or state can be correlated to changes in the mRNA abundance of several genes (Alizadeh, Eisen et al. 2000) (DeRisi. Iyer et al. 1997) (Schena, Shalon et al. 1995) (Schena Shalon et al. 1996).

DNA microarray analysis has been used to study diffuse large B-cell lymphoma (DLBCL) where microarrays were used to expand the diagnosis of DLBCL (Alizadeh, Eisen et al. 2000). While standard histological and morphological techniques had defined subsets of DLBCL, array analysis revealed two clinically distinct classes. These two newly discovered classes were indistinguishable by standard pathology, but expression analysis showed a differential expression of hundreds of genes. Correlation of these molecular differences with differences in the progression of the disease and clinical outcome has revealed that these two classes of DLBCL could be considered separate diseases (Alizadeh, Eisen et al. 2000).

Nucleic acid arrays have been described, e.g., in U.S. Pat. No. 5,837,832, U.S. Pat. No. 5,807,522, U.S. Pat. No. 6,007,987, U.S. Pat. No. 6,110,426, WO 99/05324, 99/05591, WO 00/58516, WO 95/11995, WO 95/35505A1, WO 99/42813, JP10503841T2, GR3030430T3, ES2134481T3, EP804731B1, DE69509925C0, CA2192095AA, AU2862995A1, AU709276B2, AT180570, EP 1066506, and AU 2780499. Such arrays can be incorporated into computerized methods for analyzing hybridization results when the arrays are contacted with prepared sample nucleotides, e.g., as described in PCT Publication WO 99/05574, and U.S. Pat. Nos. 5,754,524; 6228,575; 5,593,839; and 5,856,101. Methods for screening for disease markers are also known to the art, e.g., as described in U.S. Pat. Nos. 6,228,586; 6,160,104; 6,083,698; 6,268,398; 6,228,578; and 6,265,174.

Currently, DNA microarrays are the most efficient method to monitor correlative changes in gene expression and to investigate complex traits on a molecular level. Expression profiles assembled from multiple interrelated experiments are used to determine hierarchical connections between gene expression patterns underlying complex biological traits. These patterns are used to further define the molecular basis of complex disorders.

As used herein “array” refers to an ordered set of isolated nucleic acid molecules or spots consisting of pluralities of substantially identical isolated nucleic acid molecules. Preferably the molecules are attached to a substrate. The spots or molecules are ordered so that the location of each (on the substrate) is known and the identity of each is known. Arrays on a micro scale can be called microarrays. Microarrays on solid substrates, such as glass or other ceramic slides, can be called gene chips or chips.

As used herein, an “isolated nucleic acid” is a nucleic acid outside of the context in which it is found in nature. An isolated nucleic acid is a nucleic acid the structure of which is not identical to that of any naturally occurring nucleic acid molecule. The term covers, for example: (a) a DNA which has the sequence of part of a naturally-occurring genomic DNA molecule but is not flanked by both of the coding or noncoding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally-occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein, or a modified gene having a sequence not found in nature.

As used herein “probe” refers to an isolated nucleic acid that is suitable for hybridizing to other nucleic acids when placed on a solid substrate. Probes for arrays can be as short as 20-30 nucleotides and up to as long as several thousand nucleotides. Probes can be single-stranded or double stranded. A probe usually comprises at least a partially known sequence that is used to investigate or interrogate the presence, absence, and/or amount of a complementing sequence. On the arrays of this invention, a probe is of such a sequence and the hybridization conditions of such stringency that each probe hybridizes substantially to only one type of nucleic acid per target sample.

As used herein, “target” or “target sample” refers to the collection of nucleic acids, e.g., reverse transcribed and labeled cDNA used as a prepared sample for array analysis. The target is interrogated by the probes of the array. A “target” or “target sample” may be a mixture of several prepared samples that are combined. For example, an experimental target sample may be combined with a differently labeled control sample and hybridized to an array, the combined samples being referred to as the “target” interrogated by the probes of the array. As used herein, “interrogated” means tested. Probes, targets, and hybridization conditions are chosen such that the probes are capable of interrogating the target, i.e., of hybridizing to complementary sequences in the target sample.

As used herein “printing” refers to the process of applying probes to a solid substrate, e.g., or applying arrays of probes to a solid substrate to make a gene chip. As used herein “glass slide” refers to a small piece of glass of the same dimensions as a standard microscope slide. As used herein, “prepared substrate” refers to a substrate that is prepared with a substance capable of serving as an attachment medium for attaching the probes to the substrate, such as poly Lysine.

As used herein “selective hybridization” refers to hybridization at moderate to high stringency such that only sequences of an appropriate homology can remain bound. Selective hybridization is hybridization performed at stringency conditions such that probes only hybridize to target sample nucleic acids that they are intended to hybridize with. Depending on the sequences of the probes and the target, the hybridization conditions are chosen to be appropriately selective. For example, if human sequences are used as probes for interrogating a human sample, selective hybridization could be at high stringency because, allowing for neutral polymorphism in humans, the sequences would be about 99-100% identical. When applying a chimpanzee target prepared sample to an array containing human sequence probes, selective hybridization would be at a lower stringency. Since hybridizing a target to an array is performed at one chosen hybridization stringency, probes are chosen so that they can undergo selective hybridization with the appropriate target molecules at the same hybridization stringency. As used herein “homology” refers to nucleotide sequence identity to a sequence, a molecule, or its complement.

As used herein, “clone” refers to an isolated nucleic acid molecule that may be stored in an organism such as E. coli. A clone is usually made of a vector and an insert. The insert usually contains a sequence of interest.

As used herein “physiological condition” refers to a healthy or unhealthy physiological state. As used herein “optimize an array for diagnosis” refers to selecting probes for an array such that only probes from genes necessary for diagnosis of one or more physiological conditions are included.

The microarrays or gene chips of this invention comprise probes placed in known positions on a solid substrate. A useful solid substrate is a specialized glass microscope slide. The arrays of this invention include arrays containing probes that detect some or all expressed sequences involved in mitochondrial biology in a selected species.

Arrays of this invention may contain control probes as well as probes for genes. Controls that can be included on the arrays of this invention include hybridization controls and scanning controls. The controls can be positive or negative controls. One type of hybridization control is spotting the same probe for a gene several times on one chip, each spot having different amounts of probe. This allows for the amount of probe of a given sequence to be optimized. Spotting too little probe may lead to a maximum hybridization signal resulting in a loss of data. Dimethyl sulfoxide (DMSO) can be used as a negative hybridization and scanning control. A spot of DMSO should give no signal. If there is any signal at a DMSO spot, the problem could be at hybridization or scanning steps. Plant sequences having sufficiently low homology with human and mouse sequences can also be utilized as negative hybridization and scanning controls. Plant sequences should not give any signal. A signal at a plant spot could indicate a problem with hybridization, i.e. too low a hybridization stringency was used, or with scanning, i.e., the chip was inserted into the scanner at the incorrect orientation. Poly A can be used as a positive hybridization specificity/non specificity control. A poly A spot should always give intense hybridization. No signal at a poly A spot could be the result of use of too high a hybridization stringency. Cy3 or Cy5 incorporated into a PCR product can be a positive scanning control. A spot on an array of a PCR product, or any other nucleic acid, that includes fluorescent label, should always give a signal, and if this sequence has no homology with any other sequence in the target, there should only be a signal of the label included in the nucleic acid. Control probes and probes for genes involved in mitochondrial biology can be duplicated, triplicated, etc. on the chip as printing controls. Controls for arrays can be purchased from Stratagene (SpotReport™, La Jolla, Calif., USA).

Standard targets and reference targets are also useful with the arrays of this invention, as is known in the art. When a prepared sample target to be interrogated is applied to an array of this invention, the results of the test are measured, i.e. by scanning, and recorded. These results can be compared directly to other test results using a similar array. However, it is much more accurate to include a differently labeled standard target in the hybridization mix with the prepared sample target. The results of the experimental sample target are then standardized, so that they can be compared accurately to the results of hybridizations of other sample targets. If ten different prepared sample targets are hybridized to arrays of this invention, simultaneously with the same prepared standard target, then the results of the ten sample targets can be accurately compared to each other. A prepared reference or control target for comparison can also be particularly pertinent to the experiment being performed. A prepared reference target could be a target sample derived from the same cell type from an animal of the same sex, age, and nuclear background as the experimental target sample, except for one difference, such as a different phenotype or treatment. Comparing the results of the experimental target with the results of an appropriate reference target yields a profile associated with the one difference being tested. When the hybridization results of a first sample are compared to the hybridization results of a second sample, the comparison can occur while the hybridization results of the first sample are being measured and recorded, or afterwards, by comparing the measured and recorded hybridization results of the two samples.

Probes on an array may be as short as about 20-30 nucleotides long or as long as the entire gene or clone from which they are derived, which may be up to several kilobases. A probe sequence may be identical (have 100% homology) to the portion of the gene it hybridizes to or it may be a mutated sequence. Mutated probes have less than 100% homology, such as about 98% homology, about 95% homology, about 90% homology, about 80% homology, or about 75% homology, or less, with the portions of the genes to which they hybridize. Arrays are designed such that all probes on an array can hybridize to their corresponding genes at about the same hybridization stringency. Probes for arrays should be unique at the hybridization stringencies used. Statistically, to be unique in the total human genome, probes should be at least about fifteen nucleotides long. A unique probe is only able to hybridize with one type of nucleic acid per target. A probe is not unique if at the hybridization stringency used, it hybridizes with nucleic acids derived from two different genes, i.e. related genes. The homology of the sequence of the probe to the gene and the hybridization stringency used help determine whether a probe is unique when testing a selected sample. Probes also may not hybridize with different nucleic acids derived from the same gene, i.e., splice variants. The location in the gene of the sequence used for the probe also helps determines whether a probe is unique when testing a selected sample. If the splice variants of a gene are known, ideally several different probes sequences are chosen from that gene for an array, such that each probe can only hybridize to nucleic acid derived from one of the splice variants. Arrays of this invention are used at hybridization conditions allowing for selective hybridization. At conditions of selective hybridization, probes hybridize with nucleic acid from only one gene. When an array is simultaneously hybridized with two targets or two prepared samples, each probe may hybridize with a nucleic acid in each prepared sample or target. When these two nucleic acids are from the same unigene cluster, the probe is said to hybridize with one gene, despite the fact that these nucleic acids may contain different labels.

The arrays of this invention can be utilized to determine profiles for related species by modifying the hybridization stringency appropriately. Sequence homology between organisms is known in the art. For example, human and chimpanzee sequences are about 98% identical. Consequently, human arrays are useful for profiling chimpanzees, with an appropriate lowering of the hybridization stringency. Hybridization stringency can be lowered by modifying hybridization components such as salt concentrations and hybridization and/or wash temperatures, as is known in the art.

The sequences useful for the arrays of this invention are useful for designing arrays for other species as well. To create an array for a new organism, the known sequences from the new organism, including expressed sequence tags (ESTs), are compared, by methods known to the art, with the sequences known to already be useful for other arrays. Sequence comparisons may be performed at the nucleic acid or polypeptide level. Homologous and analogous sequences from the new organism are thereby identified and selected for the new organism's mitochondrial array. The probes on the arrays of this invention are also useful as probes for identifying candidates for the new organism's array using molecular biology techniques that are standard in the art such as screening libraries.

All sequences given herein are meant to encompass the complementary strand, as well as double-stranded polynucleotides comprising the given sequence.

Microarrays of this invention can contain as few as two probes to as many as all the probes diagnostic of the selected physiological condition to be tested. Microarrays of this invention may also contain probes for all genes. The arrays of this invention may contain probes for at least about five genes, at least about ten genes, at least about twenty-five genes, at least about fifty genes, or all genes useful in differentiating between the conditions described herein. Arrays of this invention may comprise more than about five spots, more than about ten spots, more than about twenty-five spots, or all spots useful in differentiating between the conditions described herein.

Using microarrays may require amplification of target sequences (generation of multiple copies of the same sequence) of sequences of interest, such as by PCR or reverse transcription. As the nucleic acid is copied, it is tagged with a fluorescent label that emits light like a light bulb. The labeled nucleic acid is introduced to the microarray and allowed to react for a period of time. This nucleic acid sticks to, or hybridizes, with the probes on the array when the probe is sufficiently complementary to the labeled, amplified, sample nucleic acid. The extra nucleic acid is washed off of the array, leaving behind only the nucleic acid that has bound to the probes. By obtaining an image of the array with a fluorescent scanner and using software to analyze the hybridized array image, it can be determined if, and to what extent, genes are switched on and off, or whether or not sequences are present, by comparing fluorescent intensities at specific locations on the array. The intensity of the signal indicates to what extent a sequence is present. In expression arrays, high fluorescent signals indicate that many copies of a gene are present in a sample, and lower fluorescent signal shows a gene is less active. By selecting appropriate hybridization conditions and probes, this technique is useful for detecting single nucleotide polymorphisms (SNPs) and for sequencing. Methods of designing and using microarrays are continuously being improved (Relogio, Schwager et al. 2002) (Iwasaki, Ezura et al. 2002) (Lindroos, Sigurdsson et al. 2002).

Arrays of this invention may be made by any array synthesis methods known in the art such as spotting technology or solid phase synthesis. Preferably the arrays of this invention are synthesized by solid phase synthesis using a combination of photolithography and combinatorial chemistry. Some of the key elements of probe selection and array design are common to the production of all arrays. Strategies to optimize probe hybridization, for example, are invariably included in the process of probe selection. Hybridization under particular pH, salt, and temperature conditions can be optimized by taking into account melting temperatures and by using empirical rules that correlate with desired hybridization behaviors. Computer models may be used for predicting the intensity and concentration-dependence of probe hybridization.

Arrays, also called DNA microarrays or DNA chips, are fabricated by high-speed robotics, generally on glass but sometimes on nylon substrates, for which probes (Phimister 1999) with known identity are used to determine complementary binding. An experiment with a single DNA chip can provide researchers information on thousands of genes simultaneously. There are several steps in the design and implementation of a DNA array experiment. Many strategies have been investigated at each of these steps: 1) DNA types; 2) Chip fabrication; 3) Sample preparation; 4) Assay; 5) Readout; and 6) Software (informatics).

There are two major application forms for the array technology: 1) Determination of expression level (abundance) of genes; and 2) Identification of sequence (gene/gene mutation). There appear to be two variants of the array technology, in terms of intellectual property, of arrayed DNA sequence with known identity: Format I consists of probe cDNA (500˜5,000 bases long) immobilized to a solid surface such as glass using robot spotting and exposed to a set of targets either separately or in a mixture. This method, “traditionally” called DNA microarray, is widely considered as having been developed at Stanford University (Ekins and Chu 1999). Format II consists of an array of oligonucleotide (20˜80-mer oligos) or peptide nucleic acid (PNA) probes synthesized either in situ (on-chip) or by conventional synthesis followed by on-chip immobilization. The array is exposed to labeled sample DNA, hybridized, and the identity/abundance of complementary sequences is determined. This method, “historically” called DNA chips, was developed at Affymetrix, Inc., which sells its photolithographically fabricated products under the GeneChip® trademark. Many companies are manufacturing oligonucleotide-based chips using alternative in-situ synthesis or depositioning technologies.

Probes on arrays can be hybridized with fluorescently-labeled target polynucleotides and the hybridized array can be scanned by means of scanning fluorescence microscopy. The fluorescence patterns are then analyzed by an algorithm that determines the extent of mismatch content, identifies polymorphisms, and provides some general sequencing information (Chee, Yang et al. 1996). Selectivity is afforded in this system by low stringency washes to rinse away non-selectively adsorbed materials. Subsequent analysis of relative binding signals from array elements determines where base-pair mismatches may exist. This method then relies on conventional chemical methods to maximize stringency, and automated pattern recognition processing is used to discriminate between fully complementary and partially complementary binding.

Devices such as standard nucleic acid microarrays or gene chips, require data processing algorithms and the use of sample redundancy (i.e., many of the same types of array elements for statistically significant data interpretation and avoidance of anomalies) to provide semi-quantitative analysis of polymorphisms or levels of mismatch between the target sequence and sequences immobilized on the device surface. Such algorithms and software useful for statistical analysis are known to the art.

Using microarrays first requires amplification (generation of multiple copies of the same gene) of genes of interest, such as by reverse transcription. As the nucleic acid is copied, it is tagged with a fluorescent label that emits light like a light bulb. The labeled nucleic acid is introduced to the microarray and allowed to react for a period of time. This nucleic acid sticks to, or hybridizes, with the probes on the array when the probe is sufficiently complementary to the nucleic acid in the prepared sample. The extra nucleic acid is washed off of the array, leaving behind only the nucleic acid that has bound to the probes. By obtaining an image of the array with a fluorescent scanner and using software to analyze the hybridized array image, it can be determined if and to what extent genes are switched on and off, or whether or not sequences are present, by comparing fluorescent intensities at specific locations on the array. High fluorescent signals indicate that many copies of a gene are present in a prepared sample, and lower fluorescent signal shows a gene is less active. Expression levels for various genes under different conditions can be directly compared, such as for a cancer cell and a normal cell. Similarly, it can be determined what genes are turned on and off in response to certain stimuli such as a drug. Such information is valuable because it identifies genes in disease pathways and also is predictive of either efficacy or toxicity of drugs.

Probes fixed on solid substrates and targets (nucleotide sequences in the sample) are combined in a hybridization buffer solution and held at an appropriate temperature until annealing occurs. Thereafter, the substrate is washed free of extraneous materials, leaving the nucleic acids on the target bound to the fixed probe molecules allowing for detection and quantitation by methods known in the art such as by autoradiograph, liquid scintillation counting, and/or fluorescence. As improvements are made in hybridization and detection techniques, they can be readily applied by one of ordinary skill in the art. As is well known in the art, if the probe molecules and target molecules hybridize by forming a strong non-covalent bond between the two molecules, it can be reasonably assumed that the probe and target nucleic acid are essentially identical, or almost completely complementary if the annealing and washing steps are carried out under conditions of high stringency. The detectable label provides a means for determining whether hybridization has occurred.

When using oligonucleotides or polynucleotides as hybridization probes, the probes may be labeled. In arrays of this invention, the target may instead be labeled by means known to the art. Target may be labeled with radioactive or non-radioactive labels. Targets preferably contain fluorescent labels.

Various degrees of stringency of hybridization can be employed. The more stringent the conditions are, the greater the complementarity that is required for duplex formation. Stringency can be controlled by temperature, probe concentration, probe length, ionic strength, time, and the like. Hybridization experiments are often conducted under moderate to high stringency conditions by techniques well know in the art, as described, for example in Keller, G. H., and M. M. Manak (1987) DNA Probes, Stockton Press, New York, N.Y., pp. 169-170, hereby incorporated by reference. However, sequencing arrays typically use lower hybridization stringencies, as is known in the art.

Moderate to high stringency conditions for hybridization are known to the art. An example of high stringency conditions for a blot are hybridizing at 68° C. in 5×SSC/5× Denhardt's solution/0.1% SDS, and washing in 0.2×SSC/0.1% SDS at room temperature. An example of conditions of moderate stringency are hybridizing at 68° C. in 5×SSC/5× Denhardt's solution/0.1% SDS and washing at 42° C. in 3×SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, NY, N.Y., for further guidance on hybridization conditions.

The melting temperature is described by the following formula (Beltz, G. A. et al., [1983]Methods of Enzymology, R. Wu, L. Grossman and K. Moldave [Eds.] Academic Press, New York 100:266-285).

Tm=81.5° C.+16.6 Log[Na+]+0.41(+G+C)−0.61(% formamide)-600/length of duplex in base pairs.

Washes can typically be carried out as follows: twice at room temperature for 15 minutes in 1×SSPE, 0.1% SDS (low stringency wash), and once at TM-20° C. for 15 minutes in 0.2×SSPE, 0.1% SDS (moderate stringency wash).

Nucleic acid useful in this invention can be created by Polymerase Chain Reaction (PCR) amplification. PCR products can be confirmed by agarose gel electrophoresis. PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. [1985] Science 230:1350-1354). PCR is used to enzymatically amplify a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Since the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes that can be used are known to those skilled in the art.

Polynucleotide sequences of the present invention can be truncated and/or mutated such that certain of the resulting fragments and/or mutants of the original full-length sequence can retain the desired characteristics of the full-length sequence. A wide variety of restriction enzymes that are suitable for generating fragments from larger nucleic acid molecules are well known. In addition, it is well known that Bal31 exonuclease can be conveniently used for time-controlled limited digestion of DNA. See, for example, Maniatis (1982) Molecular Cloning: A Laboratory Manual, Cold Spring Harbor Laboratory, New York, pages 135-139, incorporated herein by reference. See also Wei et al. (1983) J. Biol. Chem. 258:13006-13512. By use of Bal31 exonuclease (commonly referred to as “erase-a-base” procedures), the ordinarily skilled artisan can remove nucleotides from either or both ends of the subject nucleic acids to generate a wide spectrum of fragments that are functionally equivalent to the subject nucleotide sequences. One of ordinary skill in the art can, in this manner, generate hundreds of fragments of controlled, varying lengths from locations all along the original molecule. The ordinarily skilled artisan can routinely test or screen the generated fragments for their characteristics and determine the utility of the fragments as taught herein. It is also well known that the mutant sequences can be easily produced with site-directed mutagenesis. See, for example, Larionov, O. A. and Nikiforov, V. G. (1982) Genetika 18(3):349-59; and Shortle, D. et al., (1981) Annu. Rev. Genet. 15:265-94, both incorporated herein by reference. The skilled artisan can routinely produce deletion-, insertion-, or substitution-type mutations and identify those resulting mutants that contain the desired characteristics of wild-type sequences, or fragments thereof.

Thus, mutational, insertional, and deletional variants of the disclosed nucleotide sequences can be readily prepared by methods which are well known to those skilled in the art. These variants can be used in the same manner as the exemplified primer sequences so long as the variants have substantial sequence homology with the original sequence. As used herein, substantial sequence homology refers to homology that is sufficient to enable the variant polynucleotide to function in the same capacity as the polynucleotide from which the probe was derived. Homology is greater than 80%, greater than 85%, greater than 90%, or greater than 95%. The degree of homology or identity needed for the variant to function in its intended capacity depends upon the intended use of the sequence. It is well within the skill of a person trained in this art to make mutational, insertional, and deletional mutations that are equivalent in function or are designed to improve the function of the sequence or otherwise provide a methodological advantage.

Percent sequence identity of two nucleic acids may be determined using the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See http://www.ncbi.nih.gov.

Standard techniques for cloning, DNA isolation, amplification and purification, for enzymatic reactions involving DNA ligase, DNA polymerase, restriction endonucleases and the like, and various separation techniques useful herein are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkeley; Schleif and Wensink (1982) Practical Methods in Molecular Biology; Glover (Ed.) (1985) DNA Cloning Vol. I and II, IRL Press, Oxford, UK; Hames and Higgins (Eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

Arrays can be printed on solid substrates, e.g., glass microscope slides. Before printing, slides are prepared to provide a substrate for binding, as known in the art. Arrays can be printed using any printing techniques and machines known in the art. Printing involves placing the probes on the substrate, attaching the probes to the substrate, and blocking the substrate to prevent non-specific hybridization, as known in the art.

Samples useful for analyses using the arrays of this invention include total RNA samples and m-RNA samples. RNA samples can be prepared as known in the art. An RNA sample is reverse transcribed into cDNA and simultaneously labeled, i.e. with one member of a two-color fluorescent system, such as Cy3-dCTP/Cy5-dCTP as known in the art. The arrays are hybridized with the prepared sample and washed at appropriate stringencies accounting for the choices of sample and probes of the array. The hybridization stringency can be higher when the probe sequence has higher homology with the gene it interrogates and when the probe is larger. A reference target, standard target, or other sample target for direct comparison may be prepared and hybridized simultaneously to the same array. A prepared sample will not degrade during hybridization and is labeled. Prepared samples are reverse transcribed and fluorescently labeled.

Hybridization results can be measured and analyzed using equipment and software available in the art. Before finalizing data, preliminary results are preferably normalized by methods known in the art. Analysis includes determination of statistical significance. Measurement may include normalization and analysis, including statistical analysis. Resulting data are typically stored in computer files.

Monoclonal or polyclonal antibodies, preferably monoclonal, specifically reacting with a protein of interest can be made by methods well known in the art. See, e.g., Harlow and Lane (1988) Antibodies: A Laboratory Manual, Cold Spring Harbor Laboratories; Goding (1996) Monoclonal Antibodies: Principles and Practice, 3rd ed., Academic Press, San Diego, Calif., and Ausubel et al. (1993) Current Protocols in Molecular Biology, Wiley Interscience/Greene Publishing, New York, N.Y.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

The following examples are provided for illustrative purposes, and are not intended to limit the scope of the invention as claimed herein. Any variations in the exemplified articles which occur to the skilled artisan are intended to fall within the scope of the present invention.

Materials and Methods

Clinical material. Lymph node and lymphoma specimens were obtained from the University of Washington (UW) Hematopathology Laboratory tissue bank. Freshly excised tonsils were obtained from the Seattle Children's Hospital and Medical Center. All studies were approved by the University of Washington and Children's Hospital and Medical Center Institutional Review Boards. Between 1989 and 1996, lymph node and lymphoma specimens were surgically removed from patients in the course of their medical care at the UW Medical Center or one of several referral medical facilities in western Washington, Idaho, Montana, and Alaska. Tissues not needed for diagnostic testing were frozen in water-soluble tissue freezing medium (O.C.T.; Tissue-Tek, Naperville, Ill.) and transferred to a −70° C. freezer where they were maintained until processing. Each specimen was stripped of patient identifier information with the exception of final diagnosis and anatomic source and catalogued in a FileMaker Pro (FileMaker, Inc., Santa Clara, Calif.) database. From this frozen tissue archive, the following tissues were randomly selected: 18 benign reactive lymph node (RN), 21 grade I FL, 25 SLL, and 11 MCL specimens.

RNA Isolation and cDNA synthesis. Lymphoma and lymph node tissues were transferred on dry ice from −70° C. freezer to a −20° C. Tissue-Tek II Microtome/Cryostat. Using the cryostat, approximately fifty 10 μm tissue sections (representing ˜250 mg of tissue) were cut from each specimen and placed in a 15-mL conical tube on ice. Fresh tonsil specimens, each in ˜20 mL RPMI medium (Life Technologies, Rockville, Md.), were oriented in plastic petri dishes with the epithelium-containing side down. Using a plastic scalpel, the tissue was finely chopped against the underside of the epithelial layer to free lymphoid cells into the medium. The cells were pelleted by centrifugation for 20 minutes at 800×g in a room temperature IEC CentraCL centrifuge (International Equipment Company, Needham Heights, Mass.). A sufficient volume (typically 1-5 mL for lymphoma specimens and 5 mL for pelleted tonsil cells) of phenol/guanidine isothiocyanate (TRIzol; Invitrogen Life Technologies, Carlsbad, Calif.) was added. Samples were vortexed thoroughly and placed on ice for up to several hours. Total RNA was isolated according to the TRIzol manufacturer's instructions. Total RNA was quantified by spectrophotometry (J. Sambrook, E F Fritsche, and T Maniatis, Molecular Cloning A Laboratory Manual, Second Edition, Cold Spring Harbor Laboratory Press 1989, Plainview, N.Y. 11803; page E.5) using a Hewlett Packard 8452A Diode Array spectrophotometer (Hewlett Packard, Palo Alto, Calif.). Poly(A)+RNA was purified from total RNA using oligo(dT)₂₅-linked magnetic beads (Dynal, Oslo, Norway) according to manufacturer's instructions. Poly(A)+RNA was labeled with RiboGreen (Molecular Probes, Eugene, Oreg.) according to manufacturer's instructions and quantified using a Versafluor fluorometer (Bio-Rad Laboratories, Hercules, Calif.) by comparison to a standard curve generated using known concentrations of RNA (Molecular Probes). A typical yield from each ˜250 mg tissue specimen was 0.5-4 μg of poly (A)+RNA. Poly(A)+RNA from seven tonsils was pooled. poly(A)+RNA was analyzed using an Agilent 2100 bioanalyzer (Agilent Technologies, Palo Alto, Calif.). In each case, rRNA contamination was ≦14% and poly(A)+RNA migrated as a typical population of poly(A)+RNA species predominantly ranging in size from 1.3 to 4.4 kb (data not shown). Two labeled cDNA populations were prepared from each poly(A)+RNA pool. In one reaction, 2 μg poly(A)+RNA were reverse transcribed into cDNA labeled with Cy3-dCTP (AP Biotech, Little Chalfont, Buckinghamshire, United Kingdom) as previously described (Geiss GK JV 75:4321); in a second reaction, 2 μg poly(A)+RNA were reverse transcribed into cDNA labeled with Cy5-dCTP (AP Biotech). Labeled cDNAs were purified as previously described (Geiss GK JV 75:4321) and dissolved in 100 μl 10 mM Tris, pH 8.0. The efficiency of Cy3/Cy5-dCTP incorporation was determined using an HP 8452A diode array spectrophotometer (Hewlett Packard, Palo Alto, Calif.) and the following formulae:

A₅₅₀×probe volume/0.15=pmol Cy3 probe, and

A₆₅₀×probe volume/0.25=pmol Cy5 probe.

A typical yield for each Cy3-labeled cDNA was 100 pmol and for each Cy5-labeled cDNA was 75 pmol.

Microarray construction and hybridization. The spotted cDNA microarrays used in this study, containing ˜15,000 Homo sapiens sequence-verified Integrated Molecular Analysis of Genomes and their Expression (IMAGE) consortium clones (UniGene Build 19, plates 1 to 44; Lennon G Genomics 33:151) obtained from Research Genetics (Huntsville, Ala.), were obtained from the University of Washington (UW) Center for Expression Arrays (UW-CEA). These microarrays were constructed as described previously (Geiss GK JV 75:4321). Two sets of slides were used. Human HD-1 and HD-2 arrays each contained nearly unique sets of 7488 cloned human genes and ESTs spotted in duplicate. A complete list of the genes contained on these arrays is available at http://ra.microslu.washington.edu/Website/genelist/genelist.html. Each array was rinsed 10 times in sterile H₂O and then immediately dried using compressed air (Dust-Off, Falcon Safety Products, Branchburg, N.J.). Fluorescently labeled cDNAs were combined as described in the results section and concentrated by drying and were resuspended in 20 μL of hybridization solution (50% deionized formamide [Sigma, St. Louis, Mo.], 5×SSC [0.75M sodium chloride, 75 mM sodium citrate; Ambion, Austin, Tex.], 5×Denhardts solution [Fisher Scientific, Houston, Tex.], 0.1% sodium dodecyl sulfate [SDS; Ambion, Austin, Tex.], 100 μg/μL CotI DNA [Invitrogen], and 20 μg/μL polyA [5′-A(75)-3′] primer [Invitrogen]), denatured by boiling for 3 minutes, chilled on ice for 30 seconds, briefly centrifuged, and placed at room temperature. Labeled cDNAs were added to the each array and covered with 64- by 25-mm cover slips. Microarrays were hybridized for 14 to 16 hours at 42° C. in a humidified chamber (Genetix Limited, Hampshire, United Kingdom). Following hybridization, the microarrays were washed briefly in 1×SSC/0.2% SDS (pre-warmed to 54° C.) to remove the cover slips. The arrays were transferred to rectangular glass dishes (Wheaton Science Products, Millville, N.J.) in which they were washed by gentle rocking in 1×SSC/0.2% SDS (pre-warmed to 54° C.) for 10 minutes, 1×SSC/0.2% SDS (pre-warmed to 54° C.) for 10 minutes, 0.1×SSC/0.2% SDS (pre-warmed to 54° C.) for 10 minutes, 0.2×SSC (rt) for 1 minute, and 0.1×SSC (rt) for 1 minute. Finally, the arrays were dipped twice in distilled H₂O and dried with compressed air. The microarrays were scanned at 532 and 633 nm using a Molecular Dynamics Avalanche dual-laser confocal scanner.

Microarray data analysis. Duplicate Human HD1 and HD2 slides were hybridized with the same cDNAs but with the fluorescent labels reversed to dampen dye-specific effects (Methods 24: 289 AK Kenworthy; Ramdas L Genome Biol 2001;2(11):RESEARCH0047). Each slide contained two identical sets of spots on sides A and B of the slide. Two images, corresponding to sides A and B, were obtained for each slide. Using Spot-On software developed at the UW Center for Expression arrays (Geiss GK JV 75:4321; http://ra.microslu.washington.edu/Website/analysis/analysis.html), the intensity of each spot (subtracted for local background) in both channels was obtained from each image and exported as a text file. The Spot-On software program divides each spot into four quadrants and provides the average signal intensity for each quadrant as well as the background signal intensity surrounding the spot. Using GeneSifter.Net™ (VizX Labs, Seattle, Wash.), the intensity value of each spot in an image was normalized to the mean intensity of all spots in that image. Clones were selected if the mean spot intensities showed ≧4-fold differential expression in pairwise comparisons among RN, FL, MCL, and SLL tissues and showed a corresponding p-value of ≦0.05 (derived from Student t-test analysis).

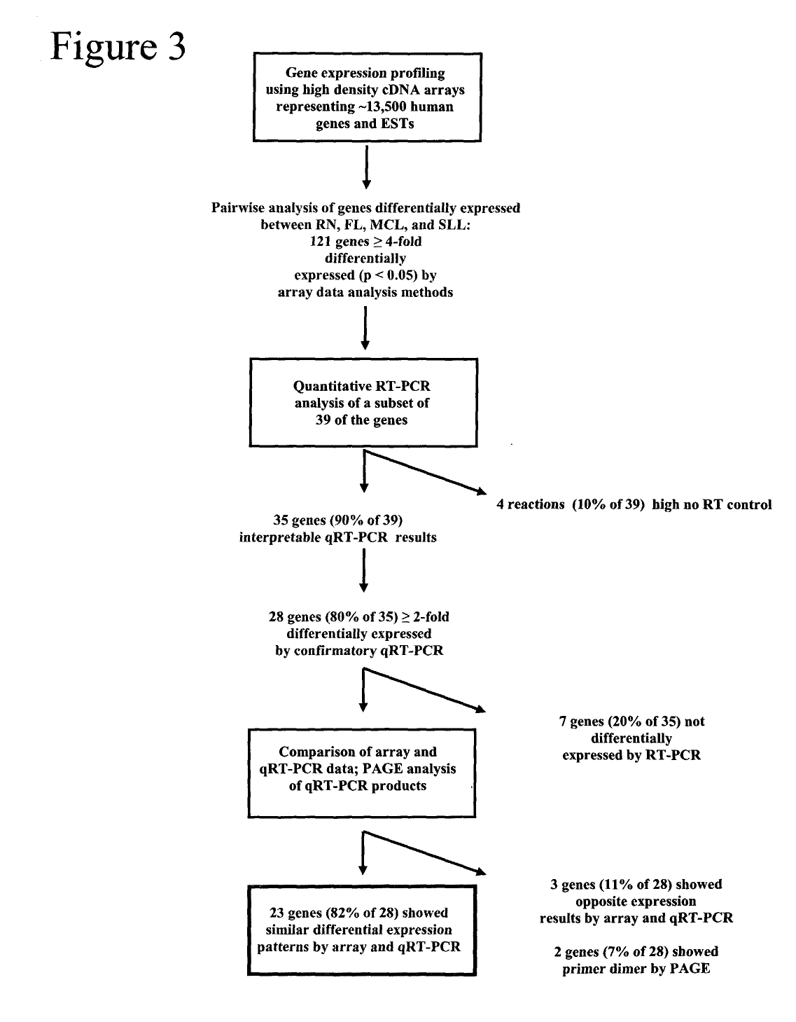

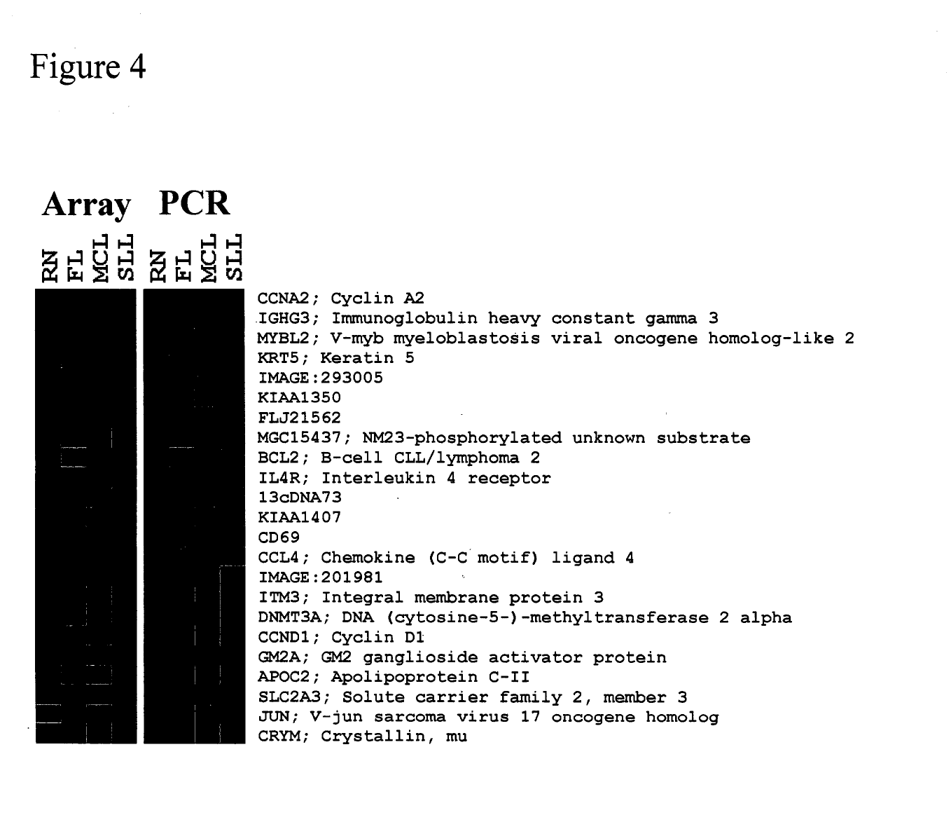

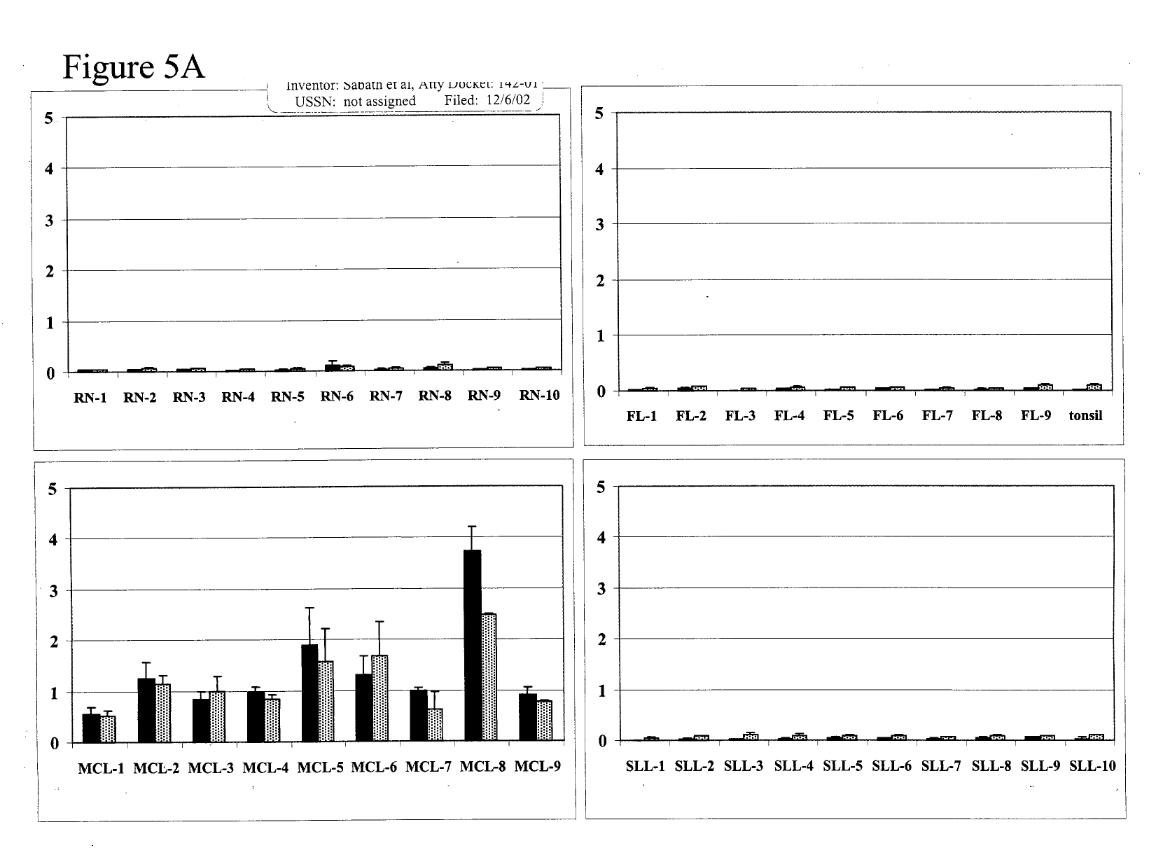

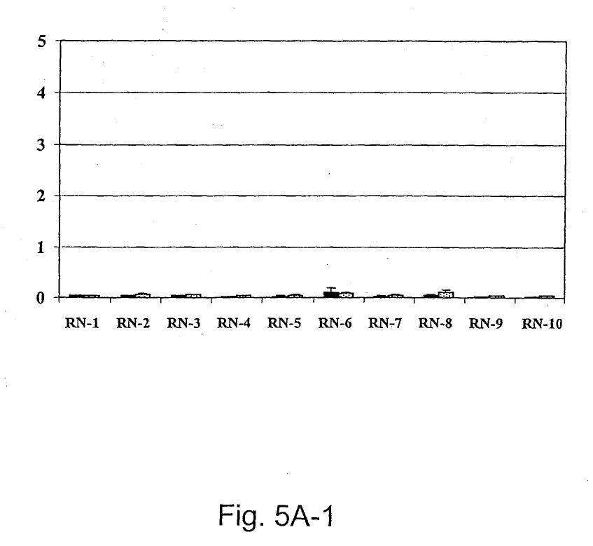

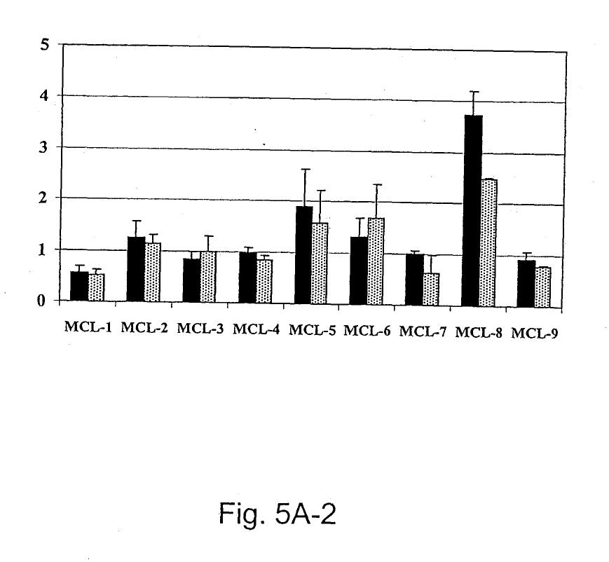

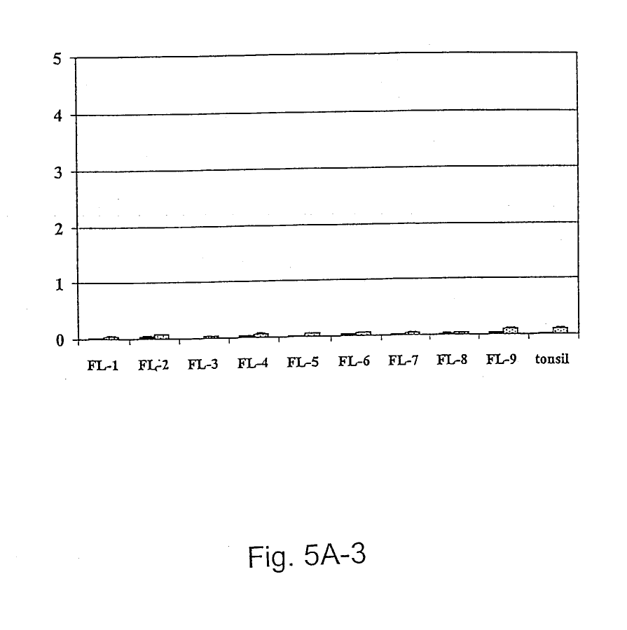

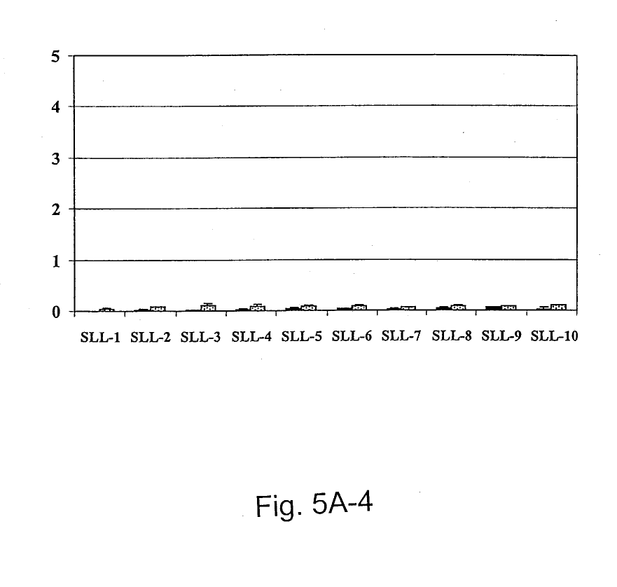

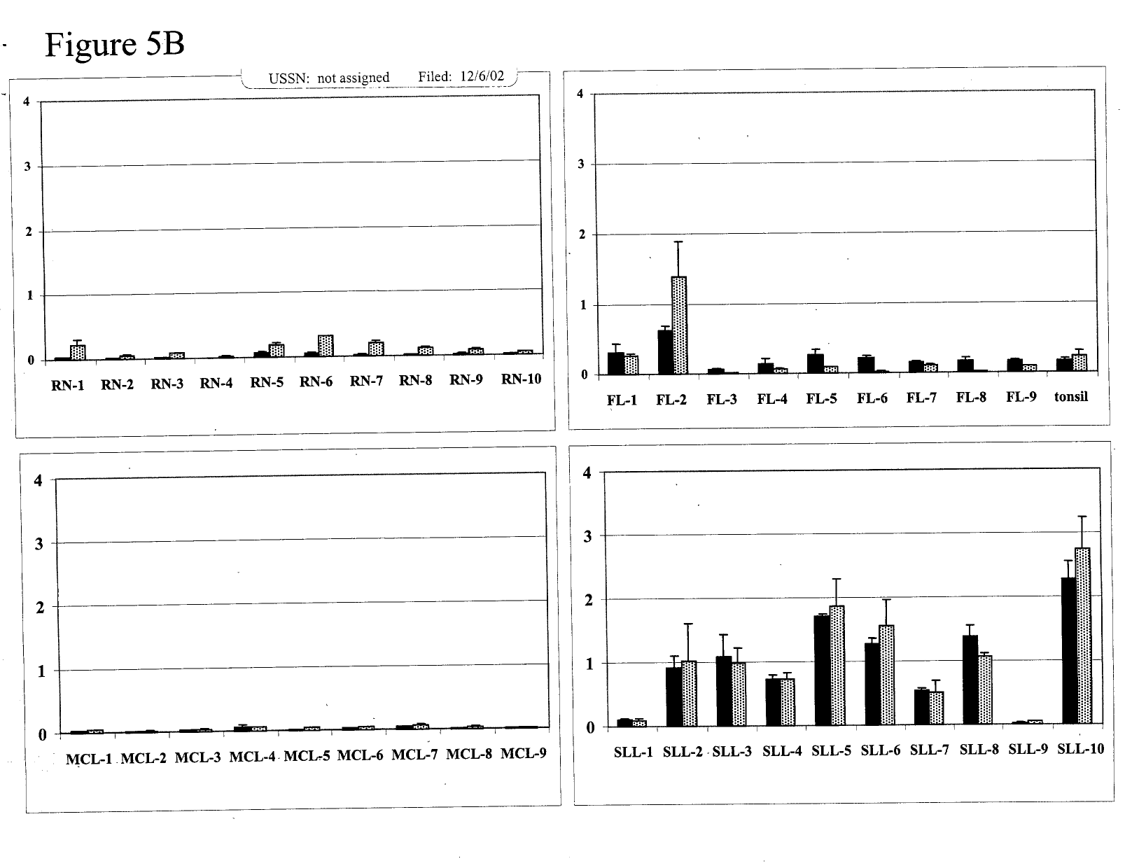

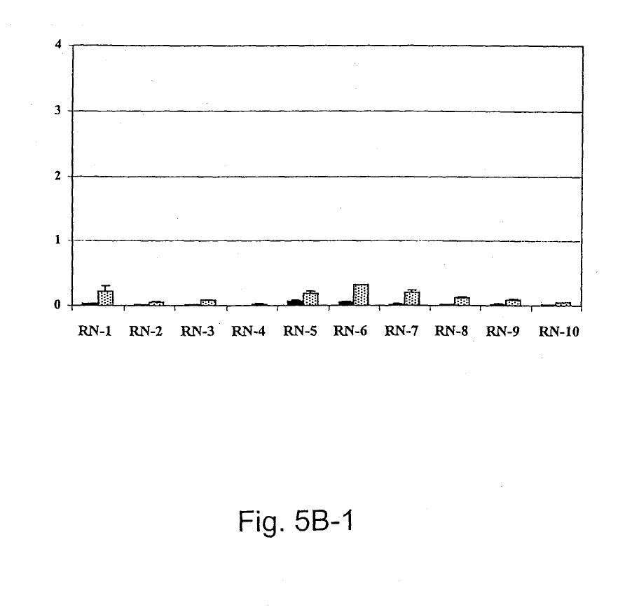

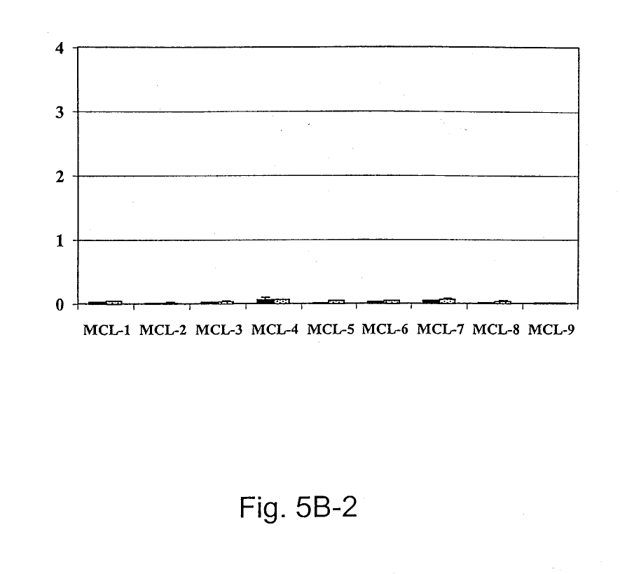

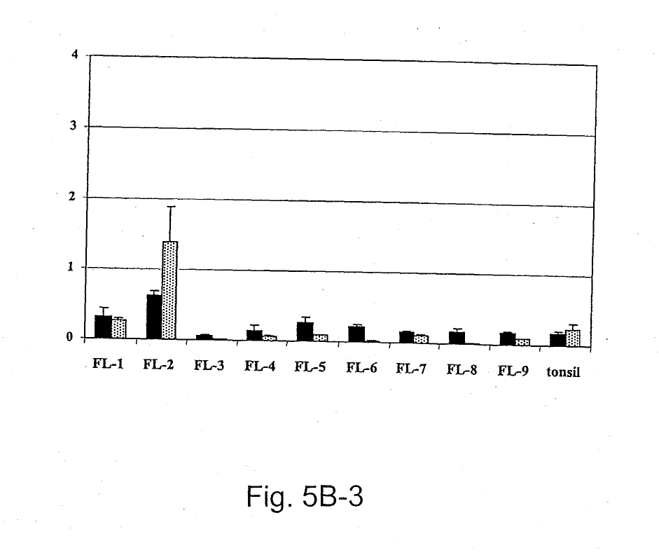

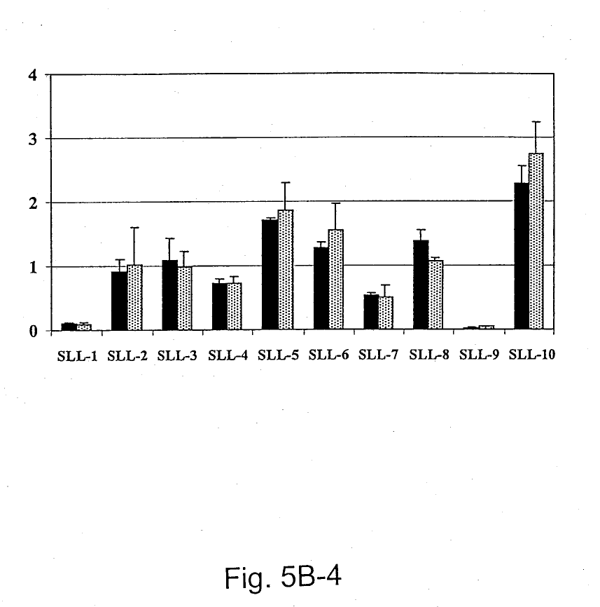

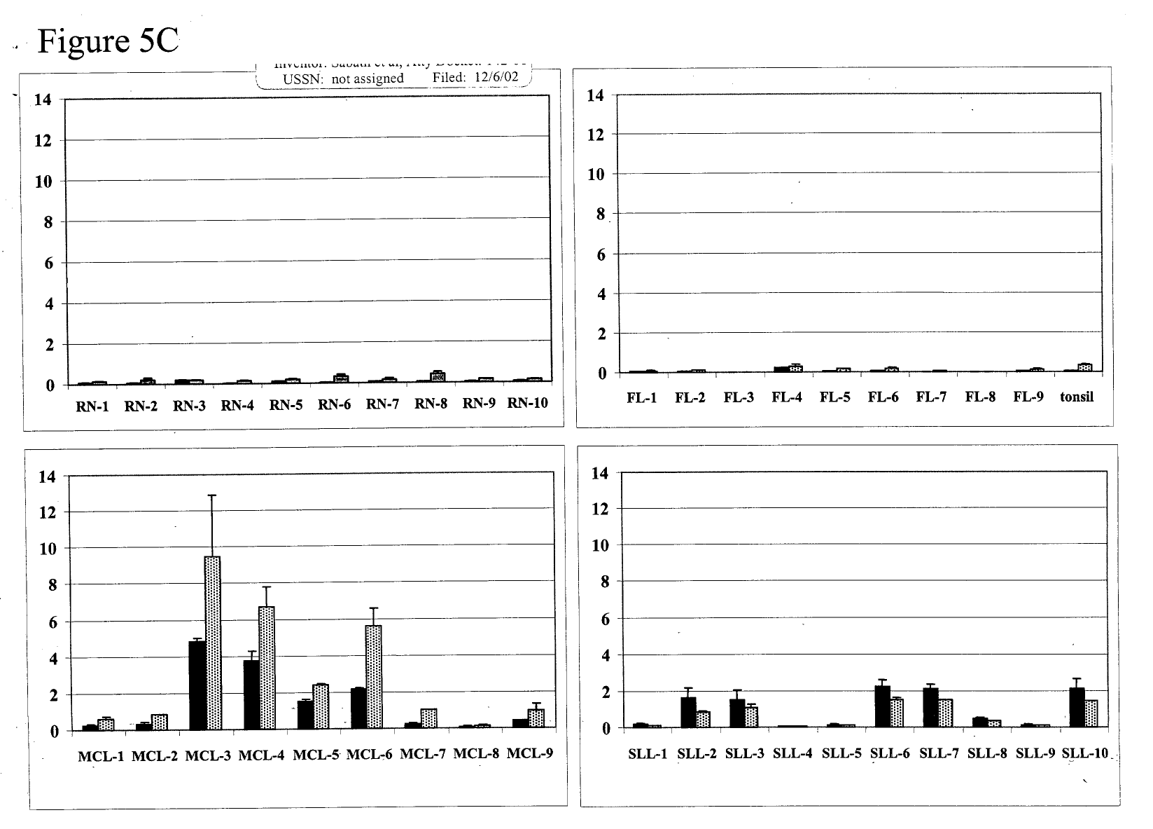

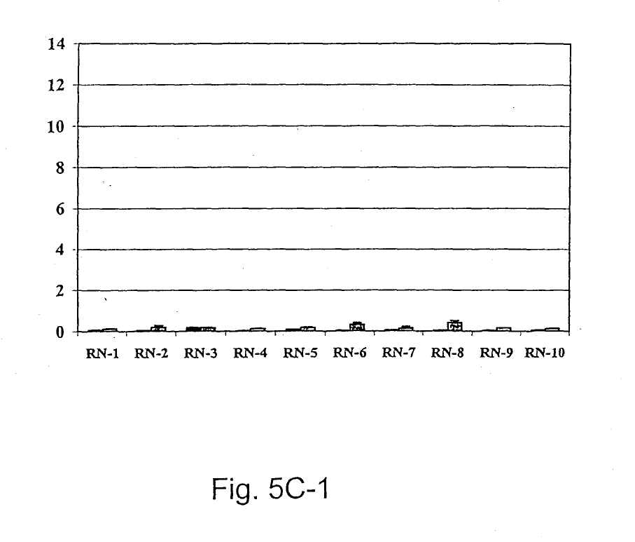

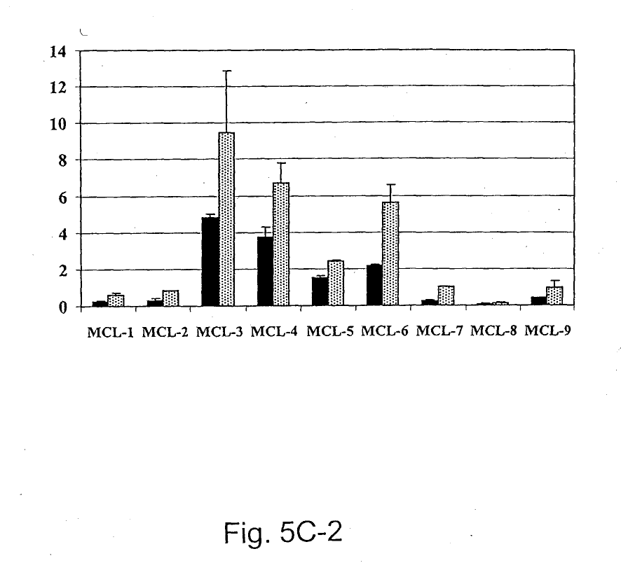

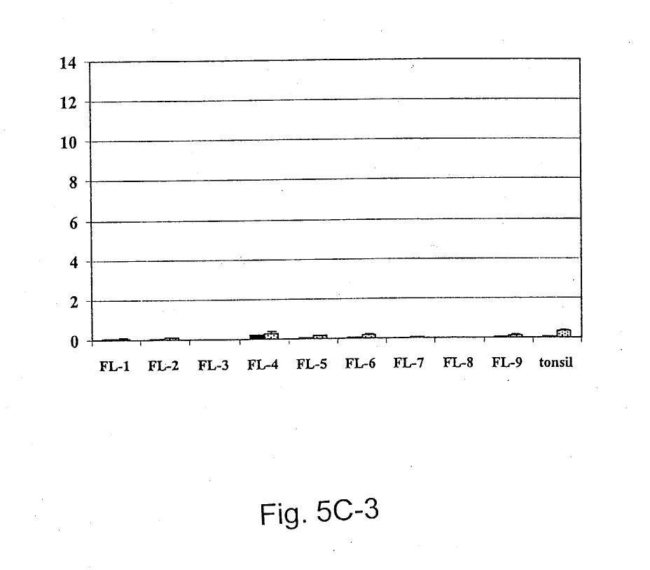

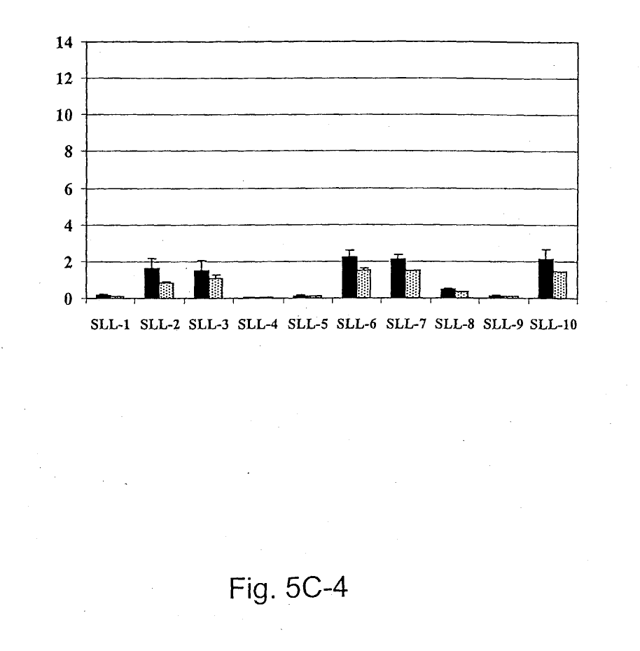

qRT-PCR. 40 μL PCR mixtures contained 1× AmpliTaq Gold Buffer (Applied Biosystems, Foster City, Calif.), 4 mM MgCl₂, 0.025 U/μL AmpliTaq Gold (Applied Biosystems), 0.25 U/μL Moloney leukemia virus reverse transcriptase (Invitrogen, Carlsbad, Calif.), 0.4 U/μL RNase inhibitor (Invitrogen), 0.5 μg/μL BSA (Ambion, Austin, Tex.), 0.33× SYBR Green I (obtained as a 10,000× solution from Molecular Probes, Eugene, Oreg.), 0.8 μM passive reference DNA oligohexamer, 5′-(6-carboxyrhodamine)-GATTAG-PO₄-3′ (Rox Standard I, Synthegen, Houston, Tex.), 200 μM dNTPs (Amersham Biosciences, Piscataway, N.J.), 50 nM gene-specific primers (Invitrogen), and 5 ng poly(A)+RNA. For qRT-PCR validation of array results (FIGS. 3 and 4), an equal amount of poly(A)+RNA was pooled from multiple specimens as described in the footnote of Table 1. For qRT-PCR analysis of individual specimens (FIG. 5), variable amounts (≦5 ng) of poly(A)+RNA were used since 5 ng were not available for all cases; subsequently, the level of cyclin D1, 13cDNA73, and KIAA1407 poly(A)+RNA was normalized to the level of expression of cyclophilin. Gene-specific primers were designed using the computer program Primer Express 1.5 (Applied Biosystems) using default settings for RT-PCR primer selection. Using an ABI 7700 sequence detector (Applied Biosystems), the reactions were subjected to the following cycling conditions: 30 min at 48° C., 10 min at 95° C., and 40 cycles comprised of 15 seconds at 95° C. and 1 minute at 60° C. SYBR Green I is fluorescent when bound to double-stranded DNA. Messenger RNA can be semi-quantified based on the assumption that amplicon concentration doubles with each round of PCR [Wittner et al Clin Chem 48:1178]. Samples with a high poly(A)+RNA copy number, subjected to RT-PCR, produce a threshold level of fluorescent signal after fewer PCR cycles (Ct, or cycle number resulting in threshold fluorescent signal) than samples with a low poly(A)+RNA copy number. Thus, the relative amount of poly(A)+RNA in two samples, sample 1 and sample 2, can be semi-quantified based on the following formula:

([RNA]_{Sample 1}/[RNA]_{Sample 2)}≈E^{(Ct Sample 2-Ct Sample 1)}≈2^{(Ct Sample 2-Ct Sample 1)},

where E is PCR efficiency (approximately 2 in exponential phase).

Results

cDNA array analysis. cDNA microarrays constructed at the UW-CEA were used to identify genes that differ in expression among RN, FL, MCL and SLL specimens. These arrays contained duplicate spots of PCR-amplified insert cDNAs from 14,976 sequence verified IMAGE clones (G Lennon Genomics 33:151) representing ˜13,500 individual UniGene clusters. Poly(A)+RNA was purified from archival tissue specimens that had been frozen shortly after their surgical removal and maintained at −70° C. For array analysis, equal amounts of poly(A)+RNA from multiple specimens representing the same tissue type were pooled. Poly(A)+RNA from 17 RN specimens was pooled to generate a single pool of RN poly(A)+RNA. Similarly, poly(A)+RNA from 14 Grade I FL specimens, 12 MCL specimens, and 16 SLL specimens was pooled. Cy3- and Cy5-labeled first-strand cDNA was generated from each poly(A)+RNA pool. Additionally, Cy3- and Cy5-labeled tonsil cDNA was generated for use as a reference cDNA population. Sample and reference cDNAs were combined and hybridized to microarrays. All array experiments were done in duplicate where the labeling scheme was reversed to compensate for potential dye-specific incorporation effects and for dye-dependent non-linearity in signal intensity (Methods 24: 289 AK Kenworthy; Ramdas L Genome Biol 2001;2(11):RESEARCH0047). For example, Cy3-labeled lymphoma cDNA and Cy5-labeled tonsil cDNA were hybridized to one array whereas Cy5-labeled lymphoma cDNA and Cy3-labeled tonsil cDNA were hybridized to a second array. After hybridization, microarray slides were washed under conditions of increasing stringency and scanned in the Cy3 and Cy5 channels using a laser confocal scanner.

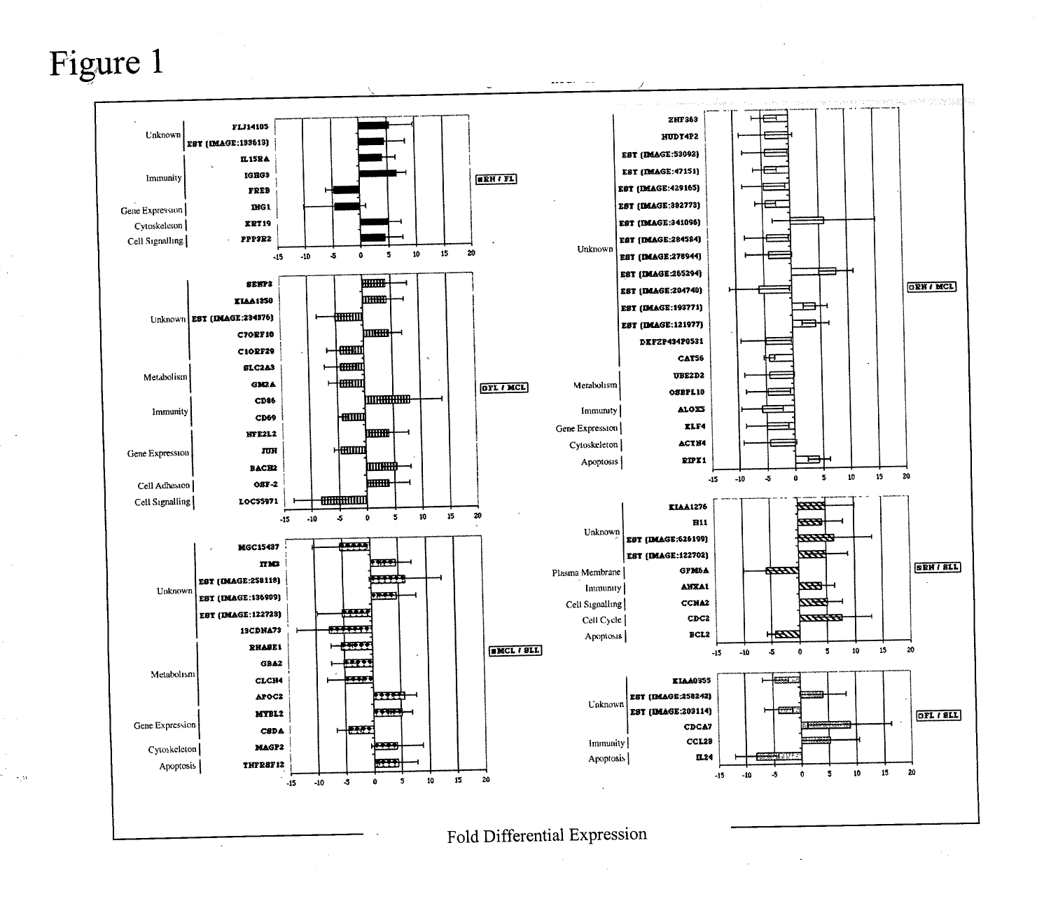

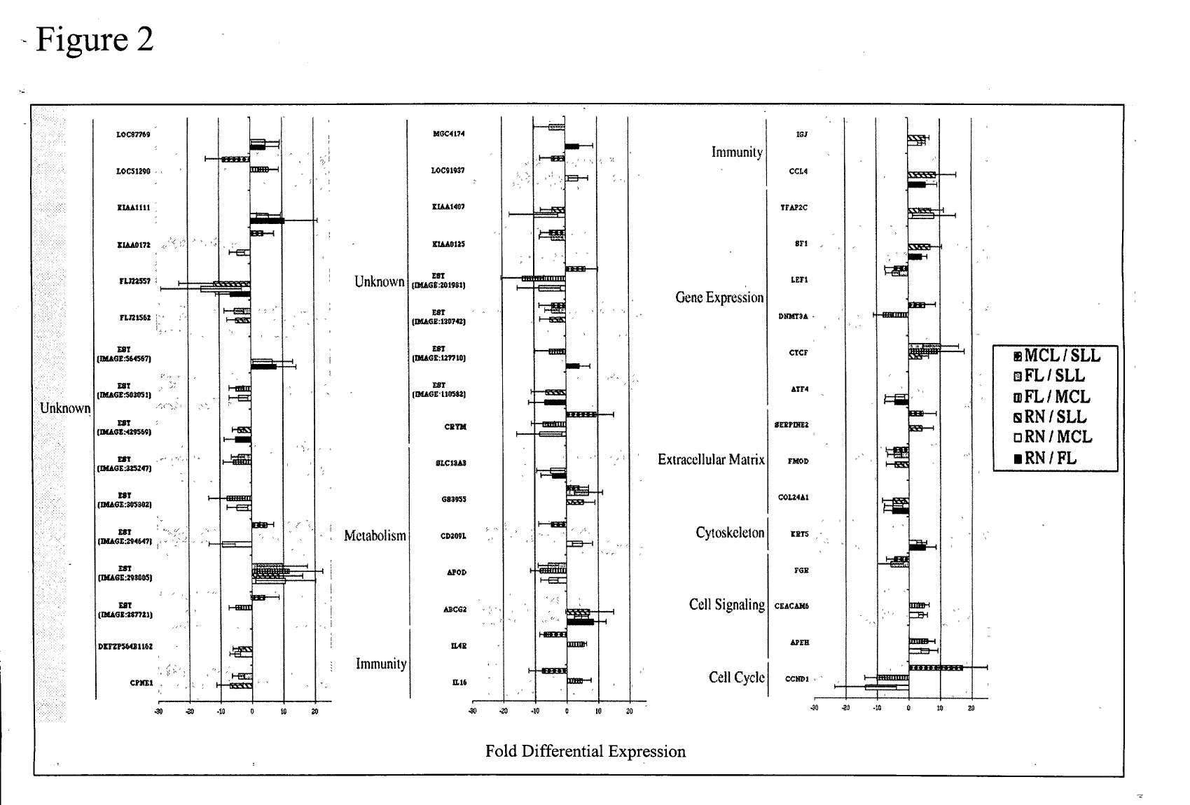

Signal and local background intensities were quantified for each spot using the Spot-On software package developed at the UW-CEA. At least four separate measurements were obtained for each IMAGE clone. Since the vast majority of IMAGE clones were spotted in duplicate and since two arrays per sample were used, four measurements were obtained for most clones. However, some image clones were spotted more than twice per array; alternatively, some genes were represented by more than one IMAGE clone. In these cases, more than four measurements were obtained. The mean (+/− standard error of the mean) expression intensity was calculated based on all available intensity measurements for each gene represented on the array. Ninety-one genes that were ≧4-fold differentially expressed (p≦0.05 based on t-test analysis) between any two tissue types were selected by pairwise analysis using the GeneSifter™ array data analysis program (VizX Labs, LLC, Seattle, Wash.). 39 of these genes were differentially expressed in two or more pairwise comparisons (FIG. 1). 8, 11, 6, 12, 4, and 11 genes were uniquely differentially expressed between RN and FL, RN and MCL, RN and SLL, FL and MCL, FL and SLL, and MCL and SLL, respectively (FIG. 2). Table 1 lists the genes, IMAGE clone numbers, representative accession numbers, chromosomal locations, and functional information for these 120 genes.

Validation of array results using qRT-PCR. The confirmatory method of qRT-PCR with SYBR Green I dye detection (M Rajeevan J Mol Diag 3:26) was used to quantify the relative RNA expression for a subset of 38 of the 120 genes. qRT-PCR was performed in duplicate on pooled poly(A)+RNA from multiple RN, FL, MCL, or SLL specimens. A general analysis of the present array and qRT-PCR data is depicted in FIG. 3. Data for 4 genes (10% of 39) genes was not informative due to lack of amplification or high no-RT background signal. Of the remaining 35 genes, 23 (66% of 35) were found by qRT-PCR to be ≧2-fold differentially expressed in the same direction as the microarray data and to give amplicons that migrated as a single band of expected size by polyacrylamide gel electrophoresis (data not shown). FIG. 4 compares the array and qRT-PCR results for these 23 genes in pseudocolor graphics where the expression level of each gene in RN, FL, MCL, and SLL is displayed as a ratio of its expression level relative to the reference RNA (tonsil) pool.

qRT-PCR analysis of gene expression in individual specimens. Pooled poly(A)+RNA was used to identify and confirm differential gene expression patterns. The level of expression of selected genes in individual specimens was also investigated. qRT-PCR was performed in duplicate for several genes of interest for 10 RN specimens, 9 FL specimens, pooled tonsil RNA, 10 MCL specimens, and 10 SLL specimens. Data for one of the MCL specimens (MCL-14) were discarded due to high amplification signals in no-RT controls (data not shown). Selected results for the remaining specimens are shown in FIG. 5. As expected, cyclin D1 was more highly expressed in all MCL specimens than in any other specimens (FIG. 5A). There was considerable variability (˜6-fold range) in cyclin D1 expression in individual MCL cases. 13cDNA73 was not appreciably expressed in RN, MCL, or tonsil, but was variably expressed in FL specimens (˜16 fold range) and SLL specimens (˜80 fold range) (FIG. 5B). KIAA1407 was not appreciably expressed in RN, FL, or tonsil, but there was variable expression in MCL specimens (˜21 fold range) and SLL specimens(˜20-fold range) (FIG. 5C).

Using cDNA microarray analysis, 120 genes whose expression patterns appear to distinguish among RN, FL, MCL, and SLL have been identified. The differential expression patterns of 23 of these genes were validated using the complementary approach of quantitative RT-PCR. This list includes genes previously known to be differentially expressed in LGBCL, genes known to be involved in cancer types other than lymphoma, genes not previously associated with malignancy, and partially characterized genes/ESTs of unknown function.

The results herein largely do not confirm previously published results obtained using oligonucleotide microarrays to examine gene expression in RN and MCL (Hofmann, de Vos et al. 2001) In that study, 92 genes were identified to be ≧3-fold differentially expressed between MCL and RN (Hofmann, de Vos et al. 2001). The cDNA arrays used herein contained probes for 55 of these genes. However, the results herein showed that only 11 (20%) of these 55 genes were significantly (p≦0.05) differentially expressed between RN and MCL and that, among these genes, only cyclin D1 showed ≧3-fold differential expression (data not shown). The reason for discrepancy between the present data set and these published results is unclear. However, a recent report suggests that microarray data obtained using oligonucleotide and cDNA array platforms may not be directly comparable (Kuo, Jenssen et al. 2002).

The differential expression of several genes previously known to be involved in LGBCL was validated. The finding that cyclin D1 was significantly over-expressed in MCL was not surprising since the expression of this cell cycle regulatory protein is known to be involved in MCL pathogenesis (Tsujimoto, Yunis et al. 1984) (Tsujimoto, Jaffe et al. 1985). By qRT-PCR analysis of individual cases considerable variability (˜6-fold range) in cyclin D1 expression in individual MCL cases was found. Variable expression levels of cyclin D1 in MCL specimens have also been obtained by other researchers using qRT-PCR (Specht, Kremer et al. 2002) (Elenitoba-Johnson, Bohling et al. 2002). This variability may reflect different proportions of neoplastic cells within tissue specimens sampled or an underlying difference in the neoplastic cells themselves. A transcript binding to a v-jun cDNA probe was significantly over-expressed in MCL relative to FL. The transcript was presumably c-jun since the amplicon generated in qRT-PCR analysis using primers directed against c-jun sequence was of a size expected from the c-jun sequence (data not shown). c-Jun is an AP-1 transcription factor component known to be over-expressed in the malignant Reed-Sternberg cells that characterize Hodgkin's disease (Mathas, Hinz et al. 2002). c-Jun was recently found by array analysis to be over-expressed in MCL relative to RN (Hofmann, de Vos et al. 2001). Lastly, we found that BCL-2 was significantly over-expressed in SLL versus RN and was over-expressed in FL versus RN, although not at the p<0.05 level of significance (data not shown). Since BCL-2 is known to be involved in the pathogenesis of FL and SLL (Hockenbery, Nunez et al. 1990) (Vaux, Cory et al. 1988), we expected to identify this gene as being over-expressed in these specimen types.

Several of the genes identified have well-established roles in cancer types other than lymphoma. A transcript binding to the v-myb cDNA probe (presumably c-myb since the qRT-PCR product was of size expected for c-myb [data not shown]) was significantly under-expressed in MCL versus SLL. c-myb is a member of the myb family of transcription factors that regulate the proliferation, differentiation, and apoptosis of hematopoietic cells and are frequently over-expressed in human myeloid and lymphoid leukemias (Davies, Badiani et al. 1999). c-myb expression in LGBCL lymphomas is believed to not have been previously studied. DNA methyltransferase 3A (DNMT3A) was significantly over-expressed in MCL relative to FL and CLL. DNMT3A and DNMT3B are thought to establish cytosine methylation patterns that influence the expression of genes containing upstream CpG islands (Jones and Laird 1999). DNA from malignant cells often shows global hypomethylation but localized CpG island hypermethylation resulting in the down-regulated expression of tumor suppressor genes (Melki and Clark 2002). DNMT over-expression may contribute to altered DNA methylation patterns in cancer and CLL cells were recently shown to have increased DNMT3A expression relative to normal lymphocytes (Melki and Clark 2002). However, no published studies have directly compared methylation patterns between FL, MCL, and SLL.

The expression patterns of several of the genes identified have been studied in cancer but have no known role in carcinogenesis. CD69 was over-expressed in MCL relative to RN. CD69 is best known as a T lymphocyte antigen and was previously shown to be expressed by benign activated B lymphocytes as well as by malignant FL, MCL, and SLL cells (Erlanson, Gronlund et al. 1998). However, a role for CD69 in lymphoma is unclear and the findings may be explained by the expression of this gene product in T cells contained within lymphoma specimens. We found that keratin 5 was under-expressed in FL and MCL relative to RN. Keratin 5 is not normally expressed in lymphocytes but are expressed in lymph node reticular cells (Doglioni, Dell'Orto et al. 1990). Decreased keratin 5 expression in lymphoma may reflect replacement of the normal lymph node architecture with neoplastic cells. We found that crystallin mu was over-expressed in MCL relative to RN and FL. Crystallin mu was originally cloned as a structural component of kangaroo lens (Kim, Gasser et al. 1992) and was more recently cloned as a nicotinamide-adenine dinucleotide phosphate-regulated thyroid hormone binding protein (Vie, Evrard et al. 1997). The role of this protein in lymphoma is unclear. A previous study of thyroxine (T3) and triiodothyronine (T4) metabolism in a small number of euthyroid patients with lymphoma and other hematopoietic disorders found that these patients showed increased levels of T3/T4 per body mass unit (Kirkegaard, Hasselbalch et al. 1989). This result suggests that hematopoictic tumors contain concentrated thyroid hormone binding sites. Over-expressed crystallin mu may serve as a T3/T4 sink in lymphoma patients.

Several of the genes identified are partially characterized genes/expressed sequence tags (ESTs) of unknown function. IMAGE:293005 was over-expressed in RN and FL relative to MCL and SLL. This gene shares 72% identity over ˜460 nucleotides with a mouse homologue (encoding the murine M17 protein) which is known to be highly expressed in the germinal centers of mice (Christoph, Rickert et al. 1994). Because RN specimens contain expanded germinal centers and FL specimens are largely comprised of neoplastic cells of germinal center origin, the expression patterns identified are consistent with germinal expression of IMAGE:293005. Further, this gene falls within the germinal center cluster identified by Alizadeh and colleagues through the microarray-based analysis of a large number of normal and malignant lymphocyte samples (Alizadeh, Eisen et al. 2000) (data not shown).

13cDNA73 was brightly over-expressed in SLL and moderately over-expressed in FL relative to RN and MCL. Individual case qRT-PCR analysis showed that expression of this gene product varied markedly among SLL (˜80 fold range) and FL (˜16 fold range) specimens. KIAA1407 was over-expressed in MCL and SLL relative to RN and FL. QRT-PCR analysis of individual cases showed that expression varied markedly among MCL (˜˜21 fold range) and SLL (˜16 fold range).

Although this description contains many specificities, these should not be construed as limiting the scope of the invention, but as merely providing illustrations of some of the preferred embodiments of the invention. All references cited herein are hereby incorporated by reference to the extent not inconsistent with the disclosure herewith.

TABLE 1

| 120 Genes Identified by cDNA Microarray Analysis¹ to be Differentially Expressed (≧4-Fold, p ≦ 0.05) | | | | | | | | |
|---|---|---|---|---|---|---|---|---|
| among RN, FL, MCL, and SLL¹ | | | | | | | | |
| | | I.M.A.G.E. | | | | | | |
| | | Clone | | Representative | Chromosomal | qRT-PCR | | |
| Gene | | Number(s) | Function | Sequence | Location | Results | Forward Primer | Reverse Primer |
| 13CDNA73 | EST | 46284 | Unknown | NM_023037 | 13q13.3 | Confirmed² | GATGACGACAGG | TGACCAGGACTG |
| | | | | | | | CCGATGATT | CGTTCCATT |
| | | | | | | | SEQ ID NO: 224 | SEQ ID NO: 225 |
| ABCG2 | ATP-binding | 288736 | Small | NM_004827 | 4q22 | ND | | |
| | cassette, sub- | | Molecule | | | | | |
| | family G | | Transport | | | | | |
| ACTN4 | Actinin, alpha 4 | 140951 | Cytoskeleton | NM_004924 | 19q13 | ND | | |
| ALOX5 | Arachidonate 5- | 179890 | Immune | NM_000698 | 10q11.2 | ND | | |
| | lipoxygenase | | | | | | | |
| ANXA1 | Annexin A1 | 208718 | Immune | NM_000700 | 9q12-q21.2 | ND | | |
| APEH | N- | 813279 | Metabolism | NM_001640 | 3p21 | ND | | |
| | acylaminoacyl- | | | | | | | |
| | peptide | | | | | | | |
| | hydrolase | | | | | | | |
| APOC2 | Apolipoprotein | 809523 | Metabolism | NM_000483 | 19q13.2 | Confirmed³ | CCCGCTGTAGAT | TCTCCCTTCAGC |
| | C-II | | | | | | GAGAAACTCA | ACAGAAAGAA |
| | | | | | | | SEQ ID NO: 226 | SEQ ID NO: 227 |
| APOD | Apolipoprotein | 159608 | Metabolism | NM_001647 | 3q26.2-qter | ND | | |
| | D | | | | | | | |
| ATF4⁴ | Activating | 949971 | Gene | NM_001675 | 22q13.1 | ND | | |
| | transcription | | Expression | | | | | |
| | factor 4 | | Regulation | | | | | |
| BACH2 | BTB and CNC | 296483 | Unknown | NM_021813 | 6q15 | ND | | |
| | homology 1, | | | | | | | |
| | basic leucine | | | | | | | |
| | zipper | | | | | | | |
| | transcription | | | | | | | |
| | factor 2 | | | | | | | |
| BCL2⁵ | B-cell | 232714 | Apoptosis | NM_000633 | 18q21.3 | Confirmed³ | ATGACTGAGTAC | CAGAGACAGCC |
| | CLL/lymphoma | | | | | | CTGAACCGGC | AGGAGAAATCA |
| | 2 | | | | | | SEQ ID NO: 228 | SEQ ID NO: 229 |
| C1ORF29 | Chromosome 1 | 754479 | Unknown | NM_006820 | 1p31.1 ND | | | |
| | open reading | | | | | | | |
| | frame 29 | | | | | | | |
| C7ORF10 | Chromosome 7 | 309499 | Unknown | NM_024728 | 7p15.2 | ND | | |
| | open reading | | | | | | | |
| | frame 10 | | | | | | | |
| CAT56⁵ | Guanine | 60201 | Unknown | NM_025263 | 6p21.3 | ND | | |
| | nucleotide | | | | | | | |
| | binding protein- | | | | | | | |
| | like 1 | | | | | | | |
| CCL28 | Chemokine(C-C | 136919 | Immune | NM_019846 | 5p12 | ND | | |
| | motif) ligand 28 | | | | | | | |
| CCL4⁴ | Chemokine (C-C | 205633 | Immune | NM_002984 | 17q12 | Confirmed² | CCAGCTGTGGTA | TGAGCAGCTCAG |
| | motif) ligand 4 | | | | | | TTCCAAACCA | TTCAGTTCCA |
| | | | | | | | SEQ ID NO: 230 | SEQ ID NO: 231 |
| CCNA2⁵ | Cyclin A2 | 950690 | Cell | NM_001237 | 4q25-q31 | Confirmed² | GCTGGCCTGAAT | GCATGCTGTGGT |
| | | | Signalling | | | | CATTAATACG | GCTTTGA |
| | | | | | | | SEQ ID NO: 232 | SEQ ID NO: 233 |
| CCND1 | Cyclin D1 | 841641 | Cell Cycle | NM_053056 | 11q13 | Confinned² | AGGTCTGCGAGGA | TGCAGGCGGCTC |
| | | | Regulation | | | | ACAGAAGTG | TTTTTCA |
| | | | | | | | SEQ ID NO: 234 | SEQ ID NO: 235 |
| CD209L | CD209 antigen- | 782758 | Metabolism | NM_014257 | 19p13 | Not Confirmed³ | TGCTGCAACTCCT | CGTCTTGCTCGG |
| | like DC-SIGNR | | | | | | CTCCTTCAT | ATTGTTCCT |
| | | | | | | | SEQ ID NO: 236 | SEQ ID NO: 237 |
| CD69 | CD69 antigen | 704459 | Immune | NM_001781 | 12p13-p12 | Confirmed³ | CATGGTGCTACTC | CCCTGTAACGTT |
| | | | | | | | TTGCTGTCA | GAACCAGTTG |
| | | | | | | | SEQ ID NO: 238 | SEQ ID NO: 240 |
| CD86 | CD86 antigen | 50214 | Immune | NM_006889 | 3q21 | Not Confirmed² | GGAAAAGACATC | TCTGGTTGTGGT |
| | | | | | | | AACCCCCATA | CTCTGGTGTT |
| | | | | | | | SEQ ID NO: 241 | SEQ ID NO: 242 |
| CDC2 | Cell division | 898286 | Cell Cycle | NM_001786 | 10q21.1 | ND | | |
| | cycle 2, G1 to S | | Regulation | | | | | |
| | and G2 to M | | | | | | | |
| CDCA7 | c-Myc target | 244058 | Unknown | NM_031942 | 2q31 | ND | | |
| | JPO1 | | | | | | | |
| CEACAM6 | Carcinoembryonic | 509823 | Cell | NM_002483 | 19q13.2 | ND | | |
| | antigen- | | Signalling | | | | | |
| | related cell | | | | | | | |
| | adhesion | | | | | | | |
| | molecule 6 | | | | | | | |
| CLCN4 | Chloride channel | 363058 | Small | NM_001830 | Xp22.3 | High No-RT | GCGGCACTGCAG | TTCCCTTAGCCA |
| | 4 | | Molecular | | | Control² | GTGTAATTA | GTCGATGGT |
| | | | Transport | | | | SEQ ID NO: 243 | SEQ ID NO: 244 |
| COL24A1 | Collagen type | 280567 | Extracellular | NM_152890 | 1 | ND | | |
| | XXIV, alpha 1 | | Matrix | | | | | |
| CPNE1 | Copine I | 843139 | Unknown | NM_003915 | 20q11.21 | Not Confirmed³ | CTGCCTCGCAAT | CCACACCCACAA |
| | | | | | | | ACTTCATGCT | TGATCACTGA |
| | | | | | | | SEQ ID NO: 245 | SEQ ID NO: 246 |
| CRYM | Crystallin, mu | 42373 | Unknown | NM_001888 | 16p13.11- | Confirmed² | GGCAGGTGCAGA | TGGCTCCAACAG |
| | | | | | p12.3 | | TGTGATCAT | CATTGATG |
| | | | | | | | SEQ ID NO: 247 | SEQ ID NO: 248 |
| CSDA | Cold shock | 810057 | Gene | NM_003651 | 12p13.1 | ND | | |
| | domain protein | | Expression | | | | | |
| | A | | Regulation | | | | | |
| CTCF | CCCTC-binding | 240367 | Gene | NM_006565 | 16q21- | Discordant² | CACACAGGTACT | TCGCACATGGAA |
| | factor (zinc | | Expression | | q22.3 | | CGTCCTCACA | CACTTGAA |
| | finger protein) | | Regulation | | | | SEQ ID NO: 249 | SEQ ID NO: 250 |
| DKFZP434P0531 | EST | 325024 | Unknown | BC022095 | 6p21.3 | ND | | |
| DKFZP564B1162⁵ | EST | 418185 | Unknown | NM_031305 | 4q21.23- | ND | | |
| | | | | | g21.3 | | | |
| DNMT3A | DNA (cytosine- | 202514 | Gene | AF331856 | 2p23 | Confirmed³ | CCATTCCTGGTCA | TCCTGTGTGGTA |
| | 5-)- | | Expression | | | | CGCAAAAC | GGCACCTGAA |
| | methyltransferase | | Regulation | | | | SEQ ID NO: 251 | SEQ ID NO: 252 |
| | 3 alpha | | | | | | | |
| EST | EST | 47151 | Unknown | R48935 | | ND | | |
| (IMAGE:47151) | | | | | | | | |
| EST | EST | 53092 | Unknown | BG284034 | 2 | ND | | |
| (IMAGE:53092) | | | | | | | | |
| EST | EST | 110582 | Unknown | T90074 | 11 | ND | | |
| (IMAGE:110582) | | | | | | | | |
| EST | EST | 121977 | Unknown | T97780 | | ND | | |
| (IMAGE:121977) | | | | | | | | |
| EST | EST | 122702 | Unknown | BC034319 | | ND | | |
| (IMAGE:122702) | | | | | | | | |
| EST | EST | 122723 | Unknown | AA777690 | | ND | | |
| (IMAGE:122723) | | | | | | | | |
| EST | EST | 127710 | Unknown | AA579610 | 10 | ND | | |
| (IMAGE:127710) | | | | | | | | |
| EST | EST | 130742 | Unknown | H13708 | | ND | | |
| (IMAGE:130742) | | | | | | | | |
| EST | EST | 133613 | Unknown | R30836 | | ND | | |
| (IMAGE:133613) | | | | | | | | |
| EST | EST | 136909 | Unknown | BU162571 | | ND | | |
| (IMAGE:136909) | | | | | | | | |
| EST | EST | 193771 | Unknown | BQ322085 | 11 | ND | | |
| (IMAGE:193771) | | | | | | | | |
| EST | EST | 201981 | Unknown | BC025340 | 6 | Confirmed³ | CCGTCTGTCTCCT | TCCTGTCCTCTGCT |
| (IMAGE:201981) | | | | | | | TTCCTTCTG | CTGTGGAT |
| | | | | | | | SEQ ID NO: 253 | SEQ ID NO: 254 |
| EST | EST | 203114 | Unknown | BF431502 | | ND | | |
| (IMAGE:203114) | | | | | | | | |
| EST | EST | 204740 | Unknown | H57305 | | ND | | |
| (IMAGE:204740) | | | | | | | | |
| EST | EST | 234376 | Unknown | AK097411 | | ND | | |
| (IMAGE:234376) | | | | | | | | |
| EST | EST | 258118 | Unknown | N27108 | 7 | Discordant³ | TGCTCCCCTGTTT | TCCTGGAAGTAAT |
| (IMAGE:258118) | | | | | | | TTGTGACA | GCCAACTCA |
| | | | | | | | SEQ ID NO: 255 | SEQ ID NO: 256 |
| EST | EST | 258242 | Unknown | BE786990 | 1 | ND | | |
| (IMAGE:258242) | | | | | | | | |
| EST | EST | 265294 | Unknown | N20848 | | ND | | |
| (IMAGE:265294) | | | | | | | | |
| EST | EST | 278944 | Unknown | AL121338 | | ND | | |
| (IMAGE:278944) | | | | | | | | |
| EST | EST | 284584 | Unknown | N59450 | | ND | | |
| (IMAGE:284584) | | | | | | | | |
| EST | EST | 287721 | Unknown | N79323 | | ND | | |
| (IMAGE:287721) | | | | | | | | |
| EST | EST | 293005 | Unknown | NM_152785 | 3 | Confirmed² | GGCCTAGAGCCT | TTGCTCCTCTCACT |
| (IMAGE:293005) | | | | | | | CTTGATTCAA | CCATGTGT |
| | | | | | | | SEQ ID NO: 257 | SEQ ID NO: 258 |
| EST | EST | 294647 | Unknown | BE971364 | | ND | | |
| (IMAGE:294647) | | | | | | | | |
| EST | EST | 305302 | Unknown | BM906531 | 3 | ND | | |
| (IMAGE:305302)⁵ | | | | | | | | |
| EST | EST | 325247 | Unknown | BU630466 | 17 | ND | | |
| (IMAGE:325247) | | | | | | | | |
| EST | EST | 341096 | Unknown | BM546103 | 15 | ND | | |
| (IMAGE:341096)⁵ | | | | | | | | |
| EST | EST | 382773 | Unknown | AA065090 | | ND | | |
| (IMAGE:382773)⁵ | | | | | | | | |
| EST | EST | 429165 | Unknown | BF677678 | 11 | ND | | |
| (IMAGE:429165)⁵ | | | | | | | | |
| EST | EST | 429569 | Unknown | AI248013 | 19 | ND | | |
| (IMAGE:429569) | | | | | | | | |
| EST | EST | 503051 | Unknown | BU630466 | 17 | ND | | |
| (IMAGE:503051) | | | | | | | | |
| EST | EST | 564567 | Unknown | AA127395 | 3 | ND | | |
| (IMAGE:564567) | | | | | | | | |
| EST | EST | 626199 | Unknown | BG283145 | 7 | ND | | |
| (IMAGE:626199) | | | | | | | | |
| FGR | Gardner-Rasheed | 681906 | Cell | NM_005248 | 1p36.2- | ND | | |
| | feline sarcoma | | Signalling | | p36.1 | | | |
| | viral (v-fgr) | | | | | | | |
| | oncogene | | | | | | | |
| | homolog | | | | | | | |
| FLJ14105 | EST | 742904 | Unknown | BQ070901 | 2 | ND | | |
| FU21562 | EST | 212772 | Unknown | NM_025113 | 13q14.11 | Confirmed² | CAGCTGGCTCGAT | TCTAGGAGGAGCCC |
| | | | | | | | AGTCGTAAA | AGTCTTCA |
| | | | | | | | SEQ ID NO: 259 | SEQ ID NO: 260 |
| FLJ22557 | EST | 501778 | Unknown | NM_024713 | 15q13.1 | ND | | |
| FMOD | Fibromodulin | 811162 | Extracellular | NM_002023 | 1q32 | ND | | |
| | | | Matrix | | | | | |
| FREB | Fc receptor | 290749 | Immune | NM_032738 | 1q23.1 | ND | | |
| | homolog | | | | | | | |
| GBA2 | Glucosidase, beta | 796297 | Metabolism | NM_020944 | 9p11.2 | ND | | |
| | (bile acid) 2 | | | | | | | |
| GM2A | GM2 ganglioside | 795173 | Metabolism | NM_000405 | 5q31.3- | Confirmed³ | AAAAGCCATCCCA | CACATTTCCAGGAA |
| | activator protein | | | | q33.1 | | GCTCAGTAG | CGACGAT |
| | | | | | | | SEQ ID NO: 261 | SEQ ID NO: 262 |
| GPM6A | Glycoprotein | 784910 | Plasma | NM_005277 | 4q34 | ND | | |
| | M6A | | Membrane | | | | | |
| | | | Protein | | | | | |
| GS3955⁵ | GS3955 protein | 813426 | Cell | NM_021643 | 2p25.1 | Primer Dimer³ | AGGAGCTGGTGTG | CCCCATAGCTTCGC |
| | | | Signalling | | | | CAAGGTGTT | TCAAAGAA |
| | | | | | | | SEQ ID NO: 263 | SEQ ID NO: 264 |
| H11 | Protein kinase | 205049 | Unknown | NM_014365 | 12q24.23 | ND | | |
| | H11 | | | | | | | |
| IGHG3 | Immunoglobulin | 289337 | Immune | BC019046 | 14q32.33 | Confirmed² | GCAGCCGGAGAAC | TGCATCACGGAGCA |
| | heavy constant | | | | | | AACTACAAG | TGAGAA |
| | gamma 3 | | | | | | SEQ ID NO: 265 | SEQ ID NO: 266 |
| IGJ | Immunoglobulin J | 80948 | Immune | NM_144646 | 4q21 | High No-RT | TCCCATGGCAAGT | CCATGACACAGCCA |
| | polypeptide | | | | | Control² | CCTAAAGC | AACAGAAA |
| | | | | | | | SEQ ID NO: 267 | SEQ ID NO: 268 |
| IL15RA⁴ | Interleukin 15 | 488019 | Immune | NM_002189 | 10p15-p14 | ND | | |
| | receptor, alpha | | | | | | | |
| IL16⁴ | Interleukin 16 | 809776 | Immune | NM_004513 | 15q26.3 | ND | | |
| | (lymphocyte | | | | | | | |
| | chemoattractant | | | | | | | |
| | factor) | | | | | | | |
| IL24 | Interleukin 24 | 712049 | Apoptosis | NM_006850 | 1q32 | Not Confirmed³ | TCTCATCGTGTCAC | GAGCTGCTTCTACG |
| | | | | | | | AACTGCAA | TCCAACTG |
| | | | | | | | SEQ ID NO: 269 | SEQ ID NO: 270 |
| IL4R⁴ | Interleukin 4 | 714453 | Immune | NM_000418 | 16p11.2- | Confirmed³ | CAGCGTTTCCTGCA | GACCCCTGAGCATC |
| | receptor | | | | 12.1 | | TTGTCATC | CTGGATTA |
| | | | | | | | SEQ ID NO: 271 | SEQ ID NO: 272 |
| ING1³ | Inhibitor of | 810061 | Gene | NM_005537 | 13q34 | ND | | |
| | growth family, | | Expression | | | | | |
| | member 1 | | Regulation | | | | | |
| ITM3 | Integral | 471196 | Plasma | NM_030926 | 2q37 | Confirmed² | GGAGCTCCTCATG | AGGTGTCTTTCCCG |
| | membrane protein | | Membrane | | | | AACGTGAA | TTGCA |
| | 3 | | Protein | | | | SEQ ID NO: 273 | SEQ ID NO: 274 |
| JUN⁴ | v-Jun sarcoma | 358531 | Gene | NM_002228 | 1p32-p31 | Confirmed² | CTAACGCAGCAGT | TCTCCGTCGCAACT |
| | virus 17 oncogene | | Expression | | | | TGCAAACA | TGTCAA |
| | homolog | | Regulation | | | | SEQ ID NO: 275 | SEQ ID NO: 276 |
| KIAA0125 | EST | 210368 | Unknown | NM_014792 | 14q32.33 | High No-RT | ATGGCTCCTGCTGT | GTGAAGCGGTGGAC |
| | | | | | | Control^{2.} | ACCTCAAG | AAGAAACT |
| | | | | | | | SEQ ID NO: 277 | SEQ ID NO: 278 |
| KIAA0172 | EST | 812975 | Unknown | D79994 | 9p24.3 | ND | | |
| KIAA0355⁵ | EST | 784104 | Unknown | NM_014686 | 19q13.12 | | | |
| KIAA1111 | EST | 810621 | Unknown | AB029034 | X | | | |
| KIAA1276⁵ | EST | 417637 | Unknown | BQ722784 | 4 | ND | | |
| KIAA1350 | EST | 321886 | Unknown | AB037771 | 4q28.1 | Confirmed² | CGAAGCTGTTGTTC | GGCTGGTGTAGCAG |
| | | | | | | | GGAATC | ATCATACC |
| | | | | | | | SEQ ID NO: 279 | SEQ ID NO: 280 |
| KIAA1407 | EST | 121475 | Unknown | AF509494 | 3q13.31 | Confirmed² | AACCTGCCAGATG | CGGTGTCATCAATT |
| | | | | | | | CTTGTGAAT | GCTTTGG |
| | | | | | | | SEQ ID NO: 281 | SEQ ID NO: 282 |
| KLF4 | Kruppel-like | 188232 | Gene | NM_004235 | 9q31 | Discordant³ | GCTCCATTACCAA | GTGCCTGGTCAGTT |
| | factor 4 | | Expression | | | | GAGCTCATG | CATCTGA |
| | | | Regulation | | | | SEQ ID NO: 283 | SEQ ID NO: 284 |
| KRT19 | Keratin 19 | 810131 | Cytoskeleton | NM_002276 | 17q21 | Primer Dimer² | GCATGAAAGCTGC | CCTGATTCTGCCGC |
| | | | | | | | CTTGGAA | TCACTATC |
| | | | | | | | SEQ ID NO: 285 | SEQ ID NO: 286 |
| KRT5 | Keratin 5 | 592540 | Cytoskeleton | NM_000424 | 12q12-q13 | Confirmed³ | CAGAAGCCGAGTC | TGGCGCACTGTTTC |
| | | | | | | | CTGGTATCA | TTGACA |
| | | | | | | | SEQ ID NO: 287 | SEQ ID NO: 288 |
| LEF1 | Lymphoid | 347036 | Gene | NM_016269 | 4q23-q25 | ND | | |
| | enhancer-binding | | Expression | | | | | |
| | factor 1 | | Regulation | | | | | |
| LOC51290⁵ | EST | 259902 | Unknown | NM_016570 | 12p12.1 | Not Confirmed³ | AGCAGAAAGAGTG | TTGGTGGAAGAGCT |
| | | | | | | | GCAGAGGAT | GTTGATGT |
| | | | | | | | SEQ ID NO: 289 | SEQ ID NO: 290 |
| LOC55971 | Insulin receptor | 131318 | Cell | NM_018842 | 7q11.21 | ND | | |
| | tyrosine kinase | | Signalling | | | | | |
| | substrate | | | | | | | |
| LOC87769 | EST | 781088 | Unknown | BC001077 | 13q32.3 | ND | | |
| LOC91937 | EST | 202315 | Unknown | NM_138379 | 5q33.2 | ND | | |
| MAGP2⁵ | Microfibril- | 138496 | Cytoskeleton | NM_003480 | 12p13.1- | ND | | |
| | associated | | | | p12.3 | | | |
| | glycoprotein-2 | | | | | | | |
| MGC15437 | NM23- | 489047 | Unknown | NM_032873 | 11q24.1 | Confirmed² | GGTGGATCTGTCA | GCCTGTCACCTCAG |
| | phosphorylated | | | | | | GCTGCCATA | AACTCCAA |
| | unknown | | | | | | SEQ ID NO: 291 | SEQ ID NO: 292 |
| | substrate | | | | | | | |
| MGC4174 | Hypothetical | 126450 | Unknown | NM_024319 | 1q42.13 | ND | | |
| | protein MGC4174 | | | | | | | |
| MYBL2 | v-Myb | 815526 | Gene | NM_002466 | 20q13.1 | Confirmed² | CCCATCAAGAAAG | GCAGTTGTCGGCAA |
| | myeloblastosis | | Expression | | | | TCCGGAAGT | GGATAGA |
| | viral oncogene | | Regulation | | | | SEQ ID NO: 293 | SEQ ID NO: 294 |
| | homolog (avian)- | | | | | | | |
| | like 2 | | | | | | | |
| NFE2L2⁴ | Nuclear factor | 884438 | Gene | NM_006164 | 2q31 | ND | | |
| | (erythroid-derived | | Expression | | | | | |
| | 2)-like 2 | | Regulation | | | | | |
| NUDT4P2 | EST | 123735 | Unknown | AU142060 | 9 | ND | | |
| OSBPL10⁵ | Oxysterol binding | 135608 | Metabolism | NM_017784 | 3p22.3 | ND | | |
| | protein-like 10 | | | | | | | |
| OSF-2 | Osteoblast | 897910 | Cell | NM_006475 | 13q13.2 | ND | | |
| | specific factor 2 | | Adhesion | | | | | |
| PPP3R2 | Protein | 782141 | Cell | NM_147180 | | ND | | |
| | phosphatase 3, | | Signalling | | | | | |
| | regulatory subunit | | | | | | | |
| | B, beta isoform | | | | | | | |
| RIPK1 | Receptor | 592125 | Apoptosis | NM_003804 | 6p24.3 | ND | | |
| | (TNFRSF)- | | | | | | | |
| | interacting serine- | | | | | | | |
| | threonine kinase 1 | | | | | | | |
| RNASE1 | Ribonuclease, | 840493 | Metabolism | NM_002933 | 14q11.1 | Not Confirmed² | TCCACTGCATCATT | TCTCCAAAGCGAGG |
| | RNase A family, | | | | | | CAGCTTTC | TCTTCCT |
| | 1 (pancreatic) | | | | | | SEQ ID NO: 295 | SEQ ID NO: 296 |
| SENP3 | Sentrin/SUMO- | 128506 | Unknown | NM_015670 | 17p13 | ND | | |
| | specific protease 3 | | | | | | | |
| SERPINE2⁵ | Serine (or | 246722 | Extracellular | NM_006216 | 2q33-q35 | ND | | |
| | cysteine) | | Matrix | | | | | |
| | proteinase | | | | | | | |
| | inhibitor, clade E, | | | | | | | |
| | member 2 | | | | | | | |
| SF1⁴ | Splicing factor 1 | 809648 | Gene | NM_004630 | 11q13 | Not Confirmed² | AGCTCAGAGACCC | ACTGAGGATCACCA |
| | | | Expression | | | | GCAGCATTA | GGCCTTTTG |
| | | | Regulation | | | | SEQ ID NO: 297 | SEQ ID NO: 298 |
| SLC13A3⁵ | Solute carrier | 51406 | Metabolism | NM_022829 | 20q12- | ND | | |
| | family 13, | | | | q13.1 | | | |
| | member 3 | | | | | | | |
| SLC2A3⁵ | Solute carrier | 121981 | Metabolism | NM_006931 | 12p13.3 | Confirmed³ | GCCCATCATCATTTC | TGAACACCTGCAT |
| | family 2, member | | | | | | CATTGTG | CCTTGAAGA |
| | 3 | | | | | | SEQ ID NO: 299 | SEQ ID NO: 300 |
| | UniGene | | | | | | | |
| TFAP2C | Transcription | 725680 | Gene | NM_003222 | 20q13.2 | High No-RT | TCGCAAAGGTCCCA | CGTAGAGCTGAGG |
| | factor AP-2 | | Expression | | | Control² | TTTCC | AGCGACAAT |
| | gamma | | Regulation | | | | SEQ ID NO: 301 | SEQ ID NO: 302 |
| TNFRSF12⁵ | Tumor necrosis | 345586 | Apoptosis | AB018263 | 1p36.2 | ND | | |
| | factor receptor | | | | | | | |
| | superfamily, | | | | | | | |
| | member 12 | | | | | | | |
| UBE2D2⁵ | Ubiquitin- | 773617 | Metabolism | NM_003339 | 5q31.3 | ND | | |
| | conjugating | | | | | | | |
| | enzyme E2D 2 | | | | | | | |
| ZNF363⁵ | Zinc finger | 784218 | Gene | NM_015436 | 4q21.1 | ND | | |
| | protein 363 | | Expression | | | | | |
| | | | Regulation | | | | | |
| ¹Array analysis performed using cDNA synthesized from pooled polyA(+) RNA from 17 RN specimens (RN-1, RN-2, RN-3, RN-4, RN-5, RN-7, RN-8, RN-9, RN-10, RN-11, RN-12, RN-13, RN-14, RN-15, RN-17, RN-18, RN-19), 21 FL specimens (FL-1, FL-2, FL-3, FL-4, FL-5, FL-6, FL-7, FL-8, FL-9, FL-10, FL-11, FL-12, FL-13, FL-14, FL-15, FL-16, FL-17, FL-18, FL-19, FL-20, FL-21), 9 | | | | | | | | |
| #MCL specimens (MCL-1, MCL-2, MCL-3, MCL-6, MCL-7, MCL-8, MCL-9, MCL-10, MCL-11), and 25 SLL specimens (SLL-1, SLL-2, SLL-3, SLL-4, SLL-5, SLL | | | | | | | | |

²qRT-PCR analysis performed on pooled polyA(+) RNA from 12 RN specimens (RN-1, RN-2, RN-3, RN-4, RN-5, RN-9, RN-10, RN-11, RN-13, RN-15, RN-16, RN-17), 12 FL specimens (FL-2, FL-5, FL-6, FL-8, FL-9, FL-11, FL-14, FL-15, FL-16, FL-18, FL-20, FL-21), 11 MCL specimens (MCL-1, MCL-2, MCL-3, MCL-4, MCL-5, MCL-6, MCL-7, MCL-8, MCL-9, MCL-12, MCL-14), and 12 SLL specimens (SLL-2, SLL-3, SLL-5, SLL-6, SLL-7, SLL-9, SLL-10, SLL-13, SLL-14, SLL-18, SLL-19, SLL-20).
³qRT-PCR analysis performed on polyA(+) RNA derived from 11 RN specimens (RN-1, RN-2, RN-3, RN-4, RN-5, RN-9, RN-10, RN-11, RN-13, RN-15, RN-17), 9 FL specimens (FL-2, FL,-5, FL-6, FL-8, FL-9, FL-11, FL-15, FL-16, FL-20), 11 MCL specimens (MCL-1, MCL-2, MCL-3, MCL-4, MCL-5, MCL-6, MCL-7, MCL-8, MCL-9, MCL-12, MCL-14), and 11 SLL specimens (SLL-2, SLL-3, SLL-5, SLL-6, SLL-7, SLL-9, SLL-10, SLL-13, SLL-14, SLL-19, SLL-20).
⁴IMAGE clones corresponding to these genes were represented by more than one set of spots on the micorarrays; data obtained from only one set of spots gave significant (≧-fold, p≦0.05) results.
⁵Multiple IMAGE clones corresponding to these genes were represented on the microarrays; data obtained from spots corresponding to only one IMAGE clone gave significant (≧-fold, p≦0.05) results.

TABLE 2

| | | | | |
|---|---|---|---|---|
| | | | Theoretical | |
| | | | end of | |
| | | UniGene | insert with | |
| | IMAGE Clone | Accession | respect to | |
| SEQ ID NO | # | # | poly A tail | Sequence |

| | | | | |
|---|---|---|---|---|
| | | | | GCTATAACATGGCAGCCTCGCATCCCTTCCTGCTTACCACCTTT |
| | | | | CTAGATATTAAGGCTTACTTAGTTCTTACTGAATTAAATGGAGA |
| | | | | GTGACTTGACAACTCTTGGCCAGCCATTCTTAATGATATTTGTG |
| | | | | TTCCTAAGATATAGCAGTATCTGCAAATCCTAAATCTGTCTCAT |
| | | | | GAAGATTTTATGATCTTTTAGATCAGTGATTAATGGGAAGGACA |
| | | | | ATGTCCTTTATTTTTTTAAATAAAAAATAATGACCTGGAACTTT |
| | | | | CTCTGTAGGCCAATAAAGGGTGAGTGTGGATGGGGCTATCACCC |
| | | | | TTGGGTNGTGTTNGGGAGTTTAACATTTCTCTAGGTTTAAAACC |
| 1 | IMAGE:110582 | T82892 | 3′ | ATNCCTATNACCTTNCCACAANACCGGC |
| | | | | ATAAGCCCTAGATATGATTTAATTTGAAGACTAGTTCATATTTT |
| | | | | TACTTTTGANCCAATTCTAGTCTCATAAAATAAAAATTCAGGTC |
| | | | | TCTCTGGGTCACACCACACATCTAAAAGTTGACAGTATGGTCTG |
| | | | | GCACTACAGTCTCCTTCTAGGAGAAGTTTGGGAAATCATTCTAA |
| | | | | CCCCTAGTTAGCTCCATGTATCTTAAGAATCACCAATTATTTGA |
| | | | | AAGCTTGGAGGTTCTAGGAGGGGAGTGCAGCTACTCATATACCC |
| 2 | IMAGE:110582 | T90074 | 5′ | TTGACCGAGACTGGGCC |
| | | | | TTCACAATCCAAATCTCAAATTACAGAAAAATGATATACCTTTC |
| | | | | AGCTATGTTTTTTTGTGTGTGTGTTGGCTGGGAATGCCAAAAAG |
| | | | | GTTGGCAAAAGGGGCAGGAAAAAAGTAGTGGGGCTCTCTGGTGT |
| | | | | ACTCCACTCCTCACATGTCTACCATTCTGAGATTTTTGATGTCA |
| | | | | GGTTCTGCCAAGTCTCAAAACCTCAAGAGTTGCCAGAATTCAGT |
| | | | | CCCAGTGTACACATTCTACTCTAGGGAGAGGAAGGATAACAACC |
| | | | | ACCCAAGGGCCACCCACCCGAGGACAGCCCTGCCTTTTAGGTAT |
| | | | | GGGGGATGCGGGTGTTCATTCAATTTGCTTTGGGGTTTCCCTTC |
| | | | | TTGAGGTCCCAGGAAAGGAGGATTTTCGGGGGAGTTCACTTTCT |
| 3 | IMAGE:121475 | T97292 | 3′ | TGCCCTTCAGGTCCCGGGGGGGAAGGCAACAGGGTTGAT |
| | | | | ANGTCANANTNGGGTTATCAGGCATCAGTCTACCTGAGGAGGCA |
| | | | | ACAGCATTGGTGGGTCCACCAGTAAAAAATGGACAGGAGACTGC |
| | | | | TGTGCCCCCTTTGTGGGAAAAGCCTCCCTTGGGAAGCAGTGGTT |
| | | | | GTATGCTCAGTCCTCCCCTGGGAAGAACAACAACAGGCAACTTG |
| | | | | CAGGGTTCCCTTCAGAATGTCTCTCTGAGTGCACCTGGGCAATA |
| | | | | AGCAGGCACAAGACCCTGGGGTGCTGAACCCTNTTCAACAGCCT |
| | | | | GGGCAGGCAACGAGGACATTCAGGAANTACCAGCCAGGAAAGGC |
| | | | | AGGAACCGTTTTGTTTGGGGTTCNTTTCCCACAACCGCCNTGTT |
| | | | | TTTTCCCGGCAACAGTTGATTTTNAGGAAGGCAAAAGAAGGAAA |
| 4 | IMAGE:121475 | T97406 | 5′ | NTTTTCAGGG |
| | | | | TAACTGGGAATTGAGAACNTGCAGTTCACACTCAACAGTACCAG |
| | | | | GGCAGAAATGAACTAATGCATGCAATTTATTTAGCCTATCATGT |
| | | | | GGGCTGTGAGTTTTTCCTGGAACATCCGGGCTGGTTTTCTTCTC |
| | | | | TTGGNATAATGGTTTATTACATGTGAATCATATCATAACATAAA |
| | | | | CTTGTTAGTTCCTGATTCCCGATAAAAAAGACATTTTATTGAAC |
| | | | | AAATGAACAGTTCAAGGTCTAAGGCAATGATTAACCGAGCCAGT |
| 5 | IMAGE:121977 | T97780 | 3′ | ATTAAATGCTCTAGNCCTATAAGGGGAATATCCCATA |
| | | | | AGGCAGGAACATGGGTTATTTATGAAGGATGCCTGTAGAGTTCA |
| | | | | ACAAGCCTGCTTACTGCGGGTTAGTTGTGACCATTGTCTAAGGT |
| | | | | AATTTAATGGTTTTCCTATGGAGGAGCTGAAGGGAGCCNTGAAA |
| | | | | GGGGAAAAGGGTGGCTCCCAATGAGTTGGCAGCCAATGGGGAAC |
| | | | | AATTTGGATATAATAAATAGGTCTCATGTTGACTCCTTTCCAAA |
| | | | | ACGGCCTTTCAAAGGGGNAGTGTNGGCTTGGCCTGGCAAACTTC |
| | | | | TCCCCACCCACTNCACCACA |
| 6 | IMAGE:121977 | T97887 | 5′ | |
| | | | | GAATTTTTATTTTAAAACAAAGAATCAAACAAACAATAATGGAA |
| | | | | AATCCATATGGAAATATTCACAATCTTCTCAGTGAGAAATAGGA |
| | | | | AAACAACTTCCCTGCCTTACTGCCAAACTGAGGAGCCAGAAGTT |
| | | | | GACGTGAAGTTGGAAGGCCACCTTTCCAGCTAAACCCCACTCCA |
| | | | | TAGCTACGTGCATTTTTATTCAAAGGCTCCAGGGGGCAGAGGGA |
| | | | | ACAGTGAGGACTNAGGACCCAAAATACTTGTCACTGGGCAAGGG |
| 7 | IMAGE:121981 | T97782 | 3′ | TTTTGGCTTAAAGGGGTCTTGAGG |
| | | | | GAATGTTTATAGCCCAAACTTGGAATTTGTAACCTCAGCTCTGG |
| | | | | GAGAGGATTTTTTTTTGAGCGATTATTATCTAAAGTGTGTTGTT |
| | | | | GCTTTAGGCTCACGGCANGCTTGNTAATGTCTGTTACCATGTCA |
| | | | | CTGTGGTCCTATGCCGAATGCCCTCAGGGGACTTGAATCTTTCC |
| | | | | AATAAACCNGGTTTNGACAGTATGNGTCAATGTGCNGTGCAGCC |
| | | | | CACACTTNTAGANGGATGAATGTATGTGCACTGTCACTTTGGCT |
| | | | | CTGGGGTGGGAGTATGTTTATTGTTTGACTTATTTTCTCTGTGT |
| 8 | IMAGE:121981 | T97889 | 5′ | TTGTTCC |
| | | | | TTCTTGATAGCATCACATTTTATTACTAATTGCAGTTTTTGATT |
| | | | | CCACAACCCTGTATAACTTGGCATTCTGGTGAATTGGACCCGAA |
| | | | | CATCTGTGAATCTTAAAAATAGTGGTTGACTCATTATGGCTTCC |
| | | | | TTATGTATAGGATTAAGAACACAGATCCTGGGAATCAGACAGCC |
| | | | | TGGCTTCCACACTCTAGCTGGGTGACCATGACCATGAAGAAGTT |
| | | | | CCTGAATGTTCCAGTGTCAGTTTATTCACCTTTACAGAGAAATC |
| | | | | TGGCCAAACACTACCCTCAGCCAGGGTGATCCAAGTTCAATATT |
| | | | | CAGCAATTAAGGTCATGTTGTTTGTTAGGTGTGTGCTCTTGATA |
| | | | | TGGCATGATGAGGAATTGCACTTCACTTCTGTGATATTCCCCCN |
| 9 | IMAGE:122702 | T98928 | 3′ | GGGCTTTTAACTTCAGGTCCCTGNAA |
| | | | | NATTTCGGCACAGAGCGCTTCCATTGCTGACCTCTACCGACCTC |
| | | | | TACCTGTGGTCCTTCCTCTACTGCAGCAGAGACACTGTTTTCTT |
| | | | | CCTTTGTTCTTCCAACCCCATGGCACAGANACACTCTCCACTGC |
| | | | | GGCCAAGGATTGCAGGAGAGGTGGCATCAGTGATTCAAGACTGC |
| | | | | TTTTCCTACCTCTTCAGTGTTTCTTTCAGTGATCTGAAGTTAAA |
| | | | | GCCAGGGGGAATATCACAGAAGTGAAGTGCAATTCTCATCATGT |
| | | | | CATATCAAGAGCACACACTAACAAACAACATGACTTATTGCTGG |
| | | | | AATATTGGAACTTGGATCACTGGGGTTTGGGTAGTNTTTTGCCA |
| 10 | IMAGE:122702 | T98972 | 5′ | GNTTTCTCTTGTNAAGGGTGGATTA |
| | | | | GCTTCTTCTGGGCACATTGTTCTGACATAAAGGTTGCCTCCTTG |
| | | | | TGGGGGAGAAGGGGAGGATTAGTTTGTTGGCTTGGGCATTTGAT |
| | | | | CATAAATTATGGAGGTGCTGGACCGGAGAACCACCCACCAGCCC |
| | | | | ACGGAGGCTACCGGGCATTCAGGATAAGGGCCGCCTTCTTCTTC |
| | | | | AGAATAACCATACCCACTCCCTCTGAAACAAAGTGGAGAGTCTT |
| | | | | AGGTCTGAGTGGAAACTCTAAATCTTTTAATTCTTGGGTTCAAC |
| | | | | TTTCTTCATCTGTTTTCCTGGGTTCAGACTAAAACCATCTAACT |
| 11 | IMAGE:122723 | T98941 | 3′ | CAGCTGGGAGAAGTTATAACCGCTTTGTTGTTGGGC |
| | | | | TACCCCGACAGTCTTCACACACACAAAAAAAAAAAAAAAAAGAA |
| | | | | AGACAGACCAAGCAGAATNAAATAAAAGGTCTGAAGAACAAGTT |
| | | | | TTGTTAATTTGCCACAACAGACTGTACTCCAGGGGAAGCTTTGT |
| | | | | TGTCCATTAAAGTGAGTTCTCTGGGAAGACGAGTAGTAACCGAC |
| | | | | TTGCACGATTTTCCTGCCTTTTCTATATTCTCTACTTACTATGA |
| | | | | CAATACAGCACTAGGNATTTCCAAGTGCTTATTACCCGGCATAG |
| 12 | IMAGE:122723 | T98991 | 5′ | GTGCATGTATTTTAATGAGGG |
| | | | | TTGGNTNNGAAGAAATAAAACTGCCTTTATTTGCAGATAACAAT |
| | | | | CACATACATAGAAAATCCTAAGGGATTTACAAAAAAAGCTGCTA |
| | | | | AAACTAATAAGGAGATTTAACAGTATTGCAGGACACAAAGCATT |
| | | | | TCTGTATCCTAACAAAGANTAATTAAAAACTGGANTTTAAAAAA |
| | | | | TTATTTAGGCTGGGCATGGTGGCTCACACCTATAATCCCAGCAC |
| | | | | TTTGGGAGGGTAGCTGGATTAAAGGCCACACTGCCACACCCATC |
| | | | | TAATTTTTGTATTTTTAGTAGAGACGGGGTTTCACCATGTTGGG |
| | | | | CTAGGCTGGTCTCAAACTCCTGGGCCTCCGACCTCAGCCTCCCA |
| | | | | AAGTGCTGGGGATTACAGGTTTGAGGCC |
| 13 | IMAGE:123735 | R01179 | 3′ | |
| | | | | GCTTTTGCACATCAATAGGTATCCCTAGGAGGGCCTGATTCAGA |
| | | | | AGCCCTCATTTTTAAACTCAATTCTTAGATGAACAGTCTTATTC |
| | | | | ATCTGGAATGTTCCACATAATGGTCATCATAATTCTAATTTATC |
| | | | | TTTAGTAAGATTTCACCATTTTTGTAAGTATTTGCAGCTTCTAG |
| | | | | GCCCTAACACATGTAAAAGGTAAACATAGCCAGGAAGGTGAAAT |
| | | | | ACACAGTTCTTTAAAAATTTAAGGGATGCTGGCCAGGGCGAGGT |
| | | | | GGCTTCACACCTGTTAATCCCAGGCACTTTGGGGAGGGCTGAGG |
| | | | | GTCGGGAGGGCCAGGGGAGTTTTGAGGACCCAGGCCTTAGGCCC |
| | | | | AACATGGGGTGGAAACCCCCGTTCTTCTACNTTAAAATTTACCA |
| | | | | AAATTTAGGTTGGGGTTTTGGGCCGTTTTGGGCCCTTTTATTCC |
| 14 | IMAGE:123735 | R01291 | 5′ | CNGTTACCCTTNT |
| | | | | ACGGTAGTGGGTAGCGGGTCTCGGGTTGCGGGTTGCAGGTTGCA |
| | | | | AGCCNAGCCCGCAGGCAACTNCCTTCCCGGCGCCATGTTCGGCT |
| | | | | CCAGTCGTGGAGGCGTGCGCGGCGGGCAGGACCAGTTCAACTNG |
| | | | | GAGGACGTGAAGACTGACAAGCAGCGGGAGAACTACCTGGGCAA |
| | | | | CTCGCTGATGGTGCCAGTAGCNCTTGGCAGAAGGGCCGCGANCT |
| 15 | IMAGE:126450 | R06699 | 3′ | C |
| | | | | TTTTAACCCGGTCAAGTCCAAAGGTTTATTTTAAGGCACAAGGT |
| | | | | GGGNGGNCAAGGGGGATGGTAAAAGCGCAAGGGGTCGTGGCCTC |
| | | | | ATCAAGGTCCGAAGGTCCAAGGGAAGGCGGGTCCGGTCCTGTTG |
| | | | | GTCCTGGTCCCGAATTGGTAGCTGGGTGTATCTCCGGACCATGT |
| | | | | TGGGGGCGCACCATCCCTTCCTCACTGGGACCTCCTGGGCTGGT |
| | | | | CCANGCCCTTCTCCTCGGGGTCGCCTCCTTCCCGCTTGCAAGAC |
| | | | | CTCCCGCGAAGTCCTCCTTGCTCAANGCCCGTGGGTTGCTTCTT |
| 16 | IMAGE:126450 | R06700 | 5′ | CANGTTCTTGTAGCCAAGGGGGCCAANAAGCGCT |
| | | | | CTTTCTTATCTTTCAGTCCCCCATATGCCCTCCTCCAATAGAAT |
| | | | | GTTTGAAATTACAAAAGGTTCAGACAACACCATAGAAGGAAAGA |
| | | | | AATTACAAATGGNACACTATTTTGTGTATATTTGTTTTTAAAAA |
| | | | | TTTCTGAATCTGCATTTAATGAATTTTTATTGAATGATGTGTTG |
| | | | | AATATTTGTTACCNATAATTATTGAAATTATTGATAATTAATGA |
| 17 | IMAGE:127710 | R09498 | 3′ | TAATTA |
| | | | | TGTCATAGACCAATGCGAAGTTTTTGGCCATTAAATATTTTTCT |
| | | | | CTGTTCTAAATGCAGAGTCTTAGAAGCAAGACGTACTTTTCAAT |
| | | | | TCATATCTTTCTACATTATATGAATTATATTTCACAATAAACAT |
| | | | | ATTTATTTCTTTAGAGATGGAGTTCCGCTCTTGTTGCCCAGGGC |
| | | | | TGAAGTGCAATGGTACAATCTCAGCTCACCTCAACCCCCACCTC |
| | | | | CCAGGGTTCAAGGCGATTCTTCTGCCTCAGCTTACCCAGGCAGC |
| | | | | TGGGGATTACACCCGTGCGTTCACCATGCCCGGGCTTAATTTTG |
| | | | | TATTTTTAGTAGAGGACGGGGGTTTCTCCCTGTTGGGTCAGGGC |
| 18 | IMAGE:127710 | R09603 | 5′ | TGGG |
| | | | | AAAGGANCCTTTATTGACCAGAGCAGGACCGTGGCATTTTTATA |
| | | | | TATATATATATATATATAAAAGTNTGAAGACCTGGCAGGCAGTG |
| | | | | ATCCNATTGTCCGCCCACCACCCCCAGCACTGATTTCCTGCTCC |
| | | | | CTGCACGGGGAAGGGGGAGGATGACTNCTCCACCCAGGCCACAG |
| | | | | GGCACACTCCCCTGCAAACAGAGGAAGAAAGGGGCTTTTCTGTA |
| | | | | GCCACCCCCTGCACATCAGANATCAACAAGTATTCTCTCAAANN |
| | | | | AANNNNNNTACAGNNTTTGANNCATTTNNNTNTGNNANNCCNNN |
| | | | | GGGNNGTGAGTGGGGNNGNGGCNNGNGNGGNNNGGNCTGGGNGT |
| | | | | TTCTTGGGGNNGGGNCTCCCNTGTCTCCCTTCCCNTTTATGGGG |
| 19 | IMAGE:128506 | R10154 | 3′ | NTTGGGGGTCTG |
| | | | | GGTTATGATGGGGTGAAAAGGTGGACCAAAAACGTGGACATCTT |
| | | | | CAATAAGGAGCTACTGCTAATCCCCATCCACCTGGAGGTGCATT |
| | | | | GGTCCCTCATCTCTGTTGATGTGAGGCGACGCACCATCACCTAT |
| | | | | TTTGACTCGCAGCGTACCCTAAACCGCCGCTGCCCTAAGCATAT |
| | | | | TGCCAAGTATCTACAGGCAGAGGCGGTAAAGAAAGACCGACTGG |
| | | | | ATTTCCACCAGGGGCTGGGAAAGGTTACTTCAAAATGTACTGCA |
| | | | | AGCATCTGGCCCTGTCTTCAGCCATTTCAGCTTTCACCCAGCAG |
| | | | | GGACATTGCCCAAATTTCGTTCGGGCAGATCTTACAAGGGAGNT |
| | | | | GTTTTCACTTGCAAATTCATTGTTGTTNGGCCTNGTTACCCCAG |
| 20 | IMAGE:128506 | R10564 | 5′ | GACCCC |
| | | | | TAAACATAANNNNTACAAAGTATAGTCTTCGTATTCACTACACA |
| | | | | CCGCAAAGTTCTGCTACTTGAAATAAAGCAAATGAAGAAAATTA |
| | | | | CGTTTTCTGACATAAAAATAATTATTATATCCACTGGCAACAAT |
| | | | | AAGGAAAACTTAGCACTTATATATTTTATGATCAAATTGATTCA |
| | | | | AAAATTAAATTGGTTAGCTTCAGCATCTATTCTGTCTATATCTC |
| | | | | CCTGTGGGATGACAATTTAGACAATATGAACATTCTCAGGATAA |
| | | | | GGAAATCTTGTTTTAAAATGTCCCAGGCATCCCTTCCNCTGGTT |
| | | | | AAAACTCCCTATATTTGCCTTATTATAAAATTCAGGGCTTTCTT |
| 21 | IMAGE:130742 | R22024 | 3′ | CCNCCAGGTGGGCCCAATGGCCCAAGGGAC |
| | | | | CAAACATCCAAACCATTTCAGAACTCATTCTATAAAATATATAA |
| | | | | ACAGCTTTCTATTTTTTTTCTAGCTGCATAATATTCCATTGTGT |
| | | | | GGATGAGCCATAATTTATCAATTTCCTATTATTTCTAATCTTTT |
| | | | | ACAATAGATAGTGTTTCAGTCCTTAATCTTATACATATAGGTG |
| | | | | GCCATAAATTTTTAAGGTTCTTTGGGCTATTTGGCCAACATGT |
| | | | | GGGAAGGAAAGCCTTGGAATTTTATAATAAGGCAAATATAGGG |
| 22 | IMAGE:130742 | R22077 | 5′ | NGTTTTAACAGTGGGGAGGGATGCTG |
| | | | | GAACAGTTCAATCCTGGGCTGCGAAATTTAATAAACCTGGGGA |
| | | | | AAAATTATGAGAAAGCTGTAAACGCTATGATCCTGGCAGGAAA |
| | | | | AGCCTACTACGATGGAGTGGCCAAGATCGGTGAGATTGCCACT |
| | | | | GGGTCCCCCGTGTCAACTTGAACTGGGACATGTCCTCATAGAG |
| | | | | ATTTCAAGTACCCACAAGAAACTTCAACGAGAGTCTTTGATGG |
| | | | | AAAATTTTTAAAAAATTCCACAAAGAGATTATCCATTGAGCTT |
| | | | | GGAGGAGGAAGGATAGGACTTTGACGTTGAAATTTTATTGAAC |
| | | | | GGCACTTCTTTAAAAAGGTTACCCAAACCAGGNCCACAGGATT |
| 23 | IMAGE:131318 | R22950 | 3′ | NATTTGGNTTTTTTTGGGG |
| | | | | GATCAAGTTAGGAAACACACGATTGAAATCTGGAAGAGAAAAC |
| | | | | TGGCTCCTACCACATTGCTTCTCTCGATCATGGGTGAAGCCTG |
| | | | | AGGAGTTCCAGACACGGGGGTAGAGGCTGGGGTCTTTATTTCT |
| | | | | TCGATCATATTCATGATTTTCTCTGGGCACTTTGATGGCATCA |
| | | | | ACACAGGTCTCCTGCCACCGAGGCAGCTTGGGAATTCAGTAGT |
| | | | | TCTGCAGACTGTAAGTGATAATAATGTATGTGGGTTTTGCAAA |
| | | | | GCCACAGTGCTTATTCAACCCAGGAAAGCAGGAAGCGCCTCTT |
| | | | | TCTCTTTCAAGCAGGAGCCTCNTTTGGCAACCCNTCTTGGCAA |
| | | | | TGGANTTTCTGGGGATTTTCACTTCTGGACGGAGGAAGTTAAC |
| | | | | CGGGTTNTTCCCACCATACTTCCAATTTCCTTTTGGTGGTTTC |
| | | | | CAAATTTTGGAGTGGCGTTTTCGGGCCTTCCCTTGGGGNTTTT |
| 24 | IMAGE:131318 | R23056 | 5′ | CCCNTCCTGAACCTTTT |
| | | | | TACACATGTGTATGCATGAAAAATTTCTAGAGGGTCATATTAA |
| | | | | TGTAAGAAATTGTGAAGGGTGGTCTCTAGGGCATGGAGCTTAG |
| | | | | CAGCTAGTGATAAAGAAACTCACTTGTCATTACACTTACTGTT |
| | | | | TGAATTTACAATGTCATGTTTCATTTTCATAATTTAAAAAAGT |
| | | | | CAGTGCCAAAACACTTACATAACTACTTACATTTCTTATGTAT |
| | | | | GATTTGACTGCTTATTTTAAAGTTTACTGTATTTAAAGTTCAA |
| | | | | CATCAAAAGAAAGGGCTAGGAAAAGTGGGTGGGCTAGGACCTA |
| | | | | GGTTCTTTCACACTACTTCATTTCTAGGCTTCCACATGGCTCT |
| 25 | IMAGE:133613 | R27606 | 3′ | GGTAATAGCCAAGGC |
| | | | | AGTTTCTGTGTTCAAGTTTGAATACTCTTGAAGTCTTATTTTT |
| | | | | TTCATTTTCAGATTTTAAAATTTTCAAAGAAAAGGCGTTGCTG |
| | | | | ATGTCTGAAATCTCAGATGCCTGAAATTCAATTGACAATTACT |
| | | | | GAACAACAGTCTCTTATTTACATAAAGGTGGGGTTGTCAATCT |
| | | | | TGGGCTCTCAGGAATTTTCTCTTGTAGGGCACTGTGTAGGCTA |
| | | | | AAGGTTATTTAAGGTGATTTCAGAGGTAGGATAGGATTACTCA |
| | | | | GTGGATTACTACCCTGTTGCCAAGGTTAATTCCGGNAGGGGTA |
| 26 | IMAGE:133613 | R30836 | 5′ | ACCCCCGTTNCCAGTTCACGTTAGNGT |
| | | | | AGAACAGGTACTTCGTACTGGGATTTCGAGGCTGGCATCCTGC |
| | | | | AGTATTTTGTGAATGAGCAAAGCAAACACCAGAAGCCTCGAGG |
| | | | | AGTCCTGTCTTTATCTGGAGCCATAGTGTCCCTGAGCGATGAA |
| | | | | GCTCCCCACATGCTGGTGGGTGTACTCTGCTAATGGGAGAGAT |
| | | | | GTTTAAACTGAGAGCTGCTGATGCAAAAGAGAAACAATTCTGG |
| | | | | GGTGACTCAGCTTCGAGCTTGTGCCAAATACCACATGGGAAAT |
| | | | | GGAATTCTTAAGGAGTGCTCCCAAGCTCCCCGAAGCCGAAGTC |
| | | | | TTCACTTTTGCTTCCCACATGGGAACACCCATTCTTGCGTCTT |
| 27 | IMAGE:135608 | R32892 | 3′ | CCCTGTTAGGCCAGAGACACCTNAA |
| | | | | GCCTCCATAGAAAGGCTTCGTGTGGAATATCACTGTCGCTGAG |
| | | | | TACCCAGTCTTGGCACAGTTGATGCTGACTTTTCCTCCGAGCT |
| | | | | CCACCCACGGGATGGTGAGAATGGACCGGGCGTAGGCACTAGG |
| | | | | CAGGGTGAATACGTACTCCTCCCCGTGTTCCAGGAGCCTCAAC |
| | | | | ACACCTTCCCCTATCATAGAGACCCCCACGGACATGCCCATGA |
| | | | | ACTTGCTTTTGGTCCATACATGAGTGTTGACGCACAGTCTCTT |
| | | | | CTCCTCGCACTCACAGTAGAAGCAGGAGATGGGTGGGTGATGG |
| | | | | GACACTTGCTCAGCCACAAACCTTAGTTTGTAGCTTTTGGGAA |
| | | | | GGGTCATCGGCCATTTGGGTNTTCATGACAGCTGGCAGGAGAG |
| 28 | IMAGE:135608 | R31395 | 5′ | CCGGGAAGCAGTTCTCTTTAGGT |
| | | | | GCCGCATGGGAGGCATGGCCCCGGGGTCCTGGTGGCCACTCGT |
| | | | | GCCTGGTGGAGAGCGAGGGCAGCCTGACGGAGAACATCTGGGC |
| | | | | CTTCGCTGGCATCTCCAGGCCCTGTGCCCTGGCCCTGTTGCGG |
| | | | | AGAGACGTGCTGGGGGCCTTCCTGCTGTGGCCTGAGCTGGGTG |
| | | | | CTAGCGGCCAGTGGTGTCTGTCCGTGCGCACGAGNGCGNTNNG |
| | | | | TTGCCCCACCAGGTCTTCCGGAACCACCTGGGGCCGCTACTGC |
| 29 | IMAGE:136909 | R36650 | 3′ | TTGGAGCACCTGCCGGCAGAGTTCCCCAGCCTGGGANGCTTCT |
| | | | | GTCGAGTTAGGAAGGGCCCTGGTCACCCCTCTAAGCCTGCAGC |
| | | | | TCACTGCTGGCCCCTCCCTGTCAAATGGCTAAAGGAGATGAGC |
| | | | | TGGGGGTGGGGGTGCCCTGGTGATTCCTAGGGGGAAGGGGTGA |
| | | | | GCCTGCGCATCCCTTCTGAGAAAGCGGGAGTCACAGCCCTGAG |
| | | | | GTTTTGAGTGGAGACAGCATGGAGATTCTTGGCCCTGTCTGCT |
| | | | | GGTGCGCATCCCTTTCTGCACACAGGAGTCCACCGTGCGTGAN |
| | | | | GTTTAGGTACAGCCCTCTGTCCCTCCTTGCCCTCTCTTGCACC |
| | | | | TTCCCACCCCTCCTTTCACTTTTCAGATNACATATTGAGGAAA |
| | | | | CAGCCCTNGTTCTGTTCAGCNTAGATGGGGGTTCATCCCCAGC |
| 30 | IMAGE:136909 | R39730 | 5′ | CT |
| | | | | GAGTTTAATGATTACATGGNGCTGAGTCAGGAGGTAGGAGGAG |
| | | | | ATTCTTAAATCTCTGAAGAGTTCTGGGCTGGGGTTCTGGGAGG |
| | | | | CAAGGGGCTGGAAAATTTGGGCCACTGATTGGTCAGGGTAAGG |
| | | | | GAGATTGAATCATTAGGATATGGAAATTGCATTCTTTGATGAT |
| | | | | TTAGCTTCTGGTAGGGNCCTTCAGACCAGGCTGACATCAGTAG |
| | | | | TTTCATCAGTATGCAGGGNCAACCAATCATGGCCAAGTCCNCT |
| 31 | IMAGE:136919 | R36539 | 3′ | TTNAGGGANTTTGTNCCCGTAGGATTTATCCG |
| | | | | GCTGATCGAACAGCCTCACTTGTGTTGCTGTCAGTGCCAGTAG |
| | | | | GGAGGCAGGAATGCAGCAGAGAGGACTCGCCATCGTGGCCTTG |
| | | | | GCTGTCTGTGCGGCCCTACATGCCTCAGAAGCCATACTTCCCA |
| | | | | TTGCCTCCAGCTGTTGCACGGAGGTTTCACATCATATTTCCAG |
| | | | | AAGGCTCCTGGGAAAGAGTGAATATGTGTCGCATCCAGAGAGC |
| | | | | TGATGGGGATTGTGACTTGGGCTGCTGTCATCCTTCATGTTCA |
| | | | | AGCGCAGGAAGGAATCTGTGTTCAGCCCGCACAACCATANTGT |
| | | | | TTAAGGCAGTGGGATGGAAAGTGGCAAGCTTGCCCAAGGAAAA |
| | | | | NGGGTTAAAGGGAANTTTTTTGCCACAGGGAAGGAAACACCNT |
| 32 | IMAGE:136919 | R38459 | 5′ | GGGCAAGAGGGNACCATTTACCAGGGGGCA |
| | | | | ACCAGAAAATAAGACATTTTATTTTGAGAAATAAATTGGAAAA |
| | | | | AAATATTTTAAAATGTTTAATTTGCAATATACATAATACTGGA |
| | | | | ATTGAAATGCTGTCTGATGGAAATGTTGCAATGTGGAGTAGGA |
| | | | | GGGTCAAGTTCGTGAAGATATTCTTAAAATTAATCTTGGAAAC |
| | | | | TCTGTGCCTATGAGGTTTCTCTAAAGTGGCTAAAATATGCATT |
| | | | | TAATATGTTGTCTAAATGAGTACATTTAATTCTAGAGACTGTA |
| | | | | AGGAGTAGGAGATTATATGCTTTGGGGGCTTTTGTAGGCNTTT |
| 33 | IMAGE:138496 | R68635 | 3′ | TTTTTAAAATCAGTTGT |
| | | | | TAAATGCATTATTCATATTTCTTGAAGCTTAGATACAGTCTAA |
| | | | | TTCATAGCAACCATATCTGCTTTATCCTAGGTGAGGGTAGCAG |
| | | | | TCCACAATGGAATAGAAGAAAATCCCATTATAACAAATGACAA |
| | | | | ATTATATATCATGAATCCTTCTGTCTGACTAACTCAATAACTT |
| | | | | TCTATAAAAGCCAATGGAATTCAAATAGGAGCTAGGAGACAAC |
| | | | | AAGTTATATATGACAGTGGAGGTTGTATTCCTTTTATATTGCT |
| | | | | GAGAAAACTAGTTAAATGATCAGATTCTTGGCTGTTAAGGAAA |
| | | | | CAATTTTCGTTTAATGGGGATCTGTACAACTGATTTTAAAAAA |
| 34 | IMAGE:138496 | R68634 | 5′ | ATGGCTACAAAAAGGCCCCAAAGG |
| | | | | TTTTTTATCCTTCTTAANNNTTATTACATGTTTTATTATCCTG |
| | | | | TCCCCAGAGGTGGGTTTATCCAGAAACCAAGAAAAAAAATCAA |
| | | | | TCAGAATAAACTCAAAAAAAAAAGGTAGGGGGAGCAAAACCAT |
| | | | | CAACCACCAGGGCAGCCAGGCCATCAGCCCACCTCCACCTCTG |
| | | | | GAGGGTCCCCAGAGACCCACGCCCGACGCAGACCCGGAGGAGG |
| | | | | CATCAGCAAGGGGGCCCGGGCAGAGAATCGGCTATGTCTTTCA |
| | | | | TTATGAGGAGGCAGGGAGAGACGGGCAGAGATATGTTTGCTAG |
| | | | | GGTGANTATATATTTTATATTAATTAAATCCGTAAGTTTAATT |
| | | | | AAAGTAAATAGGTATTTCTCTGGAAGTTTTTTTAATTTCTTTC |
| | | | | NTTTTTTATAGTTTTTTTGGTTTTTTGTGGNTTTTTTTTTTTT |
| 35 | IMAGE:140951 | R66605 | 3′ | TTTTGGGGTTT |
| | | | | CAAGCACCCCGCTTTTGCAGCAGAGGAGCTGAGTTGGCAGACC |
| | | | | GGGCCCCCCTGAACCGCACCCCATCCCACCAGCCCCGGCCTTG |
| 36 | IMAGE:140951 | R66604 | 5′ | CTTTGTCTGGCCTCACGTGTCTCAGATTTTCTAAGAACCA |
| | | | | GGAATCTACTNCGAGCACAGCAGGTCAGCAACAAGTTTATTTT |
| | | | | GCAGCTAGCAAGGTAACAGGGTAGGGCATGGTTACATNTTTAG |
| | | | | GTCAACTTCCTTTGTCGTGGTTGATTGGTTTGTCTTTATGGGG |
| | | | | GGGGGTGGGGTAGGGGAAAGCGACAGGAAGTAACATGGAGTGG |
| | | | | GTNCAGCCTCCCTNTAGAACCTGGTTACGAGAGCTTGGGGCAN |
| | | | | TTCACCTGGTCTTTGACCNTCATTTTCTTNACATCAATNTTAT |
| | | | | TAGAAGTCAGGATATTTTTTAGAGAGTCCACTNTTTCTGGAGG |
| | | | | GAGATTAGGGTTTCTTGCCAAGNTCCAAGCAAAATCCACGTGA |
| | | | | AAAAGTTGGNTGATGCAGGTACAGGNTTACACGNGGGCATAGT |
| 37 | IMAGE:159608 | H15842 | 3′ | TTNCCATAGTCNGTTGCCAGGG |
| | | | | CCAGTCACCAAGACAGGCATCTCAAATCGGCTGATTCTGCATC |
| | | | | TGGAAACTGCCTTCATCTTGAAAGAAAAGCTCCAGGTCCCTTC |
| | | | | TCCAGCCACCCAGCCCCAAGATGGTGATGCTGCTGCTGCTGCT |
| | | | | TTCCGCACTGGCTGGCCTCTTCGGTGCGGCAGAGGGACAAGCA |
| | | | | TTTCATCTTGGGAAGTGCCCCAATCCTCCGGTGCAGGAGAATT |
| | | | | TTGACGTGAATAAGTATCTCGGAAGATGGTACGAAATTGAGAA |
| | | | | GATCCCAACAACCTTTGAGAATGGACGCTGCATCCAGGGCCAA |
| | | | | CTTACTTCACTTAATGGGAAAACGGAAAGATTCAAAGTGTTAA |
| | | | | AACCAGGGAGTTTGAGGAGCTTGATGGGAACTGTTGAATTCAA |
| 38 | IMAGE:159608 | H16152 | 5′ | ATCGAAGGGTTGAAGCCACCCCCAN |
| | | | | NTCCACGATCTGCTCANNCNGNGACCACGCCCTTGGCAGTTCG |
| | | | | CCCTCGTAGTAGATGTCTACCACCTCGGCCGTGAACGTCCTGA |
| | | | | TGGCTTCCCACACCAGGAGCCCGTCGTCCCGGTAGAAGTAGTA |
| | | | | GGGGATGTCTTCTTTGCTCTCCATGCCCCGGGCCTTGATGGCC |
| | | | | TCGGGAAAGCACAGGGAGGCATAGGTCAGGTCCTTCATGGCCC |
| | | | | TCTGCACCATCTGCACGTGCCCACCGCCCCCTGTGGCGTTGGC |
| | | | | CTTGTCAAAGAGGCCACACTCGCAGATGAGCTGCTCACGGGCC |
| | | | | TTGGGTGTTGATTGCAATGGTAAATCTCACGTGTGCCACCAGC |
| | | | | AGCTTTGAAAATGGGGGTGCACAGCAGGGCAGCTNGGCGGTAA |
| 39 | IMAGE:179890 | H51574 | 3′ | CATTTGC |
| | | | | CTGGGCGAGATCCAGCTGGTCAGAATCGAGAAGCGCAAGTACT |
| | | | | GGCTGAATGACGACTGGTACCTGAAGTACATCACGCTGAAGAC |
| | | | | GCCCCACGGGACTACATCGAGTTCCCCTGCTACCGCTGGATCA |
| | | | | CCGGCGATGTCGAGGTTGTCCTGAGGGATGGACGCGCAAAGTT |
| | | | | NGCCCGAGATGACCAAATTCACATTCTCAAGCAACACCGACGT |
| | | | | AAAGAACTGGAAACACGGCAAAAACAATATCGATGGATGGAGT |
| | | | | GGAACCCTGGCTTCCCCTTGAGCATCGATGCCAAATGCCACAA |
| | | | | GGATTTACCCCGTGATATCCAGTTTGATAGTTGAAAAAGGAGT |
| 40 | IMAGE:179890 | H50910 | 5′ | TGGACTTTGTTCTNAATTACTCCAAAG |
| | | | | TTATTTAAAACTTAATTCTCACCTTGAGTATGCAAAATACAAA |
| | | | | CTCCACAAAATGTTCATTTTACTTTGTAGTTTACAAATATACA |
| | | | | AAATAGACGTTTGCTTAAATTTATATTACATATTTATTAAGGC |
| | | | | AAGGAACTATATAGAAAAACACATTTGTTCTGCTTAAGGCATA |
| | | | | CTTGGGAATAAACCATTGTACAAATTATTGCACATCTGAAACC |
| | | | | ACAGTGCATAACAGACTGTCTGCATAAAAATGCTAAAGANGTA |
| | | | | AACCAGGGTATATTACCTGACTTAGGGTCATAAATGTTGATCG |
| | | | | GAGGACAAATATAGGATTTTCCTTGTCAAAGTATGCAGGCAGT |
| | | | | TTGAAAACTTTGGGCTTCCNTGTTTGGGNACCTTTAGGANCCA |
| 41 | IMAGE:188232 | H45668 | 3′ | AGGTCTCACCAAG |
| | | | | CTGGACTTACAAAATGCCAAGGGGGTGACTGGAAGTTGTGGAT |
| | | | | ATCAGGGTATAAATTATATCCGTGAGTTGGGGGAGGGAAGACC |
| | | | | AGAATTCCCTTGAATTGTGTATTGATGCAATATAAGCATAAAA |
| | | | | GATCACCTTGTATTCTCTTTACCTTCTAAAAGCCATTATTATG |
| | | | | ATGTTAGAAGAAGAGGAAGAAATTCAGGTACAGAAAACATGT |
| | | | | TTAAATAGCCTAAATGATGGTGCTTGGTGAGTCTTGGTTCTA |
| | | | | AAGGTACCAAACAAGGAAGCCAAAGTTTTCAAACTGCTGCAT |
| | | | | ACTTTGACAAGGAAAATCTATATTTGTCTTCCGATCCAACAT |
| | | | | TTATGACCTAAGTCAGGTAATATACCGGGGTTTANTTCTTTA |
| | | | | GGCNTTTTTATGGCAGACAGTCTGTTATGGCACGTGGGTTTC |
| | | | | AGATGTGGCATTATTTGTACATGGGGTTTNTTCCCAGNATGG |
| 42 | IMAGE:188232 | H45711 | 5′ | CCTAT |
| | | | | TGCACATTCTGTTTTTACCTCTGTCACTGACTCTGTGGGTCT |
| | | | | AGCCATGTCATTTAACCACACTTGAATTTCAGGTATTTTGTC |
| | | | | TGTAAAATGAGGATAATAACGCCTGTCTACTACATTAAACCA |
| | | | | CAAGATGGTTTAAAGGTTAGCATAATAAATTATTAGAGTATG |
| | | | | ACCTAGGAGTTACCTAATCTGACCTCTTTATTTTACAGATAG |
| | | | | AAGTACAGAAAGGTAAATTGAATTGCTCAAGGTCACCCAGTG |
| | | | | TGTGGCAAAATCAGAACTGGAAACTTAGGTCTTCTGCCAGTC |
| | | | | CCATTCAAGGGCTTTTTCCATTGTACAGTTAAATTATATGTT |
| | | | | GTGTGTAAGGCATAGTATAAACTGTAAACCATTCATGCCAAA |
| 43 | IMAGE:193771 | H47895 | 3′ | TGTTCAGGTGGATTGTTTTTCCCTCAGTTTT |
| | | | | CTCCTTCTCTTCTTGTTATTATTATCATTATTATTATTTTGA |
| | | | | GATTGATTACTTTCCCATAAAAGTGGAATATACTTTGCTTTG |
| | | | | GTTGAGTAATGCTCTAATTATCTGAGGTCTTACAGTAATTGA |
| | | | | TTCAGACTGATGACCACCTGCTGCCCATTCCACATGGGCAGG |
| | | | | GACACAGCAATAATGAGAATTAGGGTTAGGCTCATAGGGGAT |
| | | | | GGAAGCCAGCAGGGAAGGGACTAAAGCTTTGGGAGAAAGCTG |
| | | | | AAGGGTGACTACTGCCCGGGGGCTTGAACTTTCTAATGGGCC |
| | | | | ATGGCCTTNCTCTGAAAATGTAATTACTATGACCACTGGGTT |
| | | | | AGGTGATGTATTTTTCATTTCTTACCCACTCTCCATCCCTTT |
| 44 | IMAGE:193771 | H47896 | 5′ | TAAACACTGCA |
| | | | | GGCCCAGATCCTCTGGACTCCTCAGATGAGCGGATTCAGAGA |
| | | | | GAAGCTTTTCAGAGCGTGCTGGCGGAGACATTTTTCACAAAA |
| | | | | GAGCCCTTGCGNTGCTGGTGTCCGTGGCGTGCCTGGGAAGNC |
| | | | | CACCAACGCTGGCCGGCCTCCAAGCACCCGGGCCTCTGCTCA |
| | | | | TGTACAGCTCCTGAACTGCCCTGCCTCTGAGTTACTGTGGAA |
| | | | | AATGAGCTTATATATGAAGAAGTCAGCGAGTGGACAAAGCCA |
| | | | | GGCGCAATGGATAGCAAAGATGTGGGAAGTCTCCTCGATTCA |
| | | | | AGTTACAAGAAAACCGCAGCATGGAGTCTNCTCTCAGCTGTT |
| | | | | TGGGGGNATTACCGATGNCTTTGACTAAGTCAAGACTGACTT |
| | | | | TTTCCAGTAATTATCACCCAAGNGGTTAGGAGGNCGTTCCCT |
| 45 | IMAGE:201981 | R99526 | 3′ | GTTCCAAGTTTTTTGNCGTTAGCNTTTT |
| | | | | GAAGGGCATTCTCAAAACGTNNCCGCACAAGCAGACCATCCC |
| | | | | TTTTATTTTCCCCGTCTGTCTCCTTTCCTTCTGCTTTCAAAA |
| | | | | TGTCTCAAGAGTATTTACAAGAGTTGAGCAACACAGGCATCT |
| | | | | TTATCTGGGGTCTTTATCCACAGAGCAGAGGACAGGAAGTCA |
| | | | | TCACTACAGAGACGAAGCGATGTATGGTTTGACCCAGTGGAG |
| | | | | GACTTTGTTAAGGTGGAGGTNTGAGTNTGGAGTGTAAACGTG |
| | | | | GGACATCCAGGGGCAGTGGAGGGTAACCACTGGGAGAGGAAG |
| | | | | TCTGGGGGACAGTTTGGGGAGCCAGCCAAATNTAAAAATAAA |
| | | | | GCATTTCTGTTCTAAATCCAAATGAACCTTTNTACGCTGCTG |
| 46 | IMAGE:201981 | R99527 | 5′ | TCATCTTCCAGTATACCCCAGGG |
| | | | | CTAAGGAAGGGCCCATCCTCACTGCAGAATCAGAAACTGTCC |
| | | | | TCCCCAGTGATTCCTGGAGTAGTGCTGAGTCTACTTCTGCTG |
| | | | | ACACTGTCCTGCTGACATCCAAAGAGTCCAAAGTTTGGGATC |
| | | | | TCCCATCAACATCCCACGTGTCAATGTGGAAAACGAGTGATT |
| | | | | CTGTGTCTTCTCCTCAGCCTGGGAGCATCTGATACAGCAGTT |
| | | | | CCTGATCAGAACAAAACAACAAAAACAGGACAGATGGATGGA |
| | | | | ATACCCATGTCAATGAAGAATGAAATGCCCATCTCCCAACTT |
| | | | | ACTGATGATCATCGCCCCCTCCTTGGGATTTGTGCTCTTTCG |
| | | | | CATTGTTTGTGGGCGTTTCTCCTGAGGAGGGGAAANTCATGG |
| | | | | GAAACCTATTTGTTTCGCAGAAACACACAAGGGTTAGGATTA |
| | | | | CNTGGGAGATAGTTAAAATTGTTCCTCAATGACGTGGCAGGC |
| 47 | IMAGE:202315 | H53024 | 3′ | TTGGAGGGGGAGACGAAGACGGCCTT |
| | | | | TGAAACAAGGAAATCTACTAAGACTTATTTTGACACTGGAGT |
| | | | | GTCATGCCCCCATCCTCAATCTAACATGCTACTGCGTTGTTA |
| | | | | GAGGGTAAAAAGGCCGTCTTCGTCTTCCCTTCCATGCTGCAC |
| | | | | GTCATTGAGGACATTTTTACTATCTCCAATGTAGTCTAGCCT |
| | | | | TGTGTGTTTCTGCGAACAATAGGTTTCCATGAGTTTCCCTCT |
| | | | | CAGGAGAAACGCCACAAACAATGCGAAGAGCACAAATCCCAA |
| | | | | GGAGGGGGCGATGATCATCAGTAGTTGGGGAGATGGGGCATT |
| | | | | TCATTCTTCATTGACATGGGGNATTCCATCCATCTGGTCCNG |
| 48 | IMAGE:202315 | H53025 | 5′ | TTTTTTGTNGGTTTTGGTTCNGGATCCAGGGGACCGCC |
| | | | | TACGTTTTGTATGTTTTTTTATTTGCTCCAGGTGGGGTTTTG |
| | | | | ACTGTCACTTTCCCACACTCTGGATTAGTTCTGATCCCACCA |
| | | | | CAAGGAGCCCTCGAATTGGCTAAAGTGAGAAACTGGGCCTGA |
| | | | | AGACTCCGTACCCTCTGCCATCTTGCCGAGGGAGTCTCCTTT |
| | | | | TAGAAAACAATCAAAGGGTTATTGCATGAGTCTGGATGAATC |
| | | | | CCACTCTCAGCTGTCCACGGGCCCGACCACCTCATCTAGCCC |
| | | | | CCTTTTTGGCAGGGAGAACCTGGGCTCCCAAGTTCTCCTCCT |
| | | | | TCACTTCGTTACAAACCAAGGGGAAGAGCCCACCGTGAGAAC |
| | | | | GCGNCATCTGCAAGCTGTCTCCCTTTTTNCATCCTTGGTNGA |
| 49 | IMAGE:202514 | H53239 | 3′ | AACCCTT |
| | | | | TNTTTTGTTGNCTCTAGCCTGANCAGATAGGAGCACAAGCAG |
| | | | | GGGACGGAAAGAGAGAGACACTCAGGCGGCACANTTCCCTCC |
| | | | | CAGCCACTGAGCTGTCGTGCCAGCACCATTCCTGGTCACGCA |
| | | | | AAACAGAACCCAGTTAGCAGCAGGGAGACGAGAACACCACAC |
| | | | | AAGACATTTTTCTACAGTATTTCAGGTGCCTACCACACAGGA |
| | | | | AACCTTGAAGAAANTCAGTTTCTAGGAAGCCGCTGTTACCTC |
| | | | | TTGTTTACAGTTTATATATATATGATAGATATGAGATNTATA |
| | | | | TATAAAAGGTACTGTTAACTACTGTACAACCCGACTTCATAA |
| | | | | TGGGTGCTTTCAAACAGGCGAGGTGNGTAAAAACATCAGNTT |
| | | | | CCACGTTNGCCTTTTGCGCAAAGGGTTTCACCAGGTTGGGGA |
| 50 | IMAGE:202514 | H53133 | 5′ | AAGGGNGACAGCTTTTT |
| | | | | CATTAAATCAGAGTACTTAATGATACGGAAAAAATTCCTATT |
| | | | | AAGTGAAAAAAGCATTACAAAACAGCATATATTATGAGCTCT |
| | | | | ATTTTTATTTTTGAAATATATTTATGCAGAGAAATACAAAAT |
| | | | | GTTAACAATATTATCTTAAAANAAAAAAATANGGCTGGGCAC |
| | | | | AGTGGCTCACACCTGTAATCCCATACTTTTGGAGGCAAGGTG |
| | | | | GGTGGATCGCTTTGAGCCCAGGGNGTTCAAGACCAGCCTGGG |
| 51 | IMAGE:203114 | H54419 | 3′ | GCAACATGGGCGNAACCCCGA |
| | | | | TGTTCCTCCCCNNTCCCCCAGGGATAAGAACCTGTTATCCAC |
| | | | | CATCAGTAACATTTTATGAAAGATCTACTTATTTGTCTGTTT |
| | | | | TGCAGACATTTTAAAATTCATAAAGTGGGATGCTTCTTTAAT |
| | | | | TTAAATACATTTAGCTTCATGAAAAACTCACTACACAGTTCT |
| | | | | TGTTCAAGCATTATTGGGAAACCACCAGAGGGCACTCTCACC |
| 52 | IMAGE:203114 | H54509 | 5′ | CAGGGCTTAATTTGAACATCTCGCCCAAAAGTGACTTTTAA |
| | | | | ATATCGGCACAGCACTCAGGAAAGCCTAAAGCTTGAAGACTC |
| | | | | CATTTATTTATAGTGCATCCCAATCCAGATACGTAACAATTA |
| | | | | ACGAGTTATTTTTACTATAAGCAAAGTTGCCTAAAATCATAG |
| | | | | TTGATACTAACCATGGTTAACAGAGCTCTAAAGTTTGACAGA |
| | | | | AAGTGAGATTCAAATCCTTTCACTCTCATATGCTAAACCTTT |
| | | | | TGCCTTACTCTGGGTCATCAGAGAAATTTAGGTGAGAATGTA |
| | | | | TGATGAAGTCTGTGTTTTAGATTCAATGCAGATATATCATTG |
| | | | | TGGGCAGAACTCTTTCTGGTTATATCCAGTTAAGAGTAAATC |
| | | | | AGGCTTTCAGCGNGTCGCGGTGGCTTCACGCCTGTAATNCCT |
| | | | | AGGCACTTTNGGGAGGNCCGAGGCGGNGCAGGNTCCACGNAG |
| | | | | GTTCAGGNAGATCGAGACCTTNCGGGNTAGCACGGGGGTTTT |
| 53 | IMAGE:204740 | H57305 | 3′ | NACCTTGTTGNTTCAGGCTGGTTNG |
| | | | | TAAGGAAAAGNNTTAATAAGTAAATATATTTATTAAATATAA |
| | | | | AAGGTACACAGTAAATATAAATGAACTAAATGCTTTAGTTAA |
| | | | | AAGACAATAAAAATTATGAAATAAAAATGTATACACTTGAAA |
| | | | | GTATTTAAAATAAATCTAATTTTCATAATGAATTTTAAGCAT |
| 54 | IMAGE:204740 | H57306 | 5′ | TAAGGAGTTTTGTAACTGANTAGTGGAACTC |
| | | | | AAATGTTAGAGGGTGCGGGGGTGAGGACTGACCACAGATTCC |
| | | | | CTGGATAGTGTAGTGGTAGATTTCTCCACAGGATAGCGCAAT |
| | | | | TGGCAAATCATGCTTGGTTGTGTTAGGCCAAAATACTAGTTT |
| | | | | TGCTTTCTTTACCTTTTCTATCTTGATGAAAATGTTGCACAT |
| | | | | TCTATAGTTGCAAAACACATAAAAGGGGACTTAACATTTCAC |
| | | | | GTTGTATCTTACTTGCAGTGAATGCAAGGGTTACTTTTCTCT |
| | | | | GGGGACCTCCCCCATCACCCAGGTTCCTACTCTGGGCTCCCG |
| | | | | ATTCCCATGGCTCCCAAACCATGCCGCATGGTTTTGGTTAAT |
| | | | | GAAACCCAGTAGCTAACCCCACTGTGCTTNCACATGCCGGGC |
| | | | | NTAAAATGGGTGATATNACAGGTCTTATTATCCCCTATTGGG |
| | | | | ATTTATNCCTCAAACCNCTTAAAAACAAACAGTGGCCTTTTG |
| 55 | IMAGE:205049 | H57493 | 3′ | GCCCTTTG |
| | | | | GATTAAGAACGTAAGCTCCTTTATTATTATTATTATTATTAT |
| | | | | TANTCATNCCCTGTTATTTACCCCNAAACAACAGCATAACTC |
| | | | | AAATAATAATGACACACACGTCCCGCCCATATACACAATACC |
| | | | | ACTAGCCTATCTGTCAGGCTATCTGGCCTTTGCTTGGTTCCT |
| | | | | GATGGAGCTGTCTGGAGACACTCNCCNCTGTAAAAATCCCGN |
| | | | | CTTAAACACAGGGGACAGAAGAAAGGGGGGACCTAGGTCAGA |
| | | | | TCATAAACTGACAGGCTCCCAGCGTCCTTAGGGAGTGCTAAT |
| | | | | GTGGGAGACTTTGAGGACGTGCTTGGACACATTCTGGGGCAG |
| | | | | ANGGCAGNAGGCACTGT |
| 56 | IMAGE:205049 | H57494 | 5′ | TTGTTTTTATGTGGNTGATGGGGTAAATTCC |
| | | | | GTTCNNTTTTCCTTNCTCATTTNATTTTAAAGTTTTATTATG |
| | | | | AAAACACATGGAATTAACGGTGTTATCCATGTATTTGCAACA |
| | | | | GCAGAGAAAGAGTGAGAGTGGACCATCCCCATAGGANCNACT |
| | | | | TATCCTTTGGCTAAACTAATATAAATAATGGAAATAACACCT |
| | | | | AATACAATAATACAGCACATAAAAGAGATTACATTAAGAGAA |
| | | | | GAGACAGGAACTGCGGAGAGGAGTCCTGAGTATGGAGGAGAT |
| | | | | GCGGCTCATGGAGAAGCATCCAGGCTCAGGTGACCTTCCCTG |
| | | | | AAGACTTCCTGTCTCTGAGCAGCTCAGTTCAGTTCCAGGGTC |
| | | | | ATACACGTACTCCGGGACCCGGGNCTCACTGGGGGGTCAGCG |
| | | | | CAGACTTGCTTGCCTCTTTTGGGTTTGGGAATACCACAGCTG |
| 57 | IMAGE:205633 | H62864 | 3′ | GGCTNGGGGAGCAGAGGNTGCTGGGTTTC |
| | | | | CTGAGAGGAACTCCTCACTCAGCTAGCTTCAGGAGCCATGAC |
| | | | | ATCATCTCTACCATGGAAATTCCACTCACTCTCCTGTGCCCC |
| | | | | CACATTTGTCCTAGGCCTCAGAGTCCCTATAAAGAGAGATTC |
| | | | | CCAACTCAGTATCAGCACAGGACACAGCTAGGTTCTGAAGCT |
| | | | | TCTGAGTTCTGCAGCCTCACCTCTGAGAAAACCTCTTTGCCA |
| | | | | CCAATACCATGAAGCTCTGCGTGACTGTCCTGTCTCTCCTCG |
| | | | | TGCTAGTAGCTGCCTTCTGCTCTCTAGCACTCTCAGCACCAA |
| | | | | TGGGCTCAGACCCTCCCACCGCCTGCTGCTTTTCTTACACCG |
| 58 | IMAGE:205633 | H62985 | 5′ | TGAGGAAGCTTCCTCGCAACTTTGTGGTAGATTANT |
| | | | | AGCTCTGCTAAAAACTCCAGCGCAATTTGATGCTGATGAACT |
| | | | | TCGTGCTGCCATGAAGGGCCTTGGAACTGATGAAGATACTCT |
| | | | | AATTGAGATTTTGGCATCAAGAACTAACAAAGAAATCAGAGA |
| | | | | CATTAACAGGGTCTACAGAGAGGAACTGAAGAGAGATCTGGC |
| | | | | CAAAGACATAACCTCAGACACATCTGGAGATTTTCGGAACGC |
| | | | | TTTGCTTTCTCTTGCTAAGGGTGACCGATCTGAGGACTTTGG |
| | | | | NGTGAATGAAGACTTGGCTGATTCAGATGCCAGGGCCTTGTA |
| | | | | TGAAGCAGGAGANAGGAGAAAGGGGACAGACGTAANCGTGTT |
| | | | | CCAATACCATCCTTACCACCAGAAGCTATCCACAACTTCGCA |
| 59 | IMAGE:208718 | H63077 | 3′ | GAGT |
| | | | | TCTTGTGACGTCATTTTATTTTCAGCTACATAGACATCTTTC |
| | | | | TCATGTATTGTTACTAGAACAACTTGTATAGGGTTTTATGGT |
| | | | | TTGGGGAAAACATTTTTAAAAAATGGACTTATCTCTATTATA |
| | | | | CAGAGTTATAATATAAAAATGATTTAAAGGCTATATTTTTCA |
| | | | | GCATGTAGGTAGCTACACTGTAATCCTGTTGAAGANACTTTC |
| | | | | CTATTTAAGCTTATAGGATGANAATATATAATTAAAGTCTTC |
| | | | | TGATCATAGCTTGAGACCATCAAGGGANTGTTTAGTTTCCTC |
| | | | | CACAAAGAGCCACCAGGGTTTTTCTCATAATCTCCTTTGGGT |
| 60 | IMAGE:208718 | H63161 | 5′ | TTCATCCAGGGATGGCTTNGCAAAGGGGAGTTACCAT |
| | | | | AATTAAAGCAAATAGACTGTTGTAGGTACCAATTCTCAATGT |
| | | | | CACAGTGTTACATGGAAAGTAAAATACACAAGAACAGCCCAA |
| | | | | AAGATGGAAACAATGGACGTGGTCAAATGACATCAGTACAAC |
| | | | | ATCCATATGGTCCTAAGTAGCCATCTTTAAAATGGGTTAGGA |
| | | | | AATGCCTTCAATCATTCACACAGGACACATGCATTGGAACAA |
| | | | | ACTCTAAGGAAGTGTTCTTACACGGGGAAAAGGCAAGTTACA |
| | | | | GGATGCATGGGGCATGGATATGGGGTGTAGGATGTGTGGTAT |
| | | | | GGTGGCATCCCCACTTCATACACAAAATACCCCGGCATCGGC |
| 61 | IMAGE:210368 | H65343 | 3′ | CCACATGGCCTGCTGTGTGCGGTAGG |
| | | | | TAAAAAATGATCGTTATGTAGGTGATTGAGAAGTAAATGTAT |
| | | | | TCTTTTTTAAGGTAAAAATTTGGACCCTTATCATGCATACCC |
| | | | | CCCTCTGTGCTCTTCAAATCAACATCATTATTAATATCTGTA |
| | | | | CATTTTTGCTCATCTGAGCCAGCACAGGCTGAGGCTGTCAGA |
| | | | | ATGGACACCTTTTGGTTGTTGGGTTTCTGTCAGTTTCTGGGG |
| | | | | TGAAGCTGCGTGATTGAGAACGTAGCTCTTGGGCTGCCATCT |
| | | | | CGGGGATTATTAAGGACTGTGAACTCTATCCACAAGCCATGG |
| | | | | CAATATCTGTCCCACCGAATGCTNCCTCTAAACACACTCTTA |
| 62 | IMAGE:210368 | H65547 | 5′ | CTTCCCGTGGATGTGTTGTTAAGGGGTNCCGATTGANGGCTG |
| | | | | ATGGGTCATATTTTTGTTCACTGAAAGGACCAACCAGTTTCA |
| | | | | TCAAACAAGCTTTAGAGAAAGAGAAACTGAGTAATTCATCTT |
| | | | | GTCAGTTACAGTTCACATATATGCACACACATACAAACTGGC |
| | | | | TCAGCATCAGTGAAACATAACTATTCAAATACAAAAGTATAA |
| | | | | NAAACCTCTTTAAAAAACCAATAGCAGCCAAAACAGAACATT |
| | | | | TGTAAACAAAACCACAACTNTCAGCCCTGTGCTTAAACACAG |
| | | | | GGTTCTGCATTCTTTTGGAAACATTAAGGTATATGGCATTAA |
| 63 | IMAGE:212772 | H69683 | 3′ | NGGGGGTTNTAGGNCCATCTTTNTC |
| | | | | GCTTTATCATCATGAAACAAGTCATCAGAGTCTTTGAATCTT |
| | | | | GCGTAGGAATTGGAAGTCGGGGTATACCAGGATAGGTTTTCA |
| | | | | GCACCAGGTGTGGCACTCACCCTCCGGTATGCTTGGCAGAGT |
| | | | | TTGTGAAGCGGCTCCGGTACTGCGAATACCTAGGGAAGTATT |
| | | | | TCTGTGACTGCTGCCACTCATATGCAGAGTCGTGCATCCCTG |
| | | | | CCCGAATCCTGATGATGTGGGACTTCAAGAAGTACTACGTCA |
| | | | | GCAATTTCTCCAAACAGCTGCTCGACAGCATATGGGCACCAG |
| | | | | CCCATTTTCAATTTGCTGAGCATCGGCCAAAGCCTGTATTGC |
| | | | | GAAAGCCAAGGAGCTGGGACAGAGTTGAAGGAAATTCAGGAG |
| | | | | GCAGCTCTTCCATNTTCAAGGAGGTTGTTTGAAGACNGTTAG |
| | | | | GTTTTGTAAACAGTGCATTTANAGGGNGTTTCGGAGGCAGGT |
| | | | | GGCCGGGNACATTTNGATTGATGNAGTTCCACCTGTTCTTCC |
| | | | | CTTTGAGGGACNGGGTCAGGATCAGGAAAGGGGTTGTTGGCA |
| 64 | IMAGE:212772 | H70099 | 5′ | AACTAC |
| | | | | NTTCGGCACAGACTTTTTTTAAGCTACCAATTGTGCCGAGAA |
| | | | | AAGCATTTTAGCAATTTATACAATATCATCCAGTACCTTAAA |
| | | | | CCCTGATTGTGTATATTCATATATTTTGGATACGCACCCCCC |
| | | | | AACTCCCAATACTGGCTCTGTCTGAGTAAGAAACAGAATCCT |
| | | | | CTGGAACTTGAGGAAGTGAACATTTCGGTGACTTCCGCATCA |
| | | | | GGAAGGCTAGAGTTACCCAGAGCATCAGGCCGCCACAAGTGC |
| | | | | CTGCTTTTAGGAGACCGAAGTCCGCAGAACCTGCCTGTGTCC |
| | | | | CAGCTTGGAGGCCTGGGTCCTGGGAACTGAGCCGGGGCCCTC |
| | | | | ACTGGCCTTCCTTCCAGGGGATGGATCAACAGGGGCAGTGTG |
| | | | | GTCTTCCGAATGTCTGGGAAGCTGATGGGAGCTCAGANTTTC |
| | | | | CACTGTCAAGAAAGAGGCAGTTAGGAGGGGTTTGGGTGGGGC |
| | | | | TTGTTCACCTGGGGGGCCTTCCAGGTAGGGCCCTTTTTAAGT |
| 65 | IMAGE:232714 | H73130 | 3′ | GGGA |
| | | | | GTGGGNCTGTGTTGAAACAGGCCACGTAAAGCAACTCTCTAA |
| | | | | AGGTCAAACCACCATAGATTTGAATCTGCTGGTCATTCGCCA |
| | | | | TCTGGATTTTTAACTGAATGAATCTCATGGGTTTAACCAAAC |
| | | | | ATGCATGTAATCCTGAATACCATGANTTAAATGCGGANTTGC |
| | | | | CCAGGGACGAGGAAACCTTCAAGAAACAAGGTCAAAGGGACA |
| | | | | NCAGATATAACTGTCACANTAAACANTTCTGTTGACGTGGGA |
| | | | | AATGCACATGACTTGGTTGAAACAAAGCTCCTCAGTGGGCCA |
| | | | | GTGACATCCNGGGTTTTTCTTAGGGTAGGCTGAGGACTCAGG |
| 66 | IMAGE:232714 | H74208 | 5′ | GGCTTATCTCACCTTCTCAGGAATGCTTTTTGAAGG |
| | | | | TTGCTTACATGGGCATCCTTCAGCTTTTAATAATCTGAAAAA |
| | | | | CTCTATTTACCCATTGTCAATGTGTATAAATTAATCTGAGTC |
| | | | | AATTTTATACAATAAAAGGTGAACTTTTATGCATGAAACAAT |
| | | | | AATTTAACAAGAAATGTACCTGAAGAAGAATGTTCATTACAA |
| | | | | ATATAGGANACATAAATATTACCAAATATTGGCAAGCACTAA |
| | | | | AATGTTCAGAAATATAAGTCTATTACAGTTATAGCTCTCTCA |
| | | | | AGCAAAAAAACAGCAGAGAAAAACTTAGTTTACCTTAGGGGC |
| | | | | TATTTATTTACTTAGGGATTTGTTAAAAGGTCGAATGGGGTC |
| | | | | ACACAGAATACTAAGAAGAGCTGTTCACCCAGGCCTCACTAA |
| | | | | GAACTCTTCTTCATTCAGTAGCTGTATAGTAACATGACAACT |
| 67 | IMAGE:234376 | N28268 | | GGCTCCTACGACCCAA |
| | | | | ATTACTTGCAAATTAAGTTACCACAGACTCTGGTAGTGTNCT |
| | | | | AAATNGCGCCAAGGCNTGGGCNCACAGCNCAGTAGCAGNCTG |
| | | | | GNCGNCAGGGCCACTGGCCNACCAGTGACGGACATGCACGTG |
| | | | | GCAGATCATGATTTCCAGCCCACGGAGCCAGCATTTGAACCT |
| | | | | TGTATAATTAACTTTCAGTTATGATTTCCCATCGACATTTTC |
| | | | | TTTGCCCTGTTTGTAGCTGATTGTTGTGTTTTATAAATCTTC |
| | | | | TGTTAAGGCAGAAGGGTGATTATGAGTGGTTCACAGCAGCCC |
| | | | | TTATAAGCTGGGCCAGAAAATTTCACTAGGGTCAGTAATTTA |
| 68 | IMAGE:240367 | H89996 | 3′ | AACCTTGGTTCTTC |
| | | | | GTTTTTTTAGCACTTGTTAATCCGTTATGATTTATTAGCTGT |
| | | | | ACAGCAGTAGATCCTCCTCCCCAGCTTTCAACCCCATTACAT |
| | | | | ATTTTATTACAGGTCTCATGTTGGCGTCCTAAAATAATGAAA |
| | | | | AATATCACACAGTACAGCTAAGTACAAAATGCATCAACCTAG |
| | | | | AGTCTGATAGCTAACTGATGGCTCTCTTAAAAGCAATACACA |
| | | | | GANGANAAAAGTGTTTGAAATCAGTAAGACTGAGGCTCTCTA |
| | | | | AAAAACACATTTTTAAACATGTGACAGTTCATGTGNCAAGGA |
| | | | | NTCACTTTTTAGTTGGGTTTTGGCTTTCACATTATTTTATTT |
| 69 | IMAGE:240367 | H90086 | 5′ | TTTGAGGATCCAGGGTTTAAATTACTGACCTGGT |
| | | | | GTCCTTTGCATAATGCATGGCAAAATGAGCCTAAAACCTATA |
| | | | | TGGCCATTTTAATTTTGCTTTTGTAATAATACCAAGCCCAGT |
| | | | | TTCTTTCAACTTGAGAGATGAGCTATTTATTCTTTTACTTAA |
| | | | | TGAAGATGTAAGAAATGATCTTCTGTTCTAAAAAAAAAAAAA |
| | | | | TTTCTCTGATGTCTCTTGACCCTGTAGAAACACATTCAGTTT |
| | | | | CTACACTGCAAAACAGAGGGATATCTGTATGGCTTCCCTCTT |
| | | | | TCCATCTTTCCTTTCCTCAGGGAAAGCTAGGAAAAAGAAATC |
| | | | | TTTTCTATCACAGCAGACACACCAAATCTCCCTAAGTTGTAC |
| | | | | CACCTTAATTCCTCAGAATGGCAATTGTGTATGGATACCAAG |
| | | | | CTACAACTTGGATAAGAAATTGGTGATTTTCTTCTTTNAATT |
| 70 | IMAGE:244058 | N38809 | 3′ | TTCATTCTCCAATTTTAAAAACATCTATTGGCG |
| | | | | GTAGTGTTTTGGGCACACCTAAGGTCGATCTGTGTTGTATTT |
| | | | | AAAAATCTAATTTCTTTATTTGTGTGGCCTTCTAGACAAACG |
| | | | | AAGGGGACCAGAGGAAACCCCCTGACAGATCTCTGGATGATC |
| | | | | CTCCTTGAATCCTGGGCAGTTTGGTCTCTCCTTGNTGTGCTC |
| | | | | CTGTGGCANAAACTCCCTTTGATTGGTTCTTTCTTTCCTTCC |
| | | | | CAGCTAGACTAAGCCCCTCATGGGCAGGTAATGAAGATTGAA |
| | | | | AACTTTTTTCTGGTCTCCAGTGTGAGCACATTCCTCCTACAT |
| 71 | IMAGE:244058 | N45440 | 5′ | GGTAGATGTNCCAT |
| | | | | TACTACTCATAACAGTTTATTTTTACTTTGTACAAAATACAA |
| | | | | AAATGCAAATCCAAGGAGTACAGACCAGTAGTGACAGGCACA |
| | | | | CTGCACAACAGCAACCTTGTCTAGCAAGACAGGAGTTTTTTA |
| | | | | AATTTTATTTTAGTGAATAAATGCATTATATAAAACAACAAC |
| | | | | AACAACAACAACAAAAACACAAAGAGGCTAGAGATTTCACCG |
| | | | | TTTCTACCCCCAAAATAACGCTTGCTATCAAGACTTTGGAGG |
| | | | | GGGATGGGGGAAAAGAATTTAAAAGGCAAATAATTTTTTTTC |
| | | | | ATAAAAAGTAAAAGCTACCATAAAACATTTTTTTTTCTGTCA |
| | | | | CTGATTAAATTTCTTCTGAAAAGCCGCACATATAGACAAAAC |
| | | | | AAAACAAAAATTCCTGAACTGGACCAACAGCCAATACTCCCA |
| 72 | IMAGE:246722 | N57754 | 3′ | GGGGTGTTAACC |
| | | | | GCCATCATCCCACACATCAGCACCAAGACCATAGACAGCTGG |
| | | | | ATGAGCATCATGGTGCCCAAGAGGGTGCAGGTGATCCTGCCC |
| | | | | AAGTTCACAGCTGTAGCACAAACAGATTTGAAGGAGCCGCTG |
| | | | | AAAGTTCTTGGCATTACTGACATGTTTGATTCATCAAAGGCA |
| | | | | AATTTTTGCAAAAATAACAAGGTCAGAAAACCTCCATGTTTC |
| | | | | TCATATCTTGCAAAAAGCAAAAATTGAAGTCAGTGAAGATGG |
| | | | | AACCAAAGCTTCAGCAGCAACAACTGCAATTCTCATTGCCAA |
| | | | | GATCATCGCCTTCCCTGGGTTTATAGTAGACAGAACCTTTTT |
| | | | | CTGGTTTTCCATCCGGNCATTAATCCCTACANGGTGGCTGTG |
| 73 | IMAGE:246722 | N59721 | 5′ | TTATTCATGGGGCAGGTTAAACAAACCCCTGGA |
| | | | | AAGGATAAATGCTTTATTCTTTCTGTTAATTCATCGTTTTCA |
| | | | | AATGAATGAGATAATGCCCTAGAAACCTCCAAAAGGTACCAA |
| | | | | GGAGGTGAGTGTGTGTATATAATCATAAACTCAGATTTCTAT |
| | | | | ATATTTATATACATTGTGGTCATTATTTGTTTTGATGGCCAT |
| | | | | ATTGTCTCATTTTAGGTTAGTGGGAGCTCCTTAATATTGCTC |
| | | | | CCCTGTTTTTGTGACATGATTCATTAATCTTTGATAGCTTCC |
| | | | | TTGATTTCTGGAGTAAGATGGCCCAAGTTTATTTTACATATT |
| | | | | TCCTGCCCCAGACCTGGATTCAGCTATTCTCCTAAGAGCACT |
| | | | | GGTTCTTAGGAACCAGTNGAGTAATAGTATGGAGAGACCACA |
| | | | | GTCTTGGATGTTCATTGGTAACTACTGGCTACTGAGTTGGCA |
| 74 | IMAGE:258118 | N27108 | 3′ | TTACTTCCAGGAC |
| | | | | TAATCCTAGATTATCTTTATTTGTTCTATAATTTAATAGTAT |
| | | | | ACCTATAAAATAATTACATTATACTTATAGCTTTTCTTCATT |
| | | | | TATAAACAANACAAAAAAATTAAATACAATTTGAGCCATTAT |
| | | | | AAGGTAAACTTTGTACATACGNTAACCCCAGAAGGAGCTTCA |
| | | | | CACTGCAGCATATCATATTGCTTTCATTGCTACACCCACAAT |
| | | | | TGGGTTCGAAGAGAGTGTGCTCGTGTTTGCATTCTGTAAGTT |
| | | | | CTTAGCTTAATCCCTCCCCTATCTGTGTGGGTTCCATGTTAA |
| 75 | IMAGE:258242 | N30655 | 3′ | TAAAATGATAGGGGTTGGCTTTGCAGCTGGNCAGAGAC |
| | | | | CATAATACAGTTTTATAGTTTAATGGACAATGTTTAACATGG |
| | | | | TACCTCTCAAATCTGATATATCTTGTGGTGCTTACAATTTGC |
| | | | | CTTACACTTTCATTTAAAGTTACCCTGTTCTCCACTCACCAC |
| | | | | ATGTATAAAATATCCTATTTTTTCTCTTAATGTTTTACAAAC |
| | | | | CGGTAATTTTCACTATCAGTAGCGGATCTTTTTATAACTCAC |
| | | | | CCTATGTTGCCCAAAATACACCAATAATATAATGATTAGATT |
| | | | | AAAAAACTTGGCATCTTTTTTAAAAAAATGTGCTTTCTTTTC |
| | | | | CATGTATAAGATTCTACTATACCATTTGTGAATGACACCCTA |
| | | | | GTTACATAACACCTACATATCTGCCCCTGTGAGAATTTACCT |
| | | | | TAGTCTTCTAAGACTCTATCTTCAACAGTTAGATAAGTCAAT |
| | | | | AACCAGAGTTCCAAGAAAAGTAGTTACTTTTTAAGACCAAAT |
| 76 | IMAGE:259902 | N32912 | 3′ | TATTGGGATAACTGGGTC |
| | | | | TGGATCCTATAAACCTGTCAATTCTGTTCCTTTTGAGGATGG |
| | | | | CCACACAGACAACCACTTACCTCTTTTAGAAAATAATACACA |
| | | | | TTAACACCTCCCGATTGAAGGAGAAAAACTTTTTGCCTGAGA |
| | | | | CATAAAACCTTTTTTTAATAATAAAATATTGTGCAATATATT |
| | | | | CAAAGAAAAGAAAACACAAATAAGCAGAAAACATACTTATTT |
| 77 | IMAGE:259902 | N42054 | 5′ | TAAA |
| | | | | AGCAGCACCTTTTTGGCTTTTTAATGCTTGGCTTGCTTATAT |
| | | | | CTTTGTCTGTAAAAGAATCTAATAGTTTAAAGCAAGAAAAAT |
| | | | | TCCTAGTCTGCAATTAAATACGTATGGCAACTATGTGGAATA |
| | | | | CTAATCAAATCTTTGGTGTCCTTTCTAAGGTAAATTCATTTT |
| | | | | TCTACCTCAGTTCAATCTTCATTATCATTTTACATTCCACTG |
| | | | | GAGGCCCAGCTAGCACAACAATGGCCAGCTCTTGCCTGAATC |
| | | | | CCGAAAATTAGACTTATATAAATGATACCCCCAGAAAGACTC |
| | | | | GGGGTAATCTCAAAACAGGAGACCAATTTTTGATGCTGGCTT |
| | | | | GCATTCTTGCTTTCTTGGTCATTTTGCTTTTAGTAGGCCAAA |
| | | | | GCTAATACTTCTCCAGTGGGAATTTCAGATGGTTGGACATTG |
| | | | | GATGGGAACAAAGAACATATTTAAGGAAAATTAAATTTCCNG |
| | | | | GGTAGTAAAGTTTATAAACTTTGGAAATCCNTAGACTGGGCT |
| | | | | TAAACTTTCACTGGGTAAATTCNCAATAATGGNAACACCTTG |
| 78 | IMAGE:265294 | N20848 | 3′ | GCCAAAGATGCTATATAC |
| | | | | AGCATATTAGTCTATCAAATCCAACTACCCTTAATGCCAGTGA |
| | | | | ATGTTAAAAGTAAAACTTTCTTAGCACTGACAATTTAATAAGT |
| | | | | AAAAATAAGTGGTACTAAGCTTACAAAAATTAGCTGAATTGGG |
| | | | | GAAATTGTTGATAAGGCCACAAGTATTAACATGTTATACTTGC |
| | | | | TTGCTTTGAGGGTATATAGCATCTTTTGGCAAAGTGTTACATT |
| | | | | ATTGTGAATTTAACAGTGAAAGTTTAAGCCAGTCTAGGATTTC |
| | | | | AAAGTTTATAACTTTACTACCAGGAAATTAATTTACTTAAATA |
| | | | | TGTTCTTTGTTTCCATCAATGTCAACATCTGAAATTCCACTGG |
| | | | | AGAAGTATTAGCTTTGGCCTACTAAAAGCAAAATGACAAGAAG |
| | | | | CAAGAATGCAAGCCAGCATCAAAAATTGGTCTCCTGTTTTGAG |
| | | | | ATTACCCCGAGTCTTTCTGGGGGTATCATTTATATAAGTCTAA |
| 79 | IMAGE:265294 | N27686 | 5′ | TTTTCGGGATTCAGGCAAGAGCTG |
| | | | | NGACAGTTGATTATTTATTTGAATAAAAAATTCAATTAGATTT |
| | | | | CTATCACACACAATAGACACAAACAAATTAAAGGTGGATTAGG |
| | | | | GGCTAAATACATGCATATACATGTATACACACACATACACACA |
| | | | | GATATATATGCATATACATATATTCACACACATAAACACATAC |
| | | | | ATATATTTTTTAAGGGAAAAAAACAATAAAATTAAAACTTAGA |
| | | | | AGTATATATATGTAAACTGTGATCTGGTTTCAAGATTATGAAA |
| | | | | GGCTTTCTAAATAGCTTAAAGTAGAAATCACAACAGTAAAAGA |
| | | | | TAATCTGATTATAAATAAAAAAGAGGGAAAACCTTTTTATGTA |
| | | | | AAGAAGACCATAAAATTTAAAAGGCAAATAATAAACTGGGGGA |
| 80 | IMAGE:278944 | N63049 | 3′ | AATACCTGGCAAAATATATTCATATCCNAAATATACCAAGAGT |
| | | | | AGCTTCATTAAAATCTTGGGAAATTTTAATTTGCATTCACCTT |
| | | | | CTCTAAACATGAACATGAATCTGTAAAGTGATACATTCTTTCT |
| | | | | TGCTTAAGAAATTAAAGCGTTTGGGGATTTGAGTTTTTATACT |
| | | | | CTTTGAAAATTGAGTTTCTTGTGCTAAAATCATCATTCACAAA |
| | | | | ATGTCCTCTCACCTGAGGAATTCCAACACAGCAAATTCAATCT |
| | | | | GAAATAAATTGAGGCTACATTTAAGAGACGGGACTTCCAGCTA |
| | | | | AAAATAGGTATTAGAGAGCTGTTTTTGCCAAAAAATTGAATAC |
| | | | | TTAACCTTATTCTTCACTCTTGACTCATTTGTTTTGTCTCAGT |
| | | | | NTGGGTGACTGGAGGGTTTCTTCTTTGTATTTNCATTCTGTAT |
| 81 | IMAGE:278944 | W00554 | 5′ | CCATTTCTTAATGCGATTGAATTAGANACCATTTTATG |
| | | | | TATTTAAAGCACATTTTTATTATAGATAGGTTAAGTGTGGTTT |
| | | | | GCTGTGGCTAAAGATATATTTATAATGGATGAACAAGCTTTTC |
| | | | | TAGATACCAAGAGGTATAATATTTTTCTTTCAGTATTGAACTA |
| | | | | ACATTTCNCTGATAACAAGGAGACATTGAACTGGCTGAGCCTA |
| | | | | TTTTAAATGGGAAAAGACTTTTTTTTTCTGGATGTTGCTTTAA |
| 82 | IMAGE:280567 | N51674 | 3′ | AGACTGGNAAATTAAAAATTTTAAAGTACCA |
| | | | | GTCATGTCAGTAATTTATTTCAGGGTCTAACAAATATTACCAC |
| | | | | AGCAGTTTAGTCTCAAAGTGATACAAAACTGAACTCAGGGTGG |
| | | | | TTACTGGGTAGTCCCTAGTCCAAAAGATTAAGACACACCTCTA |
| | | | | ATACACACACAGGCTGTGTTCAAGGCCTTTTCCTTCCCATCTT |
| | | | | CTGGTTCTGTCTCCACCCTTTCCAACTGATAGCACTTCATTGG |
| | | | | TGTGTGTGATATATGTGATTATCTTAAGCTAGAAAGTACAACA |
| | | | | GAAGGAGAGGATGGTTGTCACTTGGGGATTAGACAGTTGAGAG |
| | | | | GATAGGAAAGGAGTTATATCCACCAATACAAGCCCTTCTTCCC |
| | | | | CTCCTACTTAGAAAGAGGGTGGGACCATTGGCATTCCTTTTCT |
| | | | | AAGAAGCCCCTCAGCAAGGAGTCTGTTCCAAGAGAATATAACC |
| 83 | IMAGE:284584 | N59450 | 3′ | CGNACTANGAAC |
| | | | | GAGTTTGTTTGAAGCACACCTTTAACTCAGAATTGAGGTTGAC |
| | | | | TGATAAAACTCAGCTTTAAGTAACCCTCTGGGCAAGTTCTGAG |
| | | | | CAGAGATCCAGTGAGCTGAATGTCAGGCACCACCTCCCTGGAG |
| | | | | TCTGTATCAGTCACATCAGCATTCTCCTCTGATTAGAATCAGG |
| | | | | TTTCAAGGGTCTTGTTCAAGAGTTTATTCTCTCCTTTAAAGAT |
| | | | | GCCACAATACCGTATAAGGAATGTCTCTTGGTCCCAACTAATC |
| | | | | TACAATAAGAGAGGAGCACGTATAGTCAGAGGGCAAGAAAACA |
| | | | | ACCGCAGTTTCTAAGTTTCAGGTTATATTCTCTTGGGAACAGA |
| | | | | CTCCTTGCTGGAGGGGGTTCNAGGAAAAGGGAATGNAATGGGT |
| 84 | IMAGE:284584 | N71839 | 5′ | CCACCTCTTTCTAAGT |
| | | | | ATTAAATAGAATTTAATACTTTATTAAATTTTATTAATGTTTA |
| | | | | CTTCTACCTGTTTAGACTATTTTTAAGGAATGTAGACATCAGT |
| | | | | ACTACTCGAAGTGTGGTCCCATATTGATCCCATATTGATCAAC |
| | | | | TGTCATTGGCTGATGGAGAGATAAGCACATAAAGTGAGCAAAC |
| | | | | ATGCATAAACATTTAGAAATGCTGATAGTAAACTGACAGTGCC |
| | | | | AATGCATTCAAGTACATGATTTTGTATTTACNAAAAGTATCCT |
| 85 | IMAGE:287721 | N62231 | 3′ | TTTATGAATGGGTTTAGAATT |
| | | | | GGCGTGAAACTGNTNCTCTACTAAAAATACAAAAAATAGTGGG |
| | | | | GCATGGTGGCGCATTCCTGTAATCTCAGCTACTCGGGAGGCTG |
| | | | | AGACAGGAGAATCACTTGAACCCGGGAGCAGAGGTTGCAGTGA |
| | | | | GCCGAGACTGCACCACTGCAGTCCAGCCTGGGCAACAGAGCGG |
| | | | | GACTCGGTCTCAAAAAAAAAAAAAAATGAATAAGACAGTAGTC |
| | | | | TCACCTCCAGGAACATAACCTAGATGNNGTANAGNCGNCGAAC |
| | | | | GGNTNAGCNGGTNTGNGNCNACTAATNTTNCACAGGGTAATTG |
| | | | | AGGCAGAGTGGGACTCTAAAGGGTCTAAGATATTTACAAGGGG |
| | | | | TGCTAGAGGAAAGAANGAGAATATATAGGGTCCAAAAGACTTT |
| | | | | ATTTTCTTAGGGGAGTTTTACATCATCTCCCCACAGGCAGAAG |
| | | | | CCCTGGGTTATGTGACTATGCCAGTAATTGAGTGGTTTAATCT |
| | | | | CCAGTTTAGGGATATGGGGTATTTAACCAGTCCCTGTTGCTAC |
| | | | | AGATTGAAAAGACATATTCTTTAATTTTGCTAACAATTAAAGG |
| | | | | TGATGTTTGATCTCCNGGAGTAACTTCTCCATCTTCAGGGGGT |
| | | | | TTCCAAATTCTGGNGGGAAATNCAGGGGTGTTNCCCATTTTTA |
| 86 | IMAGE:287721 | N79323 | 5′ | TCATTNGGATC |
| | | | | CATTTTAATTCACTGAACTATATTTTTTGGTACATTACCCTTC |
| | | | | AACTAAAAAAATAAAATTAAAACATTTCCCTATTACTGATGAA |
| | | | | GGTTAGAATGAAGAGAACATAAGGTATATAAGTAGGAAAGAAA |
| | | | | ACCTATGTAGGGACAGATGTTAATAGTTATTAAATCCTAAGTA |
| | | | | AAATTTTCAGAACTTGGAAATTACCAAATCCAGGAGTGGTCAG |
| | | | | ATTCCTTTATGAAGGTAGATCTGGAGCTACTTAGGCCAGATTT |
| | | | | TTGTATTTTAGCAAAGTTCCTCAGATGATTCTGACGCACACCT |
| | | | | GGATTATAAACCACTAAACCACTGAACTACCCCAAGAAGGTTA |
| | | | | CGTGACCTCCCAGAGCTAGAATGTNCCAGAAATGGTGCAAGAA |
| 87 | IMAGE:288736 | N59214 | 3′ | TTCNATTACTGGACTCCTGGCCC |
| | | | | GAAATCACAACAAACTGAATTAAACATGAAAGAACCCAAGACA |
| | | | | TCATGTATCGCATATTAGTTAATCTCCTCAGACAGTAACCATG |
| | | | | GGGAAGAAATCTGGTCTAATTTATTAATCTAAAAAAGGAGAAT |
| | | | | TGAATTCTGGAAACTCCTGACAAGTTATTACTCGTCTCTGGCA |
| | | | | TTTGTTTCCTCATCTTTAAAATGAATAGGTAGGTTAGTAGCCC |
| | | | | NNNAGNGTCTNAATNCTTTANGATGCTATGGTTTGCCATTATT |
| | | | | TAATAAATGACAAATGTACTTAATGCTATACTGGAAATGTAAA |
| | | | | ATTGAAAATATGTTGGAAAAAAGATTCTGTCTTATAGGGTAAA |
| | | | | AAAAGCCACCGTGATAGAAAAAAAATCTTTTTGATAAGCACAT |
| | | | | TAAAGTTAATAGAACTTACTGATATTCCTGGTCTAGTGGGTAT |
| 88 | IMAGE:288736 | N75239 | 5′ | AATA |
| | | | | CAGGTTTTTATTATTTATTATTATTGTTTGTTTTGAGATGCAA |
| | | | | TCTTGCTCTGTCACGCAGGTTGGTGTGCAATGGTGCGATCTTG |
| | | | | GCTCACTACAACCTCCGCCTCACGGGTTCAAGCAATTCTCCTG |
| | | | | CCTCAGCCTCCCAAGTAGCTGGGATGATGGGCGTCCGCGCCGT |
| | | | | GCCTGGGTAAATTTCTGCATTTTTAGTCCAGATGGGGTTTCAC |
| | | | | CATGTTGGGCAGGCTGGTCTTGAACTCCTGACCTCAGGTGATC |
| | | | | CGCCTGCCTTGGCTCCCAAAGTGCTGGGATTACAGGCGTGCAA |
| | | | | CCCGCGCCTGGCCCCAAATGTCATGTTTTTAAATAAAAACATA |
| | | | | GAAAATGATATAAAGGTTCACAGCATCATCAAGAAAACAGTTC |
| 89 | IMAGE:289337 | N92646 | 3′ | CCCCGTGTCGCGGAGGGGAGATG |
| | | | | GCAGNGGGAACGTCTTCTCATGCTCCGTGATGCATGAGGGTCT |
| | | | | GCACAACCACTACACGCAGAAGAGCCTCTCCCTGTCTCCGGGT |
| | | | | AAATGAGTGCGACGGCCGGCAAGCCCCCGCTCCCCGGGCTCTC |
| | | | | GCGGTCGCACGAGGATGCTTGGCACGTACCCCGTGTACATACT |
| | | | | TCCCGGGCGCCCATCATGGAAATAAAGCACCCAGCGCTGCCCT |
| 90 | IMAGE:289337 | N99582 | 5′ | GGGCCCTGC |
| | | | | AAAATAAAGAATCAGATTTATTGGGGTGGTTAAGTGAGATCAT |
| | | | | GGATGAACTGTTTACTTCTATTCAGCAGTAGCCTTTGTGGTCC |
| | | | | CAGGCTTCTGGTGGCCAGATAATTCCCTCAACTCCATGAGCAG |
| 91 | IMAGE:290749 | N71796 | 3′ | GTGACCGAGGAGGACTCTCACATCCTGCATGTGTTTNAGA |
| | | | | AAGTTTTGCCACTAACTTTAATGTATCATTAGGCAAATTATC |
| | | | | CTCTCTGAGCCAAAGAAGGGTAGTGGGATTACGGGATCTCCA |
| | | | | AGGATCGTTCTTCTTAACATTGTCTGATGGCATAATTGTCTT |
| | | | | ATTAAGATTTCTAGGGAGAAATACAAAGTTAAAAATAAAATC |
| | | | | ATATAGGTTAAAATTATGTAAACATCTGGCCTAGAGCCTCTT |
| | | | | GATTCAACTCACATAACTAACCAGACCATGGGGGCCAACAGG |
| | | | | TCAAAGGACACTATGTAAAAGACATGACTTAGACACATGGAG |
| | | | | TGAGAGGAGCAACACAGGCTCCCATGGGTGGGGACTGAGCTG |
| | | | | GAAGGTCACAGTAATGAGTGAACTCCCCCTTGGGCACACTTT |
| | | | | AGTATGATGAGTAAAGCTTCCCTGGTGACTTTAGAGAATGGA |
| 92 | IMAGE:293005 | N69118 | 3′ | TCATGGGAACACCTTTATAAGAAG |
| | | | | GGGGATCTGCCTGAAGCAGGGATGGGACACNAAGTCCCTCCA |
| | | | | GCTTATCTNTNCACAACAACCCTTTCCCTGCAGANATGGTTT |
| | | | | GTATACCACAAGCCCTCTTAGCACGCAAAAGCCAAAATCTAA |
| | | | | AGATCAACCATTTATCCTGAACAACACCATTTGAGAAAGAGG |
| | | | | TAACCATCTTTGGTTCTACATGGTTTGGAGAGTATAGTGGTA |
| | | | | GGAGGGGCTCCCTGATTCCCCTAAAGCTATGCACACCACAAG |
| | | | | GGGCTCTGCTCTTCTGTCTGGGATCTTCTTATAAAGTGTTCC |
| | | | | CATGATCATTCTCTAAAAGTCACAAGGAAGCTTTACTCATCA |
| | | | | TACTAAGTGTGCCCAAGGGGGAAGTTCACTCATTACTGTGAC |
| | | | | CTTCCAGCTCAGTCCCCACCCAATGGGAAGCCTGTGTTGGTT |
| | | | | CTCTCACTCCATGTGTCTAAGTCATGTCCTTTACATAGTGTC |
| | | | | NTTGAACTGGTGGGCCCCATGGTCTGGGTAGTTATGTGAGTT |
| 93 | IMAGE:293005 | N90642 | 5′ | GAATCAAA |
| | | | | CATGATCATTCTTTTTAATGTGCACCAAATTAGCAGTAAAAA |
| | | | | TAGCAGCAGATGGATCAGAGTGGTTGTCAATAAACCTTTTCT |
| | | | | CCCCAGGTTACTAATATACAATTGCCATGAAAAATAAAAAAA |
| | | | | TATATATATATATTTACACTTGACTCATCACCTCTGCTTAGG |
| | | | | ACCCTGTAAGCACAAGATATTGCTGAACTGCTGTATTTGCTA |
| | | | | CATATGGAACAATTAGACTAGCAATAAGAAGTAGTTTATGCA |
| | | | | TGTATGCTGGCCTACATGNATATACCCCTTTCCGCAATTACT |
| | | | | GAGGATTATCAACAAAGTTTGGTCTTGGTCTTGTGATTATAA |
| | | | | TNCCAATNAAATNACATNTTAAATGGGGATATCNCCGAATTN |
| | | | | TGGTTTTNATAATTACGTAATTAATTCCNAAGAAATTAAATA |
| 94 | IMAGE:294647 | N69453 | 3′ | GGTAATATAGACCCCTGTAAAAANTAACCNT |
| | | | | AGGAAGGCCAGAGTATTAATATCCCCATCTGTGTCTTTTGCC |
| | | | | TTCCATGAACCTGGGTTTTGAGCCCTCTCTTGTAAAATGGGC |
| | | | | ACAGTAATATTACCTACCTCAGGGAGTTGTGAGGATTAAACA |
| | | | | TGAAGTGCTAAGCATAGTGCCTGGTACAAAGACAGTACTCAA |
| | | | | TAAGTGCTACCTAAAACTAGTATTCATAGCAATACTGTTAGG |
| | | | | ATAAAGAATTATCATATATGAGATAGTTCCAAATTTTTGTTT |
| | | | | TTTTAAAAAAAAAAGAGTTTTATAAGTTCAAGATAATATTTT |
| | | | | CTTACTTCAAAGAAACAATCTCACAACGAGGGAATGGTAAGA |
| | | | | ATCAGGAGAGATTACTAACCTGGCAGAGGAGCTATCACAATC |
| | | | | ACAAAGGTGGTTTTTCCAGGGCACGGCTCATCCATTACACTC |
| 95 | IMAGE:294647 | W03283 | 5′ | CAGATGTGCTGACCC |
| | | | | TCAGTTTACAATGCATAATGATATGTCTTTATTTCATCAACA |
| | | | | GAAATGGTGTCTAGACAAAATTCAGTTAACACTAGCAATTCA |
| | | | | ATTGAGTGAAAACTTTTTTTGCACAATAGTGTATTTACAATG |
| | | | | AGTAAATGAAGTTTCAATTCATTAGTTCATAGCAATGCTTTT |
| | | | | TTCCCCCAAAAGGTAAAAATTCTTAGTTACAGAGAATAAGCA |
| | | | | TCAACAGCCTTTCATTTTTTACAATNAAAACCNCGGGNAAAA |
| | | | | CCNCAATCCCCTTTGGAAAAAAATTANGGGCCAGGCCTAGGA |
| | | | | CCTAGGTNCAATAAAATGGATGGCATTGGAATTAAATTTCCA |
| 96 | IMAGE:296483 | N74648 | 3′ | TTAATCGGCATAGGAATCCCGNGGTAAAANGTTTGGTAGGAA |
| | | | | ATGTCTACAAGGTTTTATTAAAATTAAGTTTAACATTAATAA |
| | | | | CACACTAATATAAAGGTAAAATTTAGCTTATCTGGTATAAAA |
| | | | | GTCATACAGGAAGCATTAGTAAATATAAAATAGCGTTTAGCT |
| | | | | TTCTTTTGTCTAAAAACTAATAAAAATTGGTGCTAAAGGAAG |
| | | | | CATTCATTTTACTAGAGGATCATAAAAGTTAAAGACTTAAAA |
| | | | | CAAACTTTGGCAATTAAGACAGCATACCAAGATGCAAATGCC |
| | | | | TGGTTGAAATGGATCAAATATTCCATCTGCAGGTTAAACAAA |
| | | | | AGCAATTAGCATGCTTGTGCACATGGCAGGCCAGAGACCCTG |
| | | | | ATTGTCCCCCTTCCACTAAGGTGGTCCTCCAGTCGGGCCAGG |
| | | | | CATGGGCTGCATGGTAGCTCTTTTCCAGGATTCTATAGCCTG |
| 97 | IMAGE:296488 | N70208 | 5′ | GAGTAATAAGTCATGCCAAGCTCTCTCCTGCTATATN |
| | | | | TTTTTTTGACTCTTATCTAACTTTACTTCCAAACAATGATTT |
| | | | | ATAAAATGTGGAGGAGAGTGGGTGTCTATGTCAAGCAGCCTT |
| | | | | ATGATAAGGCTCCGTCATATATTGTGCTTATTCAACAATACT |
| | | | | GGTGTTAATGAGGCCTGGCCTCCAAAGGACAAAGATACAGAA |
| | | | | ACAGAAAGGTTTTCCCAGGCCAGAAGTATTAGTTTACATCAC |
| | | | | AACATTAAAGCATAATACACTGTGGGCCTAAAAATTAAACTG |
| | | | | CATGTGTTCTAGCAGCAGGAACAACAACAACAACAACAAAAT |
| | | | | GCTTTCACATTTATATAAAAATGACAAAGTAAAAAGCAGAGA |
| | | | | ACACAGTGAAAAGTGTCTGGCAGTTCATTAAAATACAGTTGA |
| | | | | GTTGCTTCTATAGTCTCAAACCATTATTATATTATTTGAATG |
| | | | | AGAAAGAGTATGAGGATTTAACTGGCTGAATTCCATTCCTAC |
| | | | | CCCTTATTCATAGGGGAATAATTACCCTGATATTATTTAAAA |
| | | | | GTGTTTGCTTTACNCAAAANTAAATAACCTTAAATATTTAAA |
| 98 | IMAGE:305302 | N95059 | 3′ | AT |
| | | | | TTTTTTCCAGGAAAAAAATTAAATCTTTATTTTTAAAAATCC |
| | | | | CACAAATCCATAATGAAATCATCATCTGAAAAAAAAGATGGT |
| | | | | AGGGAACAAAACGTGGGATACATTTAAAAGGCACTAGATTCA |
| | | | | TTAATACCAGAGCCATTCTGGAGATGCCATGTAAGAAATCTG |
| | | | | GAGTTACTCTAAATCTTCTTCTTAGTGGTATCAGAACTGGGG |
| | | | | AGAAGGGTCCAAGCAAAGTGTTGCCTTTGCCAGTGTATTCGG |
| | | | | ATCGAGGTTATGAGGAAGAGCCCTTTTCCTTTGTCAGTGAGT |
| | | | | TTCATGTTGGTCCACCACTCCAGCGCTGACAGCTCCCCGATG |
| | | | | GCCCTGTCATCGTATCTCAGGACCTCCTTCAGGATGTGCGTT |
| | | | | GTGTGCTGCCGACAGGGGGGCGGCCTGGCTCTGACACTTGAN |
| 99 | IMAGE:309499 | N99256 | 3′ | TTACTGTACTCACACTGGGCTATGAAGTACACAGTTAGA |
| | | | | TCATAGGCCCAGCTGTGAGATACAGTAAGTTCAAGATGTCAG |
| | | | | AGGCCAGGCCGNCCCCCCTGCTCGGGCAGCACACAACGCACA |
| | | | | TCCTGAAGGAGGTCCTGAGATACGATGACAGGGCCATCGGGG |
| | | | | AGCTGCTCAGCGCTNGGAGTGGTGGACCAACATGAAACTCAC |
| | | | | TGACAAAGGAAAAGGGCTCTTCCTCATAACCTCGATCCGAAT |
| | | | | ACACTGGCAAAGGCAACACTTTGCTTGGACCCTTCTCCCCAG |
| | | | | TTCTGATACCACTAAGAAGAAGATTTAGAGTAACTCCAGATT |
| | | | | TCTTACATGGCATCTCCAGAATGGCTCTGGTATTAATGAATC |
| | | | | TAGTGCCTTTTAAATGTATCCCACGTTTTGTTCCCTACCATC |
| | | | | TTTTTTTTCAGATGATGATTTCATTATGGATTTGTGGGATTT |
| 100 | IMAGE:309499 | W30727 | 5′ | TTAAAAATAAAGATTTAATTTTT |
| | | | | ACATTTACTAGTTTATTGAATATGAGGTTTATCCATTTAGCA |
| | | | | ATGTAAGGAAAACTTTAGTTCTGTTTCTCAGTTATCAGGAGT |
| | | | | GAACATAAAACTATTCTAAACCACAATTAGTTTACCAGCATA |
| | | | | GTACAAAATAAAATNGACAACTAACGAAATAAAGCAATTAAA |
| | | | | GTAACTTATTTTTACTCATAAGGTTACCATAATAATAAAAAT |
| | | | | TCCTTTAATTTTCAAAGCACTCTTCATGAAAANGTAGTTGGG |
| | | | | GGAAAATTACTATTTGTTCCAANGTAGGATAAAAGGGNAGGG |
| 101 | IMAGE:321886 | W37628 | 3′ | ATGCNCCCAANTTAAACATTTTTATTNAAAAATTAAACCCCCC |
| | | | | GATTGAAATACCATCAGAGGCCCAAGCTCTCTTTTCCAGAGA |
| | | | | GCAGTGGCTTTTGTAATAATTCACTATCTTAGAGTGAAAAAG |
| | | | | GACTAGACCTGTGTTACATAATAATCTTGGTTCAAGCTGCCC |
| | | | | TTCTGAACAAAGATATAAACCTAGCATACATTGTAATAGATA |
| | | | | ACTGGTAAAACTGACAACTTTTACTTCTCAGAGGCCATTTAA |
| | | | | ATATAATAGGAACCTACTGACCAAACCTAGTGATACATAAAA |
| | | | | TTAAAGCCTGTGNACTTTTTAAAGTTGTTAATCACTATACAT |
| | | | | ATGTATGTGTATATGTGTATACACATATATAATTTTATGATC |
| | | | | AATATCTTAGATATTTTAGAAATTCCCTTTNGAATAGTCTTG |
| 102 | IMAGE:321886 | W37627 | 5′ | GCGTGCCGTGGAAAAATAGAAAATCAGGGAGATA |
| | | | | ATAATTTATTAGATCTAAAGCCCCTTCCTCCCCAGCCCCTGC |
| | | | | TTTCATTAAGGTATTTAAACTTGGGGGTTTCACTGCTCTCCC |
| | | | | CCATGATGGAGGGAGGGAGCCCCCCAACCTCAGTGAGGAGAG |
| | | | | CCCCGAGCCGGCCCCGGGGAAAGAGGGGTGCAGAGGGAGTTC |
| | | | | CCCCAGATCAGTACCCCCCACCCCTCCCCAGCTAGTAGCATG |
| | | | | ACCAGGAGACGGTTAATGAGAGCCAAGAGGAGTACCTGGTGC |
| | | | | ACCTGGTGCGGTGGTGGAGACCTGGGGGGCAGGTGGATCTGG |
| | | | | GGCTGTTCCCCCCCCTCCGTTTTTTCCACCCCACAGTTCCTC |
| | | | | CTGGGATCTGGCCCTCCAGGGNAAGTGGAGCCTCCAGCCCCT |
| | | | | AGGGGATGCATGAGGGGGGAGGGGGTGCTGAGTGGGAGGAAG |
| 103 | IMAGE:325024 | AA284236 | 3′ | AGTCAGGCTCACAGCTGGGGTGGCCTGGGGGTGGGGGT |
| | | | | GTAAAACGCTAATAATTTATTAGATCTAAAGCCCCTTCCTCC |
| | | | | CCAGCCCCTGCTTTCATTAAGGTATTTAAACTTGGGGGTTTC |
| | | | | ACTGCTCTCCCCCATGATGGANGGAGGGAGCCCCCCAACCTC |
| | | | | AGTGAGGAGAGCCCCGAGCCGGCCCCGGGGAAAGAGGGGTGC |
| | | | | AGAGGGAGTTCCCCCAGATCAGTACCCCCCACCCCTCCCCAG |
| | | | | CTAGTAGCATGACCAAGCNTAGNTTTNATGAGAGCCAAGAGG |
| | | | | AGTACCTGGTGCACCTGGTNCGGTGNTGGAAGACCTGGGGGG |
| | | | | CAGGTGGATCTGGGGCTGTTCCCCCCCCTCCCGTTTTTTCCA |
| | | | | CCCCACAAGTTCCTCCTGGGATCTGGCCCTCCAGGGAAGTGG |
| 104 | IMAGE:325024 | W49598 | 5′ | AAGCTCCAGCCCCTAGGGGATGCATG |
| | | | | CAGCAACATGAAGTTGGCAGCCTTCCTCCTCCTGTGATCCTC |
| | | | | ATCATCTTCAGCCTAGAGGTACAAGAGCTTCAGGCTGCAGGA |
| | | | | GACCGGCTTTTGGGTACCTGCGTCGAGCTCTGCACAGGTGAC |
| | | | | TGGGACTGCAACCCCGGAGACCACTGTGTCAGCAATGGGTGT |
| | | | | GGCCATGAGTGTGTTGCAGGGTAAGGACAGGTAAAAACACCA |
| | | | | GGCCCTCCCTGCTTTCTGAAACGTTGTTCAGTCTAGATGAAG |
| | | | | AGTTATCTTAAGGATCATCTTTCCCTAAGATCGTCATCCCTT |
| | | | | CCTGGAGTTCCTATCTTCCAAGATGTGACTGTCTGGAGTTCC |
| | | | | TTGACTAGGAAGATGGATGAAAACAGCAAGCCTGTGGATGGA |
| | | | | GACTACAGGGGATATGGGAGGCAGGGAAGAGGGGTTGTTTTT |
| 105 | IMAGE:325247 | AA284262 | 3′ | TTAATAAATCATCATTGTTA |
| | | | | CAGCAACATGAAGTTGGCAGCCTTCCTCCTCCTGTGATCCTC |
| | | | | ATCATCTTCAGCCTAGAGGTACAAGAGCTTCAGGCTGCAGGA |
| | | | | GACCGGCTTTTGGGTACCTGCGTCGAGCTCTGCACAGGTGAC |
| | | | | TGGGACTGCAACCCCGGAGACCACTGTGTCAGCAATGGGTGT |
| | | | | GGCCATGAGTGTGTTGCAGGGTAAGGACAGGTAAAAACACCA |
| | | | | GGCCCTCCCTGCTTTCTGAAACGTTGTTCAGTCTAGATGAAG |
| | | | | AGTTATCTTAAGGATCATCTTTCCCTAAGATCGTCATCCCTT |
| | | | | CCTGGAGTTCCTATCTTCCAAGATGTGACTGTCTGGAGTTCC |
| | | | | TTGACTAGGAAGATGGATGAAAACAGCAAGCCTGTGGATGGA |
| | | | | GACTACAGGGGATATGGGAGGCAGGGAAGAGGGGTTGTTTTT |
| 106 | IMAGE:325247 | W52431 | 5′ | TTAATAAATCATCATTGTTAAAAAGCA |
| | | | | TCTGAAGTCACAGCAGCAATACAGAACAAAGAATTTACCTTA |
| | | | | ATCTGATCTTTTTACGTGGAATTCCCTGACTCAAACTCAGTG |
| | | | | GCTTAGTTTGGAAACCTCTGAATGGCTGGGGAGAGAAAATCT |
| | | | | TTTGAAACTAAGTGAATAAATTAACACACACATACGTNGGAA |
| | | | | ATCAGCCCTTGTGCAAGTGTAACATGAACATCACTGATGAGA |
| | | | | GTGCAGAAACTCCAGGCACCCCTCTGCCTCCTCCTATCCCTG |
| | | | | GGCCTGGGGTTGTAGGGAGAAGTCACACTCAATTCATTTCTA |
| | | | | GCCACACCATGTCCCTAACAGTGCTAGTGTNAACTAGCCCTG |
| 107 | IMAGE:341096 | W58202 | 3′ | ACCTGGGTATTGGGTTTAAAGAATGGAGCCTCGTGCC |
| | | | | GCTCATTCTTTAAACCAATACCCAGGTCAGGGCTAGTTCACA |
| | | | | CTAGCACTGTTAGGGACATGGTGTGGCTAGAAATGAATTGAG |
| | | | | TGTGACTTCTCCCTACAACCCCAGGCCCAGGGATAGGAGGAG |
| | | | | GCAGAGGGGTGCCTGGAGTTTCTGCACTCTCATCAGTGATGT |
| | | | | TCATGTTACACTTGCACAAGGGCTGATTTCCACGTATGTGTG |
| | | | | TGTTAATTTATTCACTTAGTTTCAAAAGATTTTCTCTCCCCA |
| | | | | GCCATTCAGAAGGTTTCCAAACTAAGCCACTGAGTTTGAGTC |
| | | | | AGGGAATTCCACCGTAAAAAAGATCACGATTAAGGTAAATTC |
| 108 | IMAGE:341096 | W58311 | 5′ | TTTGTTCTGTATTGCTGCTGTGACTTCNGNA |
| | | | | TTGTTTTGTTTTCTTTCACAGATTTAATACCGCGATCTCAGC |
| | | | | CAAACTCCGGCCGAGAAGTTGAGAAATGTCTTCACCCCCTCT |
| | | | | CGACATTCGTTCGTGCTTCTTCGCCTTGGTGGAGCGATAGGG |
| | | | | GCGAGCAGGGGTGGGGCCGGCTGGTGCTGCTACGAGGGCCGT |
| | | | | GCAGCGNTTNAATAAGTGACATAAAATGTCTACACGCATAAG |
| | | | | TAACCGTACTTAGGGCTTCTGCAAGGGCCACCAGAGCGCCTA |
| | | | | AGGTGGCAAGTGGGCCCCGTGTCACNGGCCGCGCTGCAGGCG |
| | | | | CTTGCGCAAAGTCTTCCACGCAGCCGTCCAGCCCCATGCGCT |
| | | | | CCAGGGCCGCGTAAACGGCTCCGAGGCCCGCGGGTTGCTGCT |
| | | | | GGCGCCAGGCTTTGAGCATCTCGTACTGCTGGTCTCGGAAAC |
| | | | | GGCCGATTTCCANCTTCAAGGGCTTCGATCTCTGCCTCGCGA |
| | | | | AGCCCAGCGTGCCAACGAACTTCTTCCAAGCGCCGNCTGGGA |
| 109 | IMAGE:345586 | W71984 | 3′ | ACNGCGTCAATCAAGGTCG |
| | | | | CACAAGCCCTGGTTACTGCAGATGAAGCTGGGATGGAGGCTC |
| | | | | TGACCCCACCACCGGCCACCCATCTGTCACCCTTGGACAGCG |
| | | | | CCCACACCCTTCTAGCACCTCCTGACAGCAGTGAGAAGATCT |
| | | | | GCACCGTCCAGTTGGTGGGTAACAGCTGGACCCCTGGCTACC |
| | | | | CCGAGACCCAGGAGGCGCTCTGCCGCANGTGACATGGTCCTG |
| | | | | GGACAGTTGCCCAGCAGANTCTTGGCCCCGCTGCTGCGCCCA |
| | | | | CACTCTCGCCAGAGTCCCCAGCCGGCTCGCCAGCCAATGANT |
| | | | | GCTGCAGCCGGGCCCGCAGCTCTACGACGTGAATGGACGCGG |
| 110 | IMAGE:345586 | W76376 | 5′ | TCCCAAGCGCGGCGCTGGAAAGGAAGTTCCGTGCGC |
| | | | | CAATTTTTAAAAATGTTTTATTACAAAGCTTCTTTTAAAAAA |
| | | | | ATGCTCAGCACATTAACTCAAACTGGAATGACAAACGTTAGG |
| | | | | ATGACAGTTTTGGGCAAAGGCTGTGCTTGCTTTTTTAAAAAA |
| | | | | TGGGTACATCAATGCTCATTTTAACAACTNGGCATAAAATCC |
| | | | | CACTAATTGGCTAATAAAAACAGATACAAATACAGAACATTT |
| | | | | AAAGTAATAACAATTCAAGTGCTGGGCTTTTTACAACAAGGG |
| | | | | GGTGATAAGGAAAGAAATGAAAATTCACTGCAAACCAGTCTG |
| | | | | CTGAACGCATCTGTTAAGGTTTACTGTTTAAAAAAAAAAAAG |
| | | | | AAGAAAACAGAAGAAAAAATAAACTGAAATTAGGGCTGCCAA |
| | | | | TTGCTACCAACAGAGTGGGTTTGGCTATTACATTTATTTAGC |
| 111 | IMAGE:347036 | W81129 | 3′ | TCTACTGGAACACCTTACAAGGGCGGAGAAGCCA |
| | | | | ACTTGAATTTTTTTAATTTACACTTTTTAGTTTTAATTTTCT |
| | | | | TGTATATTTTGCTAGCTATGAGCTTTTAAATAAAATTGAAAG |
| | | | | TTCTGGAAAAGTTTGAAATAATGACATAAAAAGAAGCCTTCT |
| | | | | TTTTCTGAGACAGCTTGTCTGGTAAGTGGCTTCTCTGTGAAT |
| | | | | TGCCTGTAACACATAGTGGCTTCTCCGCCCTTGTAAGGTGTT |
| | | | | CAGTAGAGCTAAATAAATGTAATAGCCAAACCCACTCTGTTG |
| | | | | GTAGCAATTGGCAGCCCTATTTCAGTTTATTTTTTCTTCTGT |
| | | | | TTTCTTCTTNTTTTTTTTTAAACAGTAAACCTTAACAGATGC |
| | | | | GTTCAGCAGACTGGTTTGCAGTGAATTTTCATTTCTTTCCTT |
| | | | | ATCACCCCCTTGTTGTAAAAAGCCCAGCACTTGAATTGTTAT |
| 112 | IMAGE:347036 | W81128 | 5′ | TACTTTAAATGGTTCTGTAATTGGTATCNGGC |
| | | | | TTTTTTTTCGGTATTTGAATACATTTATTGTGACAAGAATGC |
| | | | | TGTTATAAATATTCATAAGCAAAGGCCATCTTTTTATCTAGG |
| | | | | AATTGTCAAAGAGAAGATTCCAAATTGGAAGGATACATCTTT |
| | | | | TGTAAAATCTGCCACCAATTCCTGCTTTGAGAATAAGCACCT |
| | | | | ATTGTAAAATTTCTACTAACATTATAAATGGTCACAGCACAT |
| | | | | GCCACTTGATACAATCCAAACTTTGAAATGTTTGACTTCTCA |
| | | | | GTGGGCTGTCCCTCTCCACTGCAACCCCCCTTCCTCCAGCCT |
| | | | | CCTGAAACATCGCACTATCCTTTCGGTAAGCAATTCCATATA |
| | | | | GATAGCTGGGGGGAGGAGGAGTATAACCTGGACCATAGCATC |
| | | | | AGGTTACATCAGGTACATTTATTTCTAAAGTCTAATAGAGAA |
| 113 | IMAGE:358531 | W96155 | 3′ | CAGTTTTTACTGCTTAATAGTAAGAAGCACTGAGAGTGA |
| | | | | GTATCCTGCCCAGTGTTGTTTGTAAATAAGAGATTTGGAGCA |
| | | | | CTCTGAGTTTACCATTTGTAATAAAGTATATAATTTTTTTAT |
| | | | | GTTTTGTTTCTGAAAATTCCAGAAAGGATATTTAAGAAAATA |
| | | | | CAATAAACTATTGGAAAGTACTCCCCTAACCTCTTTTCTGCA |
| | | | | TCATCTGTAGATACTAGCTATCTAGGTGGAGTTGAAAGAGTT |
| | | | | AAGAATGTCGATTAAAATCACTCTCAGTGCTTCTTACTATTA |
| | | | | AGCAGTAAAAACTGTTCTCTATTAGACTTTAAGAAATAAAAT |
| | | | | GTACCTGATGTACCTGAATGCTATGGTCAGGTTATACCTCCT |
| | | | | CCCTCCCCCAAGCTATCTATATGGGAATTTGCTTACCAAANGG |
| 114 | IMAGE:358531 | W96134 | 5′ | ATAGTGCCGATGTTTC |
| | | | | GGTGTAATTAGCATNGGTCAATGCGGGACGATNGAGTGGCTCT |
| | | | | GGAAACCTGATGGATTTCCTCGATGAGCCGTTCCCTGATGTGG |
| | | | | GGACGTATGAGGACTTCCACACCATCGACTGGCTAAGGGAAAA |
| 115 | IMAGE:363058 | AA019316 | 3′ | GTCACGGGACACCGACAGACACATG |
| | | | | TAAATGACACAGTCAGTGTTTTTCTGAAAATAATTGCCACCT |
| | | | | TGTTGCTAATTAAACATGATGGATTCGGGGTCCTGGTTTGCC |
| | | | | ATCTGGGCCATATGTCTCAGAACATCCTTTTTTGTGATGATG |
| | | | | CCAAGAAGTCTCCNGCTCGGGTCACAAGGAATTGCNGAAGGC |
| | | | | CCCAGTTTTCCGGGANGATATNCAACAACGGTTTCAATCGGA |
| 116 | IMAGE:363058 | AA019413 | 5′ | ATTT |
| | | | | TTTAAGCTAGAAAAAGGCCAAAAAGCAAAACCTGAGAAAACA |
| | | | | ATACGTGTTGTTTTCTCAGGAAAAGAAAAACCTTCATGACCC |
| | | | | TACTGAAGAGCATTGGAGATCAGCTTCCGCTAAGATGCTAGC |
| | | | | TTGGCCAAGTCTGTTATATTCACCTGAAAAAGTCTTAGCAGA |
| | | | | GAATTTTTGCATTCCCACCCAAAAGCCCTCTCAGCCACTCAA |
| | | | | ATGCCTATCTTCTCCAGTCTACAAGTTACATGNTCCCACCCA |
| 117 | IMAGE:382773 | AA065090 | 3′ | GCAT |
| | | | | ACCGAAGCTTAAAGTAGGACAACCATGGAGCCTTCCTGTGGC |
| | | | | AGGAGAGACAACAAAGCGCTATTATCCTAAGGTCAAGAGAAG |
| | | | | TGTCAGCCTCACCTGATTTTTATTAGTAATGAGGACTTGCCT |
| | | | | CAACTCCCTCTTTCTGGAGTGAAGCATCCGAAGAATGCTTGA |
| | | | | AGTACCCCTGGGCTTCTCTTAACATTTAAGCAAGCTGTTTTT |
| | | | | ATAGCAGCTCTTAATAATAAAGCCCAAATCTCAAGCGGTGCT |
| 118 | IMAGE:382773 | AA064973 | 5′ | TGAAGTCC |
| | | | | TAAGAGGTTGCGAACATACATATTTATTTATAATACAAAATN |
| | | | | AAGATTNGAGGGAAAAGTGCTTTAAAAAGTANCATGTAAGTG |
| | | | | TATAAATGAAATTNTNGCTTCTTCTCCGATACAATTTTGATT |
| | | | | GGGTGAGCATTATTTGCTTTTACAATAATGCTTTATTTTGTT |
| | | | | TTTTGCATTGCATTGCACTAACCTGTCCATTAATACAAACAG |
| | | | | AAAAAGAAGGTGGAGGACGTGCCCAGCCGCGTGGTCAGCGTG |
| | | | | CCGAACCTCGCCTCCTATGCAAAGAACTTTCTGAGTGGCGAT |
| | | | | CTGAGTTCCAGGATTAATGCCCCTCCAATAACTACATCACCC |
| | | | | AGCTTGGACCCAAGCCCCAGCTGTNGGCCTGGACCCTACAAA |
| | | | | CCCANACCAGTCTACAGATTGCAAAAACTGCCACAAGGTTTT |
| 119 | IMAGE:417637 | W90399 | 3′ | GGGGGGAATGTTTGG |
| | | | | AAGATTTTTGTNCCAAGTCCNGTGCTAAGCACATCCTATGGA |
| | | | | TTAATTCCTTTAGTCTCACGTCAGTCTGATGAGATAGGTGCT |
| | | | | GTATTATCTTAATTTTAAAGGCAAGGTATATGGAGACCTGGA |
| | | | | GAGGTCAAGTGACCTGTCCAAGGCCACAGAGCTAAGAATGAG |
| | | | | GAAGACTGTAATTTGAATTCAGACCTCCAGGCCAGATGGAGT |
| | | | | CCACCTTTTGTATAACCCATGCTGAAGTTTTCAGGTAAGTGA |
| | | | | TTCAGTGTCCCTTGTCTAATCATCCATGAAAAAAGGCCTTCT |
| | | | | GGAATTTGGTACCAGGTGCTAGAAAGAATCCTACTTCCCCTC |
| | | | | TNATCTACANNGNAAANACGNATAAGGGCCCCTGTCCCCAAC |
| 120 | IMAGE:417637 | W88508 | 5′ | ATCCCCCAAACCTTGTGGCAGTTTTTGCATCTGTAGACT |
| | | | | CTATCATTGTGAACTTTTTCCTCTCCTGATCCAGTTCATCAT |
| | | | | GGAGGCTCATCATTTCTGTTTCCAAAGTCAAGTTTCGCTGTT |
| | | | | CTAAGCTCTTTATCCTGGACTCATACTCTATCTTCTGTTTGG |
| | | | | TCATTTCCTGTTTCAGGCTGGAAACTAAACTGTGCAGTGCNT |
| | | | | GTGGTTGCTGCTGCTGTTGCCCACAAATGTCTCACTGTTGTC |
| | | | | ACTGCTACTGGTGGCACGACTACTTCGACCTCCCACACTCCT |
| | | | | GTGGTCACTTTTGCTTTCATAGTCCCTTGGGGTGGGAAAGGT |
| | | | | CGTCCTGCGGGGGCCCATCCAAAACAGGGTCCTCAAAGTTCC |
| | | | | CCCCAAAAAAGTCTTGCTCTGGGCAGGTGGTGGTAGAAGAGC |
| | | | | GACAGGAGTTGGAGTTCTCAGGGAGGGAGATTTCACAGGAGG |
| 121 | IMAGE:418185 | W90522 | 3′ | AAGTGGACCAGGTAGCACTGNA |
| | | | | TTTTTTTTTCACAATTGGAATGTGCTTTATTTCAGGGAAATA |
| | | | | TAAAGGGAAATGAATGCTATTATAACTTGGTAGAACAGAAGA |
| | | | | AATGGCTACCTAGCTTTGCTTTCCAACTACAAACATAAATGA |
| | | | | GGATCTCAGCATTTAAGGTAAAACATGATAAGCACAAAAGGA |
| | | | | GAGTTCACTGGGGACTGGACTCCCTCATTTACTCTAGAAATT |
| | | | | ATGAGAACCAGCAGCAATATTCCTCAAGCATCCATCTCAACA |
| | | | | TCAAGTTCCTTTGTTTTATTTACCAGATGACCAGGGAATCAT |
| | | | | AGGATGAGTTTGGGCTGCAACTGTGTCTTCCACTGCCATTCC |
| | | | | CAAAGACTTGAACACGGTGGGTCTTCTCACAGTGGGGCTGGG |
| | | | | TTCACTTCCCTTAATCACTTTTTCCCAGGTTCAGGCAAAGGN |
| | | | | TCTTGGGGCCCCTGGACCAGGCAGGGGACATTTTCCAGGATT |
| 122 | IMAGE:42373 | R59968 | 3′ | NCTTTCAGGGGGCAG |
| | | | | NCGCAGCTCCAGNCTCCTCATCCCGCCTCTAGAGACGNCCCT |
| | | | | GGCAAGCTTNTNCAGCGGTCCCGAAGNGGGGGTNATGCAGCC |
| 123 | IMAGE:42373 | R60419 | 5′ | CGTGCGCANCGTG |
| | | | | ACTGAGGTTAGAAGGCACAGGTGGCGAGATGAGCCGGGTACC |
| | | | | AGCGTTCCTGAGCGCGGCCGAGGTGGAGGAACACCTCCGCAG |
| | | | | CTCCAGCCTCCTCATCCCGCCTCTAGAGACGGCCCTGGCAAC |
| | | | | TTCTCCAGCGGTCCCGAAGAGGGGTCATGCAGCCCGTGCGCA |
| | | | | CCGTGGTCCGGTGACCAAGCACAGGGGCTACCTGGGGGTCAT |
| | | | | GCCCGCCTACAGTGCTGCAGAGGATGCACTGACCACCAAGTT |
| | | | | GGTCACCTTCTACGAGGACCGCGGCATCACCTTCGGTCGTCC |
| | | | | CTTCCCACCAGGGTAATTGTGGTTACTCTTTTGAGCCCAGCA |
| | | | | ATGGGCACCNTGCTNGGCGGTCATGGGATGGGAAATGTTCAT |
| | | | | AAATTGCAAAGAGAACAGTTGCATTTTTTGCCNTTTGCCACC |
| 124 | IMAGE:42373 | R67147 | 5′ | AATTTTTTTG |
| | | | | AAAAACAGACATAGTCTCACTGTTGTCCAGATTGGAGTACAG |
| | | | | TGACACAATCATAGCTCACTGCAGCCTCAAACTAATGGGATC |
| | | | | AAGTGATCCTCCTGCCTCAGCCTCCCAAGTAGCTAAGCCTAC |
| | | | | TGGATGCACTACTATGCCCAGCTCACACAGAAGGTTTCTGAG |
| | | | | TAATCTGTTGCTCTTTTTCCCTACAATTTGTCTTCCATATAA |
| | | | | CTCAAACTGACAAGGCTATGGCTTACATAAAGAAATATATTA |
| | | | | TAAATCAACAACACTCATGATAAGTTTACATAAGACATGAGA |
| | | | | ATACACCTGAATCACCAACCGGGAAAAATGATTGAAGAGCTT |
| | | | | GAAATTAAGCCTAAGTGTAAGTCTCTGTTAAGCTTACAACAT |
| 125 | IMAGE:429165 | AA005108 | 3′ | TACAATAGTTAAATCG |
| | | | | TCTAACCTTCGATTTAACTATTGTAATGTTGTAAGCTTAACA |
| | | | | GAGACTTACACTTAGGCTTAATTTCAAGCTCTTCAATCATTT |
| | | | | TTCCCGGTTGGTGATTCAGGTGTATTCTCATGTCTTATGTAA |
| | | | | ACTTATCATGAGTGTTGTTGATTTATAATATATTTCTTTATG |
| | | | | TAAGCCATAGCCTTGTCAGTTTGAGTTATATGGAAGACAAAT |
| | | | | TGTAGGGAAAAAGAGCAACAGATTACTCAGAAACCTTCTGTG |
| | | | | TGAGCTGGGCATAGTAGTGCATGCCAGTAGNCTTAGCTACTT |
| | | | | GGGAGGCTGAGGCAGGAGGATCACTTGATCCCATTAGTTTGA |
| | | | | GGCTGCAGTGAGCTATGATTGTGTCACTGTACTCCAATCTGG |
| 126 | IMAGE:429165 | AA005107 | 5′ | ACAACAGTGAGACT |
| | | | | GATCATTCCATCATGTATTGATGCATACAAATATCACATTGT |
| | | | | ACCATATAAATTATACAATTATTGTACAAATATATACATCAA |
| | | | | TATACAATTGTACATACAATACATACAATTGTTGTACAAATA |
| | | | | TATACAATTATTACTTGTCAATTAAAAATTTTAAAAAAGAAA |
| | | | | TCTGAAATAACAGTTGCCCCCTATGAGCATCTCACGATAAAT |
| | | | | CCCTTTAATCTCCTCTACATATACTGAGTATTAAAAAACAGA |
| | | | | ATCGTCTAGAACATTGTTGCTGTTCTGAGACCTGTCTTTCTC |
| | | | | ATTTAACACAAGTGAACATTTTTCTTTGTCAGCAAGTAGCGG |
| | | | | TAAACATCATCCATTCTAATGGCTGTATTTTTTAATAGGTGG |
| | | | | AGTTGTATCTTCAGGGCAGATTCCTAACAGTGGAATGGCTGG |
| 127 | IMAGE:429569 | AA011448 | 3′ | GTCACAAGGGAAATGTGTAGGTAGTTTTTGGA |
| | | | | GTGACAAGCAACCTTAAAAGAGACACAAGGAGACTGGCAGAC |
| | | | | AGAGGAAGAAGAGGCAGCAATGTGACCCCGGANGTGGAAATC |
| | | | | TCAGTGATGGGGCCAGGAATGTCAAGGAATGGTCAAGGAATG |
| | | | | GCTACAGCACCAGAAAAAGAGGCAAAGTGAGGCTTCTCCCCT |
| | | | | AGAATCTCTAGGAGCGCTCCAGCCCTGCTGATGTCTAGATTT |
| | | | | TTGGAGTTCTGGCCTCCAGAATGTGAGAGAGTAAACTATTGT |
| | | | | TTAAAGCTACCAAGTTTGTGGTAACTTGTTAGAGCAGCCACA |
| | | | | GGAATGAATGTACAGGGAATCAGGGCAGTCTCATACACTGAT |
| | | | | GGTGGGAAAACAAACCGGCACAACCCTTATGGTGGGAAATTT |
| | | | | GACAACATTGTACAAAAACTACCTACACATTTCCCTTGTGAC |
| | | | | CCAGCCATTCCACTGTTTAGGAATCTGCCCTGAAGATACAAC |
| 128 | IMAGE:429569 | AA011447 | 5′ | TCCACCTA |
| | | | | TTTTTTTTTTTAGTCTAAAGAAAGTTCTGAACAGAATATCAA |
| | | | | TTAAGCTTACATCACAAAAACTTTAAATGTATTTACAGAGTG |
| | | | | AATAAGTTACATAGATAAACTCTGAATATGTTTCTGCAGTGC |
| | | | | AACAAGTTCACATGCACACATCTAACACTTGACAGCATTAAG |
| | | | | TTTAAGGAGAGAACTTAAGAATGGCCCTTTACATATATATTA |
| | | | | CACATAAAATATGACATCGAAGAAACAAAGTAACAACTCATA |
| | | | | TTTTACCTTTATGATTCTACTTCTGACTATCCAAACAGGATA |
| | | | | TTAAAATATGGCATGCCTGGACAGGGTGAAAAGACTTGGGGA |
| | | | | TTTATCTTGTGGAATAGTTTTCTCTACAAAACGGGCAAAGTT |
| | | | | TAATTAAATTTAACNCTTCATTCCTTCCGGCGGTTTNAAATA |
| | | | | TGGCTCNTTAAAGGCNACCTTCTGGTTAAAAGGCCGGCCCGG |
| 129 | IMAGE:46284 | H09111 | 3′ | TTCCCTTNAAAAGG |
| | | | | GCCACACTCTCTTNGCTTGCAAATTGTAAGGCAACATTTGCA |
| | | | | GGGGGATCAAGAGATGGAGTAATTACCTGTCAACCAGGGGAC |
| | | | | TCCGAAGAAAAGCAAATGGAATCTCTTGCACAATTGGAACTG |
| | | | | TGTCAGAGATTATATAAGCTACACTTCCAGCTGCTATTGCTT |
| | | | | TTTCAGTCCTACTGTAAGCTCATCGGCCAGGGTGCACGAAGT |
| | | | | TAGCTCCATGCCAGAGCTGCTGAATATGTCCAGGGGAACTGA |
| | | | | GTGACCTAAAGAAACACCTGAAGGAAGCCAGTGCAGTCATTG |
| | | | | CAGCTGACCCTCTCTATTTCAGACGGCGCGTNGGTCCGAGCC |
| | | | | CACCTTTCACGTNCACTGAAGCAGGCCATCCAGTTCCATGCT |
| 130 | IMAGE:46284 | H09461 | 5′ | TGGGAGTTGCCTTGAAGGGACCAACGGACT |
| | | | | GAAGTAAAAGATTTTTATTGTTCTATAGACACTTCTGAAAAG |
| | | | | AGATCTAATTGAGAAAATATACAAAGCATTTAAGAGTTTCAT |
| | | | | CCCCAGAGACTGACTGAAGGCGTTACAGCCCTCCTCTCCAAG |
| | | | | GCTCAGGGCTGAGAACGGTTAGCATATCGAATGATCAGTAAA |
| | | | | AACATGCAAAAGTGAGAAGGAAAGGGAAAAAGGTGCATTCCC |
| | | | | CTAAGCTGAGGGGGATGGAATTTCAGAACAGAGGANGCAGGG |
| | | | | TGGACAAGTACCAAGGTGGCTCTCCCTTTCCCTCTGTGTNAT |
| 131 | IMAGE:471196 | AA034213 | 3′ | CTTTCAAAACCANTTCCAAGCNTGGATNAAAGCAA |
| | | | | TTTTTTTTTTAGCACACCACAGCCACCATACAGACAGGAGTG |
| | | | | CAGCCCCTCCTCCCTAGGAACCCCCACCCCTACTCTTCACTA |
| | | | | GGCAGGGCCCATGGCTCATGAATGCAGAACAGTCACCCCAGC |
| | | | | CATGGCTGAGCATACCCACTGTTAGTGACACAGAGTTTCCCT |
| | | | | GAGAAGAGGCTCCCAAAGGCATACGACAGCCCCTTGGCCACT |
| | | | | GCCACAGTAACAGTGCTATCCCTCCTGCCCTTGGANTAGGGG |
| | | | | AGGACACAAAGAGCCTAAGGGCTACACTTCAAACTTAGGAGT |
| | | | | ACATCACAGCCACCATATGGGAGAGGAGACCAACCTCTTCCT |
| | | | | CCCTGTGAGGCCTTTCAACTNCCTGCTCCCCAACAAACAGAA |
| 132 | IMAGE:47151 | H10995 | 3′ | CCCCAA |
| | | | | CAGTACTGCGGCCNNCNCTCCTNTCCNAACCTCGCTCTCGCG |
| 133 | IMAGE:47151 | H10727 | 5′ | GCCTACCTTTANCCGCCCGCCTGC |
| | | | | ATTGAAAATAGATGTTTTATTTTGTTTATACAAGGTACAATG |
| | | | | TCAAAATACAAATAATATATAATGTATAGATATAATAGACAA |
| | | | | GGAAGTATAAATATAAACGCATATATTCGTAAAATGGCACTG |
| | | | | AGTTGAGTTTTCTTCTTCCTGAATCCTTCAATGGAGAGGATT |
| | | | | CNCTGGGCTCAGCATCTCTCCCACCTTTCCCAGGTCCCTGTC |
| | | | | CATGTGTGCAGAGAGCTGGAGACAGGGTGGTTAGAAGCCCAA |
| | | | | ACGCTGGTGTCTTCCCTGTAGACGTCTCCCACGCCAGGAGAA |
| | | | | GCCTTGTAATTGACAGAGAGCTTTGGGTATGTCACTTTTCTC |
| | | | | TGTGAACTGAAAGTTTAGGATGAGGGCNCGGAANATTCGGGG |
| 134 | IMAGE:488019 | AA054754 | 3′ | CAGGGTTTT |
| | | | | ACNAGCATCCGCCTCCCACCAGCCGCCAGTGTNGTATCCACA |
| | | | | GGGCCACAGCGACACCACTGTGGCTATCTCCACGTCCACTGT |
| | | | | CCTGCTGTGTNGGCTGAGCGCTGTGTCTCTCCTGGCATGCTA |
| | | | | CCTCAAGTCAAGGCAAACTCCCCCGCTGGCCAGCGTTTGAAA |
| 135 | IMAGE:488019 | AA053285 | 5′ | TGGAAGCCATGGAGGCTCTG |
| | | | | CCTTCTTGTTCACTNGGTGTGGTTTATTCTTGAAGCAAGGTC |
| | | | | TCTCTCCAGTTGAAGCCCCCAGTTGGTCCATGGGTAAGAGGA |
| | | | | AGGATTGGTGGATCTGTCAGCTGCCATATTCCAGTTTCTCCT |
| | | | | AATTCTTCACAGGAACAAAATCCCAGATATGGGATCTTTCGG |
| | | | | ACCATTTGTACGAAGTCCTTGGAGTTCTGAGGTGACAGGCCC |
| | | | | TGAAGTTGGCAGGTACACGCTTCAAGGGAAGATGCGTGGGCC |
| | | | | ACAATCAGGATGTTATTTCCTTTACTTTTACATTCACTTATT |
| | | | | ATTNCTTTTGTTACTTGGGAAACTTCTACTNGATATAAGTAT |
| | | | | CATAGGATTCTGGAAACAACTAATTTNGCTCGATTGGAATGT |
| 136 | IMAGE:489047 | AA047190 | 3′ | GAGGNCTGGTAGGTTGTATCAACACTCAGGTT |
| | | | | CNTTCGGCACGATGGGGAGTATTGGAGAGGCGGCCTTATGAN |
| | | | | GNCCANGNGCTCGGGGAGACGACTCCTCTTACTATCATCTGC |
| | | | | CAGCCCATGCAGCCGCTGAGGGTCAACANCCANCCCGGCCCC |
| | | | | CAGAAGCGATGCCTTTTTGTGTGTCGGCATGGTGAGAGGATG |
| 137 | IMAGE:489047 | AA047189 | 5′ | GATGTTGT |
| | | | | TTTTTTTTTTTCTTCCTTTTTTTTCTTTTAGAAATATTCAAA |
| | | | | TTTTAAAACAACAATTAAGTGGATTATGGGAACAGGAAAACC |
| | | | | ATCTTACTTTGGTTCCAGGATATACTGGTAATATAGCTAAGG |
| | | | | ATGTAGATGCTTATTTATTACAGTTACATTGAGAGATTTCAT |
| | | | | CTACTAAAGAGCATTTGGTTTTTCAAAACATCCCTGAACTGT |
| | | | | ATAATTTACAAAAAAAAAAAGTCTCGTCTGAGAACTGTGAAC |
| | | | | TGTGGAAGAAATCAAAACTATTTTTNCTTTTAAAAAGCCACG |
| | | | | TAATGAAACCNCTAATGAAATCCCAGCAATCTGCTTCACATT |
| | | | | GAAGTGGAAAAATATCCAAAAGGAGCAGCTTCAATTTTCATT |
| | | | | GAGGTGAAAGTGCACTATGAAGATTGTTCACCTTTGGCTGCA |
| | | | | TTTGGGAGTTATATGGTTATTTGGTAACNTTAAGAACTNCTG |
| 138 | IMAGE:501778 | AA127879 | 3′ | GATTTTTAATGCCATCCNGGCATNAAAATATNATTTNTACC |
| | | | | TAATAAAACACAATCTTAAAAAAGTTAATGATGATTGGTCTT |
| | | | | GGTGGTTCCTAGTGGTAAGTCCTGTCTTATTTTTTCACATAG |
| | | | | TATAAATTATATTTTTATGCAGGATTGCATTAAAATCCAGTA |
| | | | | GTTCTTAATGTTACCAAATAACCATATAACTCCCAAATGCAG |
| | | | | CAAAGGTGAACAATCTTCATAGTGCACTTTCACCTCAATGAA |
| | | | | ATTGAAGCTGCTCCTTTTGGATATTTTTCCACTTCAATGTGA |
| | | | | AGCAGATTGCTGGGATTTCATTAGTGGTTTCATTACGTGGCT |
| | | | | TTTTAAAAGAAAAAATAGTTTTGATTTCTTCCACAGTTCACA |
| | | | | GTTCTCAGACCGAGACTTTTTTTTTTTGTAAATTATACAGTT |
| | | | | CAGGGATGTTTTGAAAAACCAAATGCTCTTTAGTAGATGAAA |
| | | | | TCCCTCAATGGTAACTGGAAATANATAAGCATCCACATCCCT |
| | | | | AGCNATATTACCNGGTATATCCNGGAACCCAAGTAAGATGGG |
| | | | | TTTCCCGGTCCCCATAATCCNCCTAAATGGTGGTTTAAAAAT |
| 139 | IMAGE:501778 | AA127929 | 5′ | TGGANTATTCCCAAAGG |
| | | | | TTTTTTTTTTAAATGAATGTAACAAGCATTTATTAAAAACTG |
| | | | | TGCTGCACAAAACATGTTAGAAACTAGACCAGGTGCTAGGAG |
| | | | | TCTAATCAAGGCAGGGGCAGGGTAAAAACATGGGAATATTAC |
| | | | | ATGGACAAGCTTGTCTAGCATGGCAGTCTATAACCCTTGAGG |
| | | | | GTTTACATAAAATAAAGGAACATTTTTGTGGNCTCAGGCTCC |
| | | | | CCAGAGTTCTCTTTATGTTTGGGCAGAGACTGCCCATCCCTT |
| | | | | AGTGATCCCACCTTAGAGCCAGGTTTTCAAAGTCATTTCTCC |
| | | | | CAGTATATCTGTCTCTGTATGCAAGTTTCCTCTGGTTGCCTT |
| | | | | GAGCAAAAACAATCATCCAAGTCAAATTTGCTAGCCCTATGC |
| | | | | TGGGCCAGCCCACGTTTCCCGTAGGACATCTGTAGGGTAAGT |
| 140 | IMAGE:50214 | H16746 | 3′ | TNAGCCCCG |
| | | | | TTCCAGAAGGCAAAAAGACATTACCATGAGTAATAAGGGGGC |
| | | | | TCCAGGACTCCCTCTAAGTGGAATAGCCTCCCTGTAACTCCA |
| | | | | GCTCTGCTCCGTATGCCAAGAGGAGACTTTAATTCTCTTACT |
| | | | | GCTTCTTTTCACTTCAGAGCACACTTATGGGCCAAGCCAGGC |
| | | | | TTAATGGCTCATGACCTGGAAATAAAATTTAGGACCAATACC |
| | | | | TCCTCCAGATCAGATTCTTCTCTTAATTTCATAGATTGTGTT |
| | | | | TTTTTTTAAATAGACCTCTCAATTTCTGGAAAACTGCCTTTT |
| | | | | ATCTGCCCAGAATTCTAAGCTGGTGCCCCACTGAATCTTGTG |
| | | | | TACCTGTGACTAAACAACTACCTCCTCAGTCTGGGTGGGACT |
| | | | | TATGTATTTATGACCTTATAGTGTTAATATCTTGAAACATAG |
| | | | | GAGGATCTATGTTACTGTAANTAGTGTGATTACTATGGTCTA |
| 141 | IMAGE:50214 | H16854 | 5′ | GAGAAAAGTCTACCCCTGCTAAGGAGTTCTCATCCCN |
| | | | | GCTTTTAACAATGATGATTTATTAAAAGAAACAACCCCTCTT |
| | | | | CCCTGCCTCCCATATCCCCTGTAGTCTCCATCCACAGGCTTG |
| | | | | CTGTTTTCATCCATCTTCCTAGTCAAGGAACTCCAGACAGTC |
| | | | | ACATCTTGGAAGATAGGAACTCCAGGAAGGGATGACGATCTT |
| | | | | AGGGAAAGATGATCCTTAAGATAACTCTTCATCTGTCCTTAC |
| | | | | CCTGCAACACACTCATGGCCACACCCATTGCTGACACAGTGG |
| | | | | TCTCCGGGGTTGCAGTCCCAGTCACCTGTGCAGAGCTCGACG |
| | | | | CAGGTACCCAAAAGCCGGTCTCCTGCAGCCTGAAGCTCTTGT |
| | | | | ACCTCTAGGCTTGAAGATGATGAGGATTCACAGGAGGAGGAA |
| | | | | GGCTGCCAACTTCATGTTGCTGTTGGAAGGCTTTGGAAAAAC |
| | | | | ACAGCAGGGCTGAGAGCAGCTGAAATTTATACCTTCCANCCG |
| | | | | CTGAGCTGGNATGCNAGGCCAGGGGTGGGACTAGGGGACTGC |
| | | | | AGACACCTTAAGNCCTGGCCAGAAACTTGACATTTCTNGAGA |
| 142 | IMAGE:503051 | AA149250 | 3′ | TTAGCACCACCCTGTGTACCCTGGGTCTT |
| | | | | AGGACCCAGGGTACACAGGGTGGGTGGCTATTCTCCAGAAAT |
| | | | | GTCAGTTTCTGGGCAGGGCTTAGGTGTCTGCAGTCCCTAGTC |
| | | | | CCACCCCTGGCCTTGCATTCCAGCTCAGCGNGTGGAAGGTAT |
| | | | | AAATTTCAGCTGCTCTCAGCCCTGCTGTGTTTTTCCAAAGCC |
| | | | | TTCCAACAGCAACATGAAGTTGGCAGCCTTCCTCCTCCTGTG |
| | | | | ATCCTCATCATCTTCAGCCTAGAGGTACAAGAGCTTCAGGCT |
| | | | | GCAGGAAGACCGGCTTTTGGGTACCTGCGTCGAGCTCTGCAC |
| | | | | AGGTGACTGGGACTGCAACCCCGGAGACCACTGTGTCAGCAA |
| | | | | TGGGTGTGGCCATGAGTGTGTTGCAGGGTAAGGACAGATGAA |
| | | | | GAGTTATCTTAAGGATCATCTTTCCCTAAGATCGTCATCCCT |
| | | | | TCCTGGAGTTCCTATCTTCCAAGATGTGACTGTCTGGAGTTC |
| | | | | CTTGACTAGGAAGATGGATGAAAACAGCAAGCCTGTGGATGG |
| | | | | AGACTACAGGGGGATATTGGAAGCAAGGAAGAGGGGTTGTTC |
| 143 | IMAGE:503051 | AA151535 | 5′ | TTTTAATAAATCATCATTGTTA |
| | | | | AAATTGGTTTTAATTTTTTTTAATTGGATCTATCTTCTTCCT |
| | | | | TAACATTTCAGTTGGAGTATGTAGCATTTAGCACCACTGGCT |
| | | | | CAATGCGCTCACCTAGGTGAGAGTGTGACCAAATCTTAAAGC |
| 144 | IMAGE:509823 | AA054073 | 3′ | ATTA |
| | | | | AGAAATTGACGACTTCACACTATGGACAGCTTTTCCCAAGATG |
| | | | | TCAAAACAAGACTCCTCATCATGATAAGGCTCTTACCCCCTTT |
| | | | | TAATTTGTCCTTGCTTATGCCTGCCTCTTTCGCTTGGCAGGAT |
| 145 | IMAGE:509823 | AA054457 | 5′ | GATGCTGTCATTAGTANTTTTT |
| | | | | TTTTTTTTTTTTTTTTGATTAACATTCTTTATTTCACAGTATT |
| | | | | TTTGATCAGAAGTCTTAGAAATCATGATTCATCTGGTTACAAA |
| | | | | TCCCATGAGTTTCTCTTTGAATGAACCTCTTGCTTCCAGTCCC |
| | | | | ATACAACGCATCTCCCACCAGCCCCAGTGGGTTGTAACTGTGA |
| | | | | TTCAACACTGAGTGCTCGCTTGGAAAGGAGGTGGAGCTCAACT |
| | | | | TCCAACTCAGAGGGCCTCTCCCACTGCTCTCAGGGAAATGCCC |
| | | | | ATGATTCACTTATGCTGTATCAACAACAAGTGCAGCTGGGCGC |
| | | | | TGCCTTTCCCAGCTGGGCCAAGCGGCTCCTAGGGGGGAATCTC |
| | | | | CACCCTCAGGAGGGCTTAGGGAAAGGGGAAGGTNTGAACGAGT |
| 146 | IMAGE:51406 | H18950 | 3′ | TCAGGGGCCCNGG |
| | | | | CTCAAAGGGCTGTCACCATCACCTGCTGCTAGGACACTACAAA |
| | | | | ACAATCAAATAATTCTTTTCTGTAATCCAATATGCAGCAAGCA |
| | | | | AGGGTGACCTCCAGTGGCCCACTCAAGTCCATGAGCCATTATC |
| | | | | TAGGATACTTTCTCTCTCTTTCATGCAGTTCAAAGCCCAGGTA |
| | | | | TCTCTCAGATCTGCTGCCTGAGAAATAAGCTCCTTTATCAGTT |
| | | | | AGCTGTTTTATCATTAGGATACAAGACAGCCCAGTGTCATCA |
| | | | | ACAGTGAGCAAATCTGGGCATGGTGTTTGTCTCGTACAGTTG |
| | | | | GGGATAGGGAGGCCATTCATTCCCATGGGGGCACAGCTTAAC |
| | | | | ATTATCCCCCAGTGGATTACTTTTCGATTACACTTGAAGGAG |
| | | | | GACCACCTTGTCTTTTAAAGGTTCANTTTCCNGGGGGTTGGC |
| | | | | ANTGTTTCCAACCCAGTTGTTTNCCAGCTGTTTCACAACCAG |
| 147 | IMAGE:51406 | H19393 | 5′ | TGTGNTGGATT |
| | | | | TTTTTTTTCACCTTAGGCAGCTTTTTATTTTGCATCCTTTTT |
| | | | | TTCAACTTTGTCTTCTATTAGCTGTNAAGAAATACATGTCTG |
| | | | | CTAAAGTTACACGATCTTCGCACAACAGCAACCTACACATTA |
| | | | | GTCTACAAAGGGGAACAAACCCAAATTCCTCAGAAGTCTGAG |
| | | | | TCCACTGTTGCCTTCTTTCTGGCCATCTGGAGGTTACAATAT |
| | | | | AGCACAGAATGACTATGCAAGTTAAATATTCATCTTAGACAT |
| | | | | GGACATTTGCTTTGGGACTCCTAAAGTGGAGTCAAATTTGAT |
| | | | | CTCTACAGAAACTCTACAATGTAGCAGAGCACTGTGCGTACT |
| | | | | TATTGACTCCCGGGACAAGCCGGAAACCCCGGAATTTGTCAT |
| | | | | TTCTATCAGGTTTTTATATAAATTGGTTCTTACCTACTTATT |
| 148 | IMAGE:53092 | R15785 | 3′ | GATGGCTTACAATTTGGGCCATTN |
| | | | | TTTTAGGGTGAATCCTATGTGTAGAATTGCTTGGTCAAATGG |
| | | | | TAAGCAATAAAAGCAATAAACAGTTGACGCCCTTGATTCGTT |
| | | | | TTTCTGTTCAAATGTCAATTCTTTAAAGAGGCCTTTTCTGAT |
| | | | | GACTCATGTGAAAAACAGCTCACTGTCATTCTCTGGCTCTTT |
| | | | | ACTCTGCTTTATTTTCCTTTGAAGTCCTTATTGGACATCATA |
| | | | | TTATCTATTAATTTGCTTATTGTTTATCTTTTCTACTGGACT |
| | | | | GTACACCTCATGTGTGTAGGGCATTTGTTTTGCTCACAGCTG |
| | | | | TCAGGTATTGGGGATACCCCAATATCTAACACAGTAAACAAT |
| | | | | CAAGAATTATTGGGTTGAATTAATGAGTTAATAAAATTAAAT |
| | | | | ACTGGCCTCATTGAAGGGGTTATATAGATTTTTAAAAAATAC |
| 149 | IMAGE:564567 | AA127395 | 3′ | CNGGTTTTGTGCNCCATGGACCCAAACTGG |
| | | | | TATTGCCATCTAATGCTCAGAACACACTTGTATTGCAAGAAA |
| | | | | ATATTTTTTTGCTTGTTTTTTTGAGACATAGTCTTGCTCTGT |
| | | | | TGCCCAGGCTGGAGTGCAGTGGTGATCTTGGCTCACTACAAC |
| | | | | CTCCGTCTCCCGAGTTCAAGTGATTCTGGAGCCTCCCAAGTA |
| | | | | GCTGGGACTACAGATGCATGCCACCATGCCCAGCTAATTTTT |
| | | | | GTATTTTTAGCAGAGATGGGGTTTCACTATATTGGCCAGGCT |
| | | | | GGTCTCAAACTCCTGACCTCGTGAATCCACCCACCTTTGGCC |
| | | | | TCCCAAAAGTGCCAGAGATTACCAGGCATGAAGCCACTGCAC |
| | | | | CTGGGCCTCAAGAANAATTATATATCACGTGGAATAGGGATN |
| 150 | IMAGE:564567 | AA127577 | 5′ | GTAGTCTCTGCACTGATTTNG |
| | | | | AGACTGCACGTGGTTCTTAGAGCCTACAGTGGCTGACAGAGT |
| | | | | ATTGGGTATTAACGTTAACGGATCCTGTGATGTGGCGGTGAN |
| | | | | TGCAGCTGTGATCCACGAAGTCTCTGAACAGGGCTTAGAAAC |
| | | | | TGACTGCACTTTGTTTTTAACAGGAGCCTACGTGAAGAAGAG |
| | | | | AGCACACAATTTTAAAAGTTGATTTTATATTCTCTGAGTTTT |
| | | | | TCTTCTTGCTTCAACAAAACTCTAGGAAATGCCATAAGCTGA |
| | | | | AAGAACATGACCTTCCTCAGACATCTCTTCTCTCCCTTTCCA |
| | | | | AACACAACTAGGAGTCATTTTTTTATTGGTGCTATGCCATTA |
| | | | | AGAGGTCTTCCTGCTTACGCTTTCCTCAGAGCGGATTGTTGG |
| | | | | CTGGGCGCAGTGGCTCAGTGCCTGATATCCCAGCACTTTGGA |
| 151 | IMAGE:592125 | AA150538 | 3′ | AGGCCG |
| | | | | GCATATGACTTGGAATTGGCCTGTACCAAACTCTGGGACCTG |
| | | | | CTGTTCCTGGATCCAGTGGTCTTGTTCCAACAGATGAATCTA |
| | | | | TAAAATATACCATATACAATAGTACTGGCATTCAGATTGGAG |
| | | | | CCTACAATTATATGGAGATTGGTGGGACCGAGTTCATCACTA |
| | | | | CTAGACAGCACAAATACGAACTTCAAAGAAGAGCCAAGCTGC |
| | | | | CTAAGTACCAAGCTATCTTTGATAATACCACTAGTCTGACCG |
| | | | | GATNAACANCTGGACCCAATCAGGGAAAATCTGGGAAAGCAC |
| 152 | IMAGE:592125 | AA143087 | 5′ | TTGGAAAAACT |
| | | | | ATATTATAAAAGCATTTTATTGAACACATTCTGGAGGTAGTT |
| | | | | AGAACCAAAACAAAATTTGGGATTGGGGTGGGGATTCTGTTT |
| | | | | TGATGATTTAGATTTGGGAAAACTTTGGGTTCTCGTGTCAGC |
| | | | | AGGGGCCATGCTGTGGGAAACCTGAAGGCTGATTTGAAGCAG |
| | | | | AATATAGAACTGCGGCACGGGAGACCAGGGGCTGGGAATGGG |
| | | | | GCTCTCCTGGGAACCAAAGAATGTGGTTCTGCAATTGGCTTG |
| 153 | IMAGE:592540 | AA160507 | 3′ | GTCTAGACTACTCTCCAGAAAAG |
| | | | | CAGCGTCAAATTTGTCTCCACCACCTCCTCCTCCCGGAAGAG |
| | | | | CTTCAAGAGCTAAGAACCTGCTGCAAGTCACTGCCTTCCAAG |
| | | | | TGCAGCAACCCAGCCCATGGAGATTGCCTCTTCTAGGCAGTT |
| | | | | GCTCAAGCCATGTTTTATCCTTTTCTGGAGAGTAGTCTAGAC |
| | | | | CAAGCCAATTGCAGAACCACATTCTTTGGTTCCCAGGAGAGC |
| 154 | IMAGE:592540 | AA160595 | 5′ | CCCATTCCCAGCCCCTGGTCTCCCGTGCCGCAGTTC |
| | | | | TTAAAAAAATTTTTTTTATTGAAGAACAGCATACATAAAGAC |
| | | | | ACACCAGTTTTAAGTGCACAACCCATTTCTCACAAAGTAGAC |
| | | | | ACACTTGAGTTTCCACCACCAGGTGAAGAGATAAAGCCTTAT |
| | | | | TAGCACCTCAAAAGATCCTCCCCTTGTGCCCCTTTTCCCATT |
| | | | | ACCCACCCTCCTCCCCAAAGGTAACCACTATCCTGACACCAT |
| | | | | AGGTTAGTTTTTGCCTGTTTTTAAACTTCACAAAAATGGAAT |
| | | | | CATACAGTCTGCATTCTTTAATGTCTGGCTCCTTTCGCTCAA |
| | | | | CATCATGTTTGTGAGATTCATCCAGGTTGCCTGTAGCAGCAG |
| 155 | IMAGE:60201 | T40444 | 3′ | TTCATT |
| | | | | TAAGCCCAGCACTTTAGGAGACCAAGGTGGGAGGATCACTTG |
| | | | | AGCCCAAGAGTTCAAGACCAGCCTGGGCAGTGTGGCAAGACC |
| | | | | CAATCTCTCATTAAATAAATAATAATAACCAAACAAAAAAAT |
| | | | | AACCACCACTTTTCACACTCACCATGGCAAAATTTAAAAACC |
| | | | | TAACAATTCCAAGTGTTGTCAAGGCTATAGGACAACTGCTGG |
| | | | | TGAGAGTGCAAATTGGTATAACCACTGTGAAAAAAAAGTTTG |
| 156 | IMAGE:60201 | T39159 | 5′ | GCATTATGTATGAAACT |
| | | | | AAATTTTGAGTGACTTGAGTCTCTTGCAGTCCCTGATTACAC |
| | | | | AGAACCTTTCTGGGCTACTTGGAGCATCACGAATAGTCTTTC |
| | | | | CTGTACTTACCAGATTTCAAGTATTCATAACTTGACTCCCTA |
| | | | | AGTGTACAAGTTGGGAATAGTACAGGGCCAAGTTCAAGTCGC |
| | | | | ATATGCTGTACTGTTCCTCCTGCAAATGTGGGGAAAGAAGAG |
| | | | | GGAGATACTAGAGGAACTGAGGCTCCACCCATTCATTCAGTT |
| | | | | GCTCTAAGCACCAGAGGACTTGTTTCAGAAAAGGGGAGTGGG |
| | | | | AACGCCCTCCGACTTTGCCCTCCTCCGGAGCATCTCTGGGAC |
| | | | | GCAGGGAGTCTGGCTAGCGTTAATAGGAAAGGTTGCTCGGCA |
| | | | | GAGTGGCCCTGGAGTACTGACTTGTCTCTCCCTCCTTTGTCA |
| | | | | AGGTCCATGTTTTTCTGGCTCTTCCTGCACACTCATCCCTAG |
| 157 | IMAGE:626199 | AA188775 | 3′ | ATTA |
| | | | | GACACCAATTCATAGCATTTATTGACATTTCCATTTAAAATG |
| | | | | CTAGGAAAGCTGTATNAATTGTAAACATGGAAACCAAATACT |
| | | | | TGCATAAATTATTTCAAAAACTCTACAGCACATTAGAAAACA |
| | | | | GTGCAGCTATTGAAGGATAGAAACATAAAACCGACAAATAGA |
| | | | | AGGGAGGGGCCGATTATTAAATCGTATACCCATACTGAGATT |
| | | | | TCAGTGCCTGTTTGAGGACCAGCAAACCATGATTGTCAAGTT |
| | | | | TAAGTTGCAGTATTGATGCCACAGTTGGCCTCAATTTGCTCT |
| | | | | GCACATTTCGTACATTAACGCTCATAATCTAGGGATGAGTGT |
| | | | | GCAGGAAGAGCCAGAAAAACATGGACCTTGACAAAGGAGGGA |
| | | | | GAGACAAGTCAGTACTCCAGGGCAGCTCTGCCGAAGCAACCT |
| | | | | TTCCTATTAACGCTAGCCAGACTCCCTGCGTCCCAGAGATGC |
| | | | | TCCGGAGNGGGGCAAAGTCGAGGGCGTTCCCACTCCCCCTTT |
| 158 | IMAGE:626199 | AA188785 | 5′ | TCCTGAAACCAAGTCCTN |
| | | | | TGAGGATTCATATTGTCATTTTACTTATTTACAGAATCAATA |
| | | | | AACCAACACATACACACTATTCAGAGAGGTGGGAAGTGCTCT |
| | | | | GCAACCTTCTCCCTCAAACCTGGGCCCAGACCCCAGTCCTGG |
| | | | | ACCACTGCATCCACCCAGCAGGAAAGGGGTCCAGCCAAGACT |
| | | | | TTTCCTGACTTTGTAACTTACAGACACAAGAGAATAGAGGGT |
| | | | | AGAAGGGAAATTCTTGGCACCTGGACTAGAGTGAGATAAAAG |
| | | | | GAGAGTAGGAAACCAGTGATAGGAGAGAAGTGAGGGAGGTAC |
| | | | | ATACAGTTTTATAAATAACTAGACAAGGTCTGAGCACTTTGG |
| | | | | GTGGGGATGGAGTGAGAAAGGCTACAGGCATGTAGGGGCCTA |
| | | | | AGTGGAAAAGGAAGAAATAGTGCTTGGGGCCAGAGCGGATGA |
| | | | | GAGATCAGCTCTGGGCCTTCTTTTGCCCCATCTGTAAACCAG |
| | | | | TGGTTGCCTAGGTGGTGTCAAACAGCCCGTCCCGGTTATCTA |
| 159 | IMAGE:681906 | AA256172 | 3′ | GG |
| | | | | CTCTGGCTATGGGGATAGGAGGAGAGCTCCGGAGGTCTCTGA |
| | | | | CCCCTCCCAAGGATCATGCCGCAGCCCCACTGACCCAGGAGT |
| | | | | AGGGGCCTAAGGGCAGGGAACCTGGAACTGGGCTGTGTGTTC |
| | | | | TGCAAGAAATTGGAGCCGGTGGCACGGCATATGAGGATGCTG |
| | | | | GCCTGGAAGGGGACTTCAGAAGCTACGGGGCAGCAGACCACT |
| | | | | ATGGGCCTGACCCCACTAAGGCCCGGCCTGCATCCTCATTTG |
| | | | | CCCACATCCCCAACTACAGCAACTTCTCCTCTCAGGCCATCA |
| | | | | ACCCTGGCTTCCTTGATAGTGGCACCATCAGGGGTGTGTCAG |
| | | | | GGATTGGGGTGACCTGTTCATTGCCCTGTATGACTATGAGGC |
| | | | | TCGAACTGAGGATGACGTCACCTTCACCAAGGGCGAGAAGTT |
| | | | | CCACATCTTGAACAATACCTGAAGTGACTGGTGGGAGGCTCG |
| | | | | GTCTCTCAGCTCCGGAAAAACTGGCTGCATTCCCAGCACTAC |
| | | | | GTGGCCCCTGTGACTCAACAAGCTGAGAATGGTATTTGGAAA |
| 160 | IMAGE:681906 | AA256231 | 5′ | ATTGGGA |
| | | | | GTTTATTCTACTTTTATTTCACATATATAAAAACAGCTTATA |
| | | | | ATTGTACTGAACACAAAATACAAACAAATACATTTTATTGCA |
| | | | | CATAAAAATATTTTAAATGAAGTATTGAAGTATTGCACGTAA |
| | | | | TAGAATTGATTTAGGAAAGTCACAAACCTATTATAAGACTAG |
| | | | | TATTATTCTAGGTCTGAAGATTACAGAATATTTCCTAATAGA |
| | | | | GATTTGCCACATCACATATTGCACATTTTCCAACACTATTCT |
| | | | | ATGTCTTGCAAATATTCCTCATAGTCTTTGCTTATGTCTTTT |
| | | | | CTCTGTAAGACACTGTATAAAAGATTATAAAGGCAAAGAAAT |
| | | | | ATGTACCATCGAAAAGGACCTGTCTACAGCTGAGGAAGTAAA |
| 161 | IMAGE:704459 | AA279755 | 3′ | AAAATAAATACACGATCATCCCATTCTTTTG |
| | | | | ACAGCTCTTTGCATCCGGAGAGTGGACAAGAAAATGATGCCA |
| | | | | CCAGTCCCCATTTCTCAACACGTCATGAAGGGTCCTTCCAAG |
| | | | | TTCCTGTCCTGTGTGCTGTAATGAATGTGGTCTTCATCACCA |
| | | | | TTTTAATCATAGCTCTCATTGCCTTATCAGTGGGCCAATACA |
| | | | | ATTGTCCAGGCCAATACACATTCTCAATGCCATCAGACAGCC |
| | | | | ATGTTTCTTCATGCTCTGAGGACTGGGTTGGCTACCAGAGGA |
| | | | | AATGCTACTTTATTTCTACTGTGAAGAGGAGCTGGACTTCAG |
| | | | | CCAAAATGCCTGTTCCTGACATTGTGCTAATCCTGCTGCAAT |
| 162 | IMAGE:704459 | AA279883 | 5′ | GATCCTGAAAAGGGCATTGACTTT |
| | | | | TTTTTTTTTTTTTTTTTTTTTTTTTTTTTTTTTTTTTTTTTCA |
| | | | | ACTGAAGTTCTATTTATTTGTGAGACTGTAAGTTACATGAAGG |
| | | | | CAGCAGAATATTGTGCCCCATGCTTCTTTACCCCTCACAATCC |
| | | | | TTGCCACAGTGTGGGGCAGTGGATGGGTGCTTAGTAAGTACTT |
| | | | | AATAAACTGTGGTGCTTTTTTTGGCCTGTCTTTGGATTGTTAA |
| | | | | AAAACAGAGAGGGATGCTTGGATGTAAAACTGAACTTCAGAGC |
| | | | | ATGAAAATCACACTGTCTTCTGATATCTGCAGGGACAGAGCAT |
| | | | | TGGGGTGGGGGTAAGGTGCACTGTTTGAAAAGTAAACGATAAA |
| | | | | ATGTGGATTAAAGTGCCCAGCACAAAGCAGATCCTCAATAAAC |
| 163 | IMAGE:712049 | AA281635 | 3′ | ATTTCATT |
| | | | | CAGACACTGTATCTTTAGATTGATGTCGACCACAAAGTTCAGC |
| | | | | CAGAGCTTGAGGCTAGATGCACAGCCTTGCTATTGGGAAGAAG |
| | | | | GCCTTTTCTAGCTGTACAACACAGTCTCACTGGGCATTCATCC |
| | | | | AGAAATAGAGAAGAAAGTCTGCCAGACTTGAGTTATGTTGTCT |
| | | | | TTTATTAGCAGGGAATGTCATCACAGATTGGATAGTACATCCA |
| | | | | GGTGCAATGTCACCATCAGCAAGGTCAGCTTGACACTCAAGTG |
| | | | | GAAGATTAGGGAAGAATGACTAGGATAAAAAAAAAAGGAGGGC |
| | | | | ACCAAGGGAAAGGGATGATGGGGTGAGCTGGCGAGTGTGGGTG |
| 164 | IMAGE:712049 | AA281696 | 5′ | GGAAATGAAA |
| | | | | AAAAAGACAAAGAAACTTTATTTATACAAAACTCCACCCCTTC |
| | | | | TGTTCCACTCTCCTCAGCAAACACAGATAACAGGTGATGAAAC |
| | | | | TAAAACACACAGACGAGCATTACTCAACCCAAGGTTCCCGCCT |
| | | | | TCCCTAGCACCTGAGGTCTGGGCCAACATGCAGGCTAACTGGT |
| | | | | GCCTTATGCCTGCTGTCTGGATTGCCCGGCCCACAGGGTGGCT |
| | | | | GAGCATATTTATTCTGGGGGTTCCATGCATACGAGGAGCCCCC |
| | | | | AGCCATACAGCTGGGCATGGGTGTTTGGCAGCAAATTGTCCC |
| | | | | TGCTTTAGTCACAGCAATTTTTCATGTCCTCTGTTTGCTCCC |
| 165 | IMAGE:714453 | AA293306 | 3′ | CTTAAA |
| | | | | CCAGTCCCTGTCCCCTTGTTCACCTTTGGACTGGACAGGGAG |
| | | | | CCACCTCGCAGTCCGCAGAGCTCACATCTCCCAAGCAGGCCC |
| | | | | CAGACACCTGGGTCTGGAGCAGGGGGAAAAGGTAGAGGACAT |
| | | | | GCCAAAGCCCCCACTTCCCCAGGAGCAGGCCACAGACCCCCT |
| | | | | TGTGGACAGCCTGGGCAGTGGCATTGTCTACTCAGCCCTTAC |
| | | | | CTGCCACCTGTGCGGCCACCTGAAACAGTGTCATGGCCAGGA |
| | | | | GGATGGTGGCCAGACCCCTGTCATGGCCAGTCCTTGCTGTGG |
| | | | | CTGCTGCTGTGGAGACAGGTCCTCGCCCCCTACAACCCCCCT |
| | | | | GGAGGCCCCAGACCCCTCTCCAGGTGGGGTTCCACTGGAGGC |
| | | | | CAGTCTGTGTCCGGCCTCCCTGGCACCCTCGGGCATCTCAGG |
| 166 | IMAGE:714453 | AA292025 | 5′ | AAGAGTAATCCTCATCA |
| | | | | CAGATTCTAACAAGAATACTTTTATTATACACGTATCATACA |
| | | | | CACAACAATTATTTGGGGAACATTTACAGGCAGAGAGTTCAA |
| | | | | TTCCAAATCTCCATTTCACCCACACACACTGTACTGCACACT |
| | | | | CACCTTAGGGTTCAGCCCAACAGGAACGAGACAAAGTTATTG |
| | | | | CTTTCTGAACAGAGAGTTTCAATTAAATAGAATCTTCCAAGC |
| | | | | CAAGAACAGAGCCCAGCATCCTCTTAATTCTTAATACCCTGT |
| | | | | ATATATATGAATAAAACCTTATGATGTGTTATAGATTACCCC |
| | | | | ATCACCATTAAAAGTTAATATTAAAATTGGATCCCATGTCTC |
| | | | | AAAAAAGTCGTAAGAAGTGCACCAGTATTTACAGACCCCATT |
| | | | | AAATTACGCATAAATAAAATCTGTACACTCAACGCACTGTTT |
| 167 | IMAGE:725680 | AA394236 | 3′ | C |
| | | | | CCTGATTGTCATAGACAAATCCTACATGAACCCTGGAGACCA |
| | | | | GAGTCCAGCTGATTCTAACAAAACCCTGGAGAAAATGGAGAA |
| | | | | ACACAGGAAATAAAATTGGAACGAAGAAAGGTTAGGAGAGTA |
| | | | | GGGAAGGAACAGGACTGCAAAAATCCTTCTCCACCGCACAGA |
| | | | | CTGGGAACCCCTCCTGGCCTGGGGGAAGAGTTTGTTACCTAC |
| | | | | CTTACTATTTAAAGAGCCTTCACTGGTTCTGCATCACCCGCC |
| | | | | CCTGGACTTCTTAGTTGTTTCTCTAGCGCTGAGCTATCTCCT |
| | | | | AACTTTGGACCTATTATCAGAAGGTGACAAGTACTGGCTCTT |
| | | | | TATTCATTAAGCTTTTTTTTTTTGAACCCCATTCTTTCCTTC |
| | | | | TCTGAAAGTGGTGCTATAAGTTTTAGAATCTTTTAAATACAT |
| 168 | IMAGE:725680 | AA399334 | 5′ | TCCCTGGGCCAACAGACCCACACACTTAGCCATTGAAATGT |
| | | | | TTTTTTTTTGCATTTGTAACATGCACATTTATTCAGAACAAA |
| | | | | CAACTCATTAATTTATTCCAAAATAATTTCACTTGATAACTT |
| | | | | GAAATACAGAGTAAAACAAATTGGTCAGGTAAATATACATGT |
| | | | | AACTTAAAAAGAAACAGTCATGTACTTTAGGCATAAGGACAA |
| | | | | TGCTTTTCTCTTTTACAAATTCTTAGTTAGGTCAAATTCTCT |
| | | | | GGAAGTCACTACATTTCTTTACTGTGATGTGTTTTGGGTGAA |
| | | | | GTTACAACCTATTTGCAAATCACATCACTGGTTTGTCCAAGC |
| | | | | AGAGGTAGATGAGAGGTAAGCTCCTCTCCTGCTAAAAGTCTC |
| | | | | CTAAAAACAGCAAGAAAATATTTTTATGTGTTCAAAAATGCT |
| | | | | CATTTATTTATATTCCTAAATTTTCTTTTACTCAGTATAATA |
| | | | | TAGATAATTTAAAAGTAAGTAGAATATTTTTATTATATATGT |
| 169 | IMAGE:742904 | AA405815 | 3′ | TTTATTTTTATACTTCTAGTTAAAT |
| | | | | GGACTCACGGGCGGGGCATGATGGTGGTGGGTACGGGCACCT |
| | | | | CGCTGGCGCTCTCCTCCCTCCTGTCCCTGCTGCTCTTTGCTG |
| | | | | GGATGCAGATGTACAGCCGTCAGCTGGCCTCCACCGAGTGGC |
| | | | | TCACCATCCAGGGCGGCCTGCTTGGTTCGGGTCTCTTCGTGT |
| | | | | TCTCGCTCACTGCCTTCAATAATCTGGAGAATCTTGTCTTTG |
| | | | | GCAAAGGATTCCAAGCAAAGATCTTCCCTGAGATTCTCCTGT |
| | | | | GCCTCCTGTTGGCTCTCTTTGCATCTGGCCTCATCCACCGAG |
| | | | | TCTGTGTCACCACCTGCTTCATCTTCTCCATGGTTGGTCTGT |
| | | | | ACTACATCAACAAGATCTCCTCCACCCTGTACCAGGCAGCAG |
| | | | | CTCCAGTCCTCACACCAGCCAAGGTCACAGGCAAGAGCAAGA |
| 170 | IMAGE:742904 | AA405814 | 5′ | AGAGAAACTGACCC |
| | | | | TTGCAGTGGAGATGGGGTTTCATCATGTTGCCCAGGCTAGTT |
| | | | | TTCCTTTCTATATACAGAAAAATTTAAAGTGAATGTGATGTT |
| | | | | GGAGAGAGTGGGAAGGAAAAGTAATGGCAAGTATGCTTGCTC |
| | | | | ATTACCAGGCACTGTGCTAAGCTCTGTGAATACACAGATAAG |
| | | | | TAAAATCCACGCTGTTTCTCAAAGAACTCACAATCTGTTTAA |
| | | | | GAAGCAGATGTCTATACAATAATTTTATAACTATTATTCAAT |
| | | | | GTGATTAGTACTCACATAGCTCTATATAGAGTGTTATAGAAG |
| | | | | AATAAATTAGAGAATATCTCATTTTTCCTCCAGTGGTTTAAA |
| 171 | IMAGE:754479 | AA410188 | 3′ | AAGATGTCACAGAAACTGAATTGTAAATGGTACGGAAATA |
| | | | | GGAGATAAGTTGCCTTGATTCTGACATTTGGCCCAGCCTGTA |
| | | | | CTGGTGTGCCGCAATGAGAGTCAATCTCTATTGACAGCCTGC |
| | | | | TTCAGATTTTGCTTTTGTTCGTTTTGCCTTCTGTCCTTGGAA |
| | | | | CAGTCATATCTCAAGTTCAAAGGCCAAAACCTGAGAAGCGGT |
| | | | | GGGCTAAGATAGGTCCTACTGCAAACCACCCCTCCATATTTC |
| | | | | CGTACCATTTACAATTCAGTTTCTGTGACATCTTTTTAAACC |
| | | | | ACTGGAGGAAAAATGAGATATTCTCTAATTTATTCTTCTATA |
| | | | | ACACTCTATATAGAGCTATGTGAGTACTAATCACATTGAATA |
| | | | | ATAGTTATAAAATTATTGTATAGACATCTGCTTCTTAAACAG |
| | | | | ATTGTGAGTTCTTTGAGAAACAGCGTGGATTTTACTTATCTG |
| 172 | IMAGE:754479 | AA410567 | 5′ | TGTATTCACAGAGCTTAGC |
| | | | | TTTTTTTTTTTATATGTAATGACTGTAGTAACCAGTTTATTA |
| | | | | CACAGATTAATCATTCTTGAAAGTACAAGCTCCAGAGGAGAA |
| | | | | TCTGGGTCTTTAAATATACACAAGTATTTCCATCAAATGAAT |
| | | | | TTTCACCCTTACATCCAAATAGACCTAAGCGGTTAAAAACAT |
| | | | | AAGAAAAATAAGAGCTATTAGCTATGTATTAACTGAGAAACC |
| | | | | ACATACAAACCAAAGAATATGGAAGAGAGAAAGAAGGGCTGA |
| | | | | AAGGCCAAAGGTTGAAGGGGTAGGGAGATGTAAAAGAGTTGG |
| | | | | GAACAAAGCCCATCACACTTGATGTACTAATAGCTCCAATCC |
| | | | | ATTTAAATGTTGACCAGTTAAACTTAGACCTTAAAATGCAGG |
| 173 | IMAGE:773617 | AA431869 | 3′ | ATGTGGGTAGAG |
| | | | | GAGATTGCTCGGATCTACAAAACAGATAGAGAAAAGTACAAC |
| | | | | AGAATAGCTCGGGAATGGACTCAGAAGTATGCGATGTAATTA |
| | | | | AAGAAATTATTGGATAACCTCTACAAATAAAGATAGGGGAAC |
| | | | | TCTGAAAGAGAAAGTCCTTTTGATTTCCATTTGACTGCTTTC |
| | | | | TATGAGCCCACGCCTCATCTTCCCCTGTGCACATGTTTACCT |
| | | | | GATACAGCAGTGCTGCGTGTTGTACATACTTGGAACAACAAA |
| | | | | CTAGAAATACTGTACTTCTGTACCAACATTGCCTCCTAGCAG |
| | | | | AGAAGTGTGTGTGTGACAAGCCAGTTCTACAGGCATTACCTA |
| | | | | GGTGTGAGACTAAAAGCTTTTCTTATTGACTTAAATTTGGAT |
| | | | | AACAGCAAGGTGTGAGGGGGGTGGTGGGTATGGTGTGTGCTT |
| | | | | GGATGGGAAAGAANAGGCTCCACTCACCTATAGGAGATTATT |
| 174 | IMAGE:773617 | AA431868 | 5′ | TTTAAGTGGAATCC |
| | | | | GCGCCGCGCGCCCGAAAGGCTGCGGCCGTGGGCCCGTCCCGC |
| | | | | AGACCCTGTGGTTGGGCTGACCCCGCTTCAGGGTGCCGTACA |
| | | | | CGAAGACTAGGGCCATCCGGGCAGACTGTAACTTGTTTCTTC |
| | | | | AAGGAAGTGTTGCCTTAGAATCCAGATCCACAGTAAGCCTGA |
| | | | | GAGTCTTAAAAACTTTTGACTTCAGAATCCTTCCACATGATT |
| 175 | IMAGE:781088 | AA430002 | 3′ | CAAGAAAAAGTTAAGTCCACTTCACAGGGTGAC |
| | | | | GAAAACTGGATATTTGGTCCCCATGGACTTTCTGGTCACCCT |
| | | | | GTGAAGTGGACTTAACTTTTTCTTGAATCATGTGGAAGGATT |
| | | | | CTGAAGTCAAAAGTTTTTAAGACTCTCAGGCTTACTGTGGAT |
| | | | | CTGGATTCTAAGGCAACACTTCCTTGAAGAAACAAGCTTACA |
| | | | | GCTCTGCCCGGATGGCCCTAGTCTTCGTGTACGGCACCCTGA |
| | | | | AGCGGGGTCAGCCCAACCACAGGGTCCTGCGGGACGGCGCCC |
| 176 | IMAGE:781088 | AA430230 | 5′ | ACGGCTCCGCAGCCTTTCGGG |
| | | | | TTTGCCCAGCAAAGACAAATATATTTGTCCCTGTTGCTACAA |
| | | | | TAGGAAGTTAACAATCTGGCAAGATATCTGAACACAAGCAAA |
| | | | | TGAAAACAGTTCACCTAACACCCATGCAAATTATAAATTTCC |
| | | | | TCCCATATACAAAATGATGAGAAATAACAGCAAAAATGTATA |
| | | | | CTTTCTTATTTTTGAACTTTTAAAGTTCTAGTTTGGTCTTTG |
| | | | | AATCAAAACAAAGTAAAAGATGTTTATAAAAGCCATTTCCTT |
| | | | | TTCTTTCCCCACTATGCTCATTTGACTTGCTCTTCCCCCTAT |
| | | | | AGGGTACCCTGAGTCATTCAGAGAAGGAGAATTAATAGCACT |
| | | | | GAGTTGGTGATGAAGCTCCTGTTAGGACATATGGCTTCACAA |
| | | | | AAAGAAATACTTCCAGATAAGTCAGAGAGACAGTTGGACGTC |
| | | | | TTGAGCAAATCTTGAAAGAGATAGGGAAGAAAGCAGAAGTTG |
| 177 | IMAGE:782141 | AA431190 | 3′ | TTGGGTGGT |
| | | | | GTGCTCCCACTTTGACAATGATGAAATTAAAAGGCTGGGCAG |
| | | | | GAGGTTTAAGAAGTTGGACTTGGACAAATCAGGGTCTCTGAG |
| | | | | CGTGGAGGAGTTCATGTCCCTGCCGGAGCTGCGCCACAACCC |
| | | | | GTTGGTGCGGCGAGTGATCGACGTCTTCGACACCGACGGTGA |
| | | | | TGGAGAAGTGGACTTCAAGGAATTCATCCCGGGGACCTCCCA |
| | | | | GTTCAGCGTCAAGGGCGACGAGGAGCAGAAGTTGAGGTTTGC |
| | | | | GTTCAGCATTTACGACATGGATAAAGATGGCTACATTTCCAA |
| | | | | CGGGGAGCTCTTCCAGGTGCTGAAGATGATGGTGGGCAACAA |
| 178 | IMAGE:782141 | AA431516 | 5′ | CCTGA |
| | | | | TTTTGCAGTTACAACATTTACCACTTTATTATAAAGGCTACA |
| | | | | ACTCAGAAACAGCCAAATGGAAGACATGTATAGGACAAAGAA |
| | | | | AGATGTGGGGGTGGAAGAGGTTGTATGGAGCCTCCATGCCCT |
| | | | | CTCTGGATGCCATTGGTTGACTGGGGGAATTAATTCCCTGGT |
| | | | | GCTTCCAGCCTGCAAGATGAGCTCCTTCAACCAGCAAGTCCC |
| | | | | CAGTCAAAAGAGTGCACGGGGTGTAGCTGGAAGTTGAGCAGA |
| | | | | TGGTAGTTTGCATGGATGAGATAAAGCCCCAGGGGACAGGGC |
| | | | | AGCTACACATGAATCCAAATAGTCTAATCTCCAAAAGGAACA |
| | | | | GAGAGTGGATTCATACAACATACCAAGCCCGCCCCCTAAATG |
| | | | | CATCCCACTCAGGTCACTTATAAAGCTCCAAGGATGGGCCAA |
| | | | | GAACACAAGCTCTACACCAGGGAAACTTGGAGGCATCAGAAG |
| | | | | GACAGAATAAGACCCAGGTTCATAGGGGATGAAAAATCGAAC |
| 179 | IMAGE:782758 | AA448002 | 3′ | AG |
| | | | | CAACAATAGCGGGAATGAAGACTGTGCGGAATTTAGTGGCAG |
| | | | | TGGCTGGAACGACAATCGATGTGACGTTGACAATTACTGGAT |
| | | | | CTGCAAAAAGCCCGCAGCCTGCTTCAGAGACGAATAGTAGTT |
| | | | | TCCCTGCTAGCCTCAGCCTCCATTGTGGTATAGCAGAACTTC |
| | | | | ACCCACTTGTAAGCCAGCGCTTCTTCTCTCCATCCTTGGACC |
| | | | | TTCACAAATGCCCTGAGACGGTTCTCTGTTCGATTTTTCATC |
| | | | | CCCTATGAACCTGGGTCTTATTCTGTCCTTCTGATGCCTCCA |
| | | | | AGTTTCCCTGGTGTAGAGCTTGTGTTCTTGGCCCATCCTTGG |
| | | | | AGCTTTATAAGTGACCTGAGTGGGATGCATTTAGGGGGCGGG |
| 180 | IMAGE:782758 | AA448145 | 5′ | CTTGGTATGTTGTATGAATCCACTCTCTGTGCC |
| | | | | TTTTATTATTAAATGTATATTTTTAATAAAGCCAATAGTTAT |
| | | | | TTTACTTATAGGAGCTTTAAAAGATACAAAATGTAGAGTTCC |
| | | | | AGTTTGGAAGCATTGTAACTATACACACAATGTCCTGCTGAT |
| | | | | GCCCTAGCAAGGCACCCACGCCCAACCATGCAAAGGACACAC |
| | | | | ACGTTCACACATGCACACACATGCGCTTTGGCGAGACCCCTC |
| 181 | IMAGE:784104 | AA432052 | 3′ | TGCCAAGCGCACACCCTGGAAT |
| | | | | GGACTTAGAAGCCTTACAAATACATCTGTGCATTCTTGCTTC |
| | | | | AGACTTTACAACTGAGGGCCAGCCCAGTCTGGAAGCATCTCT |
| | | | | TATTAATGTTACAAGGAAACCGCTACCTCAGCAAACAAAAGG |
| | | | | AATGGAGGAGGAGACTTACAACTGTTTTGTATATAGACATTT |
| 182 | IMAGE:784104 | AA446737 | 5′ | TCAGGCACGTGCTTT |
| | | | | TTTTTTTTTTTCTGCTTCAATATAATTTTATTAGCAGTTATT |
| | | | | ACATCAAAATTCACATTTAGAGGATCCAGAGGACTGTCTTAG |
| | | | | AAAATTCTAAAGCATATTTAATTAGGTTTTAACAGTAAGGGA |
| | | | | GAACTTAATATAACACAGCCCTTAAAAAGTCAAGACTACTAC |
| | | | | TGAAAATTAAGTGCAGTTCTATCAAGAACTAGAAATGAACTG |
| | | | | CACGTGTAGTGTCACTTAAAGCAAAGCTTCATGAAAATATAA |
| | | | | TACACTTCTATGAATGTATCAGTGGCAAACATCATTGGCTTC |
| | | | | CAAAAAACTGACACTAAAGGAATTTCCAATCAAAACACAAGC |
| | | | | ACAGTGGCTTTCATTCAATATAGAGCTATGATAAGTCTATCA |
| | | | | AGAGACCCTGAATCCTTACGTACTTGTAATATGATTTTATGC |
| 183 | IMAGE:784218 | AA446867 | 3′ | TGTGACACT |
| | | | | TTTTTTTTTCACTCAATAAATTTTTATTAGAAATGCAGTTAC |
| | | | | ACTGAGAAAGGATTTCACAATGGTCAAATCAGTGCACAATAC |
| | | | | TACCTAGTTTTATACACTGAAAAAAATGTCTTGTCAGGCTAC |
| | | | | ATCATTTTAGAAGACACTTTACAGCATTCTTGTAGCATTAGA |
| | | | | AATAATGAATAGAAGAGCGTCAAGGTGAAAACAAACACCAAA |
| | | | | TTTGGTCCAATAATACTGATTGCTCTTTGTTAAAATTCCTTT |
| | | | | GATACAGGTACTTTTTATAAATGAATATGAATGAACATTCGG |
| 184 | IMAGE:784910 | AA447632 | 3′ | TTAAAATGACTTACTTGA |
| | | | | GGGTGACACCAGGCTTACCTTTTAAAGTTTAGTATACGGAGA |
| | | | | CAATTTTAATGGAAATAACTACTGTAGACTATTGAAGAATGA |
| | | | | TCTCTTTGTGATTTAAGAAGTGGCTGGATTGGAACTTTTAAT |
| | | | | ATGCTAATGTGGAAAATTAATTACCTTTATGAAGGTGGTTTA |
| | | | | TTACAAATAAGCACACTAACCCCTCGGAAGTTGTTTTACCTA |
| | | | | CTTTAAAAGTTTTAATGGATTGCACCTCTGTAAACTATTCCT |
| 185 | IMAGE:784910 | AA448033 | 5′ | AAAATGTGTATGATATATTTGAAAAGGCTTCCATTA |
| | | | | TTTTTTTTTAACTGTCCGCAAGTTAAAAAGATTTATTGCTAT |
| | | | | TCCAGGCTTCAAATGAGCCCAGAACTCAGGGCTGGTGTGTGT |
| | | | | TTCAGAAGTTGTTATGATGTAACAGGGTGGTAGAAAAATCCA |
| | | | | GGCAGTTTGATGTCGAGGCCACCCTCTCTTCCTTGGACCCCT |
| | | | | GCTCCAAAAGCAGCTGCTGGTGAGGCTCTTTCCCATCTGCCT |
| | | | | CATTCACCCAACAGGACTCCAAGACTGAGGCAGGCAGCCTTG |
| | | | | TGATCCCCACAGCTCACAGGTGAGAGGCTGCTCATACCTCTC |
| | | | | CTAGCACTGGAAGAGCCTTGTCCTTGGGACCGGACACTATGG |
| | | | | CTTTGGCCCTGTGGAGGGAGAAACGGTGCCACAGGAGTTGTC |
| | | | | TTAAGAGGACAAGGCATGCACGGTCTGAGATCAGAGGTTGTG |
| | | | | ACGTGGCCACCCATGAGCCAGTCCGTTTGGGACACATCACAC |
| | | | | TGCACAGCTTTTTAAAAAATAATTAGGCTGCAATCTTTTAAA |
| 186 | IMAGE:795173 | AA453471 | 3′ | ATGGTAAGATTTCATATACCAATC |
| | | | | AAAAAGCTGTGCAGTGTGATGTGTCCCAAACGGACTGGCTCA |
| | | | | TGGGTGGCCACGTCACAACCTCTGATCTCAGACCGTGCATGC |
| | | | | CTTGTCCTCTTAAGACAACTCCTGTGGCACCGTTTCTCCCTC |
| | | | | CACAGGGCCAAAGCCATAGTGTCCGGTCCCAAGGACAAGGCT |
| | | | | CTTCCAGTGCTAGGAGAGGTATGAGCAGCCTCTCACCTGTGA |
| | | | | GCTGTGGGGATCACAAGGCTGCCTGCCTCAGTCTTGGAGTCC |
| | | | | TGTTGGGTGAATGAGGCAGATGGGAAAGAGCCTCACCAGCAG |
| | | | | CTGCTTTTGGAGCAGGGGTCCCAGGAAGAGAGGGTGGGCTCG |
| | | | | ACATCAAACTGCCTGGATTTTTCTACCACCCTGTTACATCAT |
| | | | | AACAACTTCTGAAACACACACCAGCCCTGAGTTCTGGGCTCA |
| | | | | TTTGAAGCCTGGAATAGCAATAAACCTTTTTAGATTGCGGGC |
| 187 | IMAGE:795173 | AA453978 | 5′ | AGTT |
| | | | | TTTTTTTTTTTTTTTTTTTTTTTGGCTTTCTGGGTCTTTTAT |
| | | | | TTGTACCCATGTGTCTGTCACACCATGAATGTACCTGGGGAA |
| | | | | ATCAACTGACCTCCCTGAACATTTCACGCAGTCAGGGAACAG |
| | | | | GTGAGGAAAGAAATAAATAAGTGATTCTAATGCTGCCTAGGT |
| | | | | CACTCTCAACCCCCATTTACTGGCACAGTTGGGTGGAGAGAA |
| | | | | GGGAAGGGGTATGATTGTCCTGATGGCTCAGGGATAGAGGGC |
| | | | | ATGGTAGAAAGCAAAGTACCCACACAGGCCCCAGTTCCAGCT |
| | | | | GCGGAGGACACTTGGGCGCTCCAGGGACAGGACTTGCTGGTA |
| | | | | CACAGTCTGCCCTTCCCGACGCAGGCACACTGTGAATTGGTC |
| 188 | IMAGE:796297 | AA461304 | 3′ | AGCGATGACTGTCCGGTGCTGATACATTC |
| | | | | GAAACACCAGCTCATTTAAGCTTTCCCCAACGCCCGGCCCTC |
| | | | | CGGACGAGTACCTAACAACCACCGGCGCCCGCATCTGGAATA |
| | | | | GGCTGGCGAGATACTTAGTATCCGAGGGCTCGGGACTTGGCG |
| | | | | CCATCGAGGTCATGGGGACCCAGGATCCAGGGAACATGGGAA |
| | | | | CCGGCGTCCCAGCCTCGGAGCAGATAAGCTGTCCAAAGAGGA |
| | | | | TCACAAGTTTATTGCCCTGAAGAGACTGGCGGCACCAAGGAT |
| | | | | GTGCAGGTTACAGACTGTAAGAGTCCCGAAGACAGCCGACCC |
| | | | | CCAAAAGAGACGGACTGCTGCAATCCGGAGGACTCTGGGCAG |
| | | | | CTGATGGTTTCCTATGAGGGTAAAGCTATGGGCTACCAGGTG |
| 189 | IMAGE:796297 | AA459721 | 5′ | CCTCCC |
| | | | | TTTATTTNNTTGAATCTATTTAATTGCTCAGACTGTGCTAGA |
| | | | | GAATACGTACCATGAAATACATATATTTCATAAGGTTCAGTT |
| | | | | ACAAAATGGATTGTTTCAAATGGCAATTTCTTACACTAACCT |
| | | | | GATTATGAAAAAAAGAAGTCTGTATCATCTGCTTCCAAGTCT |
| | | | | GTTATGTCCAAATATATTTTAATTATGCATTTATTTTGCTAC |
| | | | | TTTTATAAATATTAGAGATTTCACCNTAAATTATTTTTGTAA |
| | | | | CTAGTTCTAGAACATGTTTNCCAATTATTATTNNCCTAATGG |
| | | | | GAGACATATAATTGACCNATGGTTTATGGCATATATGGTCCT |
| | | | | CTACACAGNGGAACCTNTTTTTAAAAGGAATAGGTAAAGGAA |
| 190 | IMAGE:80948 | T70057 | 3′ | AATGCGGGACGGCCTGGGCTCTCCAGGGCCAAGGGCCA |
| | | | | TTGCTCCAGTTTTTCAGAAGAAGTGAAGTCAAGATGAAGAAC |
| | | | | CATTTGCTTTTCTGGGGAGTCCTGGCGGTTTTTATTAAGGCT |
| | | | | GTTCATGTGAAAGCCCAAGAAGATGAAAGGATTGTTCTTGTT |
| | | | | GACAACAAATGTAAGTNTGCCCGGATTACTTCCAGGATCATC |
| | | | | CGTTCTTCCGAAGATCCTAATGAGGACATTNTGGAGAGAAAC |
| | | | | ATCCGAATTATTGTTCCTCTGAACAACAGGGAGAATATCTCT |
| | | | | GATCCCACCTCACCATTGAGGAACCAGATTTGTGTACCATTT |
| | | | | GTCTGACCTCTGTAAAAAATGTGGATCCTACAGAAGTGGGAG |
| | | | | CTGGGATAATCAGNTAGTTTACTGCTTACCCAGNGGCAATAT |
| | | | | CTGTGGATGGAGGNCAGTGCTACAGAGACCTGCTTACACTTT |
| 191 | IMAGE:80948 | T70123 | 5′ | TGGAC |
| | | | | GCAACTTGAATTGTATTTTTTATTGAAAAGAATTCAGGCTAG |
| | | | | AGTTGGGAGGAGGATGCAAGAGCTACTGGGAAGGGGGAGCTC |
| | | | | AGTCTGAACCTGGGGGATCAGGGGAGTAGGGGACTCTCCCCT |
| | | | | TGTCCACTGATGGGGGGTCTGGCTGTTACTCCTCTCCCTTCA |
| | | | | GCACAGAAAGAACTTGGTCAGTAAAAATGCCTGTGTAAGTGC |
| | | | | TCATGGCTGCTGTGCTTTTGCTGTACAAGTCCCTGAGTTTCT |
| | | | | CATCTACAGCGGGCAGGTATGTCTTCTCGTACAGGTTCTGGG |
| | | | | CGGCTGTCTTTGCTGACTCCCAGTAACTGGAGAGAGATTCCT |
| 192 | IMAGE:809523 | AA454580 | 3′ | TCACCTGGGTGAGGAAGGTCGGGC |
| | | | | AATTTGAGGTCCAGGGGACCGAACAGCCCCAGCAAGATGAGA |
| | | | | TGCCTAGCCCGACCTTCCTCACCCAGGTGAAGGAATCTCTCT |
| | | | | CCAGTTACTGGGAGTCAGCAAAGACAGCCGCCCAGAACCTGT |
| | | | | ACGAGAAGACATACCTGCCCGCTGTAGATGAGAAACTCAGGG |
| | | | | ACTTGTACAGCAAAAGCACAGCAGCCATGAGCACTTACACAG |
| | | | | GCATTTTTACTGACCAAGTTCTTTCTGTGCTGAAGGGAGAGG |
| | | | | AGTAACAGCCAGACCCCCCATCAGTGGACAAGGGGAGAGTCC |
| | | | | CCTACTCCCCTGATCCCCCAGGTTCAGACTGAGCTCCCCCTT |
| 193 | IMAGE:809523 | AA456474 | 5′ | CCCAGTAGCT |
| | | | | ACTGCTCTTTTATTCAATGGAACATCCCCGCTTTAGCCAGTG |
| | | | | TTGAATCTAACACCGAAAAAAGCCCAGAGAAATTTCTGCAGA |
| | | | | TAAACCAGTGAAGAGAACGCGCAGTATACATTATTGTCAACA |
| | | | | GAATCACTTCATGGAGAGGGAAGCGGGAGGAAAAAGGAAGGA |
| | | | | GAATGAACAAGGGGCTCAAACCCCTACACACTGCAAAACATT |
| 194 | IMAGE:809648 | AA454673 | 3′ | CAGACATTTGGGATTAAAAC |
| | | | | CATATGAAATTCTAATAAATCCATTTTATTTGTGGCACCACA |
| | | | | ATATTATCATTAAGCTCTCTTTTTACACAGTCTGCAATTTGT |
| | | | | ATCAGCTGCCCCAGTGTGACTCTGCCCTTATTTTAGGAACAA |
| | | | | CCTTTTGCTGGGTGGCGTCCTAGAAGGTCTGGGCCTGGGCAG |
| | | | | CAGCGACTGGGAAGCCCACCTGTGCTTTCCCCCATCTGGGTG |
| | | | | GGGCGGCACAGAGACCCTGAGAATCAGCGGTTATGGGAGCTG |
| | | | | TGTGTTAGCTGTGTGTTATTGGCTTTGGCTTCAGCATGTCCT |
| | | | | GCCTAGGAGTCTCCAGCAGCTGTGGTTTCCTTGGACTGGAGG |
| | | | | CTTTTTCTCCTGATGACAATCGTGACAGGTCCATCAGGCAGT |
| | | | | GCGTTGATGATGTTCCAGGCTTCAAACCGTGTGAGGCCCTGC |
| | | | | ATGGCAGTGCCACCCAGCTGCAAGATTTCATCTCCAGGCTGG |
| | | | | ACTGTCTCACTTTGTTCTGAGGCTGCTCCTTTGAAAATCCTG |
| | | | | TTAATGGTGAGAAGCTTGTCTCCGTGTAGGGAGCCCTTCCCT |
| | | | | CCTTCCAGGCTGTAGCCCAGCCCTGCCGACATCTTCTCCATG |
| 195 | IMAGE:809776 | AA454732 | 3′ | GTCACCGTGAAGACTGTGGCCTC |
| | | | | GCACAGGGCTGGCTCTGTGCAGGCTCCAATCTAGGACACAAT |
| | | | | TATCTTTAATCTTTGTTGGCCTAAAAATCCTCTAGCATTGAC |
| | | | | TAACCGGTTCAATCCTCCTCCAGCAAGTATGTGGACTGGACT |
| | | | | TGTGTGATTTCTGGTCCTGACTTCCTTTGGTTTGCTCAGGTT |
| | | | | CACAGAGTGTTTCCAAATGGGCTGGCCTCCCAGGAAGGGACT |
| | | | | ATTCAGAAGGGCAATGAGGTTCTTTCCATCAACGGCAAGTCT |
| | | | | CTCAAGGGGACCACGCACCATGATGCCTTGGCATCCTCCGCC |
| | | | | AAGCTCGAGAGCCCAGGCAAGCTGTGATTGTCACAAGGAAGC |
| | | | | TGACTCCAGAGGCCATGCCGACCTCAACTCCTCCACTGACTC |
| | | | | TGCAGCCTCAGCCTCTGCAGCCAGTGATGTTTCTGTAGAATC |
| | | | | TACAGAGGCCACAGTCTGCACGGTGACACTGGAGAAGATGTC |
| 196 | IMAGE:809776 | AA454784 | 5′ | GGCAGGGCTGGGCTTCAGCATGGAAGGAGGGAAGGGCTCC |
| | | | | CGCGGAGAAAAAAGTTCTCGCCACCAAAGTCCTTGGCACTGT |
| | | | | CAAATGGTTCAACGTCAGAAATGGATATGGATTTATAAATCG |
| | | | | AAATGACACCAAAGAAGATGTATTTGTACATCAGACTGCCAT |
| | | | | CAAGAAGAATAACCCACGGAAATATCTGCGCAGTGTAGGAGA |
| | | | | TGGAGAAACTGTAGAGTTTGATGTGGTTGAAGGAGAGAAGGG |
| | | | | TGCAGAAGCTGCCAATGTGACTGGCCCGGATGGAGTTCCTGT |
| | | | | GGAAGGGAGTCGTTACGCTGCAGATCGGCGCCGTTACAGACG |
| | | | | TGGCTACTATGGAAGGCGCCGTGGCCCTCCCCGGAATGCTGG |
| | | | | TGAGATTGGAGAGATGAAGGATGGAGTCCCAGAGGGAGCACA |
| 197 | IMAGE:810057 | AA455300 | 3′ | ACTTCAGGGACCGGTTCATCGAAATCCAACTTAC |
| | | | | TTTTTCTTTTAAATCATGACACTTGGTAGGTTTACCACCAGC |
| | | | | ATCCAAAATGAACAAAAACGGAAAAAAAAGCATTTACTATAT |
| | | | | ATTTCAGATTTCTTTGGTTGGGGTTCTCCCCATGTGGTATTA |
| | | | | ATATTTCTTGTTTCAATATATATATTACCAAAACAGTAAAAA |
| | | | | CCAGGAAAAAAAATAGAAACCTAGCGGTTGCTGAAACTAGAG |
| | | | | AGGCTACTCTCTTGTCTTCCGTGCAGGAATTCCCAGGTTCTC |
| | | | | AGCTTGCTGGAAAAATTTGTTGACATTTTCTTTTTGTAGCTG |
| | | | | TTTCTTAAAGAATAACAGTAAACATTCCAATGTCCAAATCTT |
| | | | | GGTTAGTCTTCCACTTTATTGCTTGGATGTTTCTTTGGTGTT |
| | | | | GGTTAAGGTTGTGGCCTGCTTTTTGCTTTATTTCTGAATGGT |
| | | | | CATTAATTCTTTAGGTCACCTGCCGATGGTGAAGGTGCCTGA |
| | | | | GGAGCCTGGTGTTACTCAGCACTGCTCTGCTGGGTGGGTGGA |
| 198 | IMAGE:810057 | AA465019 | 5′ | GCAGGGTTCTCAGTTGGTGCTTCACCTGCC |
| | | | | AGAGGAGCGGAGCGGGCAGCGGGAAGGGGCGCGCTCCGCTGG |
| | | | | CCGCCGAGCCGCACTTGTCCAACGTGGAAAACCCAAATACCA |
| | | | | GTTTCAAACACTTGGGAAACATTCAGCCCCGCTGCGCAGCGC |
| | | | | GCATGCGCCCCGGCCCCCTCCCCCGGCAACGGCCCCGCCCCC |
| | | | | CGCCGCATTCACGCCCCTCACCGTCCCAGGCCCTGGGGGCTG |
| | | | | CGGGCTCGAGGCCGGCCCTCGCGGNGGCGTGGCCTTGCCTGT |
| | | | | CACTTTTTCCAGAGGCGAGGGTCGCGGAGGGGACAGCGTCAG |
| | | | | GGCCGCTGGGGTGTGGACGGCGGGCGAGGCGCAAACTTTACT |
| | | | | AGGAGTTTTTGGCACTTGGAGGCAGAGCCTGTTGGGCGGCAC |
| | | | | AGCACGCCCGCTGGGAAACGCAGGGGAGCGGCCTGCTTCGCT |
| 199 | IMAGE:810061 | AI732774 | 3′ | GAAAACCCGACCAGGACCTAACGGGCCGCGGGACA |
| | | | | GGCTGCGGTAGTTGCTGTGTACCATGGTCTCGGAGGTTTCTG |
| | | | | TCCCGCGGCCCGTTAGGTCCTGGTCGGGTTTTCAGCGAAGCA |
| | | | | GGCCGCTCCCCTGCGTTTCCCAGCGGGCGTGCTGTGCCGCCC |
| | | | | AACAGGCTCTGCCTCCAAGTGCCAAAAACTCCTAGTAAAGTT |
| | | | | TGCGCCTCGCCCGCCGTCCACACCCCAGCGGNCCTGACGCTG |
| | | | | TCCCCTCCGCGACCCTCGCCTCTGGAAAAAGTGACAGGCAAG |
| | | | | GCCACGCCCCCGCGAGGGCCGGCCTCGAGCCCGCAGCCCCCA |
| | | | | GGGCCTGGGACGGTGAGGGGCGTGAATGCGGCGGGGGGCGGG |
| | | | | GCCCGTGCCGGGGGAGGGGGCCGGGGCGCATGCGCGCTGCGC |
| | | | | AGCGGGGCTGAATGTTTCCCAAGTGTTTGAAACTGGTATTTG |
| | | | | GGTTTTCCACGTTGGACAAGTGCGGCTCGGCGGCCAGCGGAG |
| 200 | IMAGE:810061 | AI734162 | 5′ | CGCGCCCCTTCCCGCT |
| | | | | TCCTCGTCCTCCTCGGGGGCCTACCGAGCGGCTACGGCGCTC |
| | | | | ACTGACCGCGTCCGTACGGCATGCTGGCGGGCAACGAGAAGC |
| | | | | TAACCATGCAGAACCTCAACGACCGCCTGGCCTCCTACCTGG |
| | | | | ACAAGGTGCGCGCCCTGGAGGGCACAACGCGAGCATAGAGGT |
| | | | | GAAGATCCGCGACTGGTACCAGAAGCAGGGGCCTGGGCTCAC |
| | | | | CGCGATCTACAGCCACTACTACACGACCATCCAGGACCTGCG |
| | | | | GGACAAGATTCTTGGTGCCACCATTGAGAACTCCAGGATTGT |
| | | | | CCTGCAGATCGACAATGCCCGTCTGGCTGCAGATGACTTCCG |
| | | | | AACCAAGTTTGAGACGGAACAGGCTCTGCGCAATGAGCGTGG |
| | | | | AGGCCGACATCAACGGCATGCGCAGGGTGCTGGATGAGCTGA |
| | | | | CCCTGGCCAGGACCGACCTGGAGATGCAGATCGAAGGCCTGA |
| | | | | AGGAAGAGCTGGCCTACCTGAAGAAGAACCATGAGGAGGAAA |
| | | | | TCAGTACGCTGAGGAGGCCAGTGGGAGAGCAGGTCAGTGTGG |
| | | | | AGGTAGATTCGCTCCGGCACGATCTCGCCANATCCTGAGTGA |
| 201 | IMAGE:810131 | AA464250 | 3′ | CATGCACGCAATATGAGGTCTGGCCAGCAGA |
| | | | | CTGAAAGGGTGCGCCGAGTCAGATAACCTCGGACCTGCTCAT |
| | | | | CTGGAGCTGCTCCGTGTGGCCAGCGACCTCCCGGTTCAATTC |
| | | | | TTCAGTCCGGCTGGTGAACCAGGCTTCACATCCTTCCGGTTC |
| | | | | TGCTCGGCCATGACCTCATATTGGCTTCGATGTCACTCAGGA |
| | | | | TCTTGGCGAGATCGGTGCCCGGAGCGGAATCCACCTCCACAC |
| | | | | TGACCTGGCCTCCCACTTGGCCCCTCAGCGTACTGATTTCCT |
| | | | | CCTCATGGTTCTTCTTCAGGTAGGCCAGCTCTTCCTTCAGGC |
| | | | | CTTCGATCTGCATCTCCAGGTCGGTCCTGGCCAGGGTCAGCT |
| | | | | CATCCAGCACCCTGCGCAGGCCGTTGATGTCGGCTCCACGCT |
| | | | | CATGGCAGAGCTGTTCCGTCTCAAACTTGGTTCGGAAGTCAT |
| | | | | CTGCAGCCAGACGGGCATTGTCGATCTGCAGGACAATCCTGG |
| | | | | AGTTCTCAATGGTGGCACCAGAATCTTGTCCCGCAGTCCTGG |
| 202 | IMAGE:810131 | AA464358 | 5′ | ATGGTCGTGTAGTAGTGGCTGTAG |
| | | | | ATAATTTGCCAAGATAAATCACTTTTATCTCTATAGGAAAGG |
| | | | | GAGGATCTAAAAAAAATATAAATTACATTAGTAACACAACAT |
| | | | | AAGAAAAAGACAGGGACAAAAACAACAGAGAAGTCTGAATGA |
| | | | | TGCTACCCTAACCTATTTATAAAAAGGCCCTGCATCAGAAAT |
| | | | | TCACAATCCTACCCACTTCTAAAAATATATTTAGACATGTAC |
| | | | | AGAAGCGGTGGGCTTGTTTTTAAATTGTTTGCTTTATTTGTA |
| | | | | AAAATATATTAAAGGTGAATAGAAATCCTCTCTCCCTTCCCC |
| | | | | CTGTCCAGCCCCCAGCTAGGGACTGGAGATCAGGGGTAACTA |
| 203 | IMAGE:810621 | AA464744 | 3′ | T |
| | | | | CTTTTAGCTGGCTACACATGAGGCCACTTGTTTTAGGGTGAG |
| | | | | CTCCAGGGATTTGCCTGGATTTTGAAATCATGTAGAACATTA |
| | | | | TCCACGTGGCTGTGGCTGTGGCTGTGGCTGGGCCCTGGCAGG |
| | | | | TGGAAAACCATCTCCCAGAAACCTGAAATCACCTGCCAATGA |
| | | | | CGCAGATAACCCTGGCCCTACAGCCTGCTTGCTCCGCCTATA |
| | | | | CCACAGAGCACAGCCTGGACATTATGGAGGGTGTGGCGGGAC |
| | | | | GGCCACACCTGGGTCCTCCATCGGGAACTTTTCATGCTTCTT |
| | | | | TCTCCACCTGAGGTCTTGGTCTGAAGAAGACCTCAGGACTCA |
| 204 | IMAGE:810621 | AA464036 | 5′ | CATCTT |
| | | | | GCAATCATAAAATAACTTTATTGGTCAGGTTAGCCACCACTC |
| | | | | ATGCTTTTCCTGTAATAAGGATCCTTTATAAAGGCATGATGG |
| | | | | TGTTCACATGCAGATGCTTTCTGAAGAGCCCTGGGGCAGGGG |
| | | | | GCAGCCTTGCCCCTCACATCGGAGCTCCTTTGTTGAAATGAG |
| | | | | CTGGTTTGGCTTTTGTGGATTCCAGGTCTGGAGCCAAGAACG |
| | | | | TAGTCCAAAGATCCCCTCTTCCCTTCTCAGGGAAGGTGCTTC |
| | | | | AAAGCATACACAGTATCAGGGATGTGATGGCATCTGGGCAGA |
| | | | | GCTATACTTGGGCTAACTCTCCTCCAACAGTCCTTGCCCCTG |
| | | | | ACTGCCCAGATGGCTTTGTCCCAACCTTGCCCAAAGGACGGT |
| 205 | IMAGE:811162 | AA485748 | 3′ | GGGTTAAGCCCAGGCAACATT |
| | | | | AATGTATAGGGCTATATTTTGGCAGCTGGGTAGCTCTTTGAA |
| | | | | GGTGGATAAGACTTCAGAAGAGGAAAGGCCAGACTTTGCTTA |
| | | | | CCATCAGCATCTGCAATGGGCCAAACACACCTCAAATTGGCT |
| | | | | GAGTTGAGAAAGCAGCCCCAGTAGTTCCATTCTTGCCCAGCA |
| | | | | CTTTCTGCATTCCAAACAGCATCCTACCTGGGTTTTTATCCA |
| | | | | CAAAGGTAGCGGCCACATGGTTTTTAAAGTATGAGAAACACA |
| | | | | GTTTGTCCTCTCCTGTTATCCAAGCAGGAAGATTCTATATCC |
| | | | | TGATGGTAGAGACAGACTCCAGGCAGCCCTGGACTTGCTAGC |
| | | | | CCAAAGAAGGAGGATGTGGTTAATCTGTTTCACCTGGTTTGT |
| | | | | CCTAAGGCCATAGTTAAAAAGTACCAGCTCTGGCTGTGGTCC |
| 206 | IMAGE:811162 | AA486471 | 5′ | GTGAAGCCCAGGCCAGG |
| | | | | TTAGAGCTTAATGGAATTTTATTTTGAAAATATGGCAAGAGT |
| | | | | CTAAGGCACTTCAAACATTTAAATACATAGAGGACCAAAGTA |
| | | | | AATGTGACACGGTAAAAAGGAATCCATAAATACAAAGAGAAC |
| | | | | ACTGTGTTTCTCTAGAGGCAAATACAGAGCCGATTCCTCTAA |
| | | | | CACAATCCAACCTTTAGCATTGGAGTTGTGCAATTAATACAA |
| | | | | ATGATGATGTTACGTGTAGTTCTTCATGGCTTTAGTATGGAA |
| | | | | TACAAAAGCTGAAAATACTGTGTCAAGTTCATATAGATACCC |
| | | | | TTTTTATAAAAAGTCATATATTACATCTACCTAGTTAAGACC |
| 207 | IMAGE:812975 | AA464605 | 3′ | AAATGAGAATATTCTTTTGTAAGT |
| | | | | CTGTGTCCTAATTTATTATGACTACATAGCCCACATTCCTCT |
| | | | | GCCCACGCATCCGTGGAGTCCAGAGCCCAGAAAGCCTCCTGC |
| | | | | TGCCCTGCCAGACCGTTGAGCTCCTCAAGAGCGAAGTGTGGC |
| | | | | ACAGGCTGATCAGCTCATGCAGAATGGCAGGGCTTCAGCTGC |
| | | | | CCAAGTGTGTGCGTACCAGAGCACAGCATTCATGAAGCTGTC |
| | | | | TGACTCCACCTCCACCTCTGATAATGCGTGGGTGCTTTTGGG |
| | | | | ATAGAGCAGGAGCCGAACAGGCACATTCCGGGTCTTGAGGGC |
| | | | | ACGGTAATACTCCATGCCCTGCTTGAAGGGCACACGCCGGTC |
| | | | | CTCCTGGCCCAACATCAGTAACAGTGGTGTCTTCACCTGAGG |
| 208 | IMAGE:813279 | AA455941 | 3′ | GATGTATC |
| | | | | TTCCCAGCCATGCTTTGCAAGATGGGCTTTGCGGTACATACT |
| | | | | AGTGAACTATCGTGAATCCACGGGCTTTGGCCAGGACAGCAT |
| | | | | CCTCACCCTCCCAGGCAATGTGGGACACCAGGATGAGAAGGA |
| | | | | TGTCCAGTTTGCAGTGGAACAAGGAGCACCAGGAGGAACACA |
| | | | | TTGATGCAAGCCATGTGGACCTTATGGGTGGATCCCATGGTG |
| | | | | GCATCAATACCAGCCACATGATTGGTCAGTAACCAGAGACCT |
| | | | | NCAGGGCCTGAGTGGCACGAGAACCCGTGATAAACATAGCCA |
| | | | | CCATGTAGGGCACCACTGACATCCCTGACTGGTAAGAGGTGG |
| 209 | IMAGE:813279 | AA456408 | 5′ | AGGCTGG |
| | | | | TTTTTTAGTTAAATACGCACAATTTTATTGATTGAAGAGATT |
| | | | | AGGACAAAAACATTAAACCAAATACAGGACAAAGCACCAGAG |
| | | | | GCCATAGATCCCCACCATGCATGTCACCAACCTCTCCTCCTC |
| | | | | CAAGGTACTTAAAAAATTGGGGAGAGGGGAAAAAAAAGGTCC |
| | | | | TTCTTGACACAGCACCATCTTCAGAATGTTAAAAAAAAAAAA |
| | | | | AACCTTCTCTCCTTTCTATCTTCCATTAGCAAAATAGAATCA |
| | | | | AGGGCAAATCCATGGCCGCCTTGTCTCCTGGTTACGAAGGGT |
| | | | | GAAGCCGCCCTCCTGGGAACGTGAGGACAGGGCTCCTGCTGC |
| | | | | GCAGGCATAAAGCATCCAAGAGTCTGCACATACATGCCACAC |
| 210 | IMAGE:813426 | AA458653 | 3′ | ACTATTATGA |
| | | | | AAGAGAGAGGCAATTTTATTCTTCCAAAAAAATGCACCAAGA |
| | | | | GAGGGTGAGCACAGGAGCACCCCTGGCCACATCCCCCATCCT |
| | | | | AAGCAGGGTCTGAGATGAGGCCAGGCCTGACGTGGGCTTGGG |
| | | | | AGAAGCTGACGGAGCTCCCTGTGGCCTTGGGGAGGGAACCAG |
| | | | | GCAGACCTGGAAGTGGAACTTTGTTGTTAGCACCAGGAGCCG |
| | | | | CCCACAGCTGGGCTCGGCAACAGGGCAGCACATGGCCCTGTT |
| | | | | GCTGCCACCTGAGAGTCTGGGGAGGGGCTGGTGGCAGAAGGC |
| | | | | TCCCTGCAGGAGGTCACCTGAATGACTCTCAGATTCACAGAC |
| | | | | CCCCTCTGCCCCCACAACCCCTGTAAACATGAGAATGGGCTC |
| | | | | GTGACACCCTCAACACCTCAGGACAAGATGAGGGTCCGAGAT |
| | | | | GTGTGGCTGGGCTTCAGGCGGCCCAGGAGCTGCCGGGCTTTC |
| | | | | TCCTGCATGAAAAGCTGGTCCCTGGTCCCCCCGCAGGCCACC |
| | | | | GTCTTCCAGGCACTGGACATAGGGGCAGGTGTCGTGAAGTGG |
| | | | | CTTCGGGGCTTCTGGGCCACTGCTGCCTTCTCGGGCTTGGCT |
| 211 | IMAGE:815526 | AA457034 | 3′ | GCAAGAA |
| | | | | ATTCGGAACACCGGACGCAATCAAGAAAGTCCGGAAGTCTCT |
| | | | | GGCTCTTGACATTGTGGATGAGGATATGAAGCTGATGATGTC |
| | | | | CACACTGCCCAAGTCTCTATCCTTGCCGACAACTGCCCCTTC |
| | | | | AAACTCTTCCAGCCTCACCCTGTCAGGTATCAAAGAAGACAA |
| | | | | CAGCTTGCTCAACCAGGGCTTCTTGCAGGCCAAGCCCGAGAA |
| | | | | GGCAGCAGTGGCCCAGAAGCCCCGAAGCCACTTCACGACACC |
| | | | | TGCCCCTATGTCCAGTGCCTGGAAGACGGTGGCCTGCGGGGN |
| | | | | GGACCAGGNNGACCAGCTTTTCATGCAGGAGAAAGCCCGGCA |
| | | | | GCTCCTGGGGCCGCCCTGAAGCCCANGCCACACATTCTCGGG |
| | | | | ACCCTCATCTTGGTCCTGGAGAGTGTTGGAGGGGGTGTCACG |
| 212 | IMAGE:815526 | AA456878 | 5′ | AGCCCATTTCTCATGGTTTTACAGGGGTTG |
| | | | | GAAAGAAAGAGTGGAGGGGTTAACATGGGGCCCACCTCACAA |
| | | | | CCCACTCTTCACCCCCAAAATCACGCAGGGATGGGACTCAGG |
| | | | | AAAGGGAAGCATGTGTGTGTTGAATAGGAGCCCTAACTGTAG |
| | | | | TTACTTCTTTCACAGCAGGGAAGGAAGAGGGAAGAGGCAGCT |
| | | | | GTGGAGAGGATGAGGTTGAGGGAGGTGGGGTATCTCGCTGCT |
| | | | | CTGACCTTAGGTAGAGTCCTCCACAGAAGCATCAAAGTGGAC |
| | | | | TGGCACATATGGGCTCCCTTCACAGGCCACAATGATGTGTCT |
| | | | | CTCCTTCGGGCTGGTCCGGTATGCACAGTTGGGGTACCTGGA |
| | | | | GCCGTTTGTCAGGCGGCAGTCTGTGATGTGCATGCTGGAGTT |
| 213 | IMAGE:840493 | AA485893 | 3′ | GCTCTTGTAGCAGTTGCCCTGCCCGTTCTT |
| | | | | CCTGGTAGATGTCCAGAATGTCTGTTTCCAGGAAAAGGTCAC |
| | | | | CTGCAAGAACGGGCAGGGCAACTGCTACAAGAGCAACTCCAG |
| | | | | CATGCACATCACAGACTGCCGCCTGACAAACGGCTCCAGGTA |
| | | | | CCCCAACTGTGCATACCGGACCAGCCCGAAGAGAGACACATC |
| | | | | ATTGTGGCCTGTGAAGGGAGCCCATATGTGCCAGTCCACTTT |
| | | | | GATGCTTCTGTGGAGGACTCTACCTAAGGTCAGAGCAGCGAG |
| | | | | ATACCCCACCTCCCTCAACCTCATCCTCTCCACAGCTGCCTC |
| 214 | IMAGE:840493 | AA487797 | 5′ | TTCCCTCTTCCTTCCCTGCTGTGAAAGAAGTAACTACAGT |
| | | | | GGGTTTTACCAGTTTTATTTCTAGACTTTCATGTTTGTCTTT |
| | | | | TTGTCTTCTGCTGGAAACATGCCGGTTACATGTTGGTGCTGG |
| | | | | GAAGCGCCGCGCTGCAACCAGAAATGCACAGACCCAGCCGCC |
| | | | | CGCCGCCCAGACCCTCAGACTTGCGCGTCACAGGACAGACTC |
| | | | | CGCTGTGCCCCGTGCACTTGCCACCAGCCTTTGGCGTCTCGA |
| | | | | TACACACAACATCCAGGACTTGTGCCCTTGCCCCATCACGAC |
| | | | | AGACAAAGCGTCCCTCAAGGCCCCCGCGTGGTTCAGACAGAC |
| 215 | IMAGE:841641 | AA487486 | 3′ | GCCGCAGCCAGGATGG |
| | | | | GCCAGCTCACAGTGCTGTGTGCCCCGGTCACCTAGCAAGCTG |
| | | | | CCGAACCAAAAGAATTTGCACCCCGCTGCGGGCCCACGTGGT |
| | | | | TGGGGCCCTGCCCTGGCAGGATCATCCTGTGCTCGGAGGCCA |
| | | | | TCTCGGGCACAGGCCCACCCCGCCCCACCCCTCCAGAACACG |
| | | | | GCTCACGCTTACCTCAACCATCCTGGCTGCGGCGTCTGTCTG |
| | | | | AACCACGCGGGGGCCTTGAGGGACGCTTTGTCTGTCGTGATG |
| | | | | GGGCAAGGGCACAAGTCCTGGATGTTGTGTGTATCGAGAGGC |
| | | | | CAAAGCGTGGTGGCAAGTGCACGGGGCACAGCGGAGTCTGTC |
| | | | | CTGTGACGCGCAAGTCTGAGGGTCTGGGCGGCGGGCGGCTGG |
| | | | | GTCTGTGCATTTCTGGTTGCACCGCGGCGCTTCCCAGCACCA |
| | | | | ACATGTAACCGGCATGTTTCCAGCAGAAGACAAAAAGACAAA |
| 216 | IMAGE:841641 | AA487700 | 5′ | CATGAAAGTCTAGAAATAAAACTGGTAAAAC |
| | | | | TCTTTATTGAATGAGGGTTGTCAGGAGCAAAGGTGGGATCAA |
| | | | | GAGCAGCAAAAGCAGAAACAAGTATAAAAGTATCAAAAAATA |
| | | | | CAAAGTGCTAGCACTGAGGAGAGTGAGAAGGGTTGGGTTGTG |
| | | | | GCCCAGAGGGACCTCTGGGACACAGGATTGAGGACTTGCCAC |
| | | | | AGCCTCCAAGGGAACCTAGGCCTGGGGGGCGTGTGCAGGATC |
| | | | | CTTGGCTGAGGGTGGAAGTGGCTTGAGCGGGGCCCAACCCTG |
| | | | | GGCCGTGAAGTATGAGACCAGTTGTGTGGGCACTTCTGCGAG |
| | | | | CACGGTCTGTGCCAATGCCTCCCGAGGGGCATTCTGGAACCG |
| | | | | GCGGTAGGGTACAAACTGCACAATGTCGCGGGCAGCACCTGC |
| | | | | CCAGAACGTGTATGCAGGGGTCCACCATCAGCGTCCAGCTGC |
| | | | | TCCATGGCCTCAAAGTCAGCACCAGCCACACCCACATTGATC |
| | | | | ACTGACATGGGCAGGTTCGAGGCACGCACCACAGCCTCACGT |
| 217 | IMAGE:843139 | AA485922 | 3′ | GTGGG |
| | | | | GGTCGGTCTGTTCTTTTGCGGTTCTGCTCTTGCCCTGTGTTC |
| 218 | IMAGE:843139 | AA486527 | 5′ | TCTTTGTCTC |
| | | | | CCAGAGCTAAACAATTTAATATAAAAAATGCCATTTTTTGTC |
| | | | | CATACAGTATTTATAAAAAAGTACATAGTGGTTAGTTTTGCA |
| | | | | ATAATTTCTTTTTAGCCAGATGTCATATCATCATATAAATCT |
| | | | | ATGAATATAACAAATGACATAAGAACAGTATAAATAAGTTTT |
| | | | | TGTAGTATTTACACTTACACAGAAACTAGCCCAAATGGTGTC |
| | | | | CTAAGAAATTGTTTACAGTTAAAGTGAAACTACTGATTCAAC |
| | | | | ATACTGACACTCCAATGCTTTTTAAAGTTTCGTATTATTTTC |
| | | | | TATACTAGTTTTGGCTATGATTTTGCATAGAATTACTTATAA |
| | | | | AGTATGAGCATTTCACATCACAGTAGGAGCTTTTAGTATAAT |
| | | | | AGTACAAAAAAACTAGCTACGAAAAGGTCAAATCCTCCTAAA |
| | | | | TCTAGTTTTTCTTAAAATCTGGCTTCTAACTTTGGGAAAAAG |
| | | | | AAAACATTGGCATCACTTGTTTGCTGCAGGGAGTATTCACCA |
| 219 | IMAGE:884438 | AA629687 | 3′ | GGAGAATAAGGTGTTACCTCTTCATCACG |
| | | | | TTCATTCAATTTCCTTTAATGAGTACTTGTTACAGTAAAAGA |
| | | | | GGTATAAAGTCCTGTTCCCAAGTCCAAACCACTTTTTAACTT |
| | | | | AAATCTTGAGTTTTTCTGAATTACTCAATTTGAAGTAATTCT |
| | | | | CTTTATATCTGAAAAATGGTTTTATTGAAACGTTTGAGATTA |
| | | | | AAAAATATGCATTGCAAGAAGCATATGACAAACATTCTGAGA |
| | | | | GTACAAAATTAGTTGTAAAAAATAACATAATTTACCAGTAAA |
| | | | | CCCACTCATATAGAAATGTGCAAAGCCTTTTGATATAAAAAG |
| | | | | TTTTGTACACCAAGCACCTATTTTTATAACTTAGCTTCCCAT |
| 220 | IMAGE:897910 | AA598653 | 3′ | GGAGAGA |
| | | | | ATTTGTTAAACAGTTTAATTCCCAAAGCTAGTAATTTTAGTT |
| | | | | AAATATACATTAGAGCCTTTTTAGATGGCTGCTAATAAACAC |
| | | | | TATGTCAAAATGTGTAGTTTTAAACTCAGACTCGAAAGCCAA |
| | | | | GATAAGCAACTCCTTCAGTTATTACTCTGACCAAGGCATAAG |
| | | | | AATTCACTTAGACAAAAAGCTTTCAAAACCTACCTAAAAATA |
| | | | | AGATAGTTCATAAATTTTCAAAACTGTTCTTCCCTGTTGCGG |
| | | | | ACAGCCCTTGATCTTTGTAAGACTTAGCAAATTTTGGCATGC |
| | | | | TCTCATGTTAGCTTTTTAAGTTACTGAAAACTCCTATAAATT |
| | | | | TAGCATCATTTCTCAAATCTGTATAGTTTTCTCATTCCGAAT |
| 221 | IMAGE:898286 | AA598974 | 3′ | GCTTAAACATTTAGG |
| | | | | CCCTACAAAATAATTTATTGGAACACACAGCTACAGCACTCT |
| | | | | ATGTACAAGCACATTGACGCTCCTGACTATCCTCAACTAGGG |
| | | | | GACCCTTTTCTTCCCCCTTGCCTTGCGGACCTCTTCTATCAA |
| | | | | ATCTTTCAGGTACTGGATCTCCTTGGCCAGGGAATCCGCCCT |
| | | | | CTCTTTTAGAGCCTCGTTCTTCTTTTCCAGCTCTTTGCACTC |
| | | | | ACCAGTAAGAGCCTCCTGCTCCGCCCTCTTCTTCTGGCGGTA |
| | | | | CCTAGTGGCTGCTGTCTTGTTTTGCTCCATTTTTTTCAGCTT |
| | | | | CTTATCCAGTTTCTCACCCTTTACTTTTGCTGCTACCATCTT |
| | | | | CTCTCCAGGAGGATCGTAAGGTTTGGGACGGGCAGACCCACA |
| | | | | GAGAACACCTGGAGATGGGAGGCTCCTATTTGGAGAGCCCCT |
| | | | | GGTAGAGGGGCTGTGCTGAGGAGACCCCAGATAGGACTCTGG |
| | | | | GCTCATACAGATGCCACTATCATTATCTGAAGGGGTGTCTTC |
| | | | | CTCCTTTATGCACTGAGGGATCATGGCAACGTAAGCAGTGTA |
| 222 | IMAGE:949971 | AA600217 | 3′ | GTCTGGCTTCCTATCTCCT |
| | | | | TCCAAATCAATTTATTATCCTGACAGCTGGCATCATTAATAC |
| | | | | TTTAACAAAACCACTTAAAATTAGCCAAATATCTAAGACAGA |
| | | | | TACATATACAAAAGATATACAAATTAAAACCATTTAAAAAGT |
| | | | | AATAGATACCATAATTTGTACTTGGCCACAACTTCTGTATTC |
| | | | | AGAAATGATTGTAAAATTAAAACCTAAGTTAAAAACTGTACA |
| | | | | CCATATACTTTGAGTGATTTACATCTTAGAAAACAAAGGCAG |
| | | | | TCTTTCATTGTTACAGATTTAGTGTCTCTGGTGGGTTGAGGA |
| | | | | GAGAAACACCATGATACTTTGAATTTTTGTACTTTTCTCTTA |
| | | | | TTGACTGTTGTGCATGCTGTGGTGCTTTGAGGTAGGTCTGGT |
| | | | | GAAGGTCCATGAGACAAGGCTTAAGACTTTCCAGGGTATATC |
| | | | | CAGTCTTTCGTATTAATGATTCAGGCCAGCTTTGTCCCGTGA |
| | | | | CTGTGTAGAGTGCTAAATGAAAGGCAGCTCCAGCAATAACTG |
| | | | | ATGGCAAATACTTGAGGTATGGGTCAGCATCTATCAGACTTA |
| | | | | ATTCTCCCAAAAACATTGCTAAACTTTCAACTTTGCAGTTTG |
| | | | | CAGGCTACTGATGCAGAAAGTATTGGGGAAGAAACTGATTTA |
| | | | | CTGGTGGGAGCAGCTAAGTCAAAAGTAAGGGACTTTCAAAAA |
| | | | | CTAGATGGCTCCATTGCTCAGGAACTTGTTTACCTGGGTGGA |
| 223 | IMAGE:950690 | AA608568 | 3′ | GG |

### REFERENCES

Aalto, Y., W. El-Rifa, et al. (2001). “Distinct gene expression profiling in chronic lymphocytic leukemia with 11q23 deletion.” Leukemia 15(11): 1721-8.

Adams, J. M., A. W. Harris, et al. (1999). “Transgenic models of lymphoid neoplasia and development of a pan-hematopoietic vector.” Oncogene 18(38): 5268-77.

Alizadeh, A. A., M. B. Eisen, et al. (2000). “Distinct types of diffuse large B-cell lymphoma identified by gene expression profiling.” Nature 403(6769): 503-11.

Bodrug, S. E., B. J. Warner, et al. (1994). “Cyclin D1 transgene impedes lymphocyte maturation and collaborates in lymphomagenesis with the myc gene.” Embo J 13(9): 2124-30.

Brugarolas, J., B. F. Haynes, et al. (2001). “Towards a genomic-based diagnosis.” Lancet 357(9252): 249-50.

Capello, D. and G. Gaidano (2000). “Molecular pathophysiology of indolent lymphoma.” Haematologica 85(2): 195-201.

Chan, W. C. and J. Z. Huang (2001). “Gene expression analysis in aggressive NHL.” Ann Hematol 80 Suppl 3: B38-41.

Chee, M., R. Yang, et al. (1996). “Accessing genetic information with high-density DNA arrays.” Science 274(5287): 610-4.

Christoph, T., R. Rickert, et al. (1994). “M17: a novel gene expressed in germinal centers.” Int Immunol 6(8): 1203-11.

Cousar, J., C. Sawyers, et al. (1999). The Hematopoietic-Lymphoid Neoplasms. Winetrobe's Clinical Hematology. G. Lee, J. Foerster, J. Lukenset al. Philadelphia, Lippincott, Williams, and Wilkins. 2: 1993-2032.

Dalla-Favera, R. and G. Gaidano (2001). Molecular Biology of Lymphoma. Cancer: Principles and Practice of Oncology. V. DeVita, S. Hellman and S. Rosenberg. Philadelphia, Lippincott, Williams, and Wilkins. 2: 2215-2235.

Davies, J., P. Badiani, et al. (1999). “Cooperation of Myb and Myc proteins in T cell lymphomagenesis.” Oncogene 18(24): 3643-7.

Davis, R. E., K. D. Brown, et al. (2001). “Constitutive nuclear factor kappaB activity is required for survival of activated B cell-like diffuse large B cell lymphoma cells.” J Exp Med 194(12): 1861-74.

DeRisi, J., V. Iyer, et al. (1997). “Exploring the metabolic and genetic control of gene expression on a genomic scale.” Science 278(5338): 680-6.

Doglioni, C., P. Dell'Orto, et al. (1990). “Cytokeratin-immunoreactive cells of human lymph nodes and spleen in normal and pathological conditions. An immunocytochemical study.” Virchows Arch A Pathol Anat Histopathol 416(6): 479-90.

Ekins, R. and F. Chu (1999). “Microarrays: their origins and applications.” Trends Biotechnol 17(6): 217-8.

Elenitoba-Johnson, K. and C. Kjeldsberg (2000). Non-Hodgkin's Lymphoma. Practical Diagnosis of Hematologic Disorders. C. Kjeldsberg. Chicago, American Society of Clinical Pathologists: 559-641.

Elenitoba-Johnson, K. S., S. D. Bohling, et al. (2002). “Fluorescence PCR quantification of cyclin D1 expression.” J Mol Diagn 4(2): 90-6.

Erlanson, M., E. Gronlund, et al. (1998). “Expression of activation markers CD23 and CD69 in B-cell non-Hodgkin's lymphoma.” Eur J Haematol 60(2): 125-32.

Hamblin, T. J., Z. Davis, et al. (1999). “Unmutated Ig V(H) genes are associated with a more aggressive form of chronic lymphocytic leukemia.” Blood 94(6): 1848-54.

Harris, N. L., E. S. Jaffe, et al. (1999). “World Health Organization classification of neoplastic diseases of the hematopoietic and lymphoid tissues: report of the Clinical Advisory Committee meeting-Airlie House, Virginia, November 1997.” J Clin Oncol 17(12): 3835-49.

Hockenbery, D., G. Nunez, et al. (1990). “Bcl-2 is an inner mitochondrial membrane protein that blocks programmed cell death.” Nature 348(6299): 334-6.

Hofmann, W. K., S. de Vos, et al. (2001). “Altered apoptosis pathways in mantle cell lymphoma detected by oligonucleotide microarray.” Blood 98(3): 787-94.

Homing, S. J. (2000). “Follicular lymphoma: have we made any progress?” Ann Oncol 11 Suppl 1: 23-7.

Husson, H., E. G. Carideo, et al. (2002). “Gene expression profiling of follicular lymphoma and normal germinal center B cells using cDNA arrays.” Blood 99(1): 282-9.

Iwasaki, H., Y. Ezura, et al. (2002). “Accuracy of genotyping for single nucleotide polymorphisms by a microarray-based single nucleotide polymorphism typing method involving hybridization of short allele-specific oligonucleotides.” DNA Res 9(2): 59-62.

Jemal, A., A. Thomas, et al. (2002). “Cancer statistics, 2002.” CA Cancer J Clin 52(1): 23-47.

Jones, P. A. and P. W. Laird (1999). “Cancer epigenetics comes of age.” Nat Genet 21(2): 163-7.

Kim, R. Y., R. Gasser, et al. (1992). “mu-crystallin is a mammalian homologue of Agrobacterium ornithine cyclodeaminase and is expressed in human retina.” Proc Natl Acad Sci USA 89(19): 9292-6.

Kirkegaard, C., H. Hasselbalch, et al. (1989). “Thyroid hormone metabolism in hypermetabolic patients with haematological disorders.” Horm Metab Res 21(3): 145-7.

Klein, G. (1993). “Multistep evolution of B-cell-derived tumors in humans and rodents.” Gene 135(1-2): 189-96.

Knutsen, T. (1997). “Cytogenetic mechanisms in the pathogenesis and progression of follicular lymphoma.” Cancer Surv 30: 163-92.

Kuo, W. P., T. K. Jenssen, et al. (2002). “Analysis of matched mRNA measurements from two different microarray technologies.” Bioinformatics 18(3): 405-12.

Limpens, J., D. de Jong, et al. (1991). “Bcl-2/JH rearrangements in benign lymphoid tissues with follicular hyperplasia.” Oncogene 6(12): 2271-6.

Limpens, J., R. Stad, et al. (1995). “Lymphoma-associated translocation t(14;18) in blood B cells of normal individuals.” Blood 85(9): 2528-36.

Lindroos, K., S. Sigurdsson, et al. (2002). “Multiplex SNP genotyping in pooled DNA samples by a four-colour microarray system.” Nucleic Acids Res 30(14): e70.

listed, N. a. (1997). “A clinical evaluation of the International Lymphoma Study Group classification of non-Hodgkin's lymphoma. The Non-Hodgkin's Lymphoma Classification Project.” Blood 89(11): 3909-18.

Liu, Y., A. M. Hernandez, et al. (1994). “BCL2 translocation frequency rises with age in humans.” Proc Natl Acad Sci USA 91(19): 8910-4.

Lovec, H., A. Grzeschiczek, et al. (1994). “Cyclin D1/bcl-1 cooperates with myc genes in the generation of B-cell lymphoma in transgenic mice.” Embo J 13(15): 3487-95.

Mathas, S., M. Hinz, et al. (2002). “Aberrantly expressed c-Jun and JunB are a hallmark of Hodgkin lymphoma cells, stimulate proliferation and synergize with NF-kappa B.” Embo J 21(15): 4104-13.

Melki, J. R. and S. J. Clark (2002). “DNA methylation changes in leukaemia.” Semin Cancer Biol 12(5): 347-57.

Montserrat, E., R. Bosch, et al. (1997). “Treatment of B-cell chronic lymphocytic leukaemia: current status and future perspectives.” J Intern Med Suppl 740: 63-7.

Naylor, M. and J. D. Capra (1999). “Mutational status of Ig V(H) genes provides clinically valuable information in B-cell chronic lymphocytic leukemia.” Blood 94(6): 1837-9.

Norton, A. J., J. Matthews, et al. (1995). “Mantle cell lymphoma: natural history defined in a serially biopsied population over a 20-year period.” Ann Oncol 6(3): 249-56.

Phimister, B. (1999). “Going global.” Nature Genetics 21s: 1.

Relogio, A., C. Schwager, et al. (2002). “Optimization of oligonucleotide-based DNA microarrays.” Nucleic Acids Res 30(11): e51.

Ries, L., B. Miller, et al. (1994). SEER Cancer Statistics Review, 1973-1991: Tables and Graphs. Bethesda, Md., National Cancer Institute.

Rosenwald, A., G. Wright, et al. (2002). “The use of molecular profiling to predict survival after chemotherapy for diffuse large-B-cell lymphoma.” N Engl J Med 346(25): 1937-47.

Schena, M., D. Shalon, et al. (1995). “Quantitative monitoring of gene expression patterns with a complementary DNA microarray.” Science 270(5235): 467-70.

Schena, M., D. Shalon, et al. (1996). “Parallel human genome analysis: microarray-based expression monitoring of 1000 genes.” Proc Natl Acad Sci USA 93(20): 10614-9.

Seng, J. E. and B. A. Peterson (1997). “Indolent B-cell non-Hodgkin's lymphomas.” Oncology (Huntingt) 11(12): 1883-94, 1987; discussion 1901-2, 1.

Shipp, M. A., K. N. Ross, et al. (2002). “Diffuse large B-cell lymphoma outcome prediction by gene-expression profiling and supervised machine learning.” Nat Med 8(1): 68-74.

Specht, K., M. Kremer, et al. (2002). “Identification of cyclin D1 mRNA overexpression in B-cell neoplasias by real-time reverse transcription-PCR of microdissected paraffin sections.” Clin Cancer Res 8(9): 2902-11.

Strasser, A., A. W. Harris, et al. (1993). “E mu-bcl-2 transgene facilitates spontaneous transformation of early pre-B and immunoglobulin-secreting cells but not T cells.” Oncogene 8(1): 1-9.

Stratowa, C., G. Loffler, et al. (2001). “CDNA microarray gene expression analysis of B-cell chronic lymphocytic leukemia proposes potential new prognostic markers involved in lymphocyte trafficking.” Int J Cancer 91(4): 474-80.

Tsujimoto, Y., E. Jaffe, et al. (1985). “Clustering of breakpoints on chromosome 11 in human B-cell neoplasms with the t(11;14) chromosome translocation.” Nature 315(6017): 340-3.

Tsujimoto, Y., J. Yunis, et al. (1984). “Molecular cloning of the chromosomal breakpoint of B-cell lymphomas and leukemias with the t(11;14) chromosome translocation.” Science 224(4656): 1403-6.

Vaux, D. L., S. Cory, et al. (1988). “Bcl-2 gene promotes haemopoietic cell survival and cooperates with c-myc to immortalize pre-B cells.” Nature 335(6189): 440-2.

Vie, M. P., C. Evrard, et al. (1997). “Purification, molecular cloning, and functional expression of the human nicodinamide-adenine dinucleotide phosphate-regulated thyroid hormone-binding protein.” Mol Endocrinol 11(11): 1728-36.

Voliotis, D. and V. Diehl (2002). “Challenges in treating hematologic malignancies.” Semin Oncol 29(3 Suppl 8): 30-9.

Wodicka, L., H. Dong, et al. (1997). “Genome-wide expression monitoring in Saccharomyces cerevisiae.” Nat Biotechnol 15(13): 1359-67.

We claim:

1. A method of differentiating between benign reactive lymph node tissue, follicular lymphoma tissue, mantle cell lymphoma tissue and small lymphocytic lymphoma tissue, comprising:
hybridizing labeled RNA from tissue with oligonucleotide probes that are differentially expressed by benign reactive lymph node tissue, follicular lymphoma tissue, mantle cell lymphoma tissue and small lymphocytic lymphoma tissue;
analyzing the expression of the probes.

2. An array comprising at least two isolated nucleotide molecules, each molecule having a sequence capable of uniquely hybridizing to a nucleic acid molecule having a sequence with at least 70% homology to a sequence listed in Table 2.

3. An array comprising 120 nucleic acid molecules or spots, each spot comprising a plurality of isolated nucleic acid molecules, each molecule having a sequence consisting essentially of a sequence of Table 2.

4. A method for determining the expression profile of a sample containing nucleic acid comprising:
(a) providing the sample;
(b) providing an array of claim 3;
(c) contacting said array with said sample under conditions allowing selective hybridization; and
(d) measuring hybridization of nucleic acid in said sample to said array to produce an expression profile.


SEQUENCE LISTING (302 sequences)

SEQ ID NO: 1 - DNA, 380 bp, Homo sapiens
  misc_feature (1)..(380): n is a, c, g, or t
gctataacatggcagcctcgcatcccttcctgcttaccacctttctagatattaaggcttacttagttcttactgaattaaatggagagtgacttgacaactcttggccagccattcttaatgatatttgtgttcctaagatatagcagtatctgcaaatcctaaatctgtctcatgaagattttatgatcttttagatcagtgattaatgggaaggacaatgtcctttatttttttaaataaaaaataatgacctggaactttctctgtaggccaataaagggtgagtgtggatggggctatcacccttgggtngtgttngggagtttaacatttctctaggtttaaaaccatncctatnaccttnccacaanaccggc

SEQ ID NO: 2 - DNA, 281 bp, Homo sapiens
  misc_feature (1)..(281): n is a, c, g, or t
ataagccctagatatgatttaatttgaagactagttcatatttttacttttganccaattctagtctcataaaataaaaattcaggtctctctgggtcacaccacacatctaaaagttgacagtatggtctggcactacagtctccttctaggagaagtttgggaaatcattctaacccctagttagctccatgtatcttaagaatcaccaattatttgaaagcttggaggttctaggaggggagtgcagctactcatatacccttgaccgagactgggcc

SEQ ID NO: 3 - DNA, 435 bp, Homo sapiens
ttcacaatccaaatctcaaattacagaaaaatgatatacctttcagctatgtttttttgtgtgtgtgttggctgggaatgccaaaaaggttggcaaaaggggcaggaaaaaagtagtggggctctctggtgtactccactcctcacatgtctaccattctgagatttttgatgtcaggttctgccaagtctcaaaacctcaagagttgccagaattcagtcccagtgtacacattctactctagggagaggaaggataacaaccacccaagggccacccacccgaggacagccctgccttttaggtatgggggatgcgggtgttcattcaatttgctttggggtttcccttcttgaggtcccaggaaaggaggattttcgggggagttcactttcttgcccttcaggtcccgggggggaaggcaacagggttgat

SEQ ID NO: 4 - DNA, 406 bp, Homo sapiens
  misc_feature (1)..(406): n is a, c, g, or t
angtcanantngggttatcaggcatcagtctacctgaggaggcaacagcattggtgggtccaccagtaaaaaatggacaggagactgctgtgccccctttgtgggaaaagcctcccttgggaagcagtggttgtatgctcagtcctcccctgggaagaacaacaacaggcaacttgcagggttcccttcagaatgtctctctgagtgcacctgggcaataagcaggcacaagaccctggggtgctgaaccctnttcaacagcctgggcaggcaacgaggacattcaggaantaccagccaggaaaggcaggaaccgttttgtttggggttcntttcccacaaccgccntgttttttcccggcaacagttgattttnaggaaggcaaaagaaggaaanttttcaggg

SEQ ID NO: 5 - DNA, 301 bp, Homo sapiens
  misc_feature (1)..(301): n is a, c, g, or t
taactgggaattgagaacntgcagttcacactcaacagtaccagggcagaaatgaactaatgcatgcaatttatttagcctatcatgtgggctgtgagtttttcctggaacatccgggctggttttcttctcttggnataatggtttattacatgtgaatcatatcataacataaacttgttagttcctgattcccgataaaaaagacattttattgaacaaatgaacagttcaaggtctaaggcaatgattaaccgagccagtattaaatgctctagncctataaggggaatatcccata

SEQ ID NO: 6 - DNA, 284 bp, Homo sapiens
  misc_feature (1)..(284): n is a, c, g, or t
aggcaggaacatgggttatttatgaaggatgcctgtagagttcaacaagcctgcttactgcgggttagttgtgaccattgtctaaggtaatttaatggttttcctatggaggagctgaagggagccntgaaaggggaaaagggtggctcccaatgagttggcagccaatggggaacaatttggatataataaataggtctcatgttgactcctttccaaaacggcctttcaaaggggnagtgtnggcttggcctggcaaacttctccccacccactncaccaca

SEQ ID NO: 7 - DNA, 288 bp, Homo sapiens
  misc_feature (1)..(288): n is a, c, g, or t
gaatttttattttaaaacaaagaatcaaacaaacaataatggaaaatccatatggaaatattcacaatcttctcagtgagaaataggaaaacaacttccctgccttactgccaaactgaggagccagaagttgacgtgaagttggaaggccacctttccagctaaaccccactccatagctacgtgcatttttattcaaaggctccagggggcagagggaacagtgaggactnaggacccaaaatacttgtcactgggcaagggttttggcttaaaggggtcttgagg

SEQ ID NO: 8 - DNA, 315 bp, Homo sapiens
  misc_feature (1)..(315): n is a, c, g, or t
gaatgtttatagcccaaacttggaatttgtaacctcagctctgggagaggatttttttttgagcgattattatctaaagtgtgttgttgctttaggctcacggcangcttgntaatgtctgttaccatgtcactgtggtcctatgccgaatgccctcaggggacttgaatctttccaataaaccnggtttngacagtatgngtcaatgtgcngtgcagcccacacttntaganggatgaatgtatgtgcactgtcactttggctctggggtgggagtatgtttattgtttgacttattttctctgtgtttgttcc

SEQ ID NO: 9 - DNA, 422 bp, Homo sapiens
  misc_feature (1)..(422): n is a, c, g, or t
ttcttgatagcatcacattttattactaattgcagtttttgattccacaaccctgtataacttggcattctggtgaattggacccgaacatctgtgaatcttaaaaatagtggttgactcattatggcttccttatgtataggattaagaacacagatcctgggaatcagacagcctggcttccacactctagctgggtgaccatgaccatgaagaagttcctgaatgttccagtgtcagtttattcacctttacagagaaatctggccaaacactaccctcagccagggtgatccaagttcaatattcagcaattaaggtcatgttgtttgttaggtgtgtgctcttgatatggcatgatgaggaattgcacttcacttctgtgatattcccccngggcttttaacttcaggtccctgnaa

SEQ ID NO: 10 - DNA, 377 bp, Homo sapiens
  misc_feature (1)..(377): n is a, c, g, or t
natttcggcacagagcgcttccattgctgacctctaccgacctctacctgtggtccttcctctactgcagcagagacactgttttcttcctttgttcttccaaccccatggcacaganacactctccactgcggccaaggattgcaggagaggtggcatcagtgattcaagactgcttttcctacctcttcagtgtttctttcagtgatctgaagttaaagccagggggaatatcacagaagtgaagtgcaattctcatcatgtcatatcaagagcacacactaacaaacaacatgacttattgctggaatattggaacttggatcactggggtttgggtagtnttttgccagntttctcttgtnaagggtggatta

SEQ ID NO: 11 - DNA, 344 bp, Homo sapiens
gcttcttctgggcacattgttctgacataaaggttgcctccttgtgggggagaaggggaggattagtttgttggcttgggcatttgatcataaattatggaggtgctggaccggagaaccacccaccagcccacggaggctaccgggcattcaggataagggccgccttcttcttcagaataaccatacccactccctctgaaacaaagtggagagtcttaggtctgagtggaaactctaaatcttttaattcttgggttcaactttcttcatctgttttcctgggttcagactaaaaccatctaactcagctgggagaagttataaccgctttgttgttgggc

SEQ ID NO: 12 - DNA, 285 bp, Homo sapiens
  misc_feature (1)..(285): n is a, c, g, or t
taccccgacagtcttcacacacacaaaaaaaaaaaaaaaaagaaagacagaccaagcagaatnaaataaaaggtctgaagaacaagttttgttaatttgccacaacagactgtactccaggggaagctttgttgtccattaaagtgagttctctgggaagacgagtagtaaccgacttgcacgattttcctgccttttctatattctctacttactatgacaatacagcactaggnatttccaagtgcttattacccggcataggtgcatgtattttaatgaggg

SEQ ID NO: 13 - DNA, 380 bp, Homo sapiens
  misc_feature (1)..(380): n is a, c, g, or t
ttggntnngaagaaataaaactgcctttatttgcagataacaatcacatacatagaaaatcctaagggatttacaaaaaaagctgctaaaactaataaggagatttaacagtattgcaggacacaaagcatttctgtatcctaacaaagantaattaaaaactggantttaaaaaattatttaggctgggcatggtggctcacacctataatcccagcactttgggagggtagctggattaaaggccacactgccacacccatctaatttttgtatttttagtagagacggggtttcaccatgttgggctaggctggtctcaaactcctgggcctccgacctcagcctcccaaagtgctggggattacaggtttgaggcc

SEQ ID NO: 14 - DNA, 453 bp, Homo sapiens
  misc_feature (1)..(453): n is a, c, g, or t
gcttttgcacatcaataggtatccctaggagggcctgattcagaagccctcatttttaaactcaattcttagatgaacagtcttattcatctggaatgttccacataatggtcatcataattctaatttatctttagtaagatttcaccatttttgtaagtatttgcagcttctaggccctaacacatgtaaaaggtaaacatagccaggaaggtgaaatacacagttctttaaaaatttaagggatgctggccagggcgaggtggcttcacacctgttaatcccaggcactttggggagggctgagggtcgggagggccaggggagttttgaggacccaggccttaggcccaacatggggtggaaacccccgttcttctacnttaaaatttaccaaaatttaggttggggttttgggccgttttgggcccttttattcccngttacccttnt

SEQ ID NO: 15 - DNA, 221 bp, Homo sapiens
  misc_feature (1)..(221): n is a, c, g, or t
acggtagtgggtagcgggtctcgggttgcgggttgcaggttgcaagccnagcccgcaggcaactnccttcccggcgccatgttcggctccagtcgtggaggcgtgcgcggcgggcaggaccagttcaactnggaggacgtgaagactgacaagcagcgggagaactacctgggcaactcgctgatggtgccagtagcncttggcagaagggccgcganctc

SEQ ID NO: 16 - DNA, 342 bp, Homo sapiens
  misc_feature (1)..(342): n is a, c, g, or t
ttttaacccggtcaagtccaaaggtttattttaaggcacaaggtgggnggncaagggggatggtaaaagcgcaaggggtcgtggcctcatcaaggtccgaaggtccaagggaaggcgggtccggtcctgttggtcctggtcccgaattggtagctgggtgtatctccggaccatgttgggggcgcaccatcccttcctcactgggacctcctgggctggtccangcccttctcctcggggtcgcctccttcccgcttgcaagacctcccgcgaagtcctccttgctcaangcccgtgggttgcttcttcangttcttgtagccaagggggccaanaagcgct

SEQ ID NO: 17 - DNA, 226 bp, Homo sapiens
  misc_feature (1)..(226): n is a, c, g, or t
ctttcttatctttcagtcccccatatgccctcctccaatagaatgtttgaaattacaaaaggttcagacaacaccatagaaggaaagaaattacaaatggnacactattttgtgtatatttgtttttaaaaatttctgaatctgcatttaatgaatttttattgaatgatgtgttgaatatttgttaccnataattattgaaattattgataattaatgataatta

SEQ ID NO: 18 - DNA, 356 bp, Homo sapiens
tgtcatagaccaatgcgaagtttttggccattaaatatttttctctgttctaaatgcagagtcttagaagcaagacgtacttttcaattcatatctttctacattatatgaattatatttcacaataaacatatttatttctttagagatggagttccgctcttgttgcccagggctgaagtgcaatggtacaatctcagctcacctcaacccccacctcccagggttcaaggcgattcttctgcctcagcttacccaggcagctggggattacacccgtgcgttcaccatgcccgggcttaattttgtatttttagtagaggacgggggtttctccctgttgggtcagggctggg

SEQ ID NO: 19 - DNA, 408 bp, Homo sapiens
  misc_feature (1)..(408): n is a, c, g, or t
aaaggancctttattgaccagagcaggaccgtggcatttttatatatatatatatatatataaaagtntgaagacctggcaggcagtgatccnattgtccgcccaccacccccagcactgatttcctgctccctgcacggggaagggggaggatgactnctccacccaggccacagggcacactcccctgcaaacagaggaagaaaggggcttttctgtagccaccccctgcacatcaganatcaacaagtattctctcaaannaannnnnntacagnntttganncatttnnntntgnnannccnnngggnngtgagtggggnngnggcnngngnggnnnggnctgggngtttcttggggnngggnctcccntgtctcccttcccntttatggggnttgggggtctg

SEQ ID NO: 20 - DNA, 402 bp, Homo sapiens
  misc_feature (1)..(402): n is a, c, g, or t
ggttatgatggggtgaaaaggtggaccaaaaacgtggacatcttcaataaggagctactgctaatccccatccacctggaggtgcattggtccctcatctctgttgatgtgaggcgacgcaccatcacctattttgactcgcagcgtaccctaaaccgccgctgccctaagcatattgccaagtatctacaggcagaggcggtaaagaaagaccgactggatttccaccaggggctgggaaaggttacttcaaaatgtactgcaagcatctggccctgtcttcagccatttcagctttcacccagcagggacattgcccaaatttcgttcgggcagatcttacaagggagntgttttcacttgcaaattcattgttgttnggcctngttaccccaggacccc

SEQ ID NO: 21 - DNA, 382 bp, Homo sapiens
  misc_feature (1)..(382): n is a, c, g, or t
taaacataannnntacaaagtatagtcttcgtattcactacacaccgcaaagttctgctacttgaaataaagcaaatgaagaaaattacgttttctgacataaaaataattattatatccactggcaacaataaggaaaacttagcacttatatattttatgatcaaattgattcaaaaattaaattggttagcttcagcatctattctgtctatatctccctgtgggatgacaatttagacaatatgaacattctcaggataaggaaatcttgttttaaaatgtcccaggcatcccttccnctggttaaaactccctatatttgccttattataaaattcagggctttcttccnccaggtgggcccaatggcccaagggac

SEQ ID NO: 22 - DNA, 287 bp, Homo sapiens
  misc_feature (1)..(287): n is a, c, g, or t
caaacatccaaaccatttcagaactcattctataaaatatataaacagctttctattttttttctagctgcataatattccattgtgtggatgagccataatttatcaatttcctattatttctaatcttttacaatagatagtgtttcagtccttaatcttatacatataggtggccataaatttttaaggttctttgggctatttggccaacatgtgggaaggaaagccttggaattttataataaggcaaatatagggngttttaacagtggggagggatgctg

SEQ ID NO: 23 - DNA, 363 bp, Homo sapiens
  misc_feature (1)..(363): n is a, c, g, or t
gaacagttcaatcctgggctgcgaaatttaataaacctggggaaaaattatgagaaagctgtaaacgctatgatcctggcaggaaaagcctactacgatggagtggccaagatcggtgagattgccactgggtcccccgtgtcaacttgaactgggacatgtcctcatagagatttcaagtacccacaagaaacttcaacgagagtctttgatggaaaatttttaaaaaattccacaaagagattatccattgagcttggaggaggaaggataggactttgacgttgaaattttattgaacggcacttctttaaaaaggttacccaaaccaggnccacaggattnatttggntttttttgggg

SEQ ID NO: 24 - DNA, 490 bp, Homo sapiens
  misc_feature (1)..(490): n is a, c, g, or t
gatcaagttaggaaacacacgattgaaatctggaagagaaaactggctcctaccacattgcttctctcgatcatgggtgaagcctgaggagttccagacacgggggtagaggctggggtctttatttcttcgatcatattcatgattttctctgggcactttgatggcatcaacacaggtctcctgccaccgaggcagcttgggaattcagtagttctgcagactgtaagtgataataatgtatgtgggttttgcaaagccacagtgcttattcaacccaggaaagcaggaagcgcctctttctctttcaagcaggagcctcntttggcaacccntcttggcaatggantttctggggattttcacttctggacggaggaagttaaccgggttnttcccaccatacttccaatttccttttggtggtttccaaattttggagtggcgttttcgggccttcccttggggnttttcccntcctgaacctttt

SEQ ID NO: 25 - DNA, 359 bp, Homo sapiens
tacacatgtgtatgcatgaaaaatttctagagggtcatattaatgtaagaaattgtgaagggtggtctctagggcatggagcttagcagctagtgataaagaaactcacttgtcattacacttactgtttgaatttacaatgtcatgtttcattttcataatttaaaaaagtcagtgccaaaacacttacataactacttacatttcttatgtatgatttgactgcttattttaaagtttactgtatttaaagttcaacatcaaaagaaagggctaggaaaagtgggtgggctaggacctaggttctttcacactacttcatttctaggcttccacatggctctggtaatagccaaggc

SEQ ID NO: 26 - DNA, 328 bp, Homo sapiens
  misc_feature (1)..(328): n is a, c, g, or t
agtttctgtgttcaagtttgaatactcttgaagtcttatttttttcattttcagattttaaaattttcaaagaaaaggcgttgctgatgtctgaaatctcagatgcctgaaattcaattgacaattactgaacaacagtctcttatttacataaaggtggggttgtcaatcttgggctctcaggaattttctcttgtagggcactgtgtaggctaaaggttatttaaggtgatttcagaggtaggataggattactcagtggattactaccctgttgccaaggttaattccggnaggggtaacccccgttnccagttcacgttagngt

SEQ ID NO: 27 - DNA, 369 bp, Homo sapiens
  misc_feature (1)..(369): n is a, c, g, or t
agaacaggtacttcgtactgggatttcgaggctggcatcctgcagtattttgtgaatgagcaaagcaaacaccagaagcctcgaggagtcctgtctttatctggagccatagtgtccctgagcgatgaagctccccacatgctggtgggtgtactctgctaatgggagagatgtttaaactgagagctgctgatgcaaaagagaaacaattctggggtgactcagcttcgagcttgtgccaaataccacatgggaaatggaattcttaaggagtgctcccaagctccccgaagccgaagtcttcacttttgcttcccacatgggaacacccattcttgcgtcttccctgttaggccagagacacctnaa

SEQ ID NO: 28 - DNA, 410 bp, Homo sapiens
  misc_feature (1)..(410): n is a, c, g, or t
gcctccatagaaaggcttcgtgtggaatatcactgtcgctgagtacccagtcttggcacagttgatgctgacttttcctccgagctccacccacgggatggtgagaatggaccgggcgtaggcactaggcagggtgaatacgtactcctccccgtgttccaggagcctcaacacaccttcccctatcatagagacccccacggacatgcccatgaacttgcttttggtccatacatgagtgttgacgcacagtctcttctcctcgcactcacagtagaagcaggagatgggtgggtgatgggacacttgctcagccacaaaccttagtttgtagcttttgggaagggtcatcggccatttgggtnttcatgacagctggcaggagagccgggaagcagttctctttaggt

SEQ ID NO: 29 - DNA, 301 bp, Homo sapiens
  misc_feature (1)..(301): n is a, c, g, or t
gccgcatgggaggcatggccccggggtcctggtggccactcgtgcctggtggagagcgagggcagcctgacggagaacatctgggccttcgctggcatctccaggccctgtgccctggccctgttgcggagagacgtgctgggggccttcctgctgtggcctgagctgggtgctagcggccagtggtgtctgtccgtgcgcacgagngcgntnngttgccccaccaggtcttccggaaccacctggggccgctactgcttggagcacctgccggcagagttccccagcctgggangcttct

SEQ ID NO: 30 - DNA, 389 bp, Homo sapiens
  misc_feature (1)..(389): n is a, c, g, or t
gtcgagttaggaagggccctggtcacccctctaagcctgcagctcactgctggcccctccctgtcaaatggctaaaggagatgagctgggggtgggggtgccctggtgattcctagggggaaggggtgagcctgcgcatcccttctgagaaagcgggagtcacagccctgaggttttgagtggagacagcatggagattcttggccctgtctgctggtgcgcatccctttctgcacacaggagtccaccgtgcgtgangtttaggtacagccctctgtccctccttgccctctcttgcaccttcccacccctcctttcacttttcagatnacatattgaggaaacagccctngttctgttcagcntagatgggggttcatccccagcct

SEQ ID NO: 31 - DNA, 290 bp, Homo sapiens
  misc_feature (1)..(290): n is a, c, g, or t
gagtttaatgattacatggngctgagtcaggaggtaggaggagattcttaaatctctgaagagttctgggctggggttctgggaggcaaggggctggaaaatttgggccactgattggtcagggtaagggagattgaatcattaggatatggaaattgcattctttgatgatttagcttctggtagggnccttcagaccaggctgacatcagtagtttcatcagtatgcagggncaaccaatcatggccaagtccnctttnagggantttgtncccgtaggatttatccg

SEQ ID NO: 32 - DNA, 417 bp, Homo sapiens
  misc_feature (1)..(417): n is a, c, g, or t
gctgatcgaacagcctcacttgtgttgctgtcagtgccagtagggaggcaggaatgcagcagagaggactcgccatcgtggccttggctgtctgtgcggccctacatgcctcagaagccatacttcccattgcctccagctgttgcacggaggtttcacatcatatttccagaaggctcctgggaaagagtgaatatgtgtcgcatccagagagctgatggggattgtgacttgggctgctgtcatccttcatgttcaagcgcaggaaggaatctgtgttcagcccgcacaaccatantgtttaaggcagtgggatggaaagtggcaagcttgcccaaggaaaangggttaaagggaanttttttgccacagggaaggaaacaccntgggcaagagggnaccatttaccagggggca

SEQ ID NO: 33 - DNA, 318 bp, Homo sapiens
  misc_feature (1)..(318): n is a, c, g, or t
accagaaaataagacattttattttgagaaataaattggaaaaaaatattttaaaatgtttaatttgcaatatacataatactggaattgaaatgctgtctgatggaaatgttgcaatgtggagtaggagggtcaagttcgtgaagatattcttaaaattaatcttggaaactctgtgcctatgaggtttctctaaagtggctaaaatatgcatttaatatgttgtctaaatgagtacatttaattctagagactgtaaggagtaggagattatatgctttgggggcttttgtaggcnttttttttaaaatcagttgt

SEQ ID NO: 34 - DNA, 368 bp, Homo sapiens
taaatgcattattcatatttcttgaagcttagatacagtctaattcatagcaaccatatctgctttatcctaggtgagggtagcagtccacaatggaatagaagaaaatcccattataacaaatgacaaattatatatcatgaatccttctgtctgactaactcaataactttctataaaagccaatggaattcaaataggagctaggagacaacaagttatatatgacagtggaggttgtattccttttatattgctgagaaaactagttaaatgatcagattcttggctgttaaggaaacaattttcgtttaatggggatctgtacaactgattttaaaaaaatggctacaaaaaggccccaaagg

SEQ ID NO: 35 - DNA, 441 bp, Homo sapiens
  misc_feature (1)..(441): n is a, c, g, or t
ttttttatccttcttaannnttattacatgttttattatcctgtccccagaggtgggtttatccagaaaccaagaaaaaaaatcaatcagaataaactcaaaaaaaaaaggtagggggagcaaaaccatcaaccaccagggcagccaggccatcagcccacctccacctctggagggtccccagagacccacgcccgacgcagacccggaggaggcatcagcaagggggcccgggcagagaatcggctatgtctttcattatgaggaggcagggagagacgggcagagatatgtttgctagggtgantatatattttatattaattaaatccgtaagtttaattaaagtaaataggtatttctctggaagtttttttaatttctttcnttttttatagtttttttggttttttgtggnttttttttttttttttggggttt

SEQ ID NO: 36 - DNA, 126 bp, Homo sapiens
caagcaccccgcttttgcagcagaggagctgagttggcagaccgggcccccctgaaccgcaccccatcccaccagccccggccttgctttgtctggcctcacgtgtctcagattttctaagaacca

SEQ ID NO: 37 - DNA, 409 bp, Homo sapiens
  misc_feature (1)..(409): n is a, c, g, or t
ggaatctactncgagcacagcaggtcagcaacaagtttattttgcagctagcaaggtaacagggtagggcatggttacatntttaggtcaacttcctttgtcgtggttgattggtttgtctttatgggggggggtggggtaggggaaagcgacaggaagtaacatggagtgggtncagcctccctntagaacctggttacgagagcttggggcanttcacctggtctttgaccntcattttcttnacatcaatnttattagaagtcaggatattttttagagagtccactntttctggagggagattagggtttcttgccaagntccaagcaaaatccacgtgaaaaagttggntgatgcaggtacaggnttacacgngggcatagtttnccatagtcngttgccaggg

SEQ ID NO: 38 - DNA, 412 bp, Homo sapiens
  misc_feature (1)..(412): n is a, c, g, or t
ccagtcaccaagacaggcatctcaaatcggctgattctgcatctggaaactgccttcatcttgaaagaaaagctccaggtcccttctccagccacccagccccaagatggtgatgctgctgctgctgctttccgcactggctggcctcttcggtgcggcagagggacaagcatttcatcttgggaagtgccccaatcctccggtgcaggagaattttgacgtgaataagtatctcggaagatggtacgaaattgagaagatcccaacaacctttgagaatggacgctgcatccagggccaacttacttcacttaatgggaaaacggaaagattcaaagtgttaaaaccagggagtttgaggagcttgatgggaactgttgaattcaaatcgaagggttgaagccacccccan

SEQ ID NO: 39 - DNA, 394 bp, Homo sapiens
  misc_feature (1)..(394): n is a, c, g, or t
ntccacgatctgctcanncngngaccacgcccttggcagttcgccctcgtagtagatgtctaccacctcggccgtgaacgtcctgatggcttcccacaccaggagcccgtcgtcccggtagaagtagtaggggatgtcttctttgctctccatgccccgggccttgatggcctcgggaaagcacagggaggcataggtcaggtccttcatggccctctgcaccatctgcacgtgcccaccgccccctgtggcgttggccttgtcaaagaggccacactcgcagatgagctgctcacgggccttgggtgttgattgcaatggtaaatctcacgtgtgccaccagcagctttgaaaatgggggtgcacagcagggcagctnggcggtaacatttgc

SEQ ID NO: 40 - DNA, 371 bp, Homo sapiens
  misc_feature (1)..(371): n is a, c, g, or t
ctgggcgagatccagctggtcagaatcgagaagcgcaagtactggctgaatgacgactggtacctgaagtacatcacgctgaagacgccccacgggactacatcgagttcccctgctaccgctggatcaccggcgatgtcgaggttgtcctgagggatggacgcgcaaagttngcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagttgaaaaaggagttggactttgttctnaattactccaaag

SEQ ID NO: 41 - DNA, 400 bp, Homo sapiens
  misc_feature (1)..(400): n is a, c, g, or t
ttatttaaaacttaattctcaccttgagtatgcaaaatacaaactccacaaaatgttcattttactttgtagtttacaaatatacaaaatagacgtttgcttaaatttatattacatatttattaaggcaaggaactatatagaaaaacacatttgttctgcttaaggcatacttgggaataaaccattgtacaaattattgcacatctgaaaccacagtgcataacagactgtctgcataaaaatgctaaagangtaaaccagggtatattacctgacttagggtcataaatgttgatcggaggacaaatataggattttccttgtcaaagtatgcaggcagtttgaaaactttgggcttccntgtttgggnacctttagganccaaggtctcaccaag

SEQ ID NO: 42 - DNA, 471 bp, Homo sapiens
  misc_feature (1)..(471): n is a, c, g, or t
ctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatccaacatttatgacctaagtcaggtaatataccggggtttanttctttaggcntttttatggcagacagtctgttatggcacgtgggtttcagatgtggcattatttgtacatggggtttnttcccagnatggcctat

SEQ ID NO: 43 - DNA, 409 bp, Homo sapiens
tgcacattctgtttttacctctgtcactgactctgtgggtctagccatgtcatttaaccacacttgaatttcaggtattttgtctgtaaaatgaggataataacgcctgtctactacattaaaccacaagatggtttaaaggttagcataataaattattagagtatgacctaggagttacctaatctgacctctttattttacagatagaagtacagaaaggtaaattgaattgctcaaggtcacccagtgtgtggcaaaatcagaactggaaacttaggtcttctgccagtcccattcaagggctttttccattgtacagttaaattatatgttgtgtgtaaggcatagtataaactgtaaaccattcatgccaaatgttcaggtggattgtttttccctcagtttt

SEQ ID NO: 44 - DNA, 389 bp, Homo sapiens
  misc_feature (1)..(389): n is a, c, g, or t
ctccttctcttcttgttattattatcattattattattttgagattgattactttcccataaaagtggaatatactttgctttggttgagtaatgctctaattatctgaggtcttacagtaattgattcagactgatgaccacctgctgcccattccacatgggcagggacacagcaataatgagaattagggttaggctcataggggatggaagccagcagggaagggactaaagctttgggagaaagctgaagggtgactactgcccgggggcttgaactttctaatgggccatggccttnctctgaaaatgtaattactatgaccactgggttaggtgatgtatttttcatttcttacccactctccatcccttttaaacactgca

SEQ ID NO: 45 - DNA, 448 bp, Homo sapiens
  misc_feature (1)..(448): n is a, c, g, or t
ggcccagatcctctggactcctcagatgagcggattcagagagaagcttttcagagcgtgctggcggagacatttttcacaaaagagcccttgcgntgctggtgtccgtggcgtgcctgggaagnccaccaacgctggccggcctccaagcacccgggcctctgctcatgtacagctcctgaactgccctgcctctgagttactgtggaaaatgagcttatatatgaagaagtcagcgagtggacaaagccaggcgcaatggatagcaaagatgtgggaagtctcctcgattcaagttacaagaaaaccgcagcatggagtctnctctcagctgtttgggggnattaccgatgnctttgactaagtcaagactgactttttccagtaattatcacccaagnggttaggaggncgttccctgttccaagttttttgncgttagcntttt

SEQ ID NO: 46 - DNA, 401 bp, Homo sapiens
  misc_feature (1)..(401): n is a, c, g, or t
gaagggcattctcaaaacgtnnccgcacaagcagaccatcccttttattttccccgtctgtctcctttccttctgctttcaaaatgtctcaagagtatttacaagagttgagcaacacaggcatctttatctggggtctttatccacagagcagaggacaggaagtcatcactacagagacgaagcgatgtatggtttgacccagtggaggactttgttaaggtggaggtntgagtntggagtgtaaacgtgggacatccaggggcagtggagggtaaccactgggagaggaagtctgggggacagtttggggagccagccaaatntaaaaataaagcatttctgttctaaatccaaatgaacctttntacgctgctgtcatcttccagtataccccaggg

SEQ ID NO: 47 - DNA, 488 bp, Homo sapiens
  misc_feature (1)..(488): n is a, c, g, or t
ctaaggaagggcccatcctcactgcagaatcagaaactgtcctccccagtgattcctggagtagtgctgagtctacttctgctgacactgtcctgctgacatccaaagagtccaaagtttgggatctcccatcaacatcccacgtgtcaatgtggaaaacgagtgattctgtgtcttctcctcagcctgggagcatctgatacagcagttcctgatcagaacaaaacaacaaaaacaggacagatggatggaatacccatgtcaatgaagaatgaaatgcccatctcccaacttactgatgatcatcgccccctccttgggatttgtgctctttcgcattgtttgtgggcgtttctcctgaggaggggaaantcatgggaaacctatttgtttcgcagaaacacacaagggttaggattacntgggagatagttaaaattgttcctcaatgacgtggcaggcttggagggggagacgaagacggcctt

SEQ ID NO: 48 - DNA, 374 bp, Homo sapiens
  misc_feature (1)..(374): n is a, c, g, or t
tgaaacaaggaaatctactaagacttattttgacactggagtgtcatgcccccatcctcaatctaacatgctactgcgttgttagagggtaaaaaggccgtcttcgtcttcccttccatgctgcacgtcattgaggacatttttactatctccaatgtagtctagccttgtgtgtttctgcgaacaataggtttccatgagtttccctctcaggagaaacgccacaaacaatgcgaagagcacaaatcccaaggagggggcgatgatcatcagtagttggggagatggggcatttcattcttcattgacatggggnattccatccatctggtccngttttttgtnggttttggttcnggatccaggggaccgcc

SEQ ID NO: 49 - DNA, 385 bp, Homo sapiens
  misc_feature (1)..(385): n is a, c, g, or t
tacgttttgtatgtttttttatttgctccaggtggggttttgactgtcactttcccacactctggattagttctgatcccaccacaaggagccctcgaattggctaaagtgagaaactgggcctgaagactccgtaccctctgccatcttgccgagggagtctccttttagaaaacaatcaaagggttattgcatgagtctggatgaatcccactctcagctgtccacgggcccgaccacctcatctagccccctttttggcagggagaacctgggctcccaagttctcctccttcacttcgttacaaaccaaggggaagagcccaccgtgagaacgcgncatctgcaagctgtctccctttttncatccttggtngaaaccctt

SEQ ID NO: 50 - DNA, 437 bp, Homo sapiens
  misc_feature (1)..(437): n is a, c, g, or t
tnttttgttgnctctagcctgancagataggagcacaagcaggggacggaaagagagagacactcaggcggcacanttccctcccagccactgagctgtcgtgccagcaccattcctggtcacgcaaaacagaacccagttagcagcagggagacgagaacaccacacaagacatttttctacagtatttcaggtgcctaccacacaggaaaccttgaagaaantcagtttctaggaagccgctgttacctcttgtttacagtttatatatatatgatagatatgagatntatatataaaaggtactgttaactactgtacaacccgacttcataatgggtgctttcaaacaggcgaggtgngtaaaaacatcagnttccacgttngccttttgcgcaaagggtttcaccaggttggggaaagggngacagcttttt

SEQ ID NO: 51 - DNA, 273 bp, Homo sapiens
  misc_feature (1)..(273): n is a, c, g, or t
cattaaatcagagtacttaatgatacggaaaaaattcctattaagtgaaaaaagcattacaaaacagcatatattatgagctctatttttatttttgaaatatatttatgcagagaaatacaaaatgttaacaatattatcttaaaanaaaaaaatanggctgggcacagtggctcacacctgtaatcccatacttttggaggcaaggtgggtggatcgctttgagcccagggngttcaagaccagcctggggcaacatgggcgnaaccccga

SEQ ID NO: 52 - DNA, 251 bp, Homo sapiens
  misc_feature (1)..(251): n is a, c, g, or t
tgttcctccccnntcccccagggataagaacctgttatccaccatcagtaacattttatgaaagatctacttatttgtctgttttgcagacattttaaaattcataaagtgggatgcttctttaatttaaatacatttagcttcatgaaaaactcactacacagttcttgttcaagcattattgggaaaccaccagagggcactctcacccagggcttaatttgaacatctcgcccaaaagtgacttttaa

SEQ ID NO: 53 - DNA, 487 bp, Homo sapiens
  misc_feature (1)..(487): n is a, c, g, or t
atatcggcacagcactcaggaaagcctaaagcttgaagactccatttatttatagtgcatcccaatccagatacgtaacaattaacgagttatttttactataagcaaagttgcctaaaatcatagttgatactaaccatggttaacagagctctaaagtttgacagaaagtgagattcaaatcctttcactctcatatgctaaaccttttgccttactctgggtcatcagagaaatttaggtgagaatgtatgatgaagtctgtgttttagattcaatgcagatatatcattgtgggcagaactctttctggttatatccagttaagagtaaatcaggctttcagcgngtcgcggtggcttcacgcctgtaatncctaggcactttngggaggnccgaggcggngcaggntccacgnaggttcaggnagatcgagaccttncgggntagcacgggggttttnaccttgttgnttcaggctggttng

SEQ ID NO: 54 - DNA, 199 bp, Homo sapiens
  misc_feature (1)..(199): n is a, c, g, or t
taaggaaaagnnttaataagtaaatatatttattaaatataaaaggtacacagtaaatataaatgaactaaatgctttagttaaaagacaataaaaattatgaaataaaaatgtatacacttgaaagtatttaaaataaatctaattttcataatgaattttaagcattaaggagttttgtaactgantagtggaactc

SEQ ID NO: 55 - DNA, 470 bp, Homo sapiens
  misc_feature (1)..(470): n is a, c, g, or t
aaatgttagagggtgcgggggtgaggactgaccacagattccctggatagtgtagtggtagatttctccacaggatagcgcaattggcaaatcatgcttggttgtgttaggccaaaatactagttttgctttctttaccttttctatcttgatgaaaatgttgcacattctatagttgcaaaacacataaaaggggacttaacatttcacgttgtatcttacttgcagtgaatgcaagggttacttttctctggggacctcccccatcacccaggttcctactctgggctcccgattcccatggctcccaaaccatgccgcatggttttggttaatgaaacccagtagctaaccccactgtgcttncacatgccgggcntaaaatgggtgatatnacaggtcttattatcccctattgggatttatncctcaaaccncttaaaaacaaacagtggccttttggccctttg

SEQ ID NO: 56 - DNA, 384 bp, Homo sapiens
  misc_feature (1)..(384): n is a, c, g, or t
gattaagaacgtaagctcctttattattattattattattattantcatnccctgttatttaccccnaaacaacagcataactcaaataataatgacacacacgtcccgcccatatacacaataccactagcctatctgtcaggctatctggcctttgcttggttcctgatggagctgtctggagacactcnccnctgtaaaaatcccgncttaaacacaggggacagaagaaaggggggacctaggtcagatcataaactgacaggctcccagcgtccttagggagtgctaatgtgggagactttgaggacgtgcttggacacattctggggcaganggcagnaggcactgtttgtttttatgtggntgatggggtaaattcc

SEQ ID NO: 57 - DNA, 449 bp, Homo sapiens
  misc_feature (1)..(449): n is a, c, g, or t
gttcnnttttccttnctcatttnattttaaagttttattatgaaaacacatggaattaacggtgttatccatgtatttgcaacagcagagaaagagtgagagtggaccatccccataggancnacttatcctttggctaaactaatataaataatggaaataacacctaatacaataatacagcacataaaagagattacattaagagaagagacaggaactgcggagaggagtcctgagtatggaggagatgcggctcatggagaagcatccaggctcaggtgaccttccctgaagacttcctgtctctgagcagctcagttcagttccagggtcatacacgtactccgggacccgggnctcactggggggtcagcgcagacttgcttgcctcttttgggtttgggaataccacagctgggctnggggagcagaggntgctgggtttc

SEQ ID NO: 58 - DNA, 372 bp, Homo sapiens
  misc_feature (1)..(372): n is a, c, g, or t
ctgagaggaactcctcactcagctagcttcaggagccatgacatcatctctaccatggaaattccactcactctcctgtgcccccacatttgtcctaggcctcagagtccctataaagagagattcccaactcagtatcagcacaggacacagctaggttctgaagcttctgagttctgcagcctcacctctgagaaaacctctttgccaccaataccatgaagctctgcgtgactgtcctgtctctcctcgtgctagtagctgccttctgctctctagcactctcagcaccaatgggctcagaccctcccaccgcctgctgcttttcttacaccgtgaggaagcttcctcgcaactttgtggtagattant

SEQ ID NO: 59 - DNA, 382 bp, Homo sapiens
  misc_feature (1)..(382): n is a, c, g, or t
agctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggngtgaatgaagacttggctgattcagatgccagggccttgtatgaagcaggaganaggagaaaggggacagacgtaancgtgttccaataccatccttaccaccagaagctatccacaacttcgcagagt

SEQ ID NO: 60 - DNA, 373 bp, Homo sapiens
  misc_feature (1)..(373): n is a, c, g, or t
tcttgtgacgtcattttattttcagctacatagacatctttctcatgtattgttactagaacaacttgtatagggttttatggtttggggaaaacatttttaaaaaatggacttatctctattatacagagttataatataaaaatgatttaaaggctatatttttcagcatgtaggtagctacactgtaatcctgttgaaganactttcctatttaagcttataggatganaatatataattaaagtcttctgatcatagcttgagaccatcaagggantgtttagtttcctccacaaagagccaccagggtttttctcataatctcctttgggtttcatccagggatggcttngcaaaggggagttaccat

SEQ ID NO: 61 - DNA, 362 bp, Homo sapiens
aattaaagcaaatagactgttgtaggtaccaattctcaatgtcacagtgttacatggaaagtaaaatacacaagaacagcccaaaagatggaaacaatggacgtggtcaaatgacatcagtacaacatccatatggtcctaagtagccatctttaaaatgggttaggaaatgccttcaatcattcacacaggacacatgcattggaacaaactctaaggaagtgttcttacacggggaaaaggcaagttacaggatgcatggggcatggatatggggtgtaggatgtgtggtatggtggcatccccacttcatacacaaaataccccggcatcggcccacatggcctgctgtgtgcggtagg

SEQ ID NO: 62 - DNA, 378 bp, Homo sapiens
  misc_feature (1)..(378): n is a, c, g, or t
taaaaaatgatcgttatgtaggtgattgagaagtaaatgtattcttttttaaggtaaaaatttggacccttatcatgcatacccccctctgtgctcttcaaatcaacatcattattaatatctgtacatttttgctcatctgagccagcacaggctgaggctgtcagaatggacaccttttggttgttgggtttctgtcagtttctggggtgaagctgcgtgattgagaacgtagctcttgggctgccatctcggggattattaaggactgtgaactctatccacaagccatggcaatatctgtcccaccgaatgctncctctaaacacactcttacttcccgtggatgtgttgttaaggggtnccgattganggctg

SEQ ID NO: 63 - DNA, 319 bp, Homo sapiens
  misc_feature (1)..(319): n is a, c, g, or t
atgggtcatatttttgttcactgaaaggaccaaccagtttcatcaaacaagctttagagaaagagaaactgagtaattcatcttgtcagttacagttcacatatatgcacacacatacaaactggctcagcatcagtgaaacataactattcaaatacaaaagtataanaaacctctttaaaaaaccaatagcagccaaaacagaacatttgtaaacaaaaccacaactntcagccctgtgcttaaacacagggttctgcattcttttggaaacattaaggtatatggcattaangggggttntaggnccatctttntc

SEQ ID NO: 64 - DNA, 552 bp, Homo sapiens
  misc_feature (1)..(552): n is a, c, g, or t
gctttatcatcatgaaacaagtcatcagagtctttgaatcttgcgtaggaattggaagtcggggtataccaggataggttttcagcaccaggtgtggcactcaccctccggtatgcttggcagagtttgtgaagcggctccggtactgcgaatacctagggaagtatttctgtgactgctgccactcatatgcagagtcgtgcatccctgcccgaatcctgatgatgtgggacttcaagaagtactacgtcagcaatttctccaaacagctgctcgacagcatatgggcaccagcccattttcaatttgctgagcatcggccaaagcctgtattgcgaaagccaaggagctgggacagagttgaaggaaattcaggaggcagctcttccatnttcaaggaggttgtttgaagacngttaggttttgtaaacagtgcatttanagggngtttcggaggcaggtggccgggnacatttngattgatgnagttccacctgttcttccctttgagggacngggtcaggatcaggaaaggggttgttggcaaactac

SEQ ID NO: 65 - DNA, 508 bp, Homo sapiens
  misc_feature (1)..(508): n is a, c, g, or t
nttcggcacagactttttttaagctaccaattgtgccgagaaaagcattttagcaatttatacaatatcatccagtaccttaaaccctgattgtgtatattcatatattttggatacgcaccccccaactcccaatactggctctgtctgagtaagaaacagaatcctctggaacttgaggaagtgaacatttcggtgacttccgcatcaggaaggctagagttacccagagcatcaggccgccacaagtgcctgcttttaggagaccgaagtccgcagaacctgcctgtgtcccagcttggaggcctgggtcctgggaactgagccggggccctcactggccttccttccaggggatggatcaacaggggcagtgtggtcttccgaatgtctgggaagctgatgggagctcagantttccactgtcaagaaagaggcagttaggaggggtttgggtggggcttgttcacctggggggccttccaggtagggccctttttaagtggga

SEQ ID NO: 66 - DNA, 372 bp, Homo sapiens
  misc_feature (1)..(372): n is a, c, g, or t
gtgggnctgtgttgaaacaggccacgtaaagcaactctctaaaggtcaaaccaccatagatttgaatctgctggtcattcgccatctggatttttaactgaatgaatctcatgggtttaaccaaacatgcatgtaatcctgaataccatganttaaatgcgganttgcccagggacgaggaaaccttcaagaaacaaggtcaaagggacancagatataactgtcacantaaacanttctgttgacgtgggaaatgcacatgacttggttgaaacaaagctcctcagtgggccagtgacatccngggtttttcttagggtaggctgaggactcaggggcttatctcaccttctcaggaatgctttttgaagg

SEQ ID NO: 67 - DNA, 436 bp, Homo sapiens
  misc_feature (1)..(436): n is a, c, g, or t
ttgcttacatgggcatccttcagcttttaataatctgaaaaactctatttacccattgtcaatgtgtataaattaatctgagtcaattttatacaataaaaggtgaacttttatgcatgaaacaataatttaacaagaaatgtacctgaagaagaatgttcattacaaatatagganacataaatattaccaaatattggcaagcactaaaatgttcagaaatataagtctattacagttatagctctctcaagcaaaaaaacagcagagaaaaacttagtttaccttaggggctatttatttacttagggatttgttaaaaggtcgaatggggtcacacagaatactaagaagagctgttcacccaggcctcactaagaactcttcttcattcagtagctgtatagtaacatgacaactggctcctacgacccaa

SEQ ID NO: 68 - DNA, 350 bp, Homo sapiens
  misc_feature (1)..(350): n is a, c, g, or t
attacttgcaaattaagttaccacagactctggtagtgtnctaaatngcgccaaggcntgggcncacagcncagtagcagnctggncgncagggccactggccnaccagtgacggacatgcacgtggcagatcatgatttccagcccacggagccagcatttgaaccttgtataattaactttcagttatgatttcccatcgacattttctttgccctgtttgtagctgattgttgtgttttataaatcttctgttaaggcagaagggtgattatgagtggttcacagcagcccttataagctgggccagaaaatttcactagggtcagtaatttaaaccttggttcttc

SEQ ID NO: 69 - DNA, 370 bp, Homo sapiens
  misc_feature (1)..(370): n is a, c, g, or t
gtttttttagcacttgttaatccgttatgatttattagctgtacagcagtagatcctcctccccagctttcaaccccattacatattttattacaggtctcatgttggcgtcctaaaataatgaaaaatatcacacagtacagctaagtacaaaatgcatcaacctagagtctgatagctaactgatggctctcttaaaagcaatacacaganganaaaagtgtttgaaatcagtaagactgaggctctctaaaaaacacatttttaaacatgtgacagttcatgtgncaaggantcactttttagttgggttttggctttcacattattttattttttgaggatccagggtttaaattactgacctggt

SEQ ID NO: 70 - DNA, 453 bp, Homo sapiens
  misc_feature (1)..(453): n is a, c, g, or t
gtcctttgcataatgcatggcaaaatgagcctaaaacctatatggccattttaattttgcttttgtaataataccaagcccagtttctttcaacttgagagatgagctatttattcttttacttaatgaagatgtaagaaatgatcttctgttctaaaaaaaaaaaaatttctctgatgtctcttgaccctgtagaaacacattcagtttctacactgcaaaacagagggatatctgtatggcttccctctttccatctttcctttcctcagggaaagctaggaaaaagaaatcttttctatcacagcagacacaccaaatctccctaagttgtaccaccttaattcctcagaatggcaattgtgtatggataccaagctacaacttggataagaaattggtgattttcttctttnaattttcattctccaattttaaaaacatctattggcg

SEQ ID NO: 71 - DNA, 308 bp, Homo sapiens
  misc_feature (1)..(308): n is a, c, g, or t
gtagtgttttgggcacacctaaggtcgatctgtgttgtatttaaaaatctaatttctttatttgtgtggccttctagacaaacgaaggggaccagaggaaaccccctgacagatctctggatgatcctccttgaatcctgggcagtttggtctctccttgntgtgctcctgtggcanaaactccctttgattggttctttctttccttcccagctagactaagcccctcatgggcaggtaatgaagattgaaaacttttttctggtctccagtgtgagcacattcctcctacatggtagatgtnccat

SEQ ID NO: 72 - DNA, 432 bp, Homo sapiens
tactactcataacagtttatttttactttgtacaaaatacaaaaatgcaaatccaaggagtacagaccagtagtgacaggcacactgcacaacagcaaccttgtctagcaagacaggagttttttaaattttattttagtgaataaatgcattatataaaacaacaacaacaacaacaacaaaaacacaaagaggctagagatttcaccgtttctacccccaaaataacgcttgctatcaagactttggagggggatgggggaaaagaatttaaaaggcaaataattttttttcataaaaagtaaaagctaccataaaacatttttttttctgtcactgattaaatttcttctgaaaagccgcacatatagacaaaacaaaacaaaaattcctgaactggaccaacagccaatactcccaggggtgttaacc

SEQ ID NO: 73 - DNA, 411 bp, Homo sapiens
  misc_feature (1)..(411): n is a, c, g, or t
gccatcatcccacacatcagcaccaagaccatagacagctggatgagcatcatggtgcccaagagggtgcaggtgatcctgcccaagttcacagctgtagcacaaacagatttgaaggagccgctgaaagttcttggcattactgacatgtttgattcatcaaaggcaaatttttgcaaaaataacaaggtcagaaaacctccatgtttctcatatcttgcaaaaagcaaaaattgaagtcagtgaagatggaaccaaagcttcagcagcaacaactgcaattctcattgccaagatcatcgccttccctgggtttatagtagacagaacctttttctggttttccatccggncattaatccctacanggtggctgtgttattcatggggcaggttaaacaaacccctgga

SEQ ID NO: 74 - DNA, 433 bp, Homo sapiens
  misc_feature (1)..(433): n is a, c, g, or t
aaggataaatgctttattctttctgttaattcatcgttttcaaatgaatgagataatgccctagaaacctccaaaaggtaccaaggaggtgagtgtgtgtatataatcataaactcagatttctatatatttatatacattgtggtcattatttgttttgatggccatattgtctcattttaggttagtgggagctccttaatattgctcccctgtttttgtgacatgattcattaatctttgatagcttccttgatttctggagtaagatggcccaagtttattttacatatttcctgccccagacctggattcagctattctcctaagagcactggttcttaggaaccagtngagtaatagtatggagagaccacagtcttggatgttcattggtaactactggctactgagttggcattacttccaggac

SEQ ID NO: 75 - DNA, 332 bp, Homo sapiens
  misc_feature (1)..(332): n is a, c, g, or t
taatcctagattatctttatttgttctataatttaatagtatacctataaaataattacattatacttatagcttttcttcatttataaacaanacaaaaaaattaaatacaatttgagccattataaggtaaactttgtacatacgntaaccccagaaggagcttcacactgcagcatatcatattgctttcattgctacacccacaattgggttcgaagagagtgtgctcgtgtttgcattctgtaagttcttagcttaatccctcccctatctgtgtgggttccatgttaataaaatgataggggttggctttgcagctggncagagac

SEQ ID NO: 76 - DNA, 480 bp, Homo sapiens
  misc_feature (1)..(480): n is a, c, g, or t
cataatacagttttatagtttaatggacaatgtttaacatggtacctctcaaatctgatatatcttgtggtgcttacaatttgccttacactttcatttaaagttaccctgttctccactcaccacatgtataaaatatcctattttttctcttaatgttttacaaaccggtaattttcactatcagtagcggatctttttataactcaccctatgttgcccaaaatacaccaataatataatgattagattaaaaaacttggcatcttttttaaaaaaatgtgctttcttttccatgtataagattctactataccatttgtgaatgacaccctagttacataacacctacatatctgcccctgtgagaatttaccttagtcttctaagactctatcttcaacagttagataagtcaataaccagagttccaagaaaagtagttactttttaagaccaaattattgggataactgggtc

SEQ ID NO: 77 - DNA, 214 bp, Homo sapiens
tggatcctataaacctgtcaattctgttccttttgaggatggccacacagacaaccacttacctcttttagaaaataatacacattaacacctcccgattgaaggagaaaaactttttgcctgagacataaaacctttttttaataataaaatattgtgcaatatattcaaagaaaagaaaacacaaataagcagaaaacatacttattttaaa

SEQ ID NO: 78 - DNA, 564 bp, Homo sapiens
  misc_feature (1)..(564): n is a, c, g, or t
agcagcacctttttggctttttaatgcttggcttgcttatatctttgtctgtaaaagaatctaatagtttaaagcaagaaaaattcctagtctgcaattaaatacgtatggcaactatgtggaatactaatcaaatctttggtgtcctttctaaggtaaattcatttttctacctcagttcaatcttcattatcattttacattccactggaggcccagctagcacaacaatggccagctcttgcctgaatcccgaaaattagacttatataaatgatacccccagaaagactcggggtaatctcaaaacaggagaccaatttttgatgctggcttgcattcttgctttcttggtcattttgcttttagtaggccaaagctaatacttctccagtgggaatttcagatggttggacattggatgggaacaaagaacatatttaaggaaaattaaatttccngggtagtaaagtttataaactttggaaatccntagactgggcttaaactttcactgggtaaattcncaataatggnaacaccttggccaaagatgctatatac

SEQ ID NO: 79 - DNA, 497 bp, Homo sapiens
agcatattagtctatcaaatccaactacccttaatgccagtgaatgttaaaagtaaaactttcttagcactgacaatttaataagtaaaaataagtggtactaagcttacaaaaattagctgaattggggaaattgttgataaggccacaagtattaacatgttatacttgcttgctttgagggtatatagcatcttttggcaaagtgttacattattgtgaatttaacagtgaaagtttaagccagtctaggatttcaaagtttataactttactaccaggaaattaatttacttaaatatgttctttgtttccatcaatgtcaacatctgaaattccactggagaagtattagctttggcctactaaaagcaaaatgacaagaagcaagaatgcaagccagcatcaaaaattggtctcctgttttgagattaccccgagtctttctgggggtatcatttatataagtctaattttcgggattcaggcaagagctg

SEQ ID NO: 80 - DNA, 430 bp, Homo sapiens
  misc_feature (1)..(430): n is a, c, g, or t
ngacagttgattatttatttgaataaaaaattcaattagatttctatcacacacaatagacacaaacaaattaaaggtggattaggggctaaatacatgcatatacatgtatacacacacatacacacagatatatatgcatatacatatattcacacacataaacacatacatatattttttaagggaaaaaaacaataaaattaaaacttagaagtatatatatgtaaactgtgatctggtttcaagattatgaaaggctttctaaatagcttaaagtagaaatcacaacagtaaaagataatctgattataaataaaaaagagggaaaacctttttatgtaaagaagaccataaaatttaaaaggcaaataataaactgggggaaatacctggcaaaatatattcatatccnaaatataccaagagt

SEQ ID NO: 81 - DNA, 425 bp, Homo sapiens
  misc_feature (1)..(425): n is a, c, g, or t
agcttcattaaaatcttgggaaattttaatttgcattcaccttctctaaacatgaacatgaatctgtaaagtgatacattctttcttgcttaagaaattaaagcgtttggggatttgagtttttatactctttgaaaattgagtttcttgtgctaaaatcatcattcacaaaatgtcctctcacctgaggaattccaacacagcaaattcaatctgaaataaattgaggctacatttaagagacgggacttccagctaaaaataggtattagagagctgtttttgccaaaaaattgaatacttaaccttattcttcactcttgactcatttgttttgtctcagtntgggtgactggagggtttcttctttgtatttncattctgtatccatttcttaatgcgattgaattaganaccattttatg

SEQ ID NO: 82 - DNA, 246 bp, Homo sapiens
  misc_feature (1)..(246): n is a, c, g, or t
tatttaaagcacatttttattatagataggttaagtgtggtttgctgtggctaaagatatatttataatggatgaacaagcttttctagataccaagaggtataatatttttctttcagtattgaactaacatttcnctgataacaaggagacattgaactggctgagcctattttaaatgggaaaagactttttttttctggatgttgctttaaagactggnaaattaaaaattttaaagtacca

SEQ ID NO: 83 - DNA, 442 bp, Homo sapiens
  misc_feature (1)..(442): n is a, c, g, or t
gtcatgtcagtaatttatttcagggtctaacaaatattaccacagcagtttagtctcaaagtgatacaaaactgaactcagggtggttactgggtagtccctagtccaaaagattaagacacacctctaatacacacacaggctgtgttcaaggccttttccttcccatcttctggttctgtctccaccctttccaactgatagcacttcattggtgtgtgtgatatatgtgattatcttaagctagaaagtacaacagaaggagaggatggttgtcacttggggattagacagttgagaggataggaaaggagttatatccaccaatacaagcccttcttcccctcctacttagaaagagggtgggaccattggcattccttttctaagaagcccctcagcaaggagtctgttccaagagaatataacccgnactangaac

SEQ ID NO: 84 - DNA, 403 bp, Homo sapiens
  misc_feature (1)..(403): n is a, c, g, or t
gagtttgtttgaagcacacctttaactcagaattgaggttgactgataaaactcagctttaagtaaccctctgggcaagttctgagcagagatccagtgagctgaatgtcaggcaccacctccctggagtctgtatcagtcacatcagcattctcctctgattagaatcaggtttcaagggtcttgttcaagagtttattctctcctttaaagatgccacaataccgtataaggaatgtctcttggtcccaactaatctacaataagagaggagcacgtatagtcagagggcaagaaaacaaccgcagtttctaagtttcaggttatattctcttgggaacagactccttgctggagggggttcnaggaaaagggaatgnaatgggtccacctctttctaagt

SEQ ID NO: 85 - DNA, 279 bp, Homo sapiens
  misc_feature (1)..(279): n is a, c, g, or t
attaaatagaatttaatactttattaaattttattaatgtttacttctacctgtttagactatttttaaggaatgtagacatcagtactactcgaagtgtggtcccatattgatcccatattgatcaactgtcattggctgatggagagataagcacataaagtgagcaaacatgcataaacatttagaaatgctgatagtaaactgacagtgccaatgcattcaagtacatgattttgtatttacnaaaagtatccttttatgaatgggtttagaatt

SEQ ID NO: 86 - DNA, 656 bp, Homo sapiens
  misc_feature (1)..(656): n is a, c, g, or t
ggcgtgaaactgntnctctactaaaaatacaaaaaatagtggggcatggtggcgcattcctgtaatctcagctactcgggaggctgagacaggagaatcacttgaacccgggagcagaggttgcagtgagccgagactgcaccactgcagtccagcctgggcaacagagcgggactcggtctcaaaaaaaaaaaaaaatgaataagacagtagtctcacctccaggaacataacctagatgnngtanagncgncgaacggntnagcnggtntgngncnactaatnttncacagggtaattgaggcagagtgggactctaaagggtctaagatatttacaaggggtgctagaggaaagaangagaatatatagggtccaaaagactttattttcttaggggagttttacatcatctccccacaggcagaagccctgggttatgtgactatgccagtaattgagtggtttaatctccagtttagggatatggggtatttaaccagtccctgttgctacagattgaaaagacatattctttaattttgctaacaattaaaggtgatgtttgatctccnggagtaacttctccatcttcagggggtttccaaattctggngggaaatncaggggtgttncccatttttatcattnggatc

SEQ ID NO: 87 - DNA, 410 bp, Homo sapiens
  misc_feature (1)..(410): n is a, c, g, or t
cattttaattcactgaactatattttttggtacattacccttcaactaaaaaaataaaattaaaacatttccctattactgatgaaggttagaatgaagagaacataaggtatataagtaggaaagaaaacctatgtagggacagatgttaatagttattaaatcctaagtaaaattttcagaacttggaaattaccaaatccaggagtggtcagattcctttatgaaggtagatctggagctacttaggccagatttttgtattttagcaaagttcctcagatgattctgacgcacacctggattataaaccactaaaccactgaactaccccaagaaggttacgtgacctcccagagctagaatgtnccagaaatggtgcaagaattcnattactggactcctggccc

SEQ ID NO: 88 - DNA, 434 bp, Homo sapiens
  misc_feature (1)..(434): n is a, c, g, or t
gaaatcacaacaaactgaattaaacatgaaagaacccaagacatcatgtatcgcatattagttaatctcctcagacagtaaccatggggaagaaatctggtctaatttattaatctaaaaaaggagaattgaattctggaaactcctgacaagttattactcgtctctggcatttgtttcctcatctttaaaatgaataggtaggttagtagcccnnnagngtctnaatnctttangatgctatggtttgccattatttaataaatgacaaatgtacttaatgctatactggaaatgtaaaattgaaaatatgttggaaaaaagattctgtcttatagggtaaaaaaagccaccgtgatagaaaaaaaatctttttgataagcacattaaagttaatagaacttactgatattcctggtctagtgggtataata

SEQ ID NO: 89 - DNA, 410 bp, Homo sapiens
caggtttttattatttattattattgtttgttttgagatgcaatcttgctctgtcacgcaggttggtgtgcaatggtgcgatcttggctcactacaacctccgcctcacgggttcaagcaattctcctgcctcagcctcccaagtagctgggatgatgggcgtccgcgccgtgcctgggtaaatttctgcatttttagtccagatggggtttcaccatgttgggcaggctggtcttgaactcctgacctcaggtgatccgcctgccttggctcccaaagtgctgggattacaggcgtgcaacccgcgcctggccccaaatgtcatgtttttaaataaaaacatagaaaatgatataaaggttcacagcatcatcaagaaaacagttcccccgtgtcgcggaggggagatg

SEQ ID NO: 90 - DNA, 224 bp, Homo sapiens
  misc_feature (1)..(224): n is a, c, g, or t
gcagngggaacgtcttctcatgctccgtgatgcatgagggtctgcacaaccactacacgcagaagagcctctccctgtctccgggtaaatgagtgcgacggccggcaagcccccgctccccgggctctcgcggtcgcacgaggatgcttggcacgtaccccgtgtacatacttcccgggcgcccatcatggaaataaagcacccagcgctgccctgggccctgc

SEQ ID NO: 91 - DNA, 169 bp, Homo sapiens
  misc_feature (1)..(169): n is a, c, g, or t
aaaataaagaatcagatttattggggtggttaagtgagatcatggatgaactgtttacttctattcagcagtagcctttgtggtcccaggcttctggtggccagataattccctcaactccatgagcaggtgaccgaggaggactctcacatcctgcatgtgtttnaga

SEQ ID NO: 92 - DNA, 444 bp, Homo sapiens
aagttttgccactaactttaatgtatcattaggcaaattatcctctctgagccaaagaagggtagtgggattacgggatctccaaggatcgttcttcttaacattgtctgatggcataattgtcttattaagatttctagggagaaatacaaagttaaaaataaaatcatataggttaaaattatgtaaacatctggcctagagcctcttgattcaactcacataactaaccagaccatgggggccaacaggtcaaaggacactatgtaaaagacatgacttagacacatggagtgagaggagcaacacaggctcccatgggtggggactgagctggaaggtcacagtaatgagtgaactcccccttgggcacactttagtatgatgagtaaagcttccctggtgactttagagaatggatcatgggaacacctttataagaag

SEQ ID NO: 93 - DNA, 512 bp, Homo sapiens
  misc_feature (1)..(512): n is a, c, g, or t
ggggatctgcctgaagcagggatgggacacnaagtccctccagcttatctntncacaacaaccctttccctgcaganatggtttgtataccacaagccctcttagcacgcaaaagccaaaatctaaagatcaaccatttatcctgaacaacaccatttgagaaagaggtaaccatctttggttctacatggtttggagagtatagtggtaggaggggctccctgattcccctaaagctatgcacaccacaaggggctctgctcttctgtctgggatcttcttataaagtgttcccatgatcattctctaaaagtcacaaggaagctttactcatcatactaagtgtgcccaagggggaagttcactcattactgtgaccttccagctcagtccccacccaatgggaagcctgtgttggttctctcactccatgtgtctaagtcatgtcctttacatagtgtcnttgaactggtgggccccatggtctgggtagttatgtgagttgaatcaaa

SEQ ID NO: 94 - DNA, 451 bp, Homo sapiens
  misc_feature (1)..(451): n is a, c, g, or t
catgatcattctttttaatgtgcaccaaattagcagtaaaaatagcagcagatggatcagagtggttgtcaataaaccttttctccccaggttactaatatacaattgccatgaaaaataaaaaaatatatatatatatttacacttgactcatcacctctgcttaggaccctgtaagcacaagatattgctgaactgctgtatttgctacatatggaacaattagactagcaataagaagtagtttatgcatgtatgctggcctacatgnatatacccctttccgcaattactgaggattatcaacaaagtttggtcttggtcttgtgattataatnccaatnaaatnacatnttaaatggggatatcnccgaattntggttttnataattacgtaattaattccnaagaaattaaataggtaatatagacccctgtaaaaantaaccnt

SEQ ID NO: 95 - DNA, 435 bp, Homo sapiens
aggaaggccagagtattaatatccccatctgtgtcttttgccttccatgaacctgggttttgagccctctcttgtaaaatgggcacagtaatattacctacctcagggagttgtgaggattaaacatgaagtgctaagcatagtgcctggtacaaagacagtactcaataagtgctacctaaaactagtattcatagcaatactgttaggataaagaattatcatatatgagatagttccaaatttttgtttttttaaaaaaaaaagagttttataagttcaagataatattttcttacttcaaagaaacaatctcacaacgagggaatggtaagaatcaggagagattactaacctggcagaggagctatcacaatcacaaaggtggtttttccagggcacggctcatccattacactccagatgtgctgaccc

SEQ ID NO: 96 - DNA, 378 bp, Homo sapiens
  misc_feature (1)..(378): n is a, c, g, or t
tcagtttacaatgcataatgatatgtctttatttcatcaacagaaatggtgtctagacaaaattcagttaacactagcaattcaattgagtgaaaactttttttgcacaatagtgtatttacaatgagtaaatgaagtttcaattcattagttcatagcaatgcttttttcccccaaaaggtaaaaattcttagttacagagaataagcatcaacagcctttcattttttacaatnaaaaccncgggnaaaaccncaatcccctttggaaaaaaattangggccaggcctaggacctaggtncaataaaatggatggcattggaattaaatttccattaatcggcataggaatcccgnggtaaaangtttggtaggaa

SEQ ID NO: 97 - DNA, 457 bp, Homo sapiens
  misc_feature (1)..(457): n is a, c, g, or t
atgtctacaaggttttattaaaattaagtttaacattaataacacactaatataaaggtaaaatttagcttatctggtataaaagtcatacaggaagcattagtaaatataaaatagcgtttagctttcttttgtctaaaaactaataaaaattggtgctaaaggaagcattcattttactagaggatcataaaagttaaagacttaaaacaaactttggcaattaagacagcataccaagatgcaaatgcctggttgaaatggatcaaatattccatctgcaggttaaacaaaagcaattagcatgcttgtgcacatggcaggccagagaccctgattgtcccccttccactaaggtggtcctccagtcgggccaggcatgggctgcatggtagctcttttccaggattctatagcctggagtaataagtcatgccaagctctctcctgctatatn

SEQ ID NO: 98 - DNA, 548 bp, Homo sapiens
  misc_feature (1)..(548): n is a, c, g, or t
tttttttgactcttatctaactttacttccaaacaatgatttataaaatgtggaggagagtgggtgtctatgtcaagcagccttatgataaggctccgtcatatattgtgcttattcaacaatactggtgttaatgaggcctggcctccaaaggacaaagatacagaaacagaaaggttttcccaggccagaagtattagtttacatcacaacattaaagcataatacactgtgggcctaaaaattaaactgcatgtgttctagcagcaggaacaacaacaacaacaacaaaatgctttcacatttatataaaaatgacaaagtaaaaagcagagaacacagtgaaaagtgtctggcagttcattaaaatacagttgagttgcttctatagtctcaaaccattattatattatttgaatgagaaagagtatgaggatttaactggctgaattccattcctaccccttattcataggggaataattaccctgatattatttaaaagtgtttgctttacncaaaantaaataaccttaaatatttaaaat

SEQ ID NO: 99 - DNA, 459 bp, Homo sapiens
  misc_feature (1)..(459): n is a, c, g, or t
ttttttccaggaaaaaaattaaatctttatttttaaaaatcccacaaatccataatgaaatcatcatctgaaaaaaaagatggtagggaacaaaacgtgggatacatttaaaaggcactagattcattaataccagagccattctggagatgccatgtaagaaatctggagttactctaaatcttcttcttagtggtatcagaactggggagaagggtccaagcaaagtgttgcctttgccagtgtattcggatcgaggttatgaggaagagcccttttcctttgtcagtgagtttcatgttggtccaccactccagcgctgacagctccccgatggccctgtcatcgtatctcaggacctccttcaggatgtgcgttgtgtgctgccgacaggggggcggcctggctctgacacttganttactgtactcacactgggctatgaagtacacagttaga

SEQ ID NO: 100 - DNA, 443 bp, Homo sapiens
  misc_feature (1)..(443): n is a, c, g, or t
tcataggcccagctgtgagatacagtaagttcaagatgtcagaggccaggccgncccccctgctcgggcagcacacaacgcacatcctgaaggaggtcctgagatacgatgacagggccatcggggagctgctcagcgctnggagtggtggaccaacatgaaactcactgacaaaggaaaagggctcttcctcataacctcgatccgaatacactggcaaaggcaacactttgcttggacccttctccccagttctgataccactaagaagaagatttagagtaactccagatttcttacatggcatctccagaatggctctggtattaatgaatctagtgccttttaaatgtatcccacgttttgttccctaccatcttttttttcagatgatgatttcattatggatttgtgggatttttaaaaataaagatttaattttt

SEQ ID NO: 101 - DNA, 337 bp, Homo sapiens
  misc_feature (1)..(337): n is a, c, g, or t
acatttactagtttattgaatatgaggtttatccatttagcaatgtaaggaaaactttagttctgtttctcagttatcaggagtgaacataaaactattctaaaccacaattagtttaccagcatagtacaaaataaaatngacaactaacgaaataaagcaattaaagtaacttatttttactcataaggttaccataataataaaaattcctttaattttcaaagcactcttcatgaaaangtagttgggggaaaattactatttgttccaangtaggataaaagggnagggatgcncccaanttaaacatttttattnaaaaattaaacccccc

SEQ ID NO: 102 - DNA, 412 bp, Homo sapiens
  misc_feature (1)..(412): n is a, c, g, or t
gattgaaataccatcagaggcccaagctctcttttccagagagcagtggcttttgtaataattcactatcttagagtgaaaaaggactagacctgtgttacataataatcttggttcaagctgcccttctgaacaaagatataaacctagcatacattgtaatagataactggtaaaactgacaacttttacttctcagaggccatttaaatataataggaacctactgaccaaacctagtgatacataaaattaaagcctgtgnactttttaaagttgttaatcactatacatatgtatgtgtatatgtgtatacacatatataattttatgatcaatatcttagatattttagaaattccctttngaatagtcttggcgtgccgtggaaaaatagaaaatcagggagata

SEQ ID NO: 103 - DNA, 458 bp, Homo sapiens
  misc_feature (1)..(458): n is a, c, g, or t
ataatttattagatctaaagccccttcctccccagcccctgctttcattaaggtatttaaacttgggggtttcactgctctcccccatgatggagggagggagccccccaacctcagtgaggagagccccgagccggccccggggaaagaggggtgcagagggagttcccccagatcagtaccccccacccctccccagctagtagcatgaccaggagacggttaatgagagccaagaggagtacctggtgcacctggtgcggtggtggagacctggggggcaggtggatctggggctgttcccccccctccgttttttccaccccacagttcctcctgggatctggccctccagggnaagtggagcctccagcccctaggggatgcatgaggggggagggggtgctgagtgggaggaagagtcaggctcacagctggggtggcctgggggtgggggt

SEQ ID NO: 104 - DNA, 404 bp, Homo sapiens
  misc_feature (1)..(404): n is a, c, g, or t
gtaaaacgctaataatttattagatctaaagccccttcctccccagcccctgctttcattaaggtatttaaacttgggggtttcactgctctcccccatgatgganggagggagccccccaacctcagtgaggagagccccgagccggccccggggaaagaggggtgcagagggagttcccccagatcagtaccccccacccctccccagctagtagcatgaccaagcntagntttnatgagagccaagaggagtacctggtgcacctggtncggtgntggaagacctggggggcaggtggatctggggctgttcccccccctcccgttttttccaccccacaagttcctcctgggatctggccctccagggaagtggaagctccagcccctaggggatgcatg

SEQ ID NO: 105 - DNA, 440 bp, Homo sapiens
cagcaacatgaagttggcagccttcctcctcctgtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttttttaataaatcatcattgtta

SEQ ID NO: 106 - DNA, 447 bp, Homo sapiens
cagcaacatgaagttggcagccttcctcctcctgtgatcctcatcatcttcagcctagaggtacaagagcttcaggctgcaggagaccggcttttgggtacctgcgtcgagctctgcacaggtgactgggactgcaaccccggagaccactgtgtcagcaatgggtgtggccatgagtgtgttgcagggtaaggacaggtaaaaacaccaggccctccctgctttctgaaacgttgttcagtctagatgaagagttatcttaaggatcatctttccctaagatcgtcatcccttcctggagttcctatcttccaagatgtgactgtctggagttccttgactaggaagatggatgaaaacagcaagcctgtggatggagactacaggggatatgggaggcagggaagaggggttgtttttttaataaatcatcattgttaaaaagca

SEQ ID NO: 107 - DNA, 373 bp, Homo sapiens
  misc_feature (1)..(373): n is a, c, g, or t
tctgaagtcacagcagcaatacagaacaaagaatttaccttaatctgatctttttacgtggaattccctgactcaaactcagtggcttagtttggaaacctctgaatggctggggagagaaaatcttttgaaactaagtgaataaattaacacacacatacgtnggaaatcagcccttgtgcaagtgtaacatgaacatcactgatgagagtgcagaaactccaggcacccctctgcctcctcctatccctgggcctggggttgtagggagaagtcacactcaattcatttctagccacaccatgtccctaacagtgctagtgtnaactagccctgacctgggtattgggtttaaagaatggagcctcgtgcc

SEQ ID NO: 108 - DNA, 367 bp, Homo sapiens
  misc_feature (1)..(367): n is a, c, g, or t
gctcattctttaaaccaatacccaggtcagggctagttcacactagcactgttagggacatggtgtggctagaaatgaattgagtgtgacttctccctacaaccccaggcccagggataggaggaggcagaggggtgcctggagtttctgcactctcatcagtgatgttcatgttacacttgcacaagggctgatttccacgtatgtgtgtgttaatttattcacttagtttcaaaagattttctctccccagccattcagaaggtttccaaactaagccactgagtttgagtcagggaattccaccgtaaaaaagatcacgattaaggtaaattctttgttctgtattgctgctgtgacttcngna

SEQ ID NO: 109 - DNA, 523 bp, Homo sapiens
  misc_feature (1)..(523): n is a, c, g, or t
ttgttttgttttctttcacagatttaataccgcgatctcagccaaactccggccgagaagttgagaaatgtcttcaccccctctcgacattcgttcgtgcttcttcgccttggtggagcgataggggcgagcaggggtggggccggctggtgctgctacgagggccgtgcagcgnttnaataagtgacataaaatgtctacacgcataagtaaccgtacttagggcttctgcaagggccaccagagcgcctaaggtggcaagtgggccccgtgtcacnggccgcgctgcaggcgcttgcgcaaagtcttccacgcagccgtccagccccatgcgctccagggccgcgtaaacggctccgaggcccgcgggttgctgctggcgccaggctttgagcatctcgtactgctggtctcggaaacggccgatttccancttcaagggcttcgatctctgcctcgcgaagcccagcgtgccaacgaacttcttccaagcgccgnctgggaacngcgtcaatcaaggtcg

SEQ ID NO: 110 - DNA, 372 bp, Homo sapiens
  misc_feature (1)..(372): n is a, c, g, or t
cacaagccctggttactgcagatgaagctgggatggaggctctgaccccaccaccggccacccatctgtcacccttggacagcgcccacacccttctagcacctcctgacagcagtgagaagatctgcaccgtccagttggtgggtaacagctggacccctggctaccccgagacccaggaggcgctctgccgcangtgacatggtcctgggacagttgcccagcagantcttggccccgctgctgcgcccacactctcgccagagtccccagccggctcgccagccaatgantgctgcagccgggcccgcagctctacgacgtgaatggacgcggtcccaagcgcggcgctggaaaggaagttccgtgcgc

SEQ ID NO: 111 - DNA, 454 bp, Homo sapiens
  misc_feature (1)..(454): n is a, c, g, or t
caatttttaaaaatgttttattacaaagcttcttttaaaaaaatgctcagcacattaactcaaactggaatgacaaacgttaggatgacagttttgggcaaaggctgtgcttgcttttttaaaaaatgggtacatcaatgctcattttaacaactnggcataaaatcccactaattggctaataaaaacagatacaaatacagaacatttaaagtaataacaattcaagtgctgggctttttacaacaagggggtgataaggaaagaaatgaaaattcactgcaaaccagtctgctgaacgcatctgttaaggtttactgtttaaaaaaaaaaaagaagaaaacagaagaaaaaataaactgaaattagggctgccaattgctaccaacagagtgggtttggctattacatttatttagctctactggaacaccttacaagggcggagaagcca

SEQ ID NO: 112 - DNA, 452 bp, Homo sapiens
  misc_feature (1)..(452): n is a, c, g, or t
acttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaacccactctgttggtagcaattggcagccctatttcagtttattttttcttctgttttcttcttntttttttttaaacagtaaaccttaacagatgcgttcagcagactggtttgcagtgaattttcatttctttccttatcacccccttgttgtaaaaagcccagcacttgaattgttattactttaaatggttctgtaattggtatcnggc

SEQ ID NO: 113 - DNA, 459 bp, Homo sapiens
ttttttttcggtatttgaatacatttattgtgacaagaatgctgttataaatattcataagcaaaggccatctttttatctaggaattgtcaaagagaagattccaaattggaaggatacatcttttgtaaaatctgccaccaattcctgctttgagaataagcacctattgtaaaatttctactaacattataaatggtcacagcacatgccacttgatacaatccaaactttgaaatgtttgacttctcagtgggctgtccctctccactgcaaccccccttcctccagcctcctgaaacatcgcactatcctttcggtaagcaattccatatagatagctggggggaggaggagtataacctggaccatagcatcaggttacatcaggtacatttatttctaaagtctaatagagaacagtttttactgcttaatagtaagaagcactgagagtga

SEQ ID NO: 114 - DNA, 395 bp, Homo sapiens
  misc_feature (1)..(395): n is a, c, g, or t
gtatcctgcccagtgttgtttgtaaataagagatttggagcactctgagtttaccatttgtaataaagtatataatttttttatgttttgtttctgaaaattccagaaaggatatttaagaaaatacaataaactattggaaagtactcccctaacctcttttctgcatcatctgtagatactagctatctaggtggagttgaaagagttaagaatgtcgattaaaatcactctcagtgcttcttactattaagcagtaaaaactgttctctattagactttaagaaataaaatgtacctgatgtacctgaatgctatggtcaggttatacctcctccctcccccaagctatctatatgggaatttgcttaccaaanggatagtgccgatgtttc

SEQ ID NO: 115 - DNA, 154 bp, Homo sapiens
  misc_feature (1)..(154): n is a, c, g, or t
ggtgtaattagcatnggtcaatgcgggacgatngagtggctctggaaacctgatggatttcctcgatgagccgttccctgatgtggggacgtatgaggacttccacaccatcgactggctaagggaaaagtcacgggacaccgacagacacatg

SEQ ID NO: 116 - DNA, 214 bp, Homo sapiens
  misc_feature (1)..(214): n is a, c, g, or t
taaatgacacagtcagtgtttttctgaaaataattgccaccttgttgctaattaaacatgatggattcggggtcctggtttgccatctgggccatatgtctcagaacatccttttttgtgatgatgccaagaagtctccngctcgggtcacaaggaattgcngaaggccccagttttccgggangatatncaacaacggtttcaatcggaattt

SEQ ID NO: 117 - DNA, 256 bp, Homo sapiens
  misc_feature (1)..(256): n is a, c, g, or t
tttaagctagaaaaaggccaaaaagcaaaacctgagaaaacaatacgtgttgttttctcaggaaaagaaaaaccttcatgaccctactgaagagcattggagatcagcttccgctaagatgctagcttggccaagtctgttatattcacctgaaaaagtcttagcagagaatttttgcattcccacccaaaagccctctcagccactcaaatgcctatcttctccagtctacaagttacatgntcccacccagcat

SEQ ID NO: 118 - DNA, 260 bp, Homo sapiens
accgaagcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaagaatgcttgaagtacccctgggcttctcttaacatttaagcaagctgtttttatagcagctcttaataataaagcccaaatctcaagcggtgcttgaagtcc

SEQ ID NO: 119 - DNA, 435 bp, Homo sapiens
  misc_feature (1)..(435): n is a, c, g, or t
taagaggttgcgaacatacatatttatttataatacaaaatnaagattngagggaaaagtgctttaaaaagtancatgtaagtgtataaatgaaattntngcttcttctccgatacaattttgattgggtgagcattatttgcttttacaataatgctttattttgttttttgcattgcattgcactaacctgtccattaatacaaacagaaaaagaaggtggaggacgtgcccagccgcgtggtcagcgtgccgaacctcgcctcctatgcaaagaactttctgagtggcgatctgagttccaggattaatgcccctccaataactacatcacccagcttggacccaagccccagctgtnggcctggaccctacaaacccanaccagtctacagattgcaaaaactgccacaaggttttggggggaatgtttgg

SEQ ID NO: 120 - DNA, 417 bp, Homo sapiens
  misc_feature (1)..(417): n is a, c, g, or t
aagatttttgtnccaagtccngtgctaagcacatcctatggattaattcctttagtctcacgtcagtctgatgagataggtgctgtattatcttaattttaaaggcaaggtatatggagacctggagaggtcaagtgacctgtccaaggccacagagctaagaatgaggaagactgtaatttgaattcagacctccaggccagatggagtccaccttttgtataacccatgctgaagttttcaggtaagtgattcagtgtcccttgtctaatcatccatgaaaaaaggccttctggaatttggtaccaggtgctagaaagaatcctacttcccctctnatctacanngnaaanacgnataagggcccctgtccccaacatcccccaaaccttgtggcagtttttgcatctgtagact

SEQ ID NO: 121 - DNA, 442 bp, Homo sapiens
  misc_feature (1)..(442): n is a, c, g, or t
ctatcattgtgaactttttcctctcctgatccagttcatcatggaggctcatcatttctgtttccaaagtcaagtttcgctgttctaagctctttatcctggactcatactctatcttctgtttggtcatttcctgtttcaggctggaaactaaactgtgcagtgcntgtggttgctgctgctgttgcccacaaatgtctcactgttgtcactgctactggtggcacgactacttcgacctcccacactcctgtggtcacttttgctttcatagtcccttggggtgggaaaggtcgtcctgcgggggcccatccaaaacagggtcctcaaagttccccccaaaaaagtcttgctctgggcaggtggtggtagaagagcgacaggagttggagttctcagggagggagatttcacaggaggaagtggaccaggtagcactgna

SEQ ID NO: 122 - DNA, 477 bp, Homo sapiens
  misc_feature (1)..(477): n is a, c, g, or t
tttttttttcacaattggaatgtgctttatttcagggaaatataaagggaaatgaatgctattataacttggtagaacagaagaaatggctacctagctttgctttccaactacaaacataaatgaggatctcagcatttaaggtaaaacatgataagcacaaaaggagagttcactggggactggactccctcatttactctagaaattatgagaaccagcagcaatattcctcaagcatccatctcaacatcaagttcctttgttttatttaccagatgaccagggaatcataggatgagtttgggctgcaactgtgtcttccactgccattcccaaagacttgaacacggtgggtcttctcacagtggggctgggttcacttcccttaatcactttttcccaggttcaggcaaaggntcttggggcccctggaccaggcaggggacattttccaggattnctttcagggggcag

SEQ ID NO: 123 - DNA, 97 bp, Homo sapiens
  misc_feature (1)..(97): n is a, c, g, or t
ncgcagctccagnctcctcatcccgcctctagagacgnccctggcaagcttntncagcggtcccgaagngggggtnatgcagcccgtgcgcancgtg

SEQ ID NO: 124 - DNA, 430 bp, Homo sapiens
  misc_feature (1)..(430): n is a, c, g, or t
actgaggttagaaggcacaggtggcgagatgagccgggtaccagcgttcctgagcgcggccgaggtggaggaacacctccgcagctccagcctcctcatcccgcctctagagacggccctggcaacttctccagcggtcccgaagaggggtcatgcagcccgtgcgcaccgtggtccggtgaccaagcacaggggctacctgggggtcatgcccgcctacagtgctgcagaggatgcactgaccaccaagttggtcaccttctacgaggaccgcggcatcaccttcggtcgtcccttcccaccagggtaattgtggttactcttttgagcccagcaatgggcaccntgctnggcggtcatgggatgggaaatgttcataaattgcaaagagaacagttgcattttttgccntttgccaccaatttttttg

SEQ ID NO: 125 - DNA, 394 bp, Homo sapiens
aaaaacagacatagtctcactgttgtccagattggagtacagtgacacaatcatagctcactgcagcctcaaactaatgggatcaagtgatcctcctgcctcagcctcccaagtagctaagcctactggatgcactactatgcccagctcacacagaaggtttctgagtaatctgttgctctttttccctacaatttgtcttccatataactcaaactgacaaggctatggcttacataaagaaatatattataaatcaacaacactcatgataagtttacataagacatgagaatacacctgaatcaccaaccgggaaaaatgattgaagagcttgaaattaagcctaagtgtaagtctctgttaagcttacaacattacaatagttaaatcg

SEQ ID NO: 126 - DNA, 392 bp, Homo sapiens
  misc_feature (1)..(392): n is a, c, g, or t
tctaaccttcgatttaactattgtaatgttgtaagcttaacagagacttacacttaggcttaatttcaagctcttcaatcatttttcccggttggtgattcaggtgtattctcatgtcttatgtaaacttatcatgagtgttgttgatttataatatatttctttatgtaagccatagccttgtcagtttgagttatatggaagacaaattgtagggaaaaagagcaacagattactcagaaaccttctgtgtgagctgggcatagtagtgcatgccagtagncttagctacttgggaggctgaggcaggaggatcacttgatcccattagtttgaggctgcagtgagctatgattgtgtcactgtactccaatctggacaacagtgagact

SEQ ID NO: 127 - DNA, 452 bp, Homo sapiens
gatcattccatcatgtattgatgcatacaaatatcacattgtaccatataaattatacaattattgtacaaatatatacatcaatatacaattgtacatacaatacatacaattgttgtacaaatatatacaattattacttgtcaattaaaaattttaaaaaagaaatctgaaataacagttgccccctatgagcatctcacgataaatccctttaatctcctctacatatactgagtattaaaaaacagaatcgtctagaacattgttgctgttctgagacctgtctttctcatttaacacaagtgaacatttttctttgtcagcaagtagcggtaaacatcatccattctaatggctgtattttttaataggtggagttgtatcttcagggcagattcctaacagtggaatggctgggtcacaagggaaatgtgtaggtagtttttgga

SEQ ID NO: 128 - DNA, 470 bp, Homo sapiens
  misc_feature (1)..(470): n is a, c, g, or t
gtgacaagcaaccttaaaagagacacaaggagactggcagacagaggaagaagaggcagcaatgtgaccccggangtggaaatctcagtgatggggccaggaatgtcaaggaatggtcaaggaatggctacagcaccagaaaaagaggcaaagtgaggcttctcccctagaatctctaggagcgctccagccctgctgatgtctagatttttggagttctggcctccagaatgtgagagagtaaactattgtttaaagctaccaagtttgtggtaacttgttagagcagccacaggaatgaatgtacagggaatcagggcagtctcatacactgatggtgggaaaacaaaccggcacaacccttatggtgggaaatttgacaacattgtacaaaaactacctacacatttcccttgtgacccagccattccactgtttaggaatctgccctgaagatacaactccaccta

SEQ ID NO: 129 - DNA, 476 bp, Homo sapiens
  misc_feature (1)..(476): n is a, c, g, or t
tttttttttttagtctaaagaaagttctgaacagaatatcaattaagcttacatcacaaaaactttaaatgtatttacagagtgaataagttacatagataaactctgaatatgtttctgcagtgcaacaagttcacatgcacacatctaacacttgacagcattaagtttaaggagagaacttaagaatggccctttacatatatattacacataaaatatgacatcgaagaaacaaagtaacaactcatattttacctttatgattctacttctgactatccaaacaggatattaaaatatggcatgcctggacagggtgaaaagacttggggatttatcttgtggaatagttttctctacaaaacgggcaaagtttaattaaatttaacncttcattccttccggcggtttnaaatatggctcnttaaaggcnaccttctggttaaaaggccggcccggttcccttnaaaagg

SEQ ID NO: 130 - DNA, 408 bp, Homo sapiens
  misc_feature (1)..(408): n is a, c, g, or t
gccacactctcttngcttgcaaattgtaaggcaacatttgcagggggatcaagagatggagtaattacctgtcaaccaggggactccgaagaaaagcaaatggaatctcttgcacaattggaactgtgtcagagattatataagctacacttccagctgctattgctttttcagtcctactgtaagctcatcggccagggtgcacgaagttagctccatgccagagctgctgaatatgtccaggggaactgagtgacctaaagaaacacctgaaggaagccagtgcagtcattgcagctgaccctctctatttcagacggcgcgtnggtccgagcccacctttcacgtncactgaagcaggccatccagttccatgcttgggagttgccttgaagggaccaacggact

SEQ ID NO: 131 - DNA, 329 bp, Homo sapiens
  misc_feature (1)..(329): n is a, c, g, or t
gaagtaaaagatttttattgttctatagacacttctgaaaagagatctaattgagaaaatatacaaagcatttaagagtttcatccccagagactgactgaaggcgttacagccctcctctccaaggctcagggctgagaacggttagcatatcgaatgatcagtaaaaacatgcaaaagtgagaaggaaagggaaaaaggtgcattcccctaagctgagggggatggaatttcagaacagaggangcagggtggacaagtaccaaggtggctctccctttccctctgtgtnatctttcaaaaccanttccaagcntggatnaaagcaa

SEQ ID NO: 132 - DNA, 384 bp, Homo sapiens
  misc_feature (1)..(384): n is a, c, g, or t
ttttttttttagcacaccacagccaccatacagacaggagtgcagcccctcctccctaggaacccccacccctactcttcactaggcagggcccatggctcatgaatgcagaacagtcaccccagccatggctgagcatacccactgttagtgacacagagtttccctgagaagaggctcccaaaggcatacgacagccccttggccactgccacagtaacagtgctatccctcctgcccttggantaggggaggacacaaagagcctaagggctacacttcaaacttaggagtacatcacagccaccatatgggagaggagaccaacctcttcctccctgtgaggcctttcaactncctgctccccaacaaacagaaccccaa

SEQ ID NO: 133 - DNA, 66 bp, Homo sapiens
  misc_feature (1)..(66): n is a, c, g, or t
cagtactgcggccnncnctcctntccnaacctcgctctcgcggcctacctttanccgcccgcctgc

SEQ ID NO: 134 - DNA, 387 bp, Homo sapiens
  misc_feature (1)..(387): n is a, c, g, or t
attgaaaatagatgttttattttgtttatacaaggtacaatgtcaaaatacaaataatatataatgtatagatataatagacaaggaagtataaatataaacgcatatattcgtaaaatggcactgagttgagttttcttcttcctgaatccttcaatggagaggattcnctgggctcagcatctctcccacctttcccaggtccctgtccatgtgtgcagagagctggagacagggtggttagaagcccaaacgctggtgtcttccctgtagacgtctcccacgccaggagaagccttgtaattgacagagagctttgggtatgtcacttttctctgtgaactgaaagtttaggatgagggcncggaanattcggggcagggtttt

SEQ ID NO: 135 - DNA, 188 bp, Homo sapiens
  misc_feature (1)..(188): n is a, c, g, or t
acnagcatccgcctcccaccagccgccagtgtngtatccacagggccacagcgacaccactgtggctatctccacgtccactgtcctgctgtgtnggctgagcgctgtgtctctcctggcatgctacctcaagtcaaggcaaactcccccgctggccagcgtttgaaatggaagccatggaggctctg

SEQ ID NO: 136 - DNA, 410 bp, Homo sapiens
  misc_feature (1)..(410): n is a, c, g, or t
ccttcttgttcactnggtgtggtttattcttgaagcaaggtctctctccagttgaagcccccagttggtccatgggtaagaggaaggattggtggatctgtcagctgccatattccagtttctcctaattcttcacaggaacaaaatcccagatatgggatctttcggaccatttgtacgaagtccttggagttctgaggtgacaggccctgaagttggcaggtacacgcttcaagggaagatgcgtgggccacaatcaggatgttatttcctttacttttacattcacttattattncttttgttacttgggaaacttctactngatataagtatcataggattctggaaacaactaatttngctcgattggaatgtgaggnctggtaggttgtatcaacactcaggtt

SEQ ID NO: 137 - DNA, 176 bp, Homo sapiens
  misc_feature (1)..(176): n is a, c, g, or t
cnttcggcacgatggggagtattggagaggcggccttatgangnccangngctcggggagacgactcctcttactatcatctgccagcccatgcagccgctgagggtcaacanccancccggcccccagaagcgatgcctttttgtgtgtcggcatggtgagaggatggatgttgt

SEQ ID NO: 138 - DNA, 503 bp, Homo sapiens
  misc_feature (1)..(503): n is a, c, g, or t
tttttttttttcttccttttttttcttttagaaatattcaaattttaaaacaacaattaagtggattatgggaacaggaaaaccatcttactttggttccaggatatactggtaatatagctaaggatgtagatgcttatttattacagttacattgagagatttcatctactaaagagcatttggtttttcaaaacatccctgaactgtataatttacaaaaaaaaaaagtctcgtctgagaactgtgaactgtggaagaaatcaaaactatttttncttttaaaaagccacgtaatgaaaccnctaatgaaatcccagcaatctgcttcacattgaagtggaaaaatatccaaaaggagcagcttcaattttcattgaggtgaaagtgcactatgaagattgttcacctttggctgcatttgggagttatatggttatttggtaacnttaagaactnctggatttttaatgccatccnggcatnaaaatatnatttntacc

SEQ ID NO: 139 - DNA, 563 bp, Homo sapiens
  misc_feature (1)..(563): n is a, c, g, or t
taataaaacacaatcttaaaaaagttaatgatgattggtcttggtggttcctagtggtaagtcctgtcttattttttcacatagtataaattatatttttatgcaggattgcattaaaatccagtagttcttaatgttaccaaataaccatataactcccaaatgcagcaaaggtgaacaatcttcatagtgcactttcacctcaatgaaattgaagctgctccttttggatatttttccacttcaatgtgaagcagattgctgggatttcattagtggtttcattacgtggctttttaaaagaaaaaatagttttgatttcttccacagttcacagttctcagaccgagactttttttttttgtaaattatacagttcagggatgttttgaaaaaccaaatgctctttagtagatgaaatccctcaatggtaactggaaatanataagcatccacatccctagcnatattaccnggtatatccnggaacccaagtaagatgggtttcccggtccccataatccncctaaatggtggtttaaaaattggantattcccaaagg

SEQ ID NO: 140 - DNA, 429 bp, Homo sapiens
  misc_feature (1)..(429): n is a, c, g, or t
ttttttttttaaatgaatgtaacaagcatttattaaaaactgtgctgcacaaaacatgttagaaactagaccaggtgctaggagtctaatcaaggcaggggcagggtaaaaacatgggaatattacatggacaagcttgtctagcatggcagtctataacccttgagggtttacataaaataaaggaacatttttgtggnctcaggctccccagagttctctttatgtttgggcagagactgcccatcccttagtgatcccaccttagagccaggttttcaaagtcatttctcccagtatatctgtctctgtatgcaagtttcctctggttgccttgagcaaaaacaatcatccaagtcaaatttgctagccctatgctgggccagcccacgtttcccgtaggacatctgtagggtaagttnagccccg

SEQ ID NO: 141 - DNA, 499 bp, Homo sapiens
  misc_feature (1)..(499): n is a, c, g, or t
ttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagccaggcttaatggctcatgacctggaaataaaatttaggaccaatacctcctccagatcagattcttctcttaatttcatagattgtgtttttttttaaatagacctctcaatttctggaaaactgccttttatctgcccagaattctaagctggtgccccactgaatcttgtgtacctgtgactaaacaactacctcctcagtctgggtgggacttatgtatttatgaccttatagtgttaatatcttgaaacataggaggatctatgttactgtaantagtgtgattactatggtctagagaaaagtctacccctgctaaggagttctcatcccn

SEQ ID NO: 142 - DNA, 575 bp, Homo sapiens
  misc_feature (1)..(575): n is a, c, g, or t
gcttttaacaatgatgatttattaaaagaaacaacccctcttccctgcctcccatatcccctgtagtctccatccacaggcttgctgttttcatccatcttcctagtcaaggaactccagacagtcacatcttggaagataggaactccaggaagggatgacgatcttagggaaagatgatccttaagataactcttcatctgtccttaccctgcaacacactcatggccacacccattgctgacacagtggtctccggggttgcagtcccagtcacctgtgcagagctcgacgcaggtacccaaaagccggtctcctgcagcctgaagctcttgtacctctaggcttgaagatgatgaggattcacaggaggaggaaggctgccaacttcatgttgctgttggaaggctttggaaaaacacagcagggctgagagcagctgaaatttataccttccanccgctgagctggnatgcnaggccaggggtgggactaggggactgcagacaccttaagncctggccagaaacttgacatttctngagattagcaccaccctgtgtaccctgggtctt

SEQ ID NO: 143 - DNA, 568 bp, Homo sapiens
  misc_feature (1)..(568): n is a, c, g, or t
aggacccagggtacacagggtgggtggctattctccagaaatgtcagtttctgggcagggcttaggtgtctgcagtccctagtcccacccctggccttgcattccagctcagcgngtggaaggtataaatttcagctgctctcagccctgctgtgtttttccaaagccttccaacagcaacatgaagttggcagccttcctcctcctgtgatcctcatcatcttcagcctagaggtacaagagcttcaggctgcaggaagaccggcttttgggtacctgcgtcgagctctgcacaggtgactgggactgcaaccccggagaccactgtgtcagcaatgggtgtggccatgagtgtgttgcagggtaaggacagatgaagagttatcttaaggatcatctttccctaagatcgtcatcccttcctggagttcctatcttccaagatgtgactgtctggagttccttgactaggaagatggatgaaaacagcaagcctgtggatggagactacagggggatattggaagcaaggaagaggggttgttcttttaataaatcatcattgtta

SEQ ID NO: 144 - DNA, 130 bp, Homo sapiens
aaattggttttaattttttttaattggatctatcttcttccttaacatttcagttggagtatgtagcatttagcaccactggctcaatgcgctcacctaggtgagagtgtgaccaaatcttaaagcatta

SEQ ID NO: 145 - DNA, 151 bp, Homo sapiens
  misc_feature (1)..(151): n is a, c, g, or t
agaaattgacgacttcacactatggacagcttttcccaagatgtcaaaacaagactcctcatcatgataaggctcttacccccttttaatttgtccttgcttatgcctgcctctttcgcttggcaggatgatgctgtcattagtanttttt

SEQ ID NO: 146 - DNA, 400 bp, Homo sapiens
  misc_feature (1)..(400): n is a, c, g, or t
ttttttttttttttttgattaacattctttatttcacagtatttttgatcagaagtcttagaaatcatgattcatctggttacaaatcccatgagtttctctttgaatgaacctcttgcttccagtcccatacaacgcatctcccaccagccccagtgggttgtaactgtgattcaacactgagtgctcgcttggaaaggaggtggagctcaacttccaactcagagggcctctcccactgctctcagggaaatgcccatgattcacttatgctgtatcaacaacaagtgcagctgggcgctgcctttcccagctgggccaagcggctcctaggggggaatctccaccctcaggagggcttagggaaaggggaaggtntgaacgagttcaggggcccngg

SEQ ID NO: 147 - DNA, 478 bp, Homo sapiens
  misc_feature (1)..(478): n is a, c, g, or t
ctcaaagggctgtcaccatcacctgctgctaggacactacaaaacaatcaaataattcttttctgtaatccaatatgcagcaagcaagggtgacctccagtggcccactcaagtccatgagccattatctaggatactttctctctctttcatgcagttcaaagcccaggtatctctcagatctgctgcctgagaaataagctcctttatcagttagctgttttatcattaggatacaagacagcccagtgtcatcaacagtgagcaaatctgggcatggtgtttgtctcgtacagttggggatagggaggccattcattcccatgggggcacagcttaacattatcccccagtggattacttttcgattacacttgaaggaggaccaccttgtcttttaaaggttcantttccngggggttggcantgtttccaacccagttgtttnccagctgtttcacaaccagtgtgntggatt

SEQ ID NO: 148 - DNA, 444 bp, Homo sapiens
  misc_feature (1)..(444): n is a, c, g, or t
ttttttttcaccttaggcagctttttattttgcatcctttttttcaactttgtcttctattagctgtnaagaaatacatgtctgctaaagttacacgatcttcgcacaacagcaacctacacattagtctacaaaggggaacaaacccaaattcctcagaagtctgagtccactgttgccttctttctggccatctggaggttacaatatagcacagaatgactatgcaagttaaatattcatcttagacatggacatttgctttgggactcctaaagtggagtcaaatttgatctctacagaaactctacaatgtagcagagcactgtgcgtacttattgactcccgggacaagccggaaaccccggaatttgtcatttctatcaggtttttatataaattggttcttacctacttattgatggcttacaatttgggccattn

SEQ ID NO: 149 - DNA, 450 bp, Homo sapiens
  misc_feature (1)..(450): n is a, c, g, or t
ttttagggtgaatcctatgtgtagaattgcttggtcaaatggtaagcaataaaagcaataaacagttgacgcccttgattcgtttttctgttcaaatgtcaattctttaaagaggccttttctgatgactcatgtgaaaaacagctcactgtcattctctggctctttactctgctttattttcctttgaagtccttattggacatcatattatctattaatttgcttattgtttatcttttctactggactgtacacctcatgtgtgtagggcatttgttttgctcacagctgtcaggtattggggataccccaatatctaacacagtaaacaatcaagaattattgggttgaattaatgagttaataaaattaaatactggcctcattgaaggggttatatagatttttaaaaaataccnggttttgtgcnccatggacccaaactgg

SEQ ID NO: 150 - DNA, 399 bp, Homo sapiens
  misc_feature (1)..(399): n is a, c, g, or t
tattgccatctaatgctcagaacacacttgtattgcaagaaaatatttttttgcttgtttttttgagacatagtcttgctctgttgcccaggctggagtgcagtggtgatcttggctcactacaacctccgtctcccgagttcaagtgattctggagcctcccaagtagctgggactacagatgcatgccaccatgcccagctaatttttgtatttttagcagagatggggtttcactatattggccaggctggtctcaaactcctgacctcgtgaatccacccacctttggcctcccaaaagtgccagagattaccaggcatgaagccactgcacctgggcctcaagaanaattatatatcacgtggaatagggatngtagtctctgcactgatttng

SEQ ID NO: 151 - DNA, 426 bp, Homo sapiens
  misc_feature (1)..(426): n is a, c, g, or t
agactgcacgtggttcttagagcctacagtggctgacagagtattgggtattaacgttaacggatcctgtgatgtggcggtgantgcagctgtgatccacgaagtctctgaacagggcttagaaactgactgcactttgtttttaacaggagcctacgtgaagaagagagcacacaattttaaaagttgattttatattctctgagtttttcttcttgcttcaacaaaactctaggaaatgccataagctgaaagaacatgaccttcctcagacatctcttctctccctttccaaacacaactaggagtcatttttttattggtgctatgccattaagaggtcttcctgcttacgctttcctcagagcggattgttggctgggcgcagtggctcagtgcctgatatcccagcactttggaaggccg

SEQ ID NO: 152 - DNA, 305 bp, Homo sapiens
  misc_feature (1)..(305): n is a, c, g, or t
gcatatgacttggaattggcctgtaccaaactctgggacctgctgttcctggatccagtggtcttgttccaacagatgaatctataaaatataccatatacaatagtactggcattcagattggagcctacaattatatggagattggtgggaccgagttcatcactactagacagcacaaatacgaacttcaaagaagagccaagctgcctaagtaccaagctatctttgataataccactagtctgaccggatnaacanctggacccaatcagggaaaatctgggaaagcacttggaaaaact

SEQ ID NO: 153 - DNA, 275 bp, Homo sapiens
atattataaaagcattttattgaacacattctggaggtagttagaaccaaaacaaaatttgggattggggtggggattctgttttgatgatttagatttgggaaaactttgggttctcgtgtcagcaggggccatgctgtgggaaacctgaaggctgatttgaagcagaatatagaactgcggcacgggagaccaggggctgggaatggggctctcctgggaaccaaagaatgtggttctgcaattggcttggtctagactactctccagaaaag

SEQ ID NO: 154 - DNA, 246 bp, Homo sapiens
cagcgtcaaatttgtctccaccacctcctcctcccggaagagcttcaagagctaagaacctgctgcaagtcactgccttccaagtgcagcaacccagcccatggagattgcctcttctaggcagttgctcaagccatgttttatccttttctggagagtagtctagaccaagccaattgcagaaccacattctttggttcccaggagagccccattcccagcccctggtctcccgtgccgcagttc

SEQ ID NO: 155 - DNA, 342 bp, Homo sapiens
ttaaaaaaattttttttattgaagaacagcatacataaagacacaccagttttaagtgcacaacccatttctcacaaagtagacacacttgagtttccaccaccaggtgaagagataaagccttattagcacctcaaaagatcctccccttgtgccccttttcccattacccaccctcctccccaaaggtaaccactatcctgacaccataggttagtttttgcctgtttttaaacttcacaaaaatggaatcatacagtctgcattctttaatgtctggctcctttcgctcaacatcatgtttgtgagattcatccaggttgcctgtagcagcagttcatt

SEQ ID NO: 156 - DNA, 269 bp, Homo sapiens
taagcccagcactttaggagaccaaggtgggaggatcacttgagcccaagagttcaagaccagcctgggcagtgtggcaagacccaatctctcattaaataaataataataaccaaacaaaaaaataaccaccacttttcacactcaccatggcaaaatttaaaaacctaacaattccaagtgttgtcaaggctataggacaactgctggtgagagtgcaaattggtataaccactgtgaaaaaaaagtttggcattatgtatgaaact

SEQ ID NO: 157 - DNA, 466 bp, Homo sapiens
aaattttgagtgacttgagtctcttgcagtccctgattacacagaacctttctgggctacttggagcatcacgaatagtctttcctgtacttaccagatttcaagtattcataacttgactccctaagtgtacaagttgggaatagtacagggccaagttcaagtcgcatatgctgtactgttcctcctgcaaatgtggggaaagaagagggagatactagaggaactgaggctccacccattcattcagttgctctaagcaccagaggacttgtttcagaaaaggggagtgggaacgccctccgactttgccctcctccggagcatctctgggacgcagggagtctggctagcgttaataggaaaggttgctcggcagagtggccctggagtactgacttgtctctccctcctttgtcaaggtccatgtttttctggctcttcctgcacactcatccctagatta

SEQ ID NO: 158 - DNA, 522 bp, Homo sapiens
  misc_feature (1)..(522): n is a, c, g, or t
gacaccaattcatagcatttattgacatttccatttaaaatgctaggaaagctgtatnaattgtaaacatggaaaccaaatacttgcataaattatttcaaaaactctacagcacattagaaaacagtgcagctattgaaggatagaaacataaaaccgacaaatagaagggaggggccgattattaaatcgtatacccatactgagatttcagtgcctgtttgaggaccagcaaaccatgattgtcaagtttaagttgcagtattgatgccacagttggcctcaatttgctctgcacatttcgtacattaacgctcataatctagggatgagtgtgcaggaagagccagaaaaacatggaccttgacaaaggagggagagacaagtcagtactccagggcagctctgccgaagcaacctttcctattaacgctagccagactccctgcgtcccagagatgctccggagnggggcaaagtcgagggcgttcccactcccccttttcctgaaaccaagtcctn

SEQ ID NO: 159 - DNA, 506 bp, Homo sapiens
tgaggattcatattgtcattttacttatttacagaatcaataaaccaacacatacacactattcagagaggtgggaagtgctctgcaaccttctccctcaaacctgggcccagaccccagtcctggaccactgcatccacccagcaggaaaggggtccagccaagacttttcctgactttgtaacttacagacacaagagaatagagggtagaagggaaattcttggcacctggactagagtgagataaaaggagagtaggaaaccagtgataggagagaagtgagggaggtacatacagttttataaataactagacaaggtctgagcactttgggtggggatggagtgagaaaggctacaggcatgtaggggcctaagtggaaaaggaagaaatagtgcttggggccagagcggatgagagatcagctctgggccttcttttgccccatctgtaaaccagtggttgcctaggtggtgtcaaacagcccgtcccggttatctagg

SEQ ID NO: 160 - DNA, 553 bp, Homo sapiens
ctctggctatggggataggaggagagctccggaggtctctgacccctcccaaggatcatgccgcagccccactgacccaggagtaggggcctaagggcagggaacctggaactgggctgtgtgttctgcaagaaattggagccggtggcacggcatatgaggatgctggcctggaaggggacttcagaagctacggggcagcagaccactatgggcctgaccccactaaggcccggcctgcatcctcatttgcccacatccccaactacagcaacttctcctctcaggccatcaaccctggcttccttgatagtggcaccatcaggggtgtgtcagggattggggtgacctgttcattgccctgtatgactatgaggctcgaactgaggatgacgtcaccttcaccaagggcgagaagttccacatcttgaacaatacctgaagtgactggtgggaggctcggtctctcagctccggaaaaactggctgcattcccagcactacgtggcccctgtgactcaacaagctgagaatggtatttggaaaattggga

SEQ ID NO: 161 - DNA, 409 bp, Homo sapiens
gtttattctacttttatttcacatatataaaaacagcttataattgtactgaacacaaaatacaaacaaatacattttattgcacataaaaatattttaaatgaagtattgaagtattgcacgtaatagaattgatttaggaaagtcacaaacctattataagactagtattattctaggtctgaagattacagaatatttcctaatagagatttgccacatcacatattgcacattttccaacactattctatgtcttgcaaatattcctcatagtctttgcttatgtcttttctctgtaagacactgtataaaagattataaaggcaaagaaatatgtaccatcgaaaaggacctgtctacagctgaggaagtaaaaaaataaatacacgatcatcccattcttttg

SEQ ID NO: 162 - DNA, 360 bp, Homo sapiens
acagctctttgcatccggagagtggacaagaaaatgatgccaccagtccccatttctcaacacgtcatgaagggtccttccaagttcctgtcctgtgtgctgtaatgaatgtggtcttcatcaccattttaatcatagctctcattgccttatcagtgggccaatacaattgtccaggccaatacacattctcaatgccatcagacagccatgtttcttcatgctctgaggactgggttggctaccagaggaaatgctactttatttctactgtgaagaggagctggacttcagccaaaatgcctgttcctgacattgtgctaatcctgctgcaatgatcctgaaaagggcattgacttt

SEQ ID NO: 163 - DNA, 395 bp, Homo sapiens
tttttttttttttttttttttttttttttttttttttttttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtcttctgatatctgcagggacagagcattggggtgggggtaaggtgcactgtttgaaaagtaaacgataaaatgtggattaaagtgcccagcacaaagcagatcctcaataaacatttcatt

SEQ ID NO: 164 - DNA, 354 bp, Homo sapiens
cagacactgtatctttagattgatgtcgaccacaaagttcagccagagcttgaggctagatgcacagccttgctattgggaagaaggccttttctagctgtacaacacagtctcactgggcattcatccagaaatagagaagaaagtctgccagacttgagttatgttgtcttttattagcagggaatgtcatcacagattggatagtacatccaggtgcaatgtcaccatcagcaaggtcagcttgacactcaagtggaagattagggaagaatgactaggataaaaaaaaaaggagggcaccaagggaaagggatgatggggtgagctggcgagtgtgggtgggaaatgaaa

SEQ ID NO: 165 - DNA, 348 bp, Homo sapiens
aaaaagacaaagaaactttatttatacaaaactccaccccttctgttccactctcctcagcaaacacagataacaggtgatgaaactaaaacacacagacgagcattactcaacccaaggttcccgccttccctagcacctgaggtctgggccaacatgcagggtaactggtgccttatgcctgctgtctggattgcccggcccacagggtggctgagcatatttattctgggggttccatgcatacgaggagcccccagccatacagctgggcatgggtgtttggcagcaaattgtccctgctttagtcacagcaatttttcatgtcctctgtttgctccccttaaa

SEQ ID NO: 166 - DNA, 437 bp, Homo sapiens
ccagtccctgtccccttgttcacctttggactggacagggagccacctcgcagtccgcagagctcacatctcccaagcaggccccagacacctgggtctggagcagggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctggaggccccagacccctctccaggtggggttccactggaggccagtctgtgtccggcctccctggcaccctcgggcatctcaggaagagtaatcctcatca

SEQ ID NO: 167 - DNA, 421 bp, Homo sapiens
cagattctaacaagaatacttttattatacacgtatcatacacacaacaattatttggggaacatttacaggcagagagttcaattccaaatctccatttcacccacacacactgtactgcacactcaccttagggttcagcccaacaggaacgagacaaagttattgctttctgaacagagagtttcaattaaatagaatcttccaagccaagaacagagcccagcatcctcttaattcttaataccctgtatatatatgaataaaaccttatgatgtgttatagattaccccatcaccattaaaagttaatattaaaattggatcccatgtctcaaaaaagtcgtaagaagtgcaccagtatttacagaccccattaaattacgcataaataaaatctgtacactcaacgcactgtttc

SEQ ID NO: 168 - DNA, 461 bp, Homo sapiens
cctgattgtcatagacaaatcctacatgaaccctggagaccagagtccagctgattctaacaaaaccctggagaaaatggagaaacacaggaaataaaattggaacgaagaaaggttaggagagtagggaaggaacaggactgcaaaaatccttctccaccgcacagactgggaacccctcctggcctgggggaagagtttgttacctaccttactatttaaagagccttcactggttctgcatcacccgcccctggacttcttagttgtttctctagcgctgagctatctcctaactttggacctattatcagaaggtgacaagtactggctctttattcattaagctttttttttttgaaccccattctttccttctctgaaagtggtgctataagttttagaatcttttaaatacattccctgggccaacagacccacacacttagccattgaaatgt

SEQ ID NO: 169 - DNA, 487 bp, Homo sapiens
tttttttttgcatttgtaacatgcacatttattcagaacaaacaactcattaatttattccaaaataatttcacttgataacttgaaatacagagtaaaacaaattggtcaggtaaatatacatgtaacttaaaaagaaacagtcatgtactttaggcataaggacaatgcttttctcttttacaaattcttagttaggtcaaattctctggaagtcactacatttctttactgtgatgtgttttgggtgaagttacaacctatttgcaaatcacatcactggtttgtccaagcagaggtagatgagaggtaagctcctctcctgctaaaagtctcctaaaaacagcaagaaaatatttttatgtgttcaaaaatgctcatttatttatattcctaaattttcttttactcagtataatatagataatttaaaagtaagtagaatatttttattatatatgttttatttttatacttctagttaaat

SEQ ID NO: 170 - DNA, 434 bp, Homo sapiens
ggactcacgggcggggcatgatggtggtgggtacgggcacctcgctggcgctctcctccctcctgtccctgctgctctttgctgggatgcagatgtacagccgtcagctggcctccaccgagtggctcaccatccagggcggcctgcttggttcgggtctcttcgtgttctcgctcactgccttcaataatctggagaatcttgtctttggcaaaggattccaagcaaagatcttccctgagattctcctgtgcctcctgttggctctctttgcatctggcctcatccaccgagtctgtgtcaccacctgcttcatcttctccatggttggtctgtactacatcaacaagatctcctccaccctgtaccaggcagcagctccagtcctcacaccagccaaggtcacaggcaagagcaagaagagaaactgaccc

SEQ ID NO: 171 - DNA, 376 bp, Homo sapiens
ttgcagtggagatggggtttcatcatgttgcccaggctagttttcctttctatatacagaaaaatttaaagtgaatgtgatgttggagagagtgggaaggaaaagtaatggcaagtatgcttgctcattaccaggcactgtgctaagctctgtgaatacacagataagtaaaatccacgctgtttctcaaagaactcacaatctgtttaagaagcagatgtctatacaataattttataactattattcaatgtgattagtactcacatagctctatatagagtgttatagaagaataaattagagaatatctcatttttcctccagtggtttaaaaagatgtcacagaaactgaattgtaaatggtacggaaata

SEQ ID NO: 172 - DNA, 439 bp, Homo sapiens
ggagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagc

SEQ ID NO: 173 - DNA, 390 bp, Homo sapiens
tttttttttttatatgtaatgactgtagtaaccagtttattacacagattaatcattcttgaaagtacaagctccagaggagaatctgggtctttaaatatacacaagtatttccatcaaatgaattttcacccttacatccaaatagacctaagcggttaaaaacataagaaaaataagagctattagctatgtattaactgagaaaccacatacaaaccaaagaatatggaagagagaaagaagggctgaaaggccaaaggttgaaggggtagggagatgtaaaagagttgggaacaaagcccatcacacttgatgtactaatagctccaatccatttaaatgttgaccagttaaacttagaccttaaaatgcaggatgtgggtagag

SEQ ID NO: 174 - DNA, 476 bp, Homo sapiens
  misc_feature (1)..(476): n is a, c, g, or t
gagattgctcggatctacaaaacagatagagaaaagtacaacagaatagctcgggaatggactcagaagtatgcgatgtaattaaagaaattattggataacctctacaaataaagataggggaactctgaaagagaaagtccttttgatttccatttgactgctttctatgagcccacgcctcatcttcccctgtgcacatgtttacctgatacagcagtgctgcgtgttgtacatacttggaacaacaaactagaaatactgtacttctgtaccaacattgcctcctagcagagaagtgtgtgtgtgacaagccagttctacaggcattacctaggtgtgagactaaaagcttttcttattgacttaaatttggataacagcaaggtgtgaggggggtggtgggtatggtgtgtgcttggatgggaaagaanaggctccactcacctataggagattatttttaagtggaatcc

SEQ ID NO: 175 - DNA, 243 bp, Homo sapiens
gcgccgcgcgcccgaaaggctgcggccgtgggcccgtcccgcagaccctgtggttgggctgaccccgcttcagggtgccgtacacgaagactagggccatccgggcagactgtaacttgtttcttcaaggaagtgttgccttagaatccagatccacagtaagcctgagagtcttaaaaacttttgacttcagaatccttccacatgattcaagaaaaagttaagtccacttcacagggtgac

SEQ ID NO: 176 - DNA, 273 bp, Homo sapiens
gaaaactggatatttggtccccatggactttctggtcaccctgtgaagtggacttaactttttcttgaatcatgtggaaggattctgaagtcaaaagtttttaagactctcaggcttactgtggatctggattctaaggcaacacttccttgaagaaacaagcttacagctctgcccggatggccctagtcttcgtgtacggcaccctgaagcggggtcagcccaaccacagggtcctgcgggacggcgcccacggctccgcagcctttcggg

SEQ ID NO: 177 - DNA, 471 bp, Homo sapiens
tttgcccagcaaagacaaatatatttgtccctgttgctacaataggaagttaacaatctggcaagatatctgaacacaagcaaatgaaaacagttcacctaacacccatgcaaattataaatttcctcccatatacaaaatgatgagaaataacagcaaaaatgtatactttcttatttttgaacttttaaagttctagtttggtctttgaatcaaaacaaagtaaaagatgtttataaaagccatttccttttctttccccactatgctcatttgacttgctcttccccctatagggtaccctgagtcattcagagaaggagaattaatagcactgagttggtgatgaagctcctgttaggacatatggcttcacaaaaagaaatacttccagataagtcagagagacagttggacgtcttgagcaaatcttgaaagagatagggaagaaagcagaagttgttgggtggt

SEQ ID NO: 178 - DNA, 341 bp, Homo sapiens
gtgctcccactttgacaatgatgaaattaaaaggctgggcaggaggtttaagaagttggacttggacaaatcagggtctctgagcgtggaggagttcatgtccctgccggagctgcgccacaacccgttggtgcggcgagtgatcgacgtcttcgacaccgacggtgatggagaagtggacttcaaggaattcatcccggggacctcccagttcagcgtcaagggcgacgaggagcagaagttgaggtttgcgttcagcatttacgacatggataaagatggctacatttccaacggggagctcttccaggtgctgaagatgatggtgggcaacaacctga

SEQ ID NO: 179 - DNA, 506 bp, Homo sapiens
ttttgcagttacaacatttaccactttattataaaggctacaactcagaaacagccaaatggaagacatgtataggacaaagaaagatgtgggggtggaagaggttgtatggagcctccatgccctctctggatgccattggttgactgggggaattaattccctggtgcttccagcctgcaagatgagctccttcaaccagcaagtccccagtcaaaagagtgcacggggtgtagctggaagttgagcagatggtagtttgcatggatgagataaagccccaggggacagggcagctacacatgaatccaaatagtctaatctccaaaaggaacagagagtggattcatacaacataccaagcccgccccctaaatgcatcccactcaggtcacttataaagctccaaggatgggccaagaacacaagctctacaccagggaaacttggaggcatcagaaggacagaataagacccaggttcataggggatgaaaaatcgaacag

SEQ ID NO: 180 - DNA, 411 bp, Homo sapiens
caacaatagcgggaatgaagactgtgcggaatttagtggcagtggctggaacgacaatcgatgtgacgttgacaattactggatctgcaaaaagcccgcagcctgcttcagagacgaatagtagtttccctgctagcctcagcctccattgtggtatagcagaacttcacccacttgtaagccagcgcttcttctctccatccttggaccttcacaaatgccctgagacggttctctgttcgatttttcatcccctatgaacctgggtcttattctgtccttctgatgcctccaagtttccctggtgtagagcttgtgttcttggcccatccttggagctttataagtgacctgagtgggatgcatttagggggcgggcttggtatgttgtatgaatccactctctgtgcc

SEQ ID NO: 181 - DNA, 232 bp, Homo sapiens
ttttattattaaatgtatatttttaataaagccaatagttattttacttataggagctttaaaagatacaaaatgtagagttccagtttggaagcattgtaactatacacacaatgtcctgctgatgccctagcaaggcacccacgcccaaccatgcaaaggacacacacgttcacacatgcacacacatgcgctttggcgagacccctctgccaagcgcacaccctggaat

SEQ ID NO: 182 - DNA, 183 bp, Homo sapiens
ggacttagaagccttacaaatacatctgtgcattcttgcttcagactttacaactgagggccagcccagtctggaagcatctcttattaatgttacaaggaaaccgctacctcagcaaacaaaaggaatggaggaggagacttacaactgttttgtatatagacattttcaggcacgtgcttt

SEQ ID NO: 183 - DNA, 429 bp, Homo sapiens
tttttttttttctgcttcaatataattttattagcagttattacatcaaaattcacatttagaggatccagaggactgtcttagaaaattctaaagcatatttaattaggttttaacagtaagggagaacttaatataacacagcccttaaaaagtcaagactactactgaaaattaagtgcagttctatcaagaactagaaatgaactgcacgtgtagtgtcacttaaagcaaagcttcatgaaaatataatacacttctatgaatgtatcagtggcaaacatcattggcttccaaaaaactgacactaaaggaatttccaatcaaaacacaagcacagtggctttcattcaatatagagctatgataagtctatcaagagaccctgaatccttacgtacttgtaatatgattttatgctgtgacact

SEQ ID NO: 184 - DNA, 312 bp, Homo sapiens
tttttttttcactcaataaatttttattagaaatgcagttacactgagaaaggatttcacaatggtcaaatcagtgcacaatactacctagttttatacactgaaaaaaatgtcttgtcaggctacatcattttagaagacactttacagcattcttgtagcattagaaataatgaatagaagagcgtcaaggtgaaaacaaacaccaaatttggtccaataatactgattgctctttgttaaaattcctttgatacaggtactttttataaatgaatatgaatgaacattcggttaaaatgacttacttga

SEQ ID NO: 185 - DNA, 288 bp, Homo sapiens
gggtgacaccaggcttaccttttaaagtttagtatacggagacaattttaatggaaataactactgtagactattgaagaatgatctctttgtgatttaagaagtggctggattggaacttttaatatgctaatgtggaaaattaattacctttatgaaggtggtttattacaaataagcacactaacccctcggaagttgttttacctactttaaaagttttaatggattgcacctctgtaaactattcctaaaatgtgtatgatatatttgaaaaggcttccatta

SEQ ID NO: 186 - DNA, 528 bp, Homo sapiens
tttttttttaactgtccgcaagttaaaaagatttattgctattccaggcttcaaatgagcccagaactcagggctggtgtgtgtttcagaagttgttatgatgtaacagggtggtagaaaaatccaggcagtttgatgtcgaggccaccctctcttccttggacccctgctccaaaagcagctgctggtgaggctctttcccatctgcctcattcacccaacaggactccaagactgaggcaggcagccttgtgatccccacagctcacaggtgagaggctgctcatacctctcctagcactggaagagccttgtccttgggaccggacactatggctttggccctgtggagggagaaacggtgccacaggagttgtcttaagaggacaaggcatgcacggtctgagatcagaggttgtgacgtggccacccatgagccagtccgtttgggacacatcacactgcacagctttttaaaaaataattaggctgcaatcttttaaaatggtaagatttcatataccaatc

SEQ ID NO: 187 - DNA, 466 bp, Homo sapiens
aaaaagctgtgcagtgtgatgtgtcccaaacggactggctcatgggtggccacgtcacaacctctgatctcagaccgtgcatgccttgtcctcttaagacaactcctgtggcaccgtttctccctccacagggccaaagccatagtgtccggtcccaaggacaaggctcttccagtgctaggagaggtatgagcagcctctcacctgtgagctgtggggatcacaaggctgcctgcctcagtcttggagtcctgttgggtgaatgaggcagatgggaaagagcctcaccagcagctgcttttggagcaggggtcccaggaagagagggtgggctcgacatcaaactgcctggatttttctaccaccctgttacatcataacaacttctgaaacacacaccagccctgagttctgggctcatttgaagcctggaatagcaataaacctttttagattgcgggcagtt

SEQ ID NO: 188 - DNA, 407 bp, Homo sapiens
tttttttttttttttttttttttggctttctgggtcttttatttgtacccatgtgtctgtcacaccatgaatgtacctggggaaatcaactgacctccctgaacatttcacgcagtcagggaacaggtgaggaaagaaataaataagtgattctaatgctgcctaggtcactctcaacccccatttactggcacagttgggtggagagaagggaaggggtatgattgtcctgatggctcagggatagagggcatggtagaaagcaaagtacccacacaggccccagttccagctgcggaggacacttgggcgctccagggacaggacttgctggtacacagtctgcccttcccgacgcaggcacactgtgaattggtcagcgatgactgtccggtgctgatacattc

SEQ ID NO: 189 - DNA, 384 bp, Homo sapiens
gaaacaccagctcatttaagctttccccaacgcccggccctccggacgagtacctaacaaccaccggcgcccgcatctggaataggctggcgagatacttagtatccgagggctcgggacttggcgccatcgaggtcatggggacccaggatccagggaacatgggaaccggcgtcccagcctcggagcagataagctgtccaaagaggatcacaagtttattgccctgaagagactggcggcaccaaggatgtgcaggttacagactgtaagagtcccgaagacagccgacccccaaaagagacggactgctgcaatccggaggactctgggcagctgatggtttcctatgagggtaaagctatgggctaccaggtgcctccc

SEQ ID NO: 190 - DNA, 416 bp, Homo sapiens
  misc_feature (1)..(416): n is a, c, g, or t
tttatttnnttgaatctatttaattgctcagactgtgctagagaatacgtaccatgaaatacatatatttcataaggttcagttacaaaatggattgtttcaaatggcaatttcttacactaacctgattatgaaaaaaagaagtctgtatcatctgcttccaagtctgttatgtccaaatatattttaattatgcatttattttgctacttttataaatattagagatttcaccntaaattatttttgtaactagttctagaacatgtttnccaattattattnncctaatgggagacatataattgaccnatggtttatggcatatatggtcctctacacagnggaacctntttttaaaaggaataggtaaaggaaaatgcgggacggcctgggctctccagggccaagggcca

SEQ ID NO: 191 - DNA, 425 bp, Homo sapiens
  misc_feature (1)..(425): n is a, c, g, or t
ttgctccagtttttcagaagaagtgaagtcaagatgaagaaccatttgcttttctggggagtcctggcggtttttattaaggctgttcatgtgaaagcccaagaagatgaaaggattgttcttgttgacaacaaatgtaagtntgcccggattacttccaggatcatccgttcttccgaagatcctaatgaggacattntggagagaaacatccgaattattgttcctctgaacaacagggagaatatctctgatcccacctcaccattgaggaaccagatttgtgtaccatttgtctgacctctgtaaaaaatgtggatcctacagaagtgggagctgggataatcagntagtttactgcttacccagnggcaatatctgtggatggaggncagtgctacagagacctgcttacacttttggac

SEQ ID NO: 192 - DNA, 360 bp, Homo sapiens
gcaacttgaattgtattttttattgaaaagaattcaggctagagttgggaggaggatgcaagagctactgggaagggggagctcagtctgaacctgggggatcaggggagtaggggactctccccttgtccactgatggggggtctggctgttactcctctcccttcagcacagaaagaacttggtcagtaaaaatgcctgtgtaagtgctcatggctgctgtgcttttgctgtacaagtccctgagtttctcatctacagcgggcaggtatgtcttctcgtacaggttctgggcggctgtctttgctgactcccagtaactggagagagattccttcacctgggtgaggaaggtcgggc

SEQ ID NO: 193 - DNA, 346 bp, Homo sapiens
aatttgaggtccaggggaccgaacagccccagcaagatgagatgcctagcccgaccttcctcacccaggtgaaggaatctctctccagttactgggagtcagcaaagacagccgcccagaacctgtacgagaagacatacctgcccgctgtagatgagaaactcagggacttgtacagcaaaagcacagcagccatgagcacttacacaggcatttttactgaccaagttctttctgtgctgaagggagaggagtaacagccagaccccccatcagtggacaaggggagagtcccctactcccctgatcccccaggttcagactgagctcccccttcccagtagct

SEQ ID NO: 194 - DNA, 230 bp, Homo sapiens
actgctcttttattcaatggaacatccccgctttagccagtgttgaatctaacaccgaaaaaagcccagagaaatttctgcagataaaccagtgaagagaacgcgcagtatacattattgtcaacagaatcacttcatggagagggaagcgggaggaaaaaggaaggagaatgaacaaggggctcaaacccctacacactgcaaaacattcagacatttgggattaaaac

SEQ ID NO: 195 - DNA, 611 bp, Homo sapiens
catatgaaattctaataaatccattttatttgtggcaccacaatattatcattaagctctctttttacacagtctgcaatttgtatcagctgccccagtgtgactctgcccttattttaggaacaaccttttgctgggtggcgtcctagaaggtctgggcctgggcagcagcgactgggaagcccacctgtgctttcccccatctgggtggggcggcacagagaccctgagaatcagcggttatgggagctgtgtgttagctgtgtgttattggctttggcttcagcatgtcctgcctaggagtctccagcagctgtggtttccttggactggaggctttttctcctgatgacaatcgtgacaggtccatcaggcagtgcgttgatgatgttccaggcttcaaaccgtgtgaggccctgcatggcagtgccacccagctgcaagatttcatctccaggctggactgtctcactttgttctgaggctgctcctttgaaaatcctgttaatggtgagaagcttgtctccgtgtagggagcccttccctccttccaggctgtagcccagccctgccgacatcttctccatggtcaccgtgaagactgtggcctc

SEQ ID NO: 196 - DNA, 502 bp, Homo sapiens
gcacagggctggctctgtgcaggctccaatctaggacacaattatctttaatctttgttggcctaaaaatcctctagcattgactaaccggttcaatcctcctccagcaagtatgtggactggacttgtgtgatttctggtcctgacttcctttggtttgctcaggttcacagagtgtttccaaatgggctggcctcccaggaagggactattcagaagggcaatgaggttctttccatcaacggcaagtctctcaaggggaccacgcaccatgatgccttggcatcctccgccaagctcgagagcccaggcaagctgtgattgtcacaaggaagctgactccagaggccatgccgacctcaactcctccactgactctgcagcctcagcctctgcagccagtgatgtttctgtagaatctacagaggccacagtctgcacggtgacactggagaagatgtcggcagggctgggcttcagcatggaaggagggaagggctcc

SEQ ID NO: 197 - DNA, 412 bp, Homo sapiens
cgcggagaaaaaagttctcgccaccaaagtccttggcactgtcaaatggttcaacgtcagaaatggatatggatttataaatcgaaatgacaccaaagaagatgtatttgtacatcagactgccatcaagaagaataacccacggaaatatctgcgcagtgtaggagatggagaaactgtagagtttgatgtggttgaaggagagaagggtgcagaagctgccaatgtgactggcccggatggagttcctgtggaagggagtcgttacgctgcagatcggcgccgttacagacgtggctactatggaaggcgccgtggccctccccggaatgctggtgagattggagagatgaaggatggagtcccagagggagcacaacttcagggaccggttcatcgaaatccaacttac

SEQ ID NO: 198 - DNA, 534 bp, Homo sapiens
tttttcttttaaatcatgacacttggtaggtttaccaccagcatccaaaatgaacaaaaacggaaaaaaaagcatttactatatatttcagatttctttggttggggttctccccatgtggtattaatatttcttgtttcaatatatatattaccaaaacagtaaaaaccaggaaaaaaaatagaaacctagcggttgctgaaactagagaggctactctcttgtcttccgtgcaggaattcccaggttctcagcttgctggaaaaatttgttgacattttctttttgtagctgtttcttaaagaataacagtaaacattccaatgtccaaatcttggttagtcttccactttattgcttggatgtttctttggtgttggttaaggttgtggcctgctttttgctttatttctgaatggtcattaattctttaggtcacctgccgatggtgaaggtgcctgaggagcctggtgttactcagcactgctctgctgggtgggtggagcagggttctcagttggtgcttcacctgcc

SEQ ID NO: 199 - DNA, 455 bp, Homo sapiens
  misc_feature (1)..(455): n is a, c, g, or t
agaggagcggagcgggcagcgggaaggggcgcgctccgctggccgccgagccgcacttgtccaacgtggaaaacccaaataccagtttcaaacacttgggaaacattcagccccgctgcgcagcgcgcatgcgccccggccccctcccccggcaacggccccgccccccgccgcattcacgcccctcaccgtcccaggccctgggggctgcgggctcgaggccggccctcgcggnggcgtggccttgcctgtcactttttccagaggcgagggtcgcggaggggacagcgtcagggccgctggggtgtggacggcgggcgaggcgcaaactttactaggagtttttggcacttggaggcagagcctgttgggcggcacagcacgcccgctgggaaacgcaggggagcggcctgcttcgctgaaaacccgaccaggacctaacgggccgcgggaca

SEQ ID NO: 200 - DNA, 478 bp, Homo sapiens
  misc_feature (1)..(478): n is a, c, g, or t
ggctgcggtagttgctgtgtaccatggtctcggaggtttctgtcccgcggcccgttaggtcctggtcgggttttcagcgaagcaggccgctcccctgcgtttcccagcgggcgtgctgtgccgcccaacaggctctgcctccaagtgccaaaaactcctagtaaagtttgcgcctcgcccgccgtccacaccccagcggncctgacgctgtcccctccgcgaccctcgcctctggaaaaagtgacaggcaaggccacgcccccgcgagggccggcctcgagcccgcagcccccagggcctgggacggtgaggggcgtgaatgcggcggggggcggggcccgtgccgggggagggggccggggcgcatgcgcgctgcgcagcggggctgaatgtttcccaagtgtttgaaactggtatttgggttttccacgttggacaagtgcggctcggcggccagcggagcgcgccccttcccgct

SEQ ID NO: 201 - DNA, 619 bp, Homo sapiens
  misc_feature (1)..(619): n is a, c, g, or t
tcctcgtcctcctcgggggcctaccgagcggctacggcgctcactgaccgcgtccgtacggcatgctggcgggcaacgagaagctaaccatgcagaacctcaacgaccgcctggcctcctacctggacaaggtgcgcgccctggagggcacaacgcgagcatagaggtgaagatccgcgactggtaccagaagcaggggcctgggctcaccgcgatctacagccactactacacgaccatccaggacctgcgggacaagattcttggtgccaccattgagaactccaggattgtcctgcagatcgacaatgcccgtctggctgcagatgacttccgaaccaagtttgagacggaacaggctctgcgcaatgagcgtggaggccgacatcaacggcatgcgcagggtgctggatgagctgaccctggccaggaccgacctggagatgcagatcgaaggcctgaaggaagagctggcctacctgaagaagaaccatgaggaggaaatcagtacgctgaggaggccagtgggagagcaggtcagtgtggaggtagattcgctccggcacgatctcgccanatcctgagtgacatgcacgcaatatgaggtctggccagcaga

SEQ ID NO: 202 - DNA, 528 bp, Homo sapiens
ctgaaagggtgcgccgagtcagataacctcggacctgctcatctggagctgctccgtgtggccagcgacctcccggttcaattcttcagtccggctggtgaaccaggcttcacatccttccggttctgctcggccatgacctcatattggcttcgatgtcactcaggatcttggcgagatcggtgcccggagcggaatccacctccacactgacctggcctcccacttggcccctcagcgtactgatttcctcctcatggttcttcttcaggtaggccagctcttccttcaggccttcgatctgcatctccaggtcggtcctggccagggtcagctcatccagcaccctgcgcaggccgttgatgtcggctccacgctcatggcagagctgttccgtctcaaacttggttcggaagtcatctgcagccagacgggcattgtcgatctgcaggacaatcctggagttctcaatggtggcaccagaatcttgtcccgcagtcctggatggtcgtgtagtagtggctgtag

SEQ ID NO: 203 - DNA, 337 bp, Homo sapiens
ataatttgccaagataaatcacttttatctctataggaaagggaggatctaaaaaaaatataaattacattagtaacacaacataagaaaaagacagggacaaaaacaacagagaagtctgaatgatgctaccctaacctatttataaaaaggccctgcatcagaaattcacaatcctacccacttctaaaaatatatttagacatgtacagaagcggtgggcttgtttttaaattgtttgctttatttgtaaaaatatattaaaggtgaatagaaatcctctctcccttccccctgtccagcccccagctagggactggagatcaggggtaactat

SEQ ID NO: 204 - DNA, 342 bp, Homo sapiens
cttttagctggctacacatgaggccacttgttttagggtgagctccagggatttgcctggattttgaaatcatgtagaacattatccacgtggctgtggctgtggctgtggctgggccctggcaggtggaaaaccatctcccagaaacctgaaatcacctgccaatgacgcagataaccctggccctacagcctgcttgctccgcctataccacagagcacagcctggacattatggagggtgtggcgggacggccacacctgggtcctccatcgggaacttttcatgcttctttctccacctgaggtcttggtctgaagaagacctcaggactcacatctt

SEQ ID NO: 205 - DNA, 399 bp, Homo sapiens
gcaatcataaaataactttattggtcaggttagccaccactcatgcttttcctgtaataaggatcctttataaaggcatgatggtgttcacatgcagatgctttctgaagagccctggggcagggggcagccttgcccctcacatcggagctcctttgttgaaatgagctggtttggcttttgtggattccaggtctggagccaagaacgtagtccaaagatcccctcttcccttctcagggaaggtgcttcaaagcatacacagtatcagggatgtgatggcatctgggcagagctatacttgggctaactctcctccaacagtccttgcccctgactgcccagatggctttgtcccaaccttgcccaaaggacggtgggttaagcccaggcaacatt

SEQ ID NO: 206 - DNA, 437 bp, Homo sapiens
aatgtatagggctatattttggcagctgggtagctctttgaaggtggataagacttcagaagaggaaaggccagactttgcttaccatcagcatctgcaatgggccaaacacacctcaaattggctgagttgagaaagcagccccagtagttccattcttgcccagcactttctgcattccaaacagcatcctacctgggtttttatccacaaaggtagcggccacatggtttttaaagtatgagaaacacagtttgtcctctcctgttatccaagcaggaagattctatatcctgatggtagagacagactccaggcagccctggacttgctagcccaaagaaggaggatgtggttaatctgtttcacctggtttgtcctaaggccatagttaaaaagtaccagctctggctgtggtccgtgaagcccaggccagg

SEQ ID NO: 207 - DNA, 360 bp, Homo sapiens
ttagagcttaatggaattttattttgaaaatatggcaagagtctaaggcacttcaaacatttaaatacatagaggaccaaagtaaatgtgacacggtaaaaaggaatccataaatacaaagagaacactgtgtttctctagaggcaaatacagagccgattcctctaacacaatccaacctttagcattggagttgtgcaattaatacaaatgatgatgttacgtgtagttcttcatggctttagtatggaatacaaaagctgaaaatactgtgtcaagttcatatagataccctttttataaaaagtcatatattacatctacctagttaagaccaaatgagaatattcttttgtaagt

SEQ ID NO: 208 - DNA, 386 bp, Homo sapiens
ctgtgtcctaatttattatgactacatagcccacattcctctgcccacgcatccgtggagtccagagcccagaaagcctcctgctgccctgccagaccgttgagctcctcaagagcgaagtgtggcacaggctgatcagctcatgcagaatggcagggcttcagctgcccaagtgtgtgcgtaccagagcacagcattcatgaagctgtctgactccacctccacctctgataatgcgtgggtgcttttgggatagagcaggagccgaacaggcacattccgggtcttgagggcacggtaatactccatgccctgcttgaagggcacacgccggtcctcctggcccaacatcagtaacagtggtgtcttcacctgagggatgtatc

SEQ ID NO: 209 - DNA, 343 bp, Homo sapiens
  misc_feature (1)..(343): n is a, c, g, or t
ttcccagccatgctttgcaagatgggctttgcggtacatactagtgaactatcgtgaatccacgggctttggccaggacagcatcctcaccctcccaggcaatgtgggacaccaggatgagaaggatgtccagtttgcagtggaacaaggagcaccaggaggaacacattgatgcaagccatgtggaccttatgggtggatcccatggtggcatcaataccagccacatgattggtcagtaaccagagacctncagggcctgagtggcacgagaacccgtgataaacatagccaccatgtagggcaccactgacatccctgactggtaagaggtggaggctgg

SEQ ID NO: 210 - DNA, 388 bp, Homo sapiens
ttttttagttaaatacgcacaattttattgattgaagagattaggacaaaaacattaaaccaaatacaggacaaagcaccagaggccatagatccccaccatgcatgtcaccaacctctcctcctccaaggtacttaaaaaattggggagaggggaaaaaaaaggtccttcttgacacagcaccatcttcagaatgttaaaaaaaaaaaaaaccttctctcctttctatcttccattagcaaaatagaatcaagggcaaatccatggccgccttgtctcctggttacgaagggtgaagccgccctcctgggaacgtgaggacagggctcctgctgcgcaggcataaagcatccaagagtctgcacatacatgccacacactattatga

SEQ ID NO: 211 - DNA, 595 bp, Homo sapiens
aagagagaggcaattttattcttccaaaaaaatgcaccaagagagggtgagcacaggagcacccctggccacatcccccatcctaagcagggtctgagatgaggccaggcctgacgtgggcttgggagaagctgacggagctccctgtggccttggggagggaaccaggcagacctggaagtggaactttgttgttagcaccaggagccgcccacagctgggctcggcaacagggcagcacatggccctgttgctgccacctgagagtctggggaggggctggtggcagaaggctccctgcaggaggtcacctgaatgactctcagattcacagaccccctctgcccccacaacccctgtaaacatgagaatgggctcgtgacaccctcaacacctcaggacaagatgagggtccgagatgtgtggctgggcttcaggcggcccaggagctgccgggctttctcctgcatgaaaagctggtccctggtccccccgcaggccaccgtcttccaggcactggacataggggcaggtgtcgtgaagtggcttcggggcttctgggccactgctgccttctcgggcttggctgcaagaa

SEQ ID NO: 212 - DNA, 450 bp, Homo sapiens
  misc_feature (1)..(450): n is a, c, g, or t
attcggaacaccggacgcaatcaagaaagtccggaagtctctggctcttgacattgtggatgaggatatgaagctgatgatgtccacactgcccaagtctctatccttgccgacaactgccccttcaaactcttccagcctcaccctgtcaggtatcaaagaagacaacagcttgctcaaccagggcttcttgcaggccaagcccgagaaggcagcagtggcccagaagccccgaagccacttcacgacacctgcccctatgtccagtgcctggaagacggtggcctgcggggnggaccaggnngaccagcttttcatgcaggagaaagcccggcagctcctggggccgccctgaagcccangccacacattctcgggaccctcatcttggtcctggagagtgttggagggggtgtcacgagcccatttctcatggttttacaggggttg

SEQ ID NO: 213 - DNA, 408 bp, Homo sapiens
gaaagaaagagtggaggggttaacatggggcccacctcacaacccactcttcacccccaaaatcacgcagggatgggactcaggaaagggaagcatgtgtgtgttgaataggagccctaactgtagttacttctttcacagcagggaaggaagagggaagaggcagctgtggagaggatgaggttgagggaggtggggtatctcgctgctctgaccttaggtagagtcctccacagaagcatcaaagtggactggcacatatgggctcccttcacaggccacaatgatgtgtctctccttcgggctggtccggtatgcacagttggggtacctggagccgtttgtcaggcggcagtctgtgatgtgcatgctggagttgctcttgtagcagttgccctgcccgttctt

SEQ ID NO: 214 - DNA, 334 bp, Homo sapiens
cctggtagatgtccagaatgtctgtttccaggaaaaggtcacctgcaagaacgggcagggcaactgctacaagagcaactccagcatgcacatcacagactgccgcctgacaaacggctccaggtaccccaactgtgcataccggaccagcccgaagagagacacatcattgtggcctgtgaagggagcccatatgtgccagtccactttgatgcttctgtggaggactctacctaaggtcagagcagcgagataccccacctccctcaacctcatcctctccacagctgcctcttccctcttccttccctgctgtgaaagaagtaactacagt

SEQ ID NO: 215 - DNA, 310 bp, Homo sapiens
gggttttaccagttttatttctagactttcatgtttgtctttttgtcttctgctggaaacatgccggttacatgttggtgctgggaagcgccgcgctgcaaccagaaatgcacagacccagccgcccgccgcccagaccctcagacttgcgcgtcacaggacagactccgctgtgccccgtgcacttgccaccagcctttggcgtctcgatacacacaacatccaggacttgtgcccttgccccatcacgacagacaaagcgtccctcaaggcccccgcgtggttcagacagacgccgcagccaggatgg

SEQ ID NO: 216 - DNA, 493 bp, Homo sapiens
gccagctcacagtgctgtgtgccccggtcacctagcaagctgccgaaccaaaagaatttgcaccccgctgcgggcccacgtggttggggccctgccctggcaggatcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaagcgtggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaac

SEQ ID NO: 217 - DNA, 509 bp, Homo sapiens
tctttattgaatgagggttgtcaggagcaaaggtgggatcaagagcagcaaaagcagaaacaagtataaaagtatcaaaaaatacaaagtgctagcactgaggagagtgagaagggttgggttgtggcccagagggacctctgggacacaggattgaggacttgccacagcctccaagggaacctaggcctggggggcgtgtgcaggatccttggctgagggtggaagtggcttgagcggggcccaaccctgggccgtgaagtatgagaccagttgtgtgggcacttctgcgagcacggtctgtgccaatgcctcccgaggggcattctggaaccggcggtagggtacaaactgcacaatgtcgcgggcagcacctgcccagaacgtgtatgcaggggtccaccatcagcgtccagctgctccatggcctcaaagtcagcaccagccacacccacattgatcactgacatgggcaggttcgaggcacgcaccacagcctcacgtgtggg

SEQ ID NO: 218 - DNA, 52 bp, Homo sapiens
ggtcggtctgttcttttgcggttctgctcttgccctgtgttctctttgtctc

SEQ ID NO: 219 - DNA, 533 bp, Homo sapiens
ccagagctaaacaatttaatataaaaaatgccattttttgtccatacagtatttataaaaaagtacatagtggttagttttgcaataatttctttttagccagatgtcatatcatcatataaatctatgaatataacaaatgacataagaacagtataaataagtttttgtagtatttacacttacacagaaactagcccaaatggtgtcctaagaaattgtttacagttaaagtgaaactactgattcaacatactgacactccaatgctttttaaagtttcgtattattttctatactagttttggctatgattttgcatagaattacttataaagtatgagcatttcacatcacagtaggagcttttagtataatagtacaaaaaaactagctacgaaaaggtcaaatcctcctaaatctagtttttcttaaaatctggcttctaactttgggaaaaagaaaacattggcatcacttgtttgctgcagggagtattcaccaggagaataaggtgttacctcttcatcacg

SEQ ID NO: 220 - DNA, 343 bp, Homo sapiens
ttcattcaatttcctttaatgagtacttgttacagtaaaagaggtataaagtcctgttcccaagtccaaaccactttttaacttaaatcttgagtttttctgaattactcaatttgaagtaattctctttatatctgaaaaatggttttattgaaacgtttgagattaaaaaatatgcattgcaagaagcatatgacaaacattctgagagtacaaaattagttgtaaaaaataacataatttaccagtaaacccactcatatagaaatgtgcaaagccttttgatataaaaagttttgtacaccaagcacctatttttataacttagcttcccatggagaga

SEQ ID NO: 221 - DNA, 393 bp, Homo sapiens
atttgttaaacagtttaattcccaaagctagtaattttagttaaatatacattagagcctttttagatggctgctaataaacactatgtcaaaatgtgtagttttaaactcagactcgaaagccaagataagcaactccttcagttattactctgaccaaggcataagaattcacttagacaaaaagctttcaaaacctacctaaaaataagatagttcataaattttcaaaactgttcttccctgttgcggacagcccttgatctttgtaagacttagcaaattttggcatgctctcatgttagctttttaagttactgaaaactcctataaatttagcatcatttctcaaatctgtatagttttctcattccgaatgcttaaacatttagg

SEQ ID NO: 222 - DNA, 565 bp, Homo sapiens
ccctacaaaataatttattggaacacacagctacagcactctatgtacaagcacattgacgctcctgactatcctcaactaggggacccttttcttcccccttgccttgcggacctcttctatcaaatctttcaggtactggatctccttggccagggaatccgccctctcttttagagcctcgttcttcttttccagctctttgcactcaccagtaagagcctcctgctccgccctcttcttctggcggtacctagtggctgctgtcttgttttgctccatttttttcagcttcttatccagtttctcaccctttacttttgctgctaccatcttctctccaggaggatcgtaaggtttgggacgggcagacccacagagaacacctggagatgggaggctcctatttggagagcccctggtagaggggctgtgctgaggagaccccagataggactctgggctcatacagatgccactatcattatctgaaggggtgtcttcctcctttatgcactgagggatcatggcaacgtaagcagtgtagtctggcttcctatctcct

SEQ ID NO: 223 - DNA, 716 bp, Homo sapiens
tccaaatcaatttattatcctgacagctggcatcattaatactttaacaaaaccacttaaaattagccaaatatctaagacagatacatatacaaaagatatacaaattaaaaccatttaaaaagtaatagataccataatttgtacttggccacaacttctgtattcagaaatgattgtaaaattaaaacctaagttaaaaactgtacaccatatactttgagtgatttacatcttagaaaacaaaggcagtctttcattgttacagatttagtgtctctggtgggttgaggagagaaacaccatgatactttgaatttttgtacttttctcttattgactgttgtgcatgctgtggtgctttgaggtaggtctggtgaaggtccatgagacaaggcttaagactttccagggtatatccagtctttcgtattaatgattcaggccagctttgtcccgtgactgtgtagagtgctaaatgaaaggcagctccagcaataactgatggcaaatacttgaggtatgggtcagcatctatcagacttaattctcccaaaaacattgctaaactttcaactttgcagtttgcaggctactgatgcagaaagtattggggaagaaactgatttactggtgggagcagctaagtcaaaagtaagggactttcaaaaactagatggctccattgctcaggaacttgtttacctgggtggagg

SEQ ID NO: 224 - DNA, 21 bp, Homo sapiens
gatgacgacaggccgatgatt

SEQ ID NO: 225 - DNA, 21 bp, Homo sapiens
tgaccaggactgcgttccatt

SEQ ID NO: 226 - DNA, 22 bp, Homo sapiens
cccgctgtagatgagaaactca

SEQ ID NO: 227 - DNA, 22 bp, Homo sapiens
tctcccttcagcacagaaagaa

SEQ ID NO: 228 - DNA, 22 bp, Homo sapiens
atgactgagtacctgaaccggc

SEQ ID NO: 229 - DNA, 22 bp, Homo sapiens
cagagacagccaggagaaatca

SEQ ID NO: 230 - DNA, 22 bp, Homo sapiens
ccagctgtggtattccaaacca

SEQ ID NO: 231 - DNA, 22 bp, Homo sapiens
tgagcagctcagttcagttcca

SEQ ID NO: 232 - DNA, 22 bp, Homo sapiens
gctggcctgaatcattaatacg

SEQ ID NO: 233 - DNA, 19 bp, Homo sapiens
gcatgctgtggtgctttga

SEQ ID NO: 234 - DNA, 22 bp, Homo sapiens
aggtctgcgaggaacagaagtg

SEQ ID NO: 235 - DNA, 19 bp, Homo sapiens
tgcaggcggctctttttca

SEQ ID NO: 236 - DNA, 22 bp, Homo sapiens
tgctgcaactcctctccttcat

SEQ ID NO: 237 - DNA, 21 bp, Homo sapiens
cgtcttgctcggattgttcct

SEQ ID NO: 238 - DNA, 22 bp, Homo sapiens
catggtgctactcttgctgtca

SEQ ID NO: 239 - DNA, 10 bp, Homo sapiens
  misc_feature (1)..(10): n is a, c, g, or t.  This sequence is a place holder.
nnnnnnnnnn

SEQ ID NO: 240 - DNA, 22 bp, Homo sapiens
ccctgtaacgttgaaccagttg

SEQ ID NO: 241 - DNA, 22 bp, Homo sapiens
ggaaaagacatcaacccccata

SEQ ID NO: 242 - DNA, 22 bp, Homo sapiens
tctggttgtggtctctggtgtt

SEQ ID NO: 243 - DNA, 21 bp, Homo sapiens
gcggcactgcaggtgtaatta

SEQ ID NO: 244 - DNA, 21 bp, Homo sapiens
ttcccttagccagtcgatggt

SEQ ID NO: 245 - DNA, 22 bp, Homo sapiens
ctgcctcgcaatacttcatgct

SEQ ID NO: 246 - DNA, 22 bp, Homo sapiens
ccacacccacaatgatcactga

SEQ ID NO: 247 - DNA, 21 bp, Homo sapiens
ggcaggtgcagatgtgatcat

SEQ ID NO: 248 - DNA, 20 bp, Homo sapiens
tggctccaacagcattgatg

SEQ ID NO: 249 - DNA, 22 bp, Homo sapiens
cacacaggtactcgtcctcaca

SEQ ID NO: 250 - DNA, 20 bp, Homo sapiens
tcgcacatggaacacttgaa

SEQ ID NO: 251 - DNA, 21 bp, Homo sapiens
ccattcctggtcacgcaaaac

SEQ ID NO: 252 - DNA, 22 bp, Homo sapiens
tcctgtgtggtaggcacctgaa

SEQ ID NO: 253 - DNA, 22 bp, Homo sapiens
ccgtctgtctcctttccttctg

SEQ ID NO: 254 - DNA, 22 bp, Homo sapiens
tcctgtcctctgctctgtggat

SEQ ID NO: 255 - DNA, 21 bp, Homo sapiens
tgctcccctgtttttgtgaca

SEQ ID NO: 256 - DNA, 22 bp, Homo sapiens
tcctggaagtaatgccaactca

SEQ ID NO: 257 - DNA, 22 bp, Homo sapiens
ggcctagagcctcttgattcaa

SEQ ID NO: 258 - DNA, 22 bp, Homo sapiens
ttgctcctctcactccatgtgt

SEQ ID NO: 259 - DNA, 22 bp, Homo sapiens
cagctggctcgatagtcgtaaa

SEQ ID NO: 260 - DNA, 22 bp, Homo sapiens
tctaggaggagcccagtcttca

SEQ ID NO: 261 - DNA, 22 bp, Homo sapiens
aaaagccatcccagctcagtag

SEQ ID NO: 262 - DNA, 21 bp, Homo sapiens
cacatttccaggaacgacgat

SEQ ID NO: 263 - DNA, 22 bp, Homo sapiens
aggagctggtgtgcaaggtgtt

SEQ ID NO: 264 - DNA, 22 bp, Homo sapiens
ccccatagcttcgctcaaagaa

SEQ ID NO: 265 - DNA, 22 bp, Homo sapiens
gcagccggagaacaactacaag

SEQ ID NO: 266 - DNA, 20 bp, Homo sapiens
tgcatcacggagcatgagaa

SEQ ID NO: 267 - DNA, 21 bp, Homo sapiens
tcccatggcaagtcctaaagc

SEQ ID NO: 268 - DNA, 22 bp, Homo sapiens
ccatgacacagccaaacagaaa

SEQ ID NO: 269 - DNA, 22 bp, Homo sapiens
tctcatcgtgtcacaactgcaa

SEQ ID NO: 270 - DNA, 22 bp, Homo sapiens
gagctgcttctacgtccaactg

SEQ ID NO: 271 - DNA, 22 bp, Homo sapiens
cagcgtttcctgcattgtcatc

SEQ ID NO: 272 - DNA, 22 bp, Homo sapiens
gacccctgagcatcctggatta

SEQ ID NO: 273 - DNA, 21 bp, Homo sapiens
ggagctcctcatgaacgtgaa

SEQ ID NO: 274 - DNA, 19 bp, Homo sapiens
aggtgtctttcccgttgca

SEQ ID NO: 275 - DNA, 21 bp, Homo sapiens
ctaacgcagcagttgcaaaca

SEQ ID NO: 276 - DNA, 20 bp, Homo sapiens
tctccgtcgcaacttgtcaa

SEQ ID NO: 277 - DNA, 22 bp, Homo sapiens
atggctcctgctgtacctcaag

SEQ ID NO: 278 - DNA, 22 bp, Homo sapiens
gtgaagcggtggacaagaaact

SEQ ID NO: 279 - DNA, 20 bp, Homo sapiens
cgaagctgttgttcggaatc

SEQ ID NO: 280 - DNA, 22 bp, Homo sapiens
ggctggtgtagcagatcatacc

SEQ ID NO: 281 - DNA, 22 bp, Homo sapiens
aacctgccagatgcttgtgaat

SEQ ID NO: 282 - DNA, 21 bp, Homo sapiens
cggtgtcatcaattgctttgg

SEQ ID NO: 283 - DNA, 22 bp, Homo sapiens
gctccattaccaagagctcatg

SEQ ID NO: 284 - DNA, 21 bp, Homo sapiens
gtgcctggtcagttcatctga

SEQ ID NO: 285 - DNA, 20 bp, Homo sapiens
gcatgaaagctgccttggaa

SEQ ID NO: 286 - DNA, 22 bp, Homo sapiens
cctgattctgccgctcactatc

SEQ ID NO: 287 - DNA, 22 bp, Homo sapiens
cagaagccgagtcctggtatca

SEQ ID NO: 288 - DNA, 20 bp, Homo sapiens
tggcgcactgtttcttgaca

SEQ ID NO: 289 - DNA, 22 bp, Homo sapiens
agcagaaagagtggcagaggat

SEQ ID NO: 290 - DNA, 22 bp, Homo sapiens
ttggtggaagagctgttgatgt

SEQ ID NO: 291 - DNA, 22 bp, Homo sapiens
ggtggatctgtcagctgccata

SEQ ID NO: 292 - DNA, 22 bp, Homo sapiens
gcctgtcacctcagaactccaa

SEQ ID NO: 293 - DNA, 22 bp, Homo sapiens
cccatcaagaaagtccggaagt

SEQ ID NO: 294 - DNA, 21 bp, Homo sapiens
gcagttgtcggcaaggataga

SEQ ID NO: 295 - DNA, 22 bp, Homo sapiens
tccactgcatcattcagctttc

SEQ ID NO: 296 - DNA, 21 bp, Homo sapiens
tctccaaagcgaggtcttcct

SEQ ID NO: 297 - DNA, 22 bp, Homo sapiens
agctcagagacccgcagcatta

SEQ ID NO: 298 - DNA, 22 bp, Homo sapiens
actgaggatcaccaggcctttg

SEQ ID NO: 299 - DNA, 22 bp, Homo sapiens
gcccatcatcatttccattgtg

SEQ ID NO: 300 - DNA, 22 bp, Homo sapiens
tgaacacctgcatccttgaaga

SEQ ID NO: 301 - DNA, 19 bp, Homo sapiens
tcgcaaaggtcccatttcc

SEQ ID NO: 302 - DNA, 22 bp, Homo sapiens
cgtagagctgaggagcgacaat